SEQUENCE LISTING (3 sequences)

SEQ ID NO: 1 - DNA, 113073 bp, Klebsiella pneumoniae bacteriophage Kle_F391/08
attatgcaaactaaacgaaatcatacactccactaattaaaggttgacaacatgaaacgctttgtaatatcatctaaaccggcggcaaaattgaacaaaatggcaagctcggaactggtcaaaatgcagcgcgatatggcgcggcgttaccgctcttatcagcgcggaggtcagcaaggcttaccgatgatttgggataacatgatgcgttatctaaaatctttacaaaataacgtttgacaatctgccgataacgctttattatagaatctcaataagcaacaccgctctttaaaaatttggaaaagtcgattctctcagtaagggtaattgagttttctgttaacactctagcagtaaaagagtgttaaccggataacccaataaactaaactggagtaagaaacatgcaaaacgttaaaaccccaatgatcggtgattccgtttttattcctttcgttactggcgacgtaagtaagccgggcgaaaatgaaaaaatcggatatatcaaaggggcggcgatgatcccgtttgataaaattaatgcggtatacgccgaaacggaaaaagcaaaaaacagcgacgcgaagatttacagcgtccgggtagattccggcgacgtggtgaaagtcattcgcaaagatgataaatggttggctgtcgcttaacagccaataagtttaaccccggcaaataatgggatgcttcccctatgttagacttccgatgtatattccatgcagcgcaagcaatgttatggtgcgcaaattaaacttatgtgctggaacttatatcatgctttgaacggcaattgtgagttaacacgaatctatagcgtgaaatagacgtaatagcgtaattattaaaggcagcgtatatatacaatgccgctttaaatagctttaaattgaaaactaaaataaagagttttcgttatatattctagctagaatatatagcggctaactttctagccttaacccttaaataaaactggagatatatcatgactgctactaaaaccgaaaaattcgcatggaacgaaacaaacgccgctaccgccgttgaaatgtacgaaaagatcattgcttccgatggtctggaggtggctaactcgcaaggtcttattgatattgcaaaggctgtaggtgctgaatcgcatgttaaagtgcgctctaaactggttagcgcaaaggtgtatcagaagtccgataagccgcgcaaagtcggcggcggctcctccctgcgtaaagctcactatgtgcgcgttctcactcagcacgccattgctgacggcctgattgatgatgccgatggtctggcaagtctggaacaaatgaagctcgaccaactggacgttatcgcccgtatggttggcgttcaggatgaagtaaaagaatcagcagaataattataatgcgcctccctagcggggcgcataattaaatattttgtcccccataactggagaaataaaaatggctttaattaaaggctccgttattaaattaacgggaaccgttgtggatgaattaatccaaacggggtatcaggataataaggttatgacgcccccaagcgttaaagtacctgaatatattgttttgtgggttaaccctgatgcggatactttcgggatggctattaaccgcgaagtctttaagccggaaatgctggaattatcttcccgcgaaatttaccttctcaattacgctttcagcgtagaagaaaaggaggttgtaaaatgatctttttccctactgaatccttaattcttatcgctggctttttcgccgccgcttgtctctatggttactataatttcatggaaataggcagcgaacaaacggatttattacgccgtgacttctggttcaagaaagcgagtatttgccgccgctggtcaataatctttattattctagcggtaactttcggaatatcagcaagcattatcccggcaatagtttagagtcgcaaattataacgccaccagcaacaaggcgttatatttggcaattctgcctatatcccattaactggagaatttagataatgatcattgcaattgagaaacaaaaagctattttaaccgctgctaacaacctcaatttttggggtaaacgcctgcgcgctaaaaagctggagatctgcgacgaattgagcaaagagcattacgggacggcgaaacactctagcgaaatttgcgattggctggatagtaacaagcctgttaaacccgctgctgaaaaacgcgcccaacgtgttgcggtggaagattcccgccccgttgccgctggtcagcttaattccagcgtcgaaagctggaaggtaattccgggccgccgttttctgctaacgtcgattcaaaataataccttccctcatgctaatttctggaaaactttacaggaggccgcgaaatatctcggcgctactctgttagtaagcaaatacgcctacaacaaaaaaggcttccaaaacgggcaaggcaacgatgaactgaaatatgatgatgctttctcagatttcatttgtgatgaaaacgtgtttttgggcaaccgcgaaaccggattcgcattcatggcggaaattaatattctgccaactgctgattttccgctgtccggctttggcgagactgcgaccgcctacggcctgaaagggctggctattggtcacgctaaaatcaccgccgaaagtgtcccggcaatgaagggcgacactgtgcgccgtatgtattcaactggcacagcgacgctgaaaaattatattcagcaaaaagcgggccaaaaggccgaagcgctgcataactatggcgctttgctggtagagatcgacgacaacggcaacttctttgctcgccaaatcgaaacgatggacgaaagcgggatgttttacgatcttaatcataaatttactgttaacggtggcgaggaggtaacgggccacgttgccgcgctgcaatatggtgacattcacgccgaaaagctggatcacgctgtcgcctttgcttcatgggggccgtgtgatgattctctagtgaacgtcctgcgccctcgttaccagattgtgcatgatgtgcatgactttacatcacgaaaccatcataaccgcgctagtggcgttttccttgcgaaacaatatgccgccggacgtgacaaggtaattgacgatcttatcgataccgggcgcgtactagaagcaatggaacgtgaattcagtcaaacggtcattgttgaatcgaaccacgatcttgcgctttctcgctggctggatgatagtaaagctaacattcaacaagacccggcgaacgcccatctttattatcgcctgaatgctgcgatttatgaagcaatcgaaaacaaggacgatacttttaacgtactagattacgccctgcgcaatgttgctggctgcgattttgctgcgatcttcttaaccacggatgaatctatgaaaattgcgggcatcgaatgcggttcccacggtcacaacggcattaacggcgctcgcggcaacccgaagggattccgcaagctcggcaagatgaacactggccatacgcatacgcccagcatttacggcggcgtttataccgctggcgtcgctggttccctcgatatgggttacaacatcggcgcgtctagctggtcacaaacgcatttgatcacctatgaaaacgggcaacgtaccttgattgactttaaagacggcgttttctttgcataacaaaaaagccggggtaattgccccggcaataaataacccataaataaataaataagctggagaaacaaatgaaacttactttcatctataacaatcgtaaatctttcaccgcgtccaacgtcgtagaaaatagccttgttatttcccgcgatagtgaagggcgcccgcatgttagctatcagaaagttaacacggtggacggcgacactgttttaaaagctctagccgctatcctcccgcgccccgctgaatttaaagaatccggcattgtgtcgcaattagtcgccgccgattctattctttatgaggctgatatctgcgagatcgtagagattgacgccgccgccgctggccttatgtttatcgtcgtaagcgaaaatgattatgatgatacctatctgctcggcgacgtgatggattattcttctagcgaatatacgccgccgctggcaattgtcccggtaatggctacccggattaaaccggctgaattagccgatgccttaactttattcttctgataattaacaaagcgagttataatatagctcgcttgattaattaccaacaaccccgaaaccggagaaatgaaaaatggcttttaataaactggcaattaaggcaattaagttatgggatttagacggaactgttattaattcctttgcccgcgtgttcccgtgtatggatgataaagggaacttagatttaaacatgtaccgggaaaaggcttgcgtacatgatgcaattatgaccgacactcttttaccgctggttgaatatatgcgggcatcgcttaacgatcccactgtattaaacattattgtcaccgctcgttatatgggtaagagtgactactatttcctacgcaaacaacgtatccgggcggggcgcggtggtaatatccagatactgtcccgcgatgtattgcaccgatatattggcgatgctgattataaagaggtgtactattcgaaagatggtatctataaaacgcattatttcgaaatgcttaaagctgaatatccgaacgctactatcactatgattgacgataatagaggcgtgttggctgctgctgctgctgctggccttcaaacgatggacgctaccgcaattaacgatattctatctattggtgtgcgccttgcgggtgagtcattcatagatgaagcgctggatgatgataatgattatcaatatctttgcgagcgtttagctcattgctgggaaggtatgaccgaagaagaaagaagcgactacggaattaagccgcaacaatttattcaatcgttagccatcgcatcataagaaagctaatagtaccctagcgaaatagttagggtactaatagttagctaggggggtaatcggactaccataccaccccccgaccctctaagcccacctccatgtgtaactttatgaaatttggagaaaaggctaaagggagtacttaggcaaacttagaacgagcagcccgaatcaaagctactactgctactgccgctgtcgcaataagaacttcccgagtcatacccgctatacgttgcaactgccgcagttgtagctatgattgctgcagtcgacccatcatcgctccgagaacttcccgagttcgatgaaggacttcccgaaactttccgagaattatccgagcctgcccgtttttgcttgcgtttcgcgacctgtggtgtcactggagggatgtagtacgggttgctcgggtcattccgaagatcgttcatgactcgttggtgttttgagatcgctacttgacgttctgcccgagtttgaaggatctcttgatgcagacttttgttacgaagatctgccgctactaccgcgttccgcaggtcatgtgcccgcacaccaaaccaaactgctgccaggaaaaagaatgttgtgaaaattaatacaatagtcatgttttacaccatacccttagtaatgtagattttgtttgctgattctagttctgccttgcattcagacaggtttaactgtgccttatgcagcaaacgggtttgattgtcataagacttctgcagcaccccgttagtgcgttctagctgtttgattgccttaccctgctttacggtaatgacgaatagtacgattgagagtaaaacaagtgctcctataactacttccatagtggtcctcctagatggttttaaaaaatctggtttacttggcggttgatttaatttataataatattatattataaaagtcacttgttagcaagtgagattttgaggtttttatgggcttttatgcaggaaggataggcgacaaaaaagtgttgtctcttacctctggcaacaataaagacgttaataaccacactaatccaggttgggatactatattccatagcgatatgcctcacgttgtagttttagaaactcacgaaagagacctctgggatggtggtgattggtatcgttgtactagaatgccagacagaattattcaggtactgtccgcagactacgacagggttgtcttaactgaagttgagtttgaggatggcaccagacgtttcatttatggtacatccctgggtgtgggcgctaaagcgtataacgcttactttagtaatactgtcggttcccaggcttctgcaggtactatggctagtatgaagactaacgtttgtgcttctgcagacttacacatggatattagtttctatttcgaagagacgccaggtactattaacgagaagctacgagacggtactgggtgtatgtacacctggggcgttaactcagaatggggagacagagggcctgggccgccggttggagctcctatacgtcctaactttgagactattattaaagctggatgggtgctctataggggggcttttagtggcaatatagccggttctgtgtctccgcccaatagaccccttactattggtgttgatgctatgcgccacccgtggatgcgtactactggtgttaacagtatatgtttgcgtggtgagactctcaaccgtaatatgtacggacatatggggcctagatatgggcagagctccaatcccgttggtggtccttatgctcataacatccagactgagtcttatcaggaggttcagtacaaagcaggttttttccgtggtccgcccaataactttatggggtgggaaaacacagataacaacaatgctggtagtggttggggtaacaatgccatttaccgtgacaataacttccgtgttcctaaacgtgttcgctggtatattactaatatgaaatacaatgggcaggggttctacgcagagaacgtatttggctcccgcaaccaggagattaaaatcagccctagagagttcattgtaaacggtataaacctaatgaacactgggtggaagttcataaaccagaacgatataaactacagcccaggtaataggcctgatatccgagttattgcaaccaacgtcgccagatttagtggcaaccctactgttggtaataatggatatgttcactttaatcagcctctaactcgaccagataatggtgctgagtttggacaaggtaacgtgagtgagatgcatgtaaccactgtaggggtttacaattttagatctgatgcacagtggtacgtaaaatctaacccgccggaaatcggaaaccagtggggtccagtatggtctgaatcaactcgacctctccgcctagtgggcggtaccggctctgctgatattggagggaacctacgtactagcggtaatgctagtcaccacctggctaccttgtggttaggggttaataactcccgaaatggggcttgcgtcgtcactctagactggaaaaatgatgagtggattgctgctgcagggattggatgttataaccctctagaagatcttacccagtggagcgaggtggatagtaggctgagaattttcggaaatcacttccagaaacgtgtgcatcaaatcatgtgtttgcccgttaacatgtgtgtgccgttccactttatacgcgggaccgtaacccagtgtggggttattcctgggaacaacgccatgcaaatgaaggctatgtgggcacctactactaccaactctgccactcagggcgattatgccattatctattggctgatagctagggctgacggtagtgttgaagtttgggttaacgttgagatgagcaatatcatgaatatgcgagtaattcttcctgaggttaggattgctgtgcaaaggcttgcctaaaggaggcaatatgagcaatgatttaattgtaccagatacaatgtctccggaaggcatgctagttatagaggcctacctggagtctggcagcgacgttgcgaaagcagcgttagctgttggcatggaggaacctaaattccgggagattatgcgtaaacctgaggtcaaagcgtaccttacggatatcttcatggaatctggcttccgtaaccgtgataaattcttcggcattctagacactgttctaactatgaaaatggaggaactggatgaaactggaatgggttcagagatggatattatggatatcctcaaactcatgcacaaaatgaagatggatgagatgaagatgcagatcgagtatgagaaggtgaagcaagctaaagctccaatacaccaaaataatactcagatcaacctggcagggggtcacgactctaattacacggaccttctgtcacgcattgtgggagcaggtaagtaatggaaatctcacgtagttatattaatacgactgacgtggtggattttggtgttgataaacgattctttaaattcccggtgtccggcttgctggccaccgaggggatcgttccaaatggcccgcagtgcgcaatcataaacgcactggaagacccacgtcaccgtttcgttacagcatgcgtatcacgtcgtgttggcaagtcgttcattgcgtatactctaggcttccttaaactcctagagccgaacgttaaggtgctcgtagtagctccgaactactccctggcaaacatcgggtgggcgcagatcaaaggtctgattaagaaatacgggcttcaaactgagcgtgagaacgccaaagataaagagattgaacttgctaacggttcgctctttaagctggcttctgctgcgcaggctgactccgcagttggtcgttcatacgactttattatctttgacgaagcagcgatctccgacgttggtggggacgcttttgatattcagctccgtcctacactagacaaacctaactctaaagctctgtttatctctactcctcgtggcggtaactggttcaagcgcttctatgaacagggattcagagaggatcttccgcaatgggtatcaatccacggtacataccgagataaccctcgcgtatctcttgcagatatcgaggaagcacgtaagactgtatctaagaactacttcaaacaggagtatgaggcggacttctccgtattcgaaggtcagatttacgacaccttcagcgtttccgagcacgtacaggatcttgcaggtatggggcacttctttgctgcggaccatgagttcgaaaccattctaggtatcgacgttggttacagagacccgacagcagtacttaccattaagtaccactatgaccaagatgtgtactacatccttgaggagtaccagcaggcagagaagactacagcacagcatgccatgtacatacaacactgcatagaccggtataacgtagaccgtatcttcgttgactccgcggctgcacagttccgacaggacttagcatacgagcatgaaatatcctctgctcctgccaaaaaatctgttctagatggcctggcatgcctagctgctctattccagcagggtaagatcattgtcgacgcctcgtgcactgcgttaattcacgcactccagaactacaagtgggacttccaagaaggggaggaaaagctctccagagagaaacctcgtcacgatgccaactctcacttatgtgacgcactgcgttacggcatctactcgatttcccgtggcaaataagagctgagttggaacggatagtagtgagtacaaattccgttccaactttataaaattcactttacaatttcgtgtgtggcagttataatatattcataccttgagagatctcatagacatcagggtgaatgagaggaatatttaattgctatacccgaagtagtattttcccaactaagaggaggaacactttggcttccaatgtaaaatataagagagatgcaatctccataatgcgtgacggaatcaaagcccagtacaaaagaggcaactgttgcgccatttgtgactcacaggaaaacctggagctacaccactactcgactgtggccttactagttaaaaactttgctaaagaattccaactggatttcactgactcagaagtcgtcctaagtaatcgggataagttctataaacattattggcatgagctagtagaggacacggtcaccttatgtgtctttcatcatcagaccttacacaaggtctatacgaaagaacctccgttgttttcagctaacaaacagaagatttgggttgagaaacaacgtgaaagatgtatgaatccagaggcacctcgtacaagcaacactggcgaaagatcaggctttgcgaagtggcttccgactgacgtcaagactgagaaatcaggattcgcaaggttcctataatggctattcgtgactggttagttactaaactaaaccgcggacaacgcataatcagggacttggaggatgttagtcaccgtactaacgtcaagccattcacgactggcaaagcctattcgtctattgagatccttaatagatccgcgaacatggtaattgacagcgccgctgagtgctcctacaccgtaggtgaacagtataaaacaataacaacctatggcacgatcaggagtaaaactcttgagacgctgcttaatgttcgccctaacccgtacatggattccagtacttttagacgcctaatagtgtctgaccttctattcgaagggtgcgcgtatatccattgggacggttcgtctctgtaccatctgcctgctgccctaatggaagtaaaagcagatgacaaaaaattcgttaacaaattcgtctttaataatatgatcgactatcgcgttgatgaaattatcttcatcaaagataatggccagaatggtggtattaactcccagattacgggtcaatctcgtgtggctaccgtaattaactcgcttactaagcgtgagaaaatgcttgagttcaaggagaaattcctggacaatggtacggttatcggtcttattctagaaacagatgaaatcctaaataaaaagctccgtgaacgtaaacaagaagagcttcagctggactataaccctagtaccggccaatcaactgtgctcattctagacggcgggatgaaggctaagccatactcccaaatctcctcctttaaagatctcgatttcgagaacgatatcgctcgttttaataaagacgtatgtatcgctctcggagtcccacaactattgatcgatgggggtaacaacgccaatattcgaccgaacattgagctgttctactacatgactattgtgcctatgctcaataaagtatgtagctctcttacgttcttcttcggttttaaagtaacgcccaatactaaagacgtagtagcactaactcctgacaaggagaaagaggctaaattcgtaactgcactggtcaacaacggtatccttaccggtaacgaaggtcgtatagagcttggttacgaggagctggctgacgagcagatgaagaaaattcgcatccctgccaacgtagctggatcagctaccggagtaagtggacaagaaggtggcgctcccaataaagacgaggaaaaacaatgattgactataaagcattaaaagcactattccctaatggtctccccgaggcacataacgtgtttgcaaccgttaaggcacatctcacttaccagattctgcgtaaggagtatgggtacgctgctactaacagtaaaacctgggatcagtttaaggaagcctacgcggaagcaactaagccagtaccggtagcttctgttagtatcacgggcgctcctgcatccttagactatactaagactgtacagcttgccgcaactgtcctaccaacaaacgcagataataaaactgtaacgtggaagactagtgatgctaccctagctactgtgagctcaacaggattagtaacagccctgtctaaggcaggcactgttaaaattactgccactgcaggtggtaaatctagtgaagtgtctattcaggtcaaggctcctgttgtagcagttaccggtgtcactatgtcacctaagactattacaatcgaagcaggtaagaccggcaaacttactggtaccgtagccccggctaacgcaaccaataagtctgtaacttacacttctgctgataccaccaaagctaccgtagctgcggacggtacggtaactgttcctgctaacctggctgcggatagtaccgtagttattactgttaaaacagctgacggcaataagaccgacactgcaatagtaacagttaaggttcctacagcgggtgtgtaatatctgttaactggggagcttcggctccccatttttctatgctagtgcataactattttcagtgtaaccaatttcgactacctactaattccacacaccgtgtgggattttcaaatatcttttgacattcgcgatttggtatgtaataatagtatctgaaaactgaggaacaacacgaaagtgtttggagcaaacgatgcagaatattaatcttaatgctgagattaaatctgttaaagctgtcggtgagggtgataatcctcctttaaaaatcaaagggtatgctaacacgattaccaaagaccgcgccggcgacgttattctctccgaagcgtggactaccagtaacgccctcaagaattttatgaagaacccgatcatgcttttcggccataaccatagccgcccaattggtaagatcctagacctggtaccaaccgagtccggacttatggttgagggtgaggtaagtgctgctgatctacagatttactcattaatacgtgatgaggttcttaaaacgttctcggtaggcttctatattaaagacgccgaatgggacgatatgactgaaacgttcattattaaagatctggagttactcgagatctctgtagtctcggttccctgcaaccaagactcaacttttagcctttcgaagtctgtaaaccataatgattacatggagcttcgtaagtcttttgtaaaatcttcgcaggtccagcctgttgaacaacctgaactttctaatttagagaaattcctagtagccgctggttacgctaaaggataatggagaaataataatgtcttacgatatcgcacaactgtctaaagatctgggcctgggtgacattgctgagcagcttaaaggtctaaccgcctctcagaaagctgaagaagctcgcaaatttgctgctgagcaggaagctaaagaactcaagcgtatggaagacctggttgctaaagcaactggtgaagaccgtaaaaacctggcagaagctctggagcttgtaaaaaacctggatgagaaatccaaacagtccgctgaagcgttcgttaaagcaatgaactcccagcaggaagaaatcactggtctgaaagaagaaatcaaatctctgctggccgctcgtgaaaatggtcgctctttcgtagctgatggcgttgctaaggcaatgtttggtaagcaggaagatttcgaagacgaagttgagaaactggttcttctgtcctacgtaatgcagaaagatgtattcggtactaagcgtggcgaagcccacctgaaagctgttaacggctcttcttctatcgaagtttctaccgaagcatacgaaaccatcttctccctgcgtatcctgcgtgacattcaggctaaactgattatcggtaccatgttcgaagaactgccgatgtctagcaaattgctgaccatgatggttgagccggaagctggtgaagctagctgggttgacgcctctacttacggtactcctgctactgttggtgcggaagacaaaaccaagctgtctgaaatcaccttcaagacctacaaactggctgctaaagcgtatatgaccgacgaaacagaagaagatgctatcttcactctgctgccgatcatgcgtcgtcgtctgattgaagctcacgctatcgcaatcgaaaaggcgttcctgaccggtactggtgctgcgggtactccgaaaggcctgatccagttcgctaaagacgatggtaaagtggttgctaccactgctaaagctgacggttctgttaaagttaccgctaaagaaattcacaagctgcgtcgttcgctgggccgccacggtctagacctgaacaaactggctctggttgtttcgatggatgcttactacgatctgatcgaagacgaagaattccaggacgttgcacaggttaccgctaccaccgctatcaaactgcagggtcaggttggtcgtatctacggtctgccggtactggtttctgagttcttcccagctaaggctgcaagcgctgagttctgtgtagttgtttaccgtgacaacttcatcgttcctcgtcagcgtgcaatcactgttgagaaagagcgtcaggctgaacgtcagcgcgatgcgtactacgttactcagcgtctgaacctgatgcgtttcttcgagaacggcgtagttgctggtgcttacgctgcttaatttggctatactgccgactaaaggagagcttcggctctccttttttattgggtaaaatatgcaattcatgacagattctgattggagaacatatggaggccttaaacgtcctgacttagagtcaaatatcccaatgttaatcaaagcagccaatgctctgattactcagcttctaggtattgacgacactgctaacgttgtagacgttctgcctactaaaccagcacgcaaaaagtacttcctgtcttctcccgtgcctagcacgatcactaaaattacgattaacgatcaggagatcgataagtcgcaatataagaactacccggatggtacgctcttgctgaaattctcccctccagaggggtatatggaagtagagtttactcagaccggcttcacctcgatccctgacgacctggtactagcagcctgcttcctagttgatcactgggtcaagaaggactaccgcgagtctcgtacattcggcggagaaaccgttactttcaataccactaaatctggcgtaccggaacacattcgtactataatcgaagtataccggaggctgtagtggcgttgggcgatctagctcgacagatagttaaagaacagctggacattatgagtggtggtagccactctaccaagaacaccgtgatatatagtgcggaaactatggataaccacaaagatggcaccataggcaaggtatccttccgatttaccaagccggtatcggaggatttactgaatgttcggacgtcctctattttaaaagctgtctcgtcgtcacttaatctggaaggtgacgttggtgttatcgataaccttctaaatagtatcactggtaaaaaatccaaaataggaagaaaacggtctaccggtagggtagaggttaactttggagatccttcagatgctgacaacggttacgctggtgctatttctggcgcttctgggcgtttcgtctcaaacacaaaccttagagcgttacttgaactcgtagcgaaagaatacttagtcaaggatatgaaaaaagctggggcaccccttaaatttagaacggggcgtttcgctaattccttgaagattaaagacgtgctgttaagagaagacgcaggggcaaagactcctgacctcaacatcacgtataactacatgcttaagccttactctgtgttcaaccctgccgtctctacctaccgcggactttccttacggcctttccctggtgctaggaacccgcagaagctgatcggcgaggcaatcgctaaggctgcaagagaccttatccattctagataccggataagagtaaatcagggtacataatgaattacagaacaagtattgctgatgccctagtggaacgactgaagaaggacatggatgggagtaatcccacagagttcttcactaatatgtatgggaacgtatcccgccagacttattcgtttgagcagatcaatgagttcccttacatagcagtccatgtgggtaccgaaactggaaactacctgccgtctgcacagcagtgggtttaccttgaaattcctattcttatctatgataaagaaaaggatgatattaacatgcaacttgagaaactcatagcggatgtaaaaacctctattgacactggaggaaatttacaatatactataatgaaacctgatggttcaactatcgattctgaagccactgacatgcagatcacgtcggtgtccacagacgagggtatactgtccccgtttggttttgctcaagttaacgtaacagtccggtatatgcctctgagaagagcgctggatagataagttacagctcaggagaaatttaaatgtctgtacaactattacgtaatacacgaatcttcgtgtcaaccgttactacggggttcactaaggccaacactcaggagatcctagttcaggatgatgtctcctggagccaggacagtaactccactgatattaccctaaatgaggctggccctaagccgacccgcggttcgcagcgctttaacgattcattgaacgctgctgagtggagcttctccacttatatcctaccgtatgacgatgcaggtaaacagatcctgccggactacctactgtggcacggactggcaactggagctgccgtgaatctagcaggtactactggcgtattccagaatgctactaacctggttgtcaactttaaagacaacgggtaccacgaactggccatgttgaacatctacatcctaaccgatagctcttggtctgtgatccgtaactgccaggttgggcaggctgaggttaacgtagatattgatgatatcgggcgtgtaacctggtcaggtaatggtactcgcttggagaccctagcttctcagccgttcgaccctaaaaccatagggatagacgacgctctttatgctaagattcagagttcttatatcaagaacaaactgaccattcttaagctgaagaacaacgctaccggcggaaaaacctataacatcccgatcacgggaggttctttcactatgaacaacaacgtgacgtacctgactcctaacatcatgtctcgtgttgacgttccgatcggttcattcactggttccttcgagctgactggttccttgacagcgtatatgaatgatgctgccaacggctctatccagctgtacaaagatctggtttccgacctgaaagctgtgaacgacttcgaagttgcaatcatcctgggtggagagtatgatactgctcgtccggcagctgttctggtggctaagcacgctaacctgaacatcccgtcaatcgaaactgatgacgtgctgggtgtgtctattgagttcaaggctattccgactcagatggacgcaggggatgaaggttatctgggcttctcttccaagtacaccaagacttcgatcgcgaagctgatcagctctggtgacggtaaccctgtcacaccataaggataactaatgctatactccctaatgcgggagtctagagtagtcatcgagtacgatggcagggcgtacggatttgacgccctgtctgattacactgctggaacgtcctacgaagagtttaaagcaaatcgtaggacgattcacagacgcagtaactacgcctattcgaagataactgctcagtctccttcttcaatttctctaactcttaacttctctagcaatgctctcgaaggtctatttttcgagttgatggggtttatagagatagacggaatgtatcagatgcccttgttcagtaataatattgagcctaaaatgttctccgtatatattattaacaagaacacgagcttacgtttcgataactgttttgctaccacctgcgacttttctctagataagagtgtcccggtgctaaacgttggtatcgagtcgggatactttgaggaagtaggccacccactcaacagctatacgcttgatcaaggtgaggtgctaccattttcattacctcaggtatcttcgaatgggagagtgctcccaggacttatgtcagccggtatgtcattccagcagcaatgcgaatggcgaggtgacagaagtttattcgatatcaataagatttataataatagaagggcaatcgttaacgaattgaactcatccgctttgatatcgatgtactatgcaaagagtcttcagatagactctacgcataacattaaacctgatattggcctaccggtacaaatcagaaataaatatattgtggtggatttcccttccactcgaatcacaaaacgcctagacttaaccgacgtgtacaagatcgattatgacgtaatacctactgagcaatcagatcctgtccgaatcaagctaattggagaataacaaatgagtattaacctaaaagatattgcactggataccaaacagatcaccattgcataccctggcctaccacacttcaaactgaaagttaactacgtctcccgtaagctctccaagaaaattctggaagctgcacaagagaaccagtttgttaatggtatcgctgttaaagtgcaaaacgatgacaaattcgcagaagagttcgtcaaagtggctattgcaggttgggaaggtctgaccgtcgcagatgttgagaaactaatgctgatcgaagttccagaagatcgtctggaagacaaagtcgaatttagtatcgacaacgcgatgatgttggtgcgtaactccagtgccttcgagacttggatgaacagcactgtcttccacctagacacttttcgtggctcaaaatcggaacctactgcttaaggaaatagatgcctttgcagaacgatgtgtcaaaggaggggactccaaaattacacgggagcagtatctgacgatgtgcgaatccctcggggaggaacctaaccccgaggtacttaaacgtttcgtagagatccatgacttccctgaaatcgcacagaccgctctaacaatatataacaacttatcggataactatatccccggagattatccaacctatttaggcaaggataagagtgctttactagttttcttcgatatctacggagtcgaagacgctgatgagaagagccttatactccaaatcatcaatatattcgactcacatgccgtggcagcctctcgtaaacgcgttgaagcagctattaagaagtctaaaatgaagtcttctagtaggtagtgagttacagtttcctccaacgggcgttccacgtgaggcttggcctagggttaatgccctaggcctttttattggaaaaaatcatgacagatagactaatacgagaattacttgtagatatcaagcagcgtggtggatccaaagccgctaaacaaatcagggatgttgaagctgctttagacggggctgctcagagctcagagggtctaaatacaagcttgggcaaacttcctgggtctttcacggcgctggaacgatctgtatcacgtactgctaagtcgctggagaaattatcctcgaccaccagcattacagcattagcagcgtctatcggcatgctaagcggcaagtttacctcgttcgaggttgacttggctaaatccgtactaaaaatcaacgcaaacctaaacggggtgacttccgccgctaacaaaatggcctctggttttgacactgcagccacttcttcggttgcggacttaaaccgcgtcaataaggctcttcaggagttagatgcgcacgcctcttcagtagctaaagtgttgcagaccttgaaggcgggggccgggttagaatctattagctctagtgctgctaaggctagtacggatcttagccacctagtatctggggtggaaaagataggtaaccaattagctagaatggcggagcaagccgtgctggcaggcaggtctcttcaggggctgaaagccgactccttaggggctgccggagagcatctgagtaaggctgcgtccgggatttccgtagctgtatcttctatgggcgaagaggtgaacaagctaaacaaactacttcttgagttggcagtaaaggctgatttagcgagtaaatctatagcaaatattgcaccagggaccaaactgaatagtctgggaactgaaatccagaagattaatactagtttagccactgcagccaatacctcggtagctgagatatctaagattaaagcagcccttacgtctttagtatcctctaccgctacagctgccgcttcaatgaaaaccgtaggaaccggtagtggtctgagcaagctaatctcagagatatcagcagccacctcagcttccacttcggatatctctaaagtaacagccgctttaaaacagcttaacgtagatgctacggcagcaggaaaagcactgcaaagtattaaagcaggcgcaaatctttcttctgtacctacagttgttggaaagataggtacttcaatgacgcagttgcgtgctcagttagaaggatctgtaaccggtatcgagaaaagcctaaatgatctatctagagcttttgccactatgggaggtacgggaaacctgaatccactgggtaactccatcagaggtatgatcccgtcactcacccagctggctaaggccgctgtgcaagttaactccgctctgtcaaaaatacaggcaggcaggggcgtacttcaattacctacccaattcaaagcagtaacggcctcattaaatgccctggagactaaactggcctctacgtctcaaatactagagcgcggattctccaagggatttcaggatatggcgtctaaatcaacctcgtcatctacgagaatgattaacaacttccagaaagtggtaccggagctcaacgctattgaagctgctgctatacgttctgctgctgcaatagacaagctaatagccaaacgtatacgcctcggacaagctgggggagggggtaaccctgcagcgttcaatatgggtgccttagtagcggaaatgaacaggattgtaacctccattgaagctatgggcaacaaaatgaataccaccatggctgatatggcacggagtacggacaaggtatctgacaaattaacagatctaaactcgggagttcgggatgttaatactggtttaggtgggttgaattcaacgttaacgggtacgggtagcgctgctaatagggcgtccagagcgttgggaaatacttcaggatctgctcgcggagctactaggaacttcgcagcactagctatggtgactggccctatgcctcttatctacggtgctatagcctctaacgtatacgtgcttaaagcagcgttcgatcagctaaaacttggagaccagctgaaccgcttagaacagtttggatctatcgtaggagcgaagacaggtacacctattcagtcccttgctgtggcactgcaggaagctaccggccacgcggtatcctttgaagaggcaatgcgtcaggcatctactgcggccgcgtatggtttcgacgctaaacagattagcgagtttgctctagttgcacgtagggcagcggctactcttggcgttgatatgaccgatgcactcaaccgtgtaatcaagggtgtgtccaagcaggaaattgagcttctagacgaattaggtgtaaccatccgtttaaatgatgcgtatgccgaatacgttaaaatacttaatgcggctaacactggtataacgtataacattcagggtctaacttccttccagaagcagcaggcatatgctaatgcggtagtagcagagtctaccaaacgcttcggctaccttgatgaggtactacgagcaaccccatgggagcaatttgcggctaatgcggattctgcattacgcaaggttcaacaagcagctgctaaatatctaggtccagttattgcatctataaacgcagcattctatacgtctaaggcttcggtatctgcagaggcagctactgcccagcaagagtcgattaagcaaatggacggtaaagactctaacgcagtggtcatgaaccttgaggcttctcagaaaggcttggatgatgcagtcaaagcaaaagaggaagtaaaaaataaactcgcagctcttaataaggagataatggatagagaggcgaagatggatatgtccactgcactggccacagccgccaactatagtgggttcggtaatctgcttaccctgggagcctctaaagctaacaaagaatttacacaacagactgcagatatgcgtagacaggcgtatatgttacagcaggagttagcagattctgcgggagctatccaaaaatggaaagacgccagggactccgctctatctaaggctcagaaagagaacccagaactggcggggaaacttaatatagggcagaacgttgaagcaagtaatggactatacacctttgacaacgcagcattagacggggcagttgctctacggaaggagttcaataatataaagaaaacttccggagatctgagcaacgatatccagaactttgcacaggactctaacactgcgtctcgagctactgcagcactgggtgatgcacttaaggcggttgagtcattggcgggtggatctacggaaaaagccaatcaaatgaccaaggaccttaatttgggctattccaccgtaaccgagatgaacactgcgtataaagccatgtctaactatcagaagatagtgaatgatgaggctaagtctaagctagatgttgagaaacgtatagcggaggtctacgctgccactcgtaataaggataaggcggaagaagctggtagagccctagaaatgcagcaacttagcgcgaagaaagaggcattgaaagctgtgctggcgacgaacaaggacaacaaggctattcaaaaagagttgaccctgttagagacggaagagctcaaagtgaagaaccagggcatggaagcgactaagaaggagaaattctataaggataagatagtaggcatagatcgagaaatagcactcctaaataatcgcactatgacagattctcagtataacgtggcgaaccttaaattaaatctacaagtagagaaagataggttagccttactgaagactcaggcagataaggagaaggaagccgaacagtctagacgtaacattgcctctattgaaagggaaatctggaaagagcagcttgaccgtaatgccaaaactgctgagatgcgtaaagaagaattcgagcgcaatcaaagcatgaagcctctaatgggagagtcacagaaaatgcaggagcagctagcgttctaccaagaaatgaaggaattcacgaaaggtaacgctgatgaacaggcgcgttggagcaaggaaattgctaacactactgcgcaaatggcggctctcaaagctcagcgtactgcacagatgatggatcgcgtaggacagtccttgggcgcagactatacgcctactactggcctggagggggaggataagaaattcgccgacatggaaaaccagatggcgtcatacgataccgctatcggtaagctctctcagctaaattcggaggctactgctactgctcaaagtatggggaacttagctaacgctatgatccagttctctcaaggatctctggatactacgtccatgattgcagcgggcatgcagacagttagccagatgatcagctacggtactaaccagcagatttcagcaattgatgcagctatagcggcagagcagaagcgtgacggtaaatctgagcagtctaagaacaagatcaaaaagatggaagcggagaagattaaacttcagcaggaatctgctaagaagcaaatcattatccagacagcagtagcggtaatgcaggcagcaacagctgttccgtatccgttctctattcctctgatgatcgcggctgggcttgcaggtgctctgtctctagcacaggcgtctagtgctaccggaatgaccgatatagcaggatctggtggtgaaaccgcctcttacctaaccttaggtgagcgccagaaaaacgtagacgttagcatgggcgcaaacgcaggtgaactatcttacgttcgcggcgataaaggcataggtagtgctaacggatttatccctcgtgcagaaggtggtaatacctacccaggtgtctcctataagatgggtgaacacggaacggaagttgcaactcctatggtgcctaccaaggtaactccagcggataaagtggcttctgaaacttcttctggcggtgctagaagaccggttaatctgaacatccaggcaatggacgctaagagctttatggagtacgcattggaaaaccctgcggcattccaggcagctgtagagttagccttgaatgaacaagggctgagcttaaagaacctgaattaactaaactaaagggaggacgtaattgtcctcccttctttttggaatttataaaattatcttgaaaatttttcttgatatgactataatattaacattgagaggagaaaacaaaacatgagattaccagatccctttacacatcctcagtataacggccttgggtttgataaagctacgctgatcgataatgatccagtgatcagagacgagctaccaaacggcaaggttaacgaagttaaaacagccactcagtactggggtcttaacattagttatcctgtgatgtttcccgacgagtatgctgtactttcgtcagcaattctagagtataagcgtaccagaggctatctcgacgttatactcccacattacgagtcttaccgagtaagaggggatgcgaacaactgtcgcattgccgctggacaaaaaggctccacactggttatcaccaatacgaattccttatctggagaacctaagccaggtgatttattccagttaaccacacatccaaaggtgtataagattacatcttttaaaaacgtagcaggagtatggacacttaatctataccctgatctgttgctcactaccaacggctccgagagaccacgtttcaatgggattcttttccaaactaaattaatgaacggagattcattcagcgaagagatcacagttgatggtgtatacgacggggttaacctagttctgagagaaagtctatgagacagatccttccttctgcgaaagcctaccttgccaacaatgacaagatacgattagcgtatcttgtctctatcgaactcccggggtccacgggtaataacgctgtttatgcttatatgacggactatatgagagatatcaactatggtggtatactcttccaatcagggaaaattaaaacaattagcagccacaaacaaaaccgtacgttaaccgtcggcagtttgagctttagtgttactggtacggatgccaacgaagtcattaagctcgtgcaaagtggtgtatcatttttagatcgctctatttctatatatcaggcgatcatcgacgacaatggggaaatccttccagtggacccagatactaatggccccttactcttctttaggggtaagattgtaggtggtggtatcaaagaaagtaatacagtatccggagttggtacttctgttataacctggaactgttctaatgaattctatgattttgagcgggttgctggacgcttcacagatgacgcttcccaccgaggacttgagattgtaaatggagaattactgccttctcacggtgccaaacgaccggaataccaagaagactatggattcttccacgccaacaagagcgttaacttcctagctaaatatcaagtaaaagaagaacgatacaagctagaatctaagaagaaattattcggtctctccaagagctacagccttaaaaagtattatgagactgttactaaagaagtagacctggacttcaaccttgcagccaaatttattcctgtagtatacggtactcaaaaagttcctggtatcccggttttcgcggatacagagagaaacaatccaaacgttgtgtacgtggtttacgcgttctgtgagggtgagattgaaggattcctagacttccagtttggggacgcccccatgatctgtactgatcaaactgacagtacatctcgtacatgctttggacaaaaaagggtgtcgggagatactatggcaagaatttccacagggctcccatcaacatctctctccacgcatggtcaagaatacaagtataatgacggtaacggagatatacgaatctggacattccatggcaagccagaccaaacggtagctacggtactaagagacattgctgctgctaacaatttcttccttcaaggagagaacggtaatggtccggagtactgggattctaggtacaagttattagacaccgcatacgctgtcatacgtttcactatcacggagaacagaactgatattcctgaagtatccgcagaattaagcggacgcaaagtgaaagtataccaggcagatggttctgttaaaatggataaaacgagtcagaatggtgtgtggcaaacatttgactacctaacctccaccacctttggtgcaagtatcccgatagacagaatggtgattggtgactggagaaaagaggccgatctattaaacattatagacacctcttatcaaactagttggcaacctttctggagatacgttggatgggagagctggacagccgaaaacagacaaataatgcaaatgaacacaatcctggataactccaactctgtgttcaaaaacgtgcaggagctattagaatccttccagggggcgttgaataacctatcaggtatcttccgcatcaccgtagagaaagattcaaaaactccgctagaacttaatttcctagatacttatggggacctggatctatcagatactacaggccgtaataagtacaactcagttcaggcatctctgattgatcctaccctgaactggaaaaccaactctataacgttctataattctaagtttaagaatgaagatcgtggagttgacaagaaacttcaactttcttttgctaacataaccaactactacactgctagaagcttggcagatagagagcttaagaagtctcgttactctcgctctctgagtttctctttaccttacaaattccttggtatagaacctaacgatcctgtagtattcacctacgatcgttatggctggaataagaagttcttcctagtagatgaggtggagaacacaagggatggtaagataaacgtaactcttcaggagtatggtgaggatgtatttattaactcaacgcaggtggataacagtagcgaggccgttcctgaaatatccaataacgtcctgccaccaagagactttaagtacacccctacaccaggtggaatggtaggtgatgttggcaaaaacggagagctttcatggcttcctagcttgacacctaacgtagtctattactcgatacgtaaatccgaccgcgtggatccttatattgtgcagcagactgctttcacacctaacgttagaatgttccaagatatcgtgggtgagcctgctggcctgactattttcgagatccgcgccgttgatattaatggtcgtcgtagctctccagtaacaatttctgtagatttgaactcagctaaaaaccttagtatggtggaaaacttccgagtgcttaaccttgcccctgaccctgctgaatgggtaggacctgacttagagttagggtgggacaagctgcaagaagaggggctgatttcagggattttctacacgcttgaaataagggacaacactaataaactgcttagatccgcaaagatcaccagtttatataactacagctacctgttgggctataataagctcgactacaaggccaacaactcgaatactttggggatttacagagcacttcagccaagaattcaggcggagggaccaaagggcgagaagtcggttgcatgggcatatatctaaatgatttcaaacatagcaccagctaaaatggtactgcagaatatcgtgaccggctatacgattgcgagtattcagcactcaatcttttccgattacgacgtaattggtagaactttctggctaactacgggtggggtaactacccgtagggactttaccggcgttgatacattcattgccacaattaacaatctaatcgctggcgctacctactctgcccagggtgctttctatgactcgatggttgatgcagagctgatggctgctaaagtaggtatgaacctctctagcaccattaacttcaagatgaaaactgctccgaagattaccaaagtgtcctcttttgcagaatctgttgacgtgggtgtgggtgctcctatggttgtcgtagagctttctggggaggccgaatacgttaccatcgaaatgaaacctgagggctctagcacctggactaaatactaccgtggtccaatcactgagcagatcatctttgggggtgttccagttggcagatacaatatccgggtatctggtgttgtcactatgccagatggtgttactgtggatgtttctgggtatgatacctggccgtcactgtttaacctgacctacaacttcactccaccgtctgccccaactaacctgcgtttcaaaactgcccacatccaagatggtatggagcgttttgacgttcgcctggaatgggattggactcgtggtacgggtgctaacgttcgtgaatttatcatccagtatattagtaacgatgagtttgcaaaaactggatggactaaggccaacaagctaaacgtgggtgcagctaaagctggtactatcactagcttcccttacaaaatccgccaccgcttccgtgtactatcggttgcttggggcccagatactcagtctataactaactctaacgaggttacttatattatagacgagagcaccactttcgacaatgcattcattaacgagaccggtgtagaaatgacctacgcaggtatcaagggtaaactctggaactctaacaccaaacagtgggagcagactttcttagtcgatgctgctacaggtgcagtagttcttggtacactcgatgaaaatggtaaagcgccgatttcattcgaccctgttaataagattgtaaacgtcgatggtaaagttatcactaaagacattaatgctgctaacgtaattcttactaacctgaccggtaaagataacccggcaatcttcactcagggtaagaagtacggtaataacgcaggtggtgtctggatgggtgttgacaacgttgacggtaaggccaaatttgacctcggtaataacactcagtatgtgcgctgggacggtgatactcttcgcatttctggtaacgttgtaatcggtactcctggaggcgatgtagaccttgaaaccggtatgcagggtaagcagactgtatttgcttataaacttggtacatctctgccaagtcgcccgcttgaccaagtttatccaccagctggatggtccgcattcccgcctaaccgcactgctcagaatcagaacgtgtacgttgtgcagggtactctggatcctaagaagagtcctcctgctctagtagacggtaccaactactcagctgcatcccagtggtctggtgtcccaggtactggtggtactgatggttctaacggtgattacactgttcagatttaccagatcagtgctagtaagcccacgaaaccaggcaatatcaatgaccctagcggttggagtcgtaccccaccgactggaacccctctttggatgtgttctggtagattcaatggcgatactaacgctctaactgttgagggttggtcagacccgatccgagtagacggcgagaaaggggctactggtgctactggtgctactggtcctcaaggtcctcaaggtccggcaggtggttcagtagaagtacagtggtctaaagatggtactactaactggcatgcaaactttactactggtgatatctacatgcgtcagcgtgttggtacaggtgggtggagttcagctatccgtgcagttggggaagatggtactaatggtactcctgggtctaagggtaactacattgggatgcgcttccgagtggcggctgagaaacctgccactccgactggccagaccccttcaggttggtcagatgcacctcctcagggaaaccctctatggatggttaaagctgagttcaacggtcaaaccaacgccctagtaggtacgtggtcagaaccagttcgtattgatggcgaaggtattggagtaaacctgtatccggttaagaaaacactggatcagtggaccggaatgagcaacggtactatggttaagaacccagataccctcagctttaccataaccaacacagagagcactacctcctctaccggaccgggtgcacatcctgttccgttccagggttcacaaggtcctatagttgagattccagtaaagccgaatacagcgtacatcttcacttatgaggttagtactgatagcaccagctttgttctgagagacctgttactagagttctctagcattaccggaagttttaccaacttccaagagttactgactggagccaaaggtaagcaggaagctaagattgtcactcgtgctgatactaagttcctgagcttccgtccaggtgtccgcactgccggagctacggttacttattctaatctaaaacttgaagaaggcattaaagctaccgcttacagcgtagaggcttccgacagtatcggtgagaaaggagaccaaggtactcagggtccacaagggcctcagggtaatcagggtccacagggtaatcaaggcccgcaaggtgctaagggtgataatgcaaaaggattcagcctctcttctctcggtcagacgtttacgtacgacgcagaaggtaaactgaagtccgatgcaaccatcctgttccaggctttccgtcagaatactactgcaaacgttacttggtcagccaaggacgagaaaggtgggaacatcactctgacaagtactagcaatactggtgctacactgaccgccgctaacttcaaaacatcgaagtctgtagtagttacagccgtctgtgatggtatcaccgatcagatcactattgtgcgtctagacgatggttccaacgctctcgtagggcttctgaccaacgaaggttccacagttctagcgaactatgctggttacgtgcaaaactatagtactggttctggtgacttcaaagtattctatggggcgaaggatatcacctcagaatgcaccttcagtactatggagaagaataaccttgatgctgatattacttcagcgggcaaatatactttaaaaggtatgccggctggtactgatgttatcaacggctgggtcgacttacgtgctgtacaccctacttacggtgccgtggtccgcagagtggcaactactaaatctatccttgcaaaaggttatgatcgcgttattaccacttcattcgagaacggaaataagggtacctggtcgactggtagtgtccaaggggtttctggtgccacaattgtagccgcaggtttcagcaaggccctagtgatctctgctagagactgtatagaagatgctaacgcattccctgtagtagctggtcagaaatatagactgggcatgtggataatggctagtgagtctaaagtcaatatcaacatgggaatgcgaattgtaagggcggccgacggagttgttgattggcaaggaacccttatgattgcacaaggtactgtagtacctggtggctggtcctacatcgaaaaagagtttactgttggcagttctaataccggtatagcaatgccgtggatccagatggctgggtcttctggtagtgacttaggtaaagcttacgttaccgatattcacattttcgccctagaaatggatggggagaaaggtgatactggtgcaactggtgctacaggttctcaaggtccacaaggtccacagggtaataaaggggacaaaggtgatactggtgcaactggtgctcaaggccctgctggtagctctgtaaacgtccagtggtctaaggatgggtctactaactggcatgctggtttccaacctggcgatattttcatgcgtcagcaggttaatggagtttggggcagtgctattcgtgcagttggcgaagacggtaaaaatggtgctgatggtactgacggtgactacatttcaatgaagtttatcgttcaggataccaaacctggcacacctactggtaacaacccaggtagctggagtgatgctcctccagttggtagccctttatggatgactaagggcactatgaatgctagcggtcaactacagggtacgtggtctaatccggtccgcctagacgggactatcaaccctaacctgttcgcagtacgtaagtggatggcagggatgactggtacggaagccggcacatctaagaacgatatcgagaagctagcgcataccttgactcgtacttccggaactgataatacggctccagggtgctatgcgaccccgtatctaggtagtggggcgttttcccatccagtgactccaggtaaacgctacactctaacctataatatcgacgcggctagcgaggtccagacccgagatactatattctggcaggctaatccagactcaggacaatccacctacatagaggaactgaataccgggacgtctataaaggttaagcgtacctttgtagttcctacaggtatgaactacctaaccctacgcccttctgctctgacacttaacgtagcgactacgtggagcaagattaagctggaagaaggcggagagaaaactgagtatcaagtagaatactcagacagtatcggtatagtaggtaaatcagttttagtacagtggtctaaggatagctcttctagtaactggcatgataccttccaaacaggtgacttgttcatgcgtcagaacgttgacggtgtttggggtcctgcgattcgtgctataggtgagaaaggtgagattggtcctgacggtaagaaaggtaactacaccaatatcatcttccgtatttcggatactaaaccagctaaacctactggtaacaaacctacggattggtttgatgctccacctgatggttcccctctttggatggcaacagcaacgtttaatggggatactaacgctatcattggtgcttggtctgaccctgtgcgaatcgatgcttccggcgtaggcgaaaacttcttagcgttcaaagagtggatgatgtctattcagagggctgaggggacgggctcttcagttagcaagaatccggaccagatgagattccgggtaacagctgggccatccagaaatgacgcgtacactaccccttaccaaggaaccggcacacattttatagaagtgtcgcctaatactgtgtatactttatcatttgaaatggaaactgctgtgtccactagaatgatgctgttgcagtttgataatggtaatgggggcacgcacgcacgtaacaatcaggttatatcaaccagtactggtataaacagtctgactattactacgggtgctaacactacccacctgtctatgcgtatgtcaatctctaacatgggagagactaacgtactgatgaaacctaagctggaactcggggcgtttcctacggcatacgtggcgcatcctagtgatctacttggtaaagatggtgctactggagctactggtcctcaaggtccgcagggtaacactggtgctactggtgctacaggcccacaaggtagtaaaggtgacacaggggctactggtcctcaaggtcctcaaggtccgaaaggtaacgcaggtgaaaacgctaaaggattcgccctaacgtcagattatcagtcattcgtgtatgatactgtaggggacattaaatccgctactactattctgttcaagggactaaaacagaatactactgcagggatcacgtggagtgctgtaaataatacgggagctgcagtaacactgatgaattctggagataaccgtcagctaaccgctgcgaacttcggcgcctctaagtgggttacgataacagcaacctgtgatggtttatcagatcaaattactgtggttcgtttgcaggacggtgagaacgtgttgaccgctgttatgacaaatgaggcagctacggtacttgctaactactccggatattgccagagttacgaaaacgccaaaggtcagatgagagtttggtacggaagcaccgatgttactggtcagtgcactttctctgagggcggaagaagtaacgtaactcctagcatcaactcagcaaatgggaactactccgttactggtatgttggatgggaccgatattaccgaaggttgggtggacgttaaggctactcatccaaaatatggagcaattaccaagcgttttgcggtaactaaggtattcctagccaagagctatgagatggttatcaccaataccttcgagaatggtaacaaaggttcatgggcaggagctctgcagagtgtttccggcccaacaaaccagagcatctctaaggcgctgcgtatcacagctagagataacctagagggtaggaacaccatcccagtagcaggggggcaaaaagtccgtatcagattctggtacaacccactaggattagaggaagccatttttagagtaggctttattgttcaccgcaaagatgggggcaaaggttacccctccagaactgtggttacgggccccgctcctaatagctcctgggcgtacttcgatcaagagttgactctaagtgctaacgatgagggtattgcctggccgtggttccagttagataacaaaacttctggttcttcattagggtatatgcttgttgctgacatacacttcgaagatctatccatggatggtgcagacggagctactggtcctcaaggtccgcagggtaacactggtgctacaggtcctcaaggtaacaagggggatactggtccacagggtccacaaggtcctgcaggagcttcagttggtgttcagtggtctaaaaccggcaacgcgagcgattggcacacaaactatgctactggcgacatttatatgcgtcagcaagttaacggtgtgtggtcttcggcaatccgcgcagttggcgaggatgggcgggttggtgctgatggtaaatatacctccctgagattccaagtagctgcaactaaacctgctagacctacagggaactctccggctaactggtctgattcacctccagaaggttccccactctggatggttaaaggtgagtttgactccagcaaccaacttcagggaacctggtcagatccagttcgtctggacggtgagacggtcaacctcaacctgtttgctaacaaagcatggattgcgtctataacgggtgccagtggtagtggatctgttgtagccaaaaaccctgacgaactacgcttacgcattaccgcaggttctggtgcaactgacgcgtataccatgcctagtggtggtgatggtacgttcttcactaaagttaccgcaggtaagcgctacactatgtcgttcgacacggatagtgctctggaaatgagaatgcatgtgttctttatccaggcaggagcaaatactactacctcatcattctcttggatagcgtcaacaactgctggcagaactagctggtcgttcactgttcctgcaggatgtgatagggtatctgtccgtgtgtcactgaacaacaacccaggtggaaccaacgttgtttccaatatcaagctggaagagggagatttcgccacagcgttcattaggaacgagctggatactattggtgctgatggttcacaaggcccacaaggtccacagggtagtaaaggtgataaaggggacacaggagctactggtccacaaggtccgcagggtccaaatggtactagcgccaaagcctttgccttaacatccgatagcctgtcctttagcttcgatactagtggtaacctgaaatctaatggtactatcaagatcgatagctggagacagaataccactgctgcaataacctggactgccaagaaccaagcagggagtaatataactttaggtggcactgctactaacaagactataacctctgctcagttcggaagctcagagtacgtaacagtaaccgcgacgtgtgatggaataaccgattccattacaatagttaggctgcaagacggggtgaactctctagtcgggtacttaactaacgaagcggctaacctgtcgtgtaactcctacggttttgtgcagaattgggatggcactacgggtaactttaaggtgttctacggaacggtagatgttaccagtcagtgtaccttcggagtggaggataagagcaatcttaacggcaacatagggtcaagcggttattatgcccctagcgctatgccaaacgggctggaaattacctctgggtgggtagattataaggctacgcatcctaagtacggaacacttattaagaggtttacactcaaaaagagcctgcccgggattggttacgacagagtgttcacggggtcctttgactctggtaacaatggatcgtggggacgtacggtagttgacatcgctacgggcagtcccggaggacacaccaaggctatacagtgcacctctagggacaccatggaaagtagtaactggttccctactcgtaaggggatgcgctaccgtgtaactgcatgggtgaacaactctgagggtgagtatcagctaaggttaggcctccatacccagaactcttctggcagcgttaacactggttacccaactatgctagccgcatcagctaaggattccgagggatggaaactagtaactggtattgtcacggtaggagatgggtctactgcggagactggtagggcaagaccatttatccagatgaatggtagtgctagccccttcggcaacgcttacgtagctgctatagcgatcgaagacctatctatggatggtgcagatggggcaacagggccgcagggtccacaaggtaatactggtgccactggtccgcaaggtgataaaggggatactggtcctcagggtccacagggtcctgcaggtaactctgtaaacgttcaatggtctaaggacgggtctactaactggcacagtacgttcacttcaggtgatctgtacatgcgtcagcaggtgaatggatcctggggtcctgcgatcagagcagtaggtgagaacggggctaatggtacgccaggttctaagggtaactatgtgagtatgaagttcgccgtaatggctagcactcctagtaggccttctggtagtaatccggctggctggtcagatagccctcctccaggtaacccgttatggatgattaaagcggagtttaatggggagactaatgctattataggtaattggtcagatcctattcgcctagatggggatagtattaacgagaacctgttctactttaaagcctggttagactcgattaccggagttgcaggcaacggttcatctataggtaagaactacgagttactaagggctaggataatcgcaggtacaggagttaccgatgcgtataccctcccatcagatggatccgcctccatgttcacctaccttcctccaagcaccacatatacgatgtctttcgagactgataacgctgtagaagttcgttgtcacgtattttggtacgctaagggaagcaataccactggaggagtgctgaagactattgcatctactactgcaggtttgagcagctttaccttcaccacgccggctaattcggataggatatccgttaggttctcagttaacgaatctgggggaaataacgttgtaggaaggtgtaagattgaaaagggagccttcgtaacgtcatatgttcgtaaccagtatgacgctgtaggggatcgtggtccagggttctatactcaggcgatcacaaacttaaccggatggaacgatactcaggcagcatctttcttccagtcaacatttggtggacctccggttaagtatgacgtactaactcagtataagtcgggctctccgcagaactcctggactcgtcaatggaatggctcggcatggacagctcctgcattaactgttcatggtgatatgatcgtctccggttctatcactgctgataagattattgcaaacaacgcgttcctggcgcagattggtgttgacatcctttacaatagggccgccgcactaagctctaacccagagggcacttacacaatgaaaatagacctggctaacgggtacattcatataaggtaacaaatgagcacggaaaacagagtagttgatattatccttgatcaaaacgtgtcatacggattgatgctacagttcatggatatcgatgactctgcgtacccagcaacggaaacccctgtcaatctgacaggggtaacccttaagtcttcaattaaagactctctggaatccactggggtaaaattagcagatttcgtcgtaacagtagtaaacgctacacaaggtcaggcgtcgctaggattaactgcggctaccgtggcaacaatcgttagtaaagcaagtaaagaacgagataaatataatcctagacttcggttcgcaggttactatgatgtaatcatgaccaaaggaacaggagctaccgctacctcttatagagtcatggaggggagcgtgtacgtcagtgatggagtaaccgcgtaatggctattacaacaagaattattgcacaacaggttaccgctcttgacggagctaactcaagggtaagtaagtacccaaaatttaccgtacagctaggatactcagttagttctctggctgctacagaattactagatgctgccactagatcggcagcatccgccgccgccgctaaaacttcggaaaccaatgctaaggcttctgaaaccgctagtaaaaattcccagacagcagctaagacttcagaaactaacgcggcagcatccgcccaagtagctcagaatttggcaggtaaagcgtccctagtaacaccactaggagtgatgaccggctcggcagaagctaagattgcatctataacaatagccgcaaatcagtcttctagcgttcacgtattatttgcgctctacgctacaggtaatggagccaacagggatgatatatacaatatggagatagtatccctggcattacctggtcctgtaacctctgtaaccgcggacaacatcggcagcttccttagtcatcgagtaataggtccggccaacactaatggatttatggtaggacttaaatctactattgagggtagcaacgtgacctatgatgtatacctcaaatctcgtagtagtttcagagaccctaagatggcattcttatccggctccatatctgttactccacctaccggacctttggtggacggaacatcccctgcgtggaaaactacaggttttgatactgatgttatctatgtaaatagggcgcaagtaattgatgatggcattagtttagcccgcatcaaacaactagctataactaacggtaaaaccgatagctctatacttcttttatcctatttaaatgaaacaggtatactatctaccaataagaagtctatttccctccggccaggaggtacgagtgactctagtattgcagctacggagtttctacctaacgggaatataattctgcctaatggggatactgggaaccaaactattagttggttaggtggtccccgcatacgagtcaactccaacgggtcttttgttctttctactaataatcccagtaatcaaactagtgggtttataactttcaggccacaaggtgatcaagtaacttccactgagcttcagattagggatgatggtaacattaagcagacagctccacagtcatcggcaggcaatgcacttatacgccaggatgcggctattcaacatatcatggataaggctccggctgccggtattactgctaaccccctaagcgacttgaacgtaatacctacgcctgaaggtacagatccttggggagcagacggtgtacgtgtattccaatcaggggtatcaacaaaaaatactccggacggaactactggtcggcttggaactatcctcaacgttaggcacacccagtaccgtataatgcagttcttcatgcagagtaatgctactgcacctattctgcatattagatcattaagggctgatcagggtaataccccaccggcatggtttaaagtttatacagaatactctaaacctaacattcagtctgacatagcaggtattactatagacggcaatggtttcgttaagaaagcctccccgatcgccaagctcatagccgaaattcctagcaaagaggattcattcttctggacaggcgtggaaactgtgggaggttacgtagggtgtaacgcagaggctcaaggtgtatttgctgtaaaaactggactaggcaagtatactattaaagggagtttaggctggaacacggaaggatggaaatttgagctaccaagagatgataacggcaatatgttgtgcttcgtggaatcggattggaatgaggaagagaaagagctaaacatacaggtgttcacccgtaagtttgatattaacacaggtaatatcatcgctggtgaacctatggagataccacaaggtcgttggattgatctacgtctggaaatgccaaaagtggaaataccagaagtggaatttccagaagatccagaagtataataaaataaaaccccagtcggagcaatccgctggggtttttctatttacatcttaccagaacttccgaagccaccttcgccacgatcagtgtcggtcagactatcgacaatttcaatgtcattggggttataatgaggtacgatcaccaactgacacagacgctcaaagttatcaatcggttgtgtctcgttgctcatattcaacaggttcatcttgatggttcctcgataatctgagtcaatcacccctgttgtattggcaatcattagccgacgtttccctagagaacttctcggaaccacgattcctacccagccttcgggaatctcaaccgcaactccggtatcaatcatcaatgactccccaggaggtatggcacgcaataggtcggaagcacgatctccgaagtaggcccgtagatccataccggctgcttccgagcttccaacgtgaggtttacaatcggggtgagataatttaatacgcattatacttctcctgtggcaatagccgtaatgtctttaacaaattgatcgtagatatcttgtcctgctgcggcaactgcttccccacagaaacttgggagatctactagagttaggttacgttccataagttcgcctgatttattcagagcctggacgaatttttgagttcctgggagcggtagtgcatcaataatgtctagcacgctaccatgttcccggattaggttgtagccacgtttttcgccgataccttcaacaccacgaatgttatcccccatatcgcccataatggcttttaatgagataaactgatctacagtatctacgttatggttgtcgaacatatctttctcgtgatattctttacgagtagtaaacgagaagcgggagatattcggagcaagcagggtatcccagtcaccgtctgtggagatgagccagatatgatcgtagtgatgacctatcagctgaataatgaaagccgccatgtcatctgcttctactccgcggattcggaaagtagggaattggcttgcaataagatcaaacgcatcgtctaagtattcaaagaattgacgatccgcctctacttccgcttcggagcgatcggcgtacttagcgtctcggttgcccttatactccgggaaaatattggtacggaagatactcttccctttatcccctagaacgatagtgtgcttcgcatcgtaggagtttgccaaagagttgatggtgtttgcaaaggatgctgcaataggcttaccactatctttcttgaagcggaagcctaagttcgtaccgtcaacaatcataagattacggcgtgaggcgaggcgttgttctgcctctcgtttcattgttccccaggatttactcatttaactaaatctccaatttcacatgcgtttaaccacggctcaaaaagaccgataacaatttccattccacgtttattaactatgaaatgtgctctactcatgaggttatcaatcatagggtcgctactatccagagcgataagccattgaccacgatctttcttaaagatcagggcgggtttcatgttcatttgctcaccttcacgtgcggcttgggcccaccatttttctagctgggattctccaacgtttaggatattgctattaaatttttcgtctgcgtagtgtttgatctcaaaacagtattggctcattttacctacgctaggtggcagatacacatccccctttagggagtgggattggccaaaagctcctgaaccaggaacacgttcccagtccaaggaggtatatttacgcagcatatcacggatttggtattccgcgcgttttcctttctcgcgactatctacggccattaggcctccaaaattgataagccagcttcgtcttgtttaacctcaatcttatgtgcatacggatgtgtgtgcccgtgagatacgatgattgagttcagctgatcttcttcctgtagaagttctacgagggtattaattccatccgggtcaatataacttacaacttcatcaaggaagagcaggttgatattaactttactaatcgttgaaagcaacattcgaattgctaatagcgtggaaatattgatacggctctgctgaccagtggagcagttgaccatagaagtcctgttaccatcattgtagatgacaacctgtagcttcgtctcgtctaactcgaaccctagagcaaatttaccgctggtcatgatagagagataatgattgatcatctcttcaaataccttgacactatgctccagcttataccctaccagatcttttagcccggtaattaagatatctagatccgctacttcttctgcaactaaggacagttcagcctggatcttatcaaactcctgctcggccttttgaatctgttcacgcttagcttcatatttcgcattttcacgtacaactgattcgttatgggttcttgcaagttctaccgaggatctgccttgacgaatgctagactcaatagctttgatacgctcagctaacccgctagcgtctacgatttcatcctgcgttccattgacctcatcaaattccttgaggtttttgcgagcctgttccagagcgttaaacttagagatataatccttaaacgccctatcttccttcctaagttcttcaacttctgcttccagcttttgacgttctttatagagcggatcatactcctccttagttcgtctcatagcttcttgggcggcggaggtatctaagtgagttccgcacgcagaacactgagttttacctgcggcatttttgaaatcgtagtaacgtttcttcaaatcatccgctctagttgccacgaccgttaggttacgcgtaacactcaggagttcctcagatttcgaggtgggcgcaggtaaattttcgaatggtgcaaaagttttttcggcagcttgtacggccatgtccaaagagctacgttttgcaatactggctttttgcttctgagccagtgctaccttgacttttgcttcggttaattcttcgactagtggctcttcatcgaaaacagggattgggatctctgattgaggtgcgccaagcgaggcttttgaggaaagtatagattgggcagttcgcaggctaccttccaatccggacagttttgccgagacggctttacgtcccgctttaacacgttcagaaacttccttatattgttcctgatcaaacaggttaacgaggaaagtcttgcgctgagcatccgtagtcttgaggaagtcgagactggatccaactgactgatatacgagcttagtaaatgtcgtaaaatcacaggcgagtacctcttctatcagcttatacgtttgggttgcggtgtgtccgctgatatcttcaccgtttttagtgagagttactttggcggttgacttcacaactttcttgacgttgtacacgtcaccgtcttttgtgaactgaccttctagcgtataggccttggagccagtatgccagttaaacaggtcgtccttcttgataccacgtgagttcttattataaagaagctcctctagagctgtaccaattgttgatttacccagcccgttacgcccgattaactgagttacgcggtgattatcaaattctatcaccacgttctcagcatatgacataaaatggctgattgtcaatttattaattactatcgacatactcagcggctctctttaatatgcgtgttctagacgcttcgtctagtttctgcacgtttgtgaaataagtatccagctcctgtagcagtgacatatcgtctagatctaacttagcatctttggtgacacggttattgatcttcttatctaacagttcgctgttctttaacgttttgagttgtgatacgtcgccggtgacttcgtatataactctgtcatacgcatcggcttccattggctcgccgacttcaatagtttttcgtatgagctgtggaagatctcccagttcaatccacgaaaggtagtgctcatggtctcttggcaagaccgtatcgatgataaaagctccgttagttccttttgtcctttctctgtgaaacgaagttgtaagcggagatccgggatagagcaggtcaacatccccgatcttttggctgttcttgtagctgtggagatcgcctgcaaatactttggagtacccatgttcgacgaatctttcgaggtagatttctggtacgacatgaggcgggattgcacctctgacatgcgtgaagcaaatgtctgaaactctgggcttccaactagctttgtgaaggctgctgtaagggatgatatcgaactctggagatcggtagtctttgacgactttccaaagtccatctgttgcgtcgctaattgctgctgcatagtgatccaggcaagaaatcgttttagatttcatttcgtggttcccggtgtaaataataccaggatgttggagggtagctaggaatgcaaacagtagttcaacttcttctgaagacgggtctgaaacatccatgatgtcaccaccaataatgtgaaggtcacatccagtagctccaaacacttcgtccagcttttcaccgagaagaataaaccgatttttctgccattcctgaggaacgttcttcgctcctagcttgatgtgatgatctgctgagaaaagtattttcatagtcaaaataaaagggagccgaagctccccttgtttggattaatcgaggtcagaagctgcttcgtgatcgatgttaccggagttcgcggaaccttcattacctttagattcgtcgccctcctcatctttcttaccttccagccatgcctggattgctttcttctgctcatcataggacggaaccgggtaggtaacatccagtttaggcactttttcgaagccttcgaattcgtctgcttcgttgtacagagcctcaccaatcagagcaacgtcggcttcatgcagggcggcctctttagaacccggctgatttttagccatctggaactgcattgcagcgatctgctgtacatcgtacttggtatcgaagccagtaccagatttggagatagtgatatcgatctcacccggatcttccagattcagctgggccatgatggagtggataccggtcaggatggttgccttaacttccatcagcttcagtttgttatcagtacgatcgataacaacggccaggtagtttttcttaggacgcagcggctgagccttaccatctttcttctccggatctttgaagcccatgtcgtgaaccggatcggatgctccacggatgaaacgctctttttcacggtcgaaacgcaggcactcgaacggagccggtttaccttccttattctgaatccagtacacataacgcgggagaacgccggaaacgatacgtacaacgttcttaccgttgttgaacttcatgtagtccagtttgtcgccgttagaaccgccagtagtagaaccccaagatttagtagccatatttaattttcctcgataaatgtcaattttgacttgttgattgcgattagtgggtggtgctcgataactgctctaggaacccacggtggaatatattgcatatccagtgatgtatccttcgagaagctatattcggcatagtttctaaaactcaaaagtcctagatactcagccaactgtcggtctgacagctttcgtttattataaacgattgtatcttcgttcaagataaatgatctacccgttaacataacatgagcttcgggatcttcaaccattcgtctgataatctgtacaatcagcgatgagttacctcgtgctaatatgtaaattttttcgtaatcataaaacaacatgattattatacaccattttgagagaatttagcaactaagtttttatttttctgcttcggactttccgaagattcagttgctgttcccattaatttatacagatattatatcgtatttttgaggactcagcaactgaaatttttaactatttgcttaactcttctttgtaaatctgtctctcagttcaatataaatattatatcaaactttggggttcttagcaaacagaattttatctaagtttgccatattcttctcgttaaagtcaagaacttcccacccattgagacgatataccgcttctctacccgccgcctgcttaaatcctacacctcctagcttcagatccacgaggattgggtctaacttatcatctgccatacgttggacacgacctgccaactgttcgattagagattcgttatttattaaagagcctaacactaaacacgataaagcatttaatgatacaccctccgagaagatgctttgggaagccgctaacacacacggaccgtcattggtcacgtcaatctgtacttgctccctatcatcgagcgaagttacaccggtgatcgtgtaggtcttcacacccctgagttccaaagcattcgtaactctctcaattagatctatacgatcggacacaaacaggactttatggcctgccatcgaatatagctcgcacaagtcaacaacctgttgaaagtattctggctgcgaatatacatcgttagctcttatcgcccacggcacattcatatttccagaaacctgcgtttttagcgcaaacctatggatagtaggtggcatagtgttatttaccggaggactatatactttcgttccaaaatagtccttaaacataacctgcaaaccatccttacgttttaatgtaccggatagcccgattttatatcgtgccgaagactgctctagaaacttggtaaacgtggtggcaacgcagtgatgcacttcgtcaacaataacggtgccgaattcttttgccaaagcggctccgtgtttgttaactgtctgtatattactaataacgataggagaatcgatattaaatttgccagacccaataactcctggctcaatcccaaagaacttgcgtacctccttctcccacatagctcgaatggtagtgttggtacatataactaacgttttctgcccaagtttatgggcgatagcaagagcaaggattgttttaccgaacccaggcttaccattaataatacacgtatcattacagtcttcatatatagggagctgatcccctggtcgcagggtgaacgatggctttggaatatccataggaaccagagtacgtttatctacgatctctgtcactttttgcccgaaagattctagaagatctaatctggttacgggaaaccacttgatctctttacctaccgatcctgagttctggaacattagaggatactttgccccaggtttaaagatctggtatgaagtgtgttttaataagtattcccacagttcgttgctgggtttacaatagattttattagatataactatcttcatatcttaactctaagtctaggagtttctggctcctcttcgtgcgtgtcaaaaataactggtgaaccgcgaaccaacgcgtacgaaatatagttggcaggttcactaagtaagaaaggatatggaacgttcttgacataacactggtactttccgttgaagattctagttgatcctctaacgcgttccgtgataatgttgtaataagtcgtaggtttccatgttactatcttacctgtggagtcgatgaaatgctttagtccagagtttattatctgagataagtacttgaactgtttctttaaaggatagagcttataaggtaaattttcccgctcaccataaagaaaaagccttcgctgagagaatgtcccaggaaggcttggattgtccagcacataccttgtaaatctcgtctggataacaatgtactccccctcttggaagagtacatcataagcccttaaagcatatactggtaagctaaagttaagcaccaagaaccctgcgaacgttgacaatatctttgctgatttccggcaaaaacttaatattggcgtacttatcgtgatcagggtggttttcatcgttagccgcaatagcagcgtagtcgaagtcctccataccgataatactacgtactttctcctcaaactccttgtcctcaatgcaagcaacagaaggacgctgtttcttgatcttaccacaggagtaatcacgagaaccgccagcctcggagtcagagtcgataccaataggacagcccggaatactaataccacggtctacctgaatgttacggattagcatctcgttatactgatcaactaaatcctcacgaacaatggcaaccacggagtcgtgaaccagcatgataatcttcatttcgtcctgcagacccagagagcgaatctcattatcggtatcgatagcgcctagcaggagactatcagaagatgcagactgaatgatggcgttgaaaccagaacgcagttcttcaccctgtacaccacggtcttcggagttaatgttgtgcagacgacgcttacgaccaaagtggctgtaaatgaagccgttagtttggatctgggcatgagatttatcgatccatttcttaagctgagggaatcggccaaagtaggtttcgatgtactctttcgcatcaccagtagtacattcagtgtacggtttaccggtcttcatgtgctcttctaggagagcttcgtttactgacgctgctactttagccgggccagaaccgtaaagaataccgaatgaaatagcttttgcagcctgtcgtagtgcaggatacagttttttaacctcagtcggtttacacggcagagcaaataccatgtgtgcaattgaaccgtggaagtctgagtagttttccgggtcgttctgcatgttgataaacacttgctgcatgttaatgtcaccagacagtacagcggcgtagtaaatctcagcagttgttaagtcccatgcgataatacgataacctacaggggcaacaatacaacctttaataacagactcatcacgaggtaactgctgaaggttcagcttaccagacgaactcagacgaccggatgtagtcatatggatatggaatccagtacgaatgcaaccgtcagcatcaatactgatcagcatcttctcgatatacgtcgagagcagcttggagatcttacgaatctctagcagagtcttagctatcggatgctggtccgaaagctctttcagggcatctgcgccggtggaatctgcacccgtatctgtcatgatacccgtcggggttaagccaacataatcgaacaggagcttacgaagctgtacaacagaggctgcgttaaatactgagccctgatcttcctctagcttacgaacttctggatactcatacagcttagcttttgcaagctgtagcgcagtcattaactgaacctgagcctctttcagacgatctttggagatcggtacaccacggtcttccatacgctgtaggaatacacatcccggcatcagtacatcgtaatacagagacttcagtcgagggttagcttcgacttttggcaggaagaaattggatagacgcagtgttgcatcggtatccttcgctgcgtaaggccacatgatatcaaacggaatgagatcatacgagaaatcctctttcttgatcttatgcgtcttgcaatacgtctccttgaactggtcgagttcaaaatcgtagtcccccatatcggtatacttcatcgccaaagatttcaaaccgtgtgtaccacgtcgttcatctagggcgtagtgcatcagcatggtatcgtggaggcgtttctcttctgctgccttatcaaaggatagacctaagtgatagcagtagaagtgcatatcaaacttcaagttatggaacacaacgccatgctccgggctatcgaacagtttctgtaagtaatagacggtattttctgtgatgacgtctgcatcaatatatacgccctgatactcttggtgggagattgagatccccagcatgtaaccatctcggcagtatagcgcactggtttcggagtcatatgcgatcatgccaggacacatggtatacaccatcttcacatacgcttcggcttcgtccgggcactgaatcgggcggtaatcccctgctttagagcgtttttcacgaccattgagaatggcatgaatgctctctactgtggcttcaaatacaggcttcatttccggtttaaagtggagctgggccggactgatacttgcaatccagttagcatacccatcatgctcaacacgtttacctgagtaatcggaaatgccctttttagctgcaaacttcaagaacgggtctgcacctaccagaataacgtaatcaaaatcttccggattgaacgggttctccggtgttccgatagtgatatgtttcttgagcagtcgcccggttactttctcactagccatgaagaaggtctcaacctcatggtcaaagagttcgaagtgtttctgatagcggacgttattcggtgatttgtctacaactgcgattttcattgatttctcctattaggtaagcaagtaaaacgtcagtgtttcttctcaaacttacccaattattatatcaaatcttgacatgttcagcaatcaagatttctacgtttctgatgagttggtcgacttcttcagcatctaggtctccagggtctttgccctctggaagcaagaagttacctataattggtataattctagtcttatcacggattaatttggccaacttcttagccgcctcattaccggacttatcattgtccaatatgatgactacgtgagtagtaccggcgatttggaacggcatgaatttatctgcaatgttatctagtgagaactggtgagtaccaaaacaacatgaaacgttatggcaacctttatcttctagattcaacatgtcgaatataccttcgactaggattagggctggggtgttatagcgaacagggaagattgggggggagaccttcttaggtctaatcatatatttcggaggggcagagctatccatattacggcctaagaagaataagtttctccctacggcatccgtaataggaaacactacacgaccttcccagtcctctgtttgttggaatgcaaagtatttcttgaaagtctctgctttgataccacgaaagtcttgatcgaatatcatggctgattctggaatttctagactgcgaccctcagttcggaggtcactaatcattctgcgaactctcaggagtctaggcgacgtcctgtactgttcttcattgaagtaatgaaagatgcttggtataccacgaccaaatccgcagcttaggcagtggaaaaccccggaatcggggtcaacacgcaaactaggatgagcatcctcatgatcaggattgagacagcggatgaggatgtccccacccgtatctctgtattcaatccctttgagatctaaaagctcggttattctactcataagtctcttgaggattctccggtgggattagtatcctcatccgaagcctttttcctttttggtttgtctttcggtttatcgctttcaagtgggataacaaactctgcttccatctgacgaatgtcctctagcttaaggtcagaagtctgatccatacgcagtgagttccagttgatcttcggcatgaatgtcataccctcggagttacgagtttttacgaagtcgaacttgattgcaccgtcatgcccctcgttctgtttagcagcatttaggtttgcagccatatcacaggaatcgagaatacctcttgccatacgggcgttaccctgttggtcaatctggtaaggggctacaccagcaacgttatgcttttggcacgtagacttgaatgtagaggagacaaccatctgctgcttccaatcgtacatatctagtgtcttcgtatccggcaaacgtacctggttgatgtagtctagcagggcgattgttaccttatctccatacttggaggttagcttagttagttcaacatcgatggttgctgtactaagttccggatcatacacaatgatcatcggagtgtgcaggggtctttcctgcattagcatagtctccatgtcatggaaatcactcatctcgttgagcgtataacgtgatacaaaatcgttatatacttccagaccgccttcaaacatttttgcacgagtcttcgcaagtttgttgagttcaataccttgtaaggagtcattgcgcatagccttagcggaaacaccggccataatggctaggttacgtctaaagacctcattctctggcatttcgatggagaaatatggggcgatatccccgttgtaatattgggctacttgaatgttagaacagatgattgatttacctgtaccacgccatccaccgagtagcagtgtttcttttctggcaattccgccaagttgagcatcaaattcgtttgagatgccgagagagataagatttaacatggagtcttcttttctcttgaaaacacgcatagtatcggcgttgaagaccttaccagtcgttgttactcgttcctctagcttcatgtgcagagccgctactcgatcaagtaattctccctgatccagtatggtaatatcctgcaacacatcagtttctagcagattcaggaatagatcctgtgtgtattccgactctaggacatgaatcgcatgcgatatgtcaacgtctggaatctgagtgtttgctaaaacagttagtgcttgagacaaccttgcgtttctgttagactctagcattagagcatctaacgaaggcattccgttatgctttttgtaataattctggacagcttggtagatcgcggtaaatgcgtcattgaagtgatctttgcgcagtctagagaatgtctctagggccacctgcttttgatcggaggctaggagcattttcaaaactactgcttggacgttatacataagatactctctacgccgcgttagcgtctacttaaacgaaaaaggggagaggaacaaaattcctctcccctcaggtcaaatcaattacttggcggatttagcgtcaagtttagcgcgcttagcaacaccatcgtgatccttagcagagataccacggcggctcagcatagacttaacaccacgctcggatttaccagttgcttctgcgatctcagcaacggtcatatcggcgatattcagaccttccagaacgtcttcgcgtgccttagcattagagacttcctggaccggcatagcttcgatacggccttcacgcagcaggctcagagctttaccacggatctgcttaacggtacgttcgaactgagcagcgattgcttcaacggacgcgccactggcaacggcattaatgaaagcagcttcctgatccgggctaaagctacgtacagcagcggccttttcagtcggcttaacagccgcagtcagttccaggctcaggattttaccctgtacttgtttagcggtataagcaccaccaactacagcagcagcgatttcagcgtaggtatactgacccggatgatcgttcaggaatgcaaccagttcatcttcctgacccggagtccacggagatttggttacgtcggatgctttctgtacttcgaagccttctttacgcagtttagaaccaaccgaacgggcggtaacttctttaccagtttcagcagccagttcagcagcgatgttagcaacggcttcctgagagataacggtcgcgttcagagcgttagctttagcggtcagggatgcagtgatttcttcgttccaggttaatttagccatttttatttattctccagtagaattttaatcgtcaagacttcgatgcccagcgattgtgcttttttgtatgaagaagaaccgatcttggtttcatcttcgcagattaagtagtttacagccttagtcacggaggttttcaccgtatacccaagactttccagatatttggtagcgtccgcacgatttttgaagtcgttgagcttaccagtaatgcaaagaacaactccgttggattcagtcgccggggtaacttgagcttcccgcgccccactgaatttccatggtagattaattacgttctgtcctgccggtgtattcttccacgaagctaagttttctccagctttaccttccgctttcacagattggaaatcgttgtaaagctgggataatttcttagcagcaacttcaccgataagagggatactaagagatccaagaaccgaaccaaagtcaacatcttgtctcagcttagtttccaactctagctcaagtttcgtggcgactttatcacctacagctctgaccaaatcttctttagtcaggaaaaagagttctgaaactttcgtgatttccagtttagcaatcgtctgtgggccaaagccctttaacttcatctttgagcagaagttctcgattaatttactgctttgagctggacacatagacttgttacggcagaacaactgatcgttaactaagtccagtttcgaaccacaagagtggcaattcagtggaatttcaattttcattgattttcttcctcatcaatttatataaatattatagcaagaaaatcaggactaagcaatcgaaatttttcagaatttagctcagtctctcaccaaatatttctttccctcaactgaatgataatagtatacaccctggcgatgaaaatgtcaataactactttaaaaaatcatcacggtgtatctattgaatcaagtgttactcgtaaacacgttctacgatgcgagggatgaccccaccactacgaattacgcgaatctggcaaccgatttctaggtcaagggcagtgatataaccaacgttattcagggtagcttttgaaatgacagcgtcgtcgatgactaccggggtgaagtatccgacaggagtaactttaccggaagccccaacttgccattctacccgttcgagcgtagtaacctcgccttcatcatcttctttgatggcgtacgcaccacgcgggaacttgttagtccaacctgctgcgttaaattggttgttgccatccatacgaaccaccacgccgtccgttgggaaccaatctactcgactggagatatccagacaggtaacaaaaccatcaccctggaggatatgcatatctccacagaaagtagcggtcaggccaactttgccggtttcacactgaatactatatgccgtgaacatcaggccaccttccgcaatacgggagagaaactctccagaatctttcaggttaatagcaccagaggcgaagttacgcatattttcaacttctttggtgatgtgtacttcaccagtaatctgcaccggagtcttttgggagatccgtttagggatgttcagtaacttcacattatgagttacatcattacctaagacgccgttaccacgggttaacgcagaaacgaactttccgtcaatatacagaagtgaaatagcacagccatcgagcttcggcgtttcaatgccctggaagggtacttcttcgccgcggcctgggtacactttctgtagggagaacatacggaataggtgaggtacatcacctttagggccaatttcttcctctagcggaaaacgacggattaaggcatcatactcttcatcagagataatcgacatgcctttgtagtacgcgtcttggcacagcttaataaattctttaacgtgttgcattatttggcctcgtagtaatcgaacagcatctgagtcttcccttcccagtaaaagttttcgcataccgcctggaaggtggacattaccctacgcttagattttggactgtacaattcgcatactagtacgttcttctcataccacatgcctagcatctggaagcctgacattgtccgtactacaggtttgcccttctctttctctaaccaggttaggactagctcctgaccctgaagtggcgggctggagtatgcgttaagcgttatcttttgcattctttaatgcctcgttaacaattctagacatgtgtgctctcagctgttgagtgtgagcatacgtcttagcagcttcggtcagcttattaactaattgagcgatttcatgatctttcataaagatctcctttaaactatgagaatattatatcaaggtttaaaacactaagcaattgagattttaaagaaaaagccagcaacttagtcgtcactggcttgggtcttttgagattcacggtaaacttcatgaagaacctctgaattgcttagtattttagtaaatgcacggaatagcgtgctggtcatttctaacgtgtacgggaacgagaagcctgatttagtagggaaccagttatcttcaatatctagtaaccataatcggaaaccaaagtataaattgcctctaaactccgagacagtcatacgaatctgtcgcccttcatcttcaaagataacgtgggactgatcatcaatatgtccttcgtaatcctgacaaatgttttgattcatttttattgttctcagataaacaaaaagcccccgaaggggctgattgcgtcttaacgacctactggagtagctcgatcaagttcttggttgaggctggtgatgcgcttaatattcgcaatcggaatatagcggaatttatcgctagagcggctaaagaccaggatatgatctttgtcagcgggttccaggccttcacggacgattcgctcggccagatatttatcttgcttaggatcgaactcagtcgtaccgtagaggtaagttacccccttggtacgcagtttggtgtattgcatgcaaaactcaccgtgtttctcacagatagctacgacttgtgctttattcattgcaataccctcttatttagtgattttacggatagcttcagccaggtgagaggcagctttaccggtcagtttgtcaacgatcgattcgtccaggaagccatcttccagtccagcgtcgttaaatgcagcgcgcagttcagcctgagcatcagacttagaagtacggccaccagctttaggggcatcaccaccagttttagcggtagacttaccagcttctttcttgacgtatacgccagctttggacagcttcatacgaaggccgttcggggaaacaccgtgctctttggcgatctcagctacgatctccatggaaacgccaggacgctcatcttccgggaagagttcgattttggcgacgtaggcagaagtcagttcagcttcgagttcaggagaccattgagttggagtagacatatttattacttccttatttattgttaaaaagagtttttaagttttgtttcagactatgagaatattataacaagttactgagctgcaaagcaaacagaattttctaggatttggttaacagctaccaaaccattaggggacaaacgatacatcttcagcaaaccacgatcgtatttggtaagccagccattatcttctgcttcgtccatgggaagtgcatagtgcagatcccggatagctgtagcaccctcggaatagatctgaaataagttcaggagttgatatttactcacctttacctctcttttctagtttcagggctgcttttacagcgttattaaccagttcagctacgtcttctttaccccacttgaaccccagggacttgatatcaacaccaagtgctaccagatgttcgacagacgccagatcccaatacatgtagttaacatactgctggcgggcttcggacagcaggtaaacacgatacgcgcctaccggattatccagagcttttttaacttcgccaatacactgatagccaggaacccaaacgtgttcgcctacttcgaatacctctttcattgattcttctggaatcgcaggtggaaagagcgggtcaacctcgccattaacacgaagcagagcaccgtgagcagacagaaccattctgatcatgtttgcggaacggtagaaacgctcggcaattgcctcaaaactatcacctgacaggtacgaatcgataacgttggccagttcagccccttcgattttagtaccacgtttctttttacgcatttcagcggaaacacgctggttatctttccattcctcgataaggcgttccatcgtggagttagacgctacgccgaggatttcgcaagcgcctttcttggttttgccttcctctagccactgaatcgcttccttaaacttttcgtccgggatgctattagcatgaagttttttgcggattgccatgttctttatcctctctcgaatttatgaaactattataatacagatttagcttctaagcaatcgcgattttcatagaacgtagggtcgaaagtaacgtgcatgaattcagatagactcttgtaccccatgttaagttccatgttgatgcgtatagaaaggcgtgtctttcgctgagcctctagccaatcatagatatttggcatctgtgctaagcgaataggtattactctacacaattctatctctttagctttccgtgctttcgtatcgcgtttgggaaacttccttgtttcccagcgggcatcacgcttgctcattacgtactccaagttcgttttgcgcttcatcggccagcgccttctcctcgtccgtcataaactcatacggtacagagataccctcgatagaacagtacgtgcgataccagtctacgatctgctccgtctccatttccttattaatacccatttgggttaagtacatacgggcatatttagggttggcagactgtcccgtcctaataaagaagtccttcttgtgtttcttcagggcttcaataaaggggctaatcgtcacgttagaacacttcttaatatccttccagaatatatcttgaagtttatggaagaactccgcccgcttgtgtggtttcttactaatccacatctccgcatgttcctgtgtgatactcttgatagagtccaggtggaagtacttgaccaacattagcttggcttcgtatgcgtccgcatgacgtggtgcacaccaatccggggtactactcaaaatagcagagactcgttcagcagtatactctgcggccgagctatcaatcttaccccagctttcactgaagatatcgatagttgctccctgatccagcagataataccgcagagtactgataaagttacgggactctagttcacgacgtttaacatacagctgctgagctaattctagccaaccctcatcgatgttatactgctccgaacccgcagagaatccgctggtggacttatcgatgaaatagaagatattcttagctccccggtcgcgcctgattgcctggaagcgcatgtttggcgcctggttagcggtacgagtaataacgaatacgttgtcgaagtaattaaagtcaacaccactcgttactgacggactgcacagtagacaatcaatctgctgttcaattaactcattcgttgtataatccaagatacgacgaatatctatatcagatgtagagtttgagtgaatctctttaacaatcgcacctgtattgtttcttagcgtcaggcctttctcgtttaactcatccggcccacagtcggacaccagaatagatttctctcccatctctagggacgtttggagggcaacccaaatactggattcatcagggaactcatacgcctgagcatccgagagcatcttacgatggtgtttataataagagaccggcttgtcaaaatctatgagagatccatatgcctcaatggtttctgcgctgatatccccgtcagacaggataacgacttttgcagatagcagaatttcacgaagaactgagatacactcacgacgttgtttaacaactggagcaaataacaggtcattcataactgcgtcgcactcatcgataaagataacatcgatctgcccaacaaagtttttaaacttgtggattgagtgaatagtagtagacatacgatcaatggcaccgcgcttgaagtttaacatatctacggacttatcatactgtccggcttcaaacttcttagcgttagatgaaaccagggcacgggtgttagtaactgcaaggaaattacctttcagctgatcacgttctagccacttcgtcactgcggtggttttacccgtacctagagaagccttgataaacgttaaatgcccttccggtggtggacggttgatcttcaaataattcatcccgtctgggctatcagaggtcaacttgtgtacaggaacgcctttaacattcgactcaggaatatctcgcatcgagttgttcacaaacgcttttaatgcctgcttacgaccgttattgaagtaatcctgaagactgcgggaattatccttcgtgttgatataatcggacaaagctgaggtaatttctttctcaagccaggcgaaatcaacaccgtcctccagagctctgtggtacagtttaggaataatacgcagataaacaccatcctctgcttcttccagctctgcaatggtttgttccactttctcagaggctacgcgcttgcctttaatctgattaagtagggatagaaactcttctttaaattccccacgagtggcttcataatctggcatagtattgggcagttcaatcctagcaccgttaactttaaccagacgtggcttaccctccgctttaaacggatcggtaaactgatcccggaaaatcgggtcagcgaaatagtgtagctgtacagatgaataataagctaagtcggcaatatcaaaaccatacttctgccgagagctatcattgagggacgtaaatagaaactttaactgaccctgagtaacgggaatattactctctaatagcatgtgcatacgaatccccggcttaattccagccgaagacgacgcatgggcaataaaccctgcgttaagcgggaacatgtcctcgctgatgctatttaataacttaatgacatgacgtcccatacccacaatgtcaaacctgtcgcatcctccggtatcagagattccatcgacgtccatagcaataatattgctcttatggtcgattttgaagttagtacgtttacgacgcacggccttctctgcaactaagcatgtacctcgtactgctacgagatgtggatcttgagtgagctgaaccatcagctcatacgcttcattaagatttttggggtcaactgtatccactacgttaaatttgtagggcattgaggctggcttaccttctggatgctcgttagaaaactccttcgcaaaaaggtaatcagttgtcttcacttccttccagtttccggaagcgggatcgcgggaaaaacccgcgtgtccttctagaatagaaaacattacgttttctcctatgttgaagaataaagcgatctcgaactatcaaggatagaacagcttaaaagtccactgactcaacaaatttccttatagatggaggggtggtcacttcctcattgctagaactggccgcttcgcacaactcccacacagcatgtaatcgccgctaagctccatgtggttgacccctattcgtcgctattgcgcagaggtacgttgctacttacagcttcacttcttggtcatttcagaccgcggtgctatttgctccacatgactaacgaacaaatatctgagagcctcgcgatgccaacctctcttacccgcattctagatcaattatctataattccgttcggaatcaatttcccaataagccaaacccaaccaagcatgtagcagccggactgttctgtatgctcggaagtgaatgagaggaattatgtcagttttgtaactcacatcatttctctcaacctatgaatatattatataaaaatcgtggcgagattgcaagtaaaatatttaaatacgtggtaggtaactgggtttaagcatagaaaagaccagaggataatcccctggtctaagcgttcaactaggcattccaacccataatattaattgagttagaagaggcattttgtttgccctcacgaagaacctctggagcatacgtccaaggctcatcagctaccaagaaccccggagaaatacctgcccccggttcccggaatgctgcaaaattagaggggtggaacacaacgttgttcaccagaaccattagccccagatccgagaggtcatcccaatcaaagaggatctcataatcctgggccaaactacctttggacaaaccaacacacacgtcatgctggatgatgaagcaaagctcctcgtccctcaacgcggctcgcagcagattctctgctacatcaatcaattccttggggatatccgcccaggagatacgttccattttaggaatactcatttagttaacccttcaacttggaaaccgaagcgacgcagcacctggatccaaccgttaatattttccggagcataataatcttcattacgtccgacccaaatacctgctgaaacacctgtaaccccaaccgggatataggactcacgcagcagacacacgtagcctagtgcaaacaccagccccatctcagtcagatcgccccagtctaggacttcatcttgatcttcgtccataggaacccatagccccaggtgcatgttgtcataatcgaagtagacctccatattatcgcaccacacacgaagatggtccagcagagactcttttacgtccagaggaatttcagcccagggaactagcccaaccccttgattaacaaccttttcattgaccagcatattcttcccccatttggatgaattcgtgccactgctcagagtccgctcgtaaaacctgatagtcgtccgataggtgactcatatcttgtacatcactgcgataacaccaactaccgttgggccataacaggatatcatcgggatggtacataaattttctcctccagccaagcctcaacttgacctatgaaatgtttgttgctaaatacattggcagcagtatcataccaaacggggatcccgcgttcttcgtacaaactctgctccaggtagttaataccgttgttcactacccacaccttagtacctcgactatgggctgggaatagttcccaattaaagtcgattacttggttactagcaccaacaacgattaccagatcctgggttgtcagggtatcaaacaggttgatctgccccatataccacggagcggtttccccgaagaacgtaattgcaggcttaacccactcatacttacggtaatcaaccgcagtgtacccgatatcttccacgatcacatccttacctcggcgatacattatctccgtaaggtagccatgagcatggatcacatcatcatggggaatgccagcacgctctagtagatcatccacgttagtagtaaagttaacgacttgaccagggaagcgctcataccactcagcaattctcaagtgagcaatgttaggctccaccgtacccagctcctgtcgccgcatgttgtagaactcatgggttagctcgtacaggttgcgactaggctcatcatcagatccctcagaaagggcttctaacgggggcatagggtcgttcagatagtgaaaccccttatcgaacgcctgaatattgcatacttcggttagtttgtatttatcccacaggggagtgatggcacccttacggaacgttggcactcaatccagcaccacttacgataatcaatctacgcattacaggaactccatatagtttctcatttcttctttccgcaactgctcagccttttcggacaagcactcagaggttctgggatcgactttatctttccacaagtggttacggcatcgcagaagttttaccacgtcttctcgggaaatagatccgtatgctccagagtccatcataaagtagatacgcttttcatcagttttatggattttcataaatcctccgtttctcaattacaactatcttatcaaaaacggaggtatttagcaagtgatttatttcaaattcatgtgtttgaccctttgcattacttcctgttgcttaccagggttaaagggtctactaccaggactacctaagtacccacaaacgcgacgggtaaccgagatattccccgacccacattgaggacatacaaagccgtgctctccggatacagactctcctagatatccgcactcttcacattcatcaactggaatgttaacccctatgtagtgagatttacttaatccataatcaacaacccactctagtgcagggataaacttacgcatctccggaagttccacaaaggatatattacccccattggcgatggtggtaaaattagcctcatagtcaaacttcacgttaggggcaacttttgtacggacatccaggtggtggctattagtcaggtatcccttatccgttagccagtcgtactcagggtactgttcggcaattttagtattgaacctattgcacagagactcactaggggtagcataaaggctgaatccaagattagtttcctcagcttttttgttacaacggtctttcatgtggttgagaacctgtacagcaaattcaatcgcaggtggggacatggggtcggtatcctcaaacattacttcaaccagctcgttaataccgatatatcccaaagacactgatgccctgccttcaaagatgggccatacaagatcattggctttcaaccgtaccccaaaagctccgtgcatatagagaattggtgcctgtttagcacgcacacgcttcaaccgctcaatagcccaatcgtgggcagccatcgctttatcaatgtattcgtccaacaacttccagaacctatcgaagtatccctcagactctacagcaactagcggcaggttaatggatacaacccctaggttattgcgccccgcagtctccccggactcaatagctgaaaggaaagaccgacaacccatagggaacttaaaatcacctgtaacagccgttactctttcataactcacgtaatccgggtacatcctcttagaagtgcaggtgagcgccagctgtttgatatcgtagttgggatcgctgggagatttattaagcccctctttaactgcgaaaataagtttaggaaatacagcagtatgccctgaagcacccaggcctcttatacgaacttccagcatagctttctgcaacatgcgtgcctcccaggattctcctagaccaaacccaaatgtcacaaatggctgctgaccattagagttgaacaacgtattaacttcgtactctagcccctggcacgcatcatatacttctttttctgtcatttcagtagcatacactgctgccttcttactatcgtgaagccaacgctggccaatagctaagtgcttatcataggacttacggacatacggggcaaagacctcgtcaatacgatcgatagacgtacccccatactgacacgagcttacctgggcaataacttgggcagttactgcagctgcagtagagatagattttggagtttcaatctctgctccaccaattttagttccattcttaaacatccccgctaaatctaccaggcagcagttagtatacccctgcgctcggtagtccatatcgtgaatgtgtatctctcctctattatgagccgccaataggtatgctggaagctcctgtgctaccacatacttagagacttccccagctatcatgtccctctgtgtagggaattgctcgcttgccttattggcattattaaacataaggtcttcttcaccctcgttatcaaggatactatagatattatcaatcaggtctaattcgttcatatattcctccagttaggcatatattataccaaatagacacaactatgtcaaccttattttattataatcaacagtaacttgatctggatcatcaaaacaataaaaactggaaaattttatttgcttattcaccctattttatattattcgtgtgcgcccgcgcatcgcgcgaataaaggaaaggatccgtcctccatatcatcctagaaaatttcagttgcaatccacgccaaaacttggtatactggtattctaaattgataaacatggtcgaaatttgaccctctgtagacctctaaggagatcgcaatgagaaaagcagcacgtcgtaaggagtcgcgccgtaacggtagcgcaaaacgtgaacgtcacgaaaacgtcatccccgttgattttgaagcacgggaacgttttcaaccaaccgcaaaagaactcaagccgaagaacgctgagcaaaagcactatattagcaccatccgtaactttacggtgacagttggtattggagaagcaggtacgggtaaaacgtttatcccatctgttctcgctgctcaagagttagcaacgcctggttctgtgtacgagaagttcatcctagtccgtccgaacgaacctcttggtaaatccctagggatgttgccaggagatctgaacgagaagatggcaccgtggttagaaccaatcgctgacggcttcaagtgggccttaggggaacgttcttatcaggggctggttgaacgcaaggcaatccaataccttgctatcgaacatgcacgcggtcgtaccttcaacaattcttatgtaattgttgacgaagctcaaaatatctccgtagaagcaatgaagtgtattctaacccgcgtaggtcaggattgcaaattagtaatctgtggcgacgtagcgcagaaagacattaaatccgactctggtctgcaactgatcatggatatctacgatcagtacgaacatgtgcctttctccttagtagagttgcatgataacgtgcgttccgctgaatccaaagcattccaggcaatctttaacgatatgggaatctaatatggttactgaactgatcattggttatggtgagggtattacctctgaagaaaactggggctttgtaggcttcggtgaaggtatcacttctcacgacgaacgtcctgacctctaactctaggagcgcgtatgaacaacgtatttacactgaacaacttccgcactcgtaaaacgaaagtgcatccagtatccctggcaacagtcaataaatacaatgctaactatcctgaggatgagcggcgacaccatgccgctttcaagatagcaaacgaatttcctaaccaacccctcggtactaaagagttagttagtcgaatgaaaaaattacacttttattaaagtagaccgacacacaggcagcctcgaaaggggttgcctgtttttgtctgtaaaatctcatttgcaaaatggccgaaaacaaagtagaatatcttttaaatcctgacaacttccaaaaggagttaccaatgacccaacgtattgaatacgttatcaagcgtgacggtacaaaagaaccgttcatggcccagaagctgaatgactgggcaaaatacatcggaatccgaagcgatgttccgtggtctccggtagcggtagccgcagttaagaacctgccgaaaggtgatgtacactcagacgatctgcaaacaatgctgattaagtctgccgaatcaatgatcgagcgtgatcatcgttacgaccgattcgccctagagctgcgtttagcccagctccgtaagaacctgtttgattcttacaccccgccgtctctgcgtttcttccacgaccacatggtagagctgggagcgtgggaagatatgagcggctggatctctgacgaccagttcgaagctctaaacgaagttatcgaccacagccgtgacgaactattcactaatgctggtctgaagcagttcatggataaatactctagacgtaatatctatacggaagagatctatgaaactccgcagtttgcctacatgggtatggcaatggcaatgctttctcaacctcactggagtatgctcgatgcaatcgacctttataacgcgctgtcgctacagaaaatcaacgtcccaactccgccgctcgtgggccttcggagtgctgacaggggcttcgcttcttgctgtcttgttgatggtactgacaccctggactccattgacgcagcagagcacgtcgtcttcaaaatggtcgctgcccgagcagggatcggctatcatcttgagagtagatcaattgctgatccggtgcgaaaaggcgcgttcccacactctggaaaactgccatattatcgacacatcgaccgctccgtaaaagctaacactcagcagtcccgtggcggttctgcaaccgtttacactgcgttcttcgatccggaaatcattcaggtaatggaagctaagagcaaccgttcccctgatgaaaagaaaatcgataagatggactacaacctgaagtttaacagtattctcctgaaacgcttcctgcgtaaagagaacatcacactgatgagcttcctgtacgctccagaagtatacgcagcattcgactctggtgatgtagcagaatttgaacgcctgtacatcgcagccgaaaaacgtctagcgggcgttaccaaacgtggtttcaagggtgaagttctccctgtagccccagtgattcctgccgcagaactgattgagttctggaaaactgtacgtatggaaactggccgcctgtacactatggacgctggtgaagtgaaccgcaacagccgttacaaagatccggtgcgtatgtccaacctttgtgttgagattgtacagccaacgttcccgatccctcacgtagtagatctgtaccgtacagacgaagaactggataaaatggatgtttcagagtatggggaagtatctctgtgcaacttaggcgggttcgccctaggtcgtatcaaaactctggaagagtgggagaagatctcttacatcctgttgaagttcgttgataccatcatcgagattcagcactatccgttcccagctatgaagtacacggcattacgccgccgtaacgtaggtattgggctgatgaacgccgcaggagcgatggccgctgaaggcctggcattcgaaggcgaagaagcccgtaactggattcaccgtgaagcagagaaggcatcattcttccttcataaagcgtctgtacgtctggcaaaagagatcggcccatgtgaatggttccatcgtactcacacttctgatggtacactgttaatcgatacttacaagaagactgtggatgacctggtttccgtaggtctggaaatggactgggagagcctccgtgaagagatcaaaactcacggtatgcgcaactctgttctgacggcaagtatgccgggagagtccagctctgttctgattggtgttaccaacgcagtagagcctcctcgcagtgcggtaactatcaagacttcgggcgttaacaaagtaattactgtagctccgggtctagacgattgggacaccatgcagtcctataagtatgcgttcgatattgaccgtactgagcacattaagtggttagcagttctgcagaagttcacggatcaggctattagtgccaacctgtactacgactttaacaaatatcctggaggtattattccgggtactgagattatcaaagatctccttaactccactaaatatgggattaagaacctctactacgcaaactttgacgtagataccggaggctctgcagcagagcagggttgttccagtggcggttgtactctgtaattaagctcgatccaaccgaaagcaaaattttatttgcaagtaggttggattttttgttataatattcgtatattaaatgagagaggaaattaaacaatggcaacagtatttaaccgcgaatgggatcacactgagtctaaactattcctgggccaagacctgggaatcgccgactatgtaaacgttcgctatcctcgtttagaggaacttgcattactgcaacgctcgcagttctgggttgagactgagatctccctggaagccgataagaaacaatggcctaatctgccacaacatattaaaaacaaaaccctgctgaacttagcctggcaaacccaagccgactccattatcacccgtgctccggaagatgccattctgaaactggtatctcgccctgaactggaaggtatgcttattcagtggagctactttgagaatattcatagccgtgcgtattccaacattattcgtaacgttctccctaatccgggtgagttcattgcaaccgtacaggctaacgacgaagcattcgcacgtctagcactgccggtttctgttattgatgagttggccgaaattgccgacatttggctggacgctagagccaatctggaaatcgctgagaaggaaggtactctggaatacaccgaagaggcagacttcttagcacttactgaacaagtacagcagaagattctggagttctactacgctgtatacgctctagaagccattatgttctacgcgtcgtttgcgtgcacctttgctctggcagaaaacgatatcctaaccggtattgcaaagaacttacagctgatcgctaaagatgaggcactccataccgttatggctatggaagttctccgtattcttcaaaatggggaaattcccccacatgtagtggcagcagctcaggctaacgccccgaagatcctgcgcagtattctggagacagaaattaactgggcgcattatattttcccggaaggcgaagatattccaggcctgaacgccgatttgctggtagagtacctgtactacaacgcacgcctggcatttatggcgatcaacattccgtggccggaagatctcccggtaattatggaagacccgattggctggatgaaaggttggctgaacaccaagaaccagcaagtagctccgcaagaagcacagattaccaactatcgtgttggtgcaacctctcaggctaacccggacgatctgtctgatgaatttggagagttcctgtaatgattacagctttatacgcaatgcgagttgacgccgccttcgggatttttaatccggctacaatggatgcctacggggaacttccctggggctccattcccgaggagctagaacagttctacagaatcctggatacctatcaggttgttatagtgggccataatacctacgaaacagcccctccacgactgaaaaaggcactggagaagaaatccatggtgtacgtagtaggttctaaggctccagttctgataaaaaaccctccccgtaatgtgcggtttattacccacttgggttccaaaattcgggacttctgtaatgaagttgaggtagtctgcataggagggaaagctctgctagaaaccctagcaactatgggctgtctggatgcgatttatcgctccaccatttatcctaaggccggtacagtaccaagcctagaccacatcatgtatctggaacacccgatacttacgtccactcctccggatgctgtagtaactcatatagcttctggggaaaatgagcgctatcgttttgttatggaaggagtctatctgtgattcactatattaatgaaggcaaacgcattcttgaagaaggtgtatggctggagaaccctcgaacgggggttcgttgtttaaccgtaatagggtcaaactttgaatatgatgtactcggaaagaaattcccacttatcacaactcgtaaagcgtatgctcttcaggccatcatggagcttatcggatatctgcgaggatatgattccgcagagcaattccgtgctatcggctgcaatacgtggaacgctaacgccaacgaaaacgaggcgtggttggtcaaccctaacagaaagggaaccgacgatatgggtagagtctatggagtacagggcagaacgtggcttcgaccggatggttcccactttgaccaactctataagatctatgagaatttacgacgaggcattgatgaccgcggagagattctcaccttctggaaccctggcgaatttgatcaaggatgcttacgaccgtgcatgcatacccaccagttctcgctgctcaatggaaatctgtatctcgactccttccagagatccaatgacttcctattgggacaagcctttaacatggtccaatgctacacgtttcttgcgcttatggcccagatcacagggaaccgggcaatcagagcaaaccaacgtatagtaaatatgcacatctacgagaatcagtacaaggttctcatggaacatgggcaatttgaccgcaaacctttcccggctcctcgtctagagatcaacccggagataaaaaccctagaggacgtactcacctgggtttctaaggacgactttaaaatcgtggggtataagtctcatgaccctattgcatatccgttcactgcgtgaggacttatgggattatttaacagacgccccaaaataaccttctccgaaagggaggagtctcagctgaagtttctggttcaatcctcgggattacacatagatgttatcctagggatggtaaagtacaagggcatggacgcactaatgcgtcagtttgcccctaaaccacctaaagaaaaccctccagccaagcgggactataacagtaatttactggttcctccagctaaattactataactaaggtggccttcgggccacttatattaaaaattcatttgcaaaccacgtgaatttctaatataatagattcataaatttgagagaggaaataaatatgtcttttactgatgcaaaagcaatggcagctaaagccaaaaggtctaacgatatggcagttatagcagctcgcagatctattatatcaaatattgatggatcagcttcttcaggtaaaacagaagtagattcttacgcattgaatggcctgcctatcgccgcacggtctcagattatggaagacctaaaggacgcgggctatgaagtaaaagtaaatcatccgtttgaccaacgcgatactgaatcaattaccatttcctgggggcacgcataatgtttcatgtttatactgatggtggttgccgcggtaatacccgtggggtagacaacgttggtgcttgggctatggtagtttacaactccagcgaagagcaaatcggcaccaagtctgctcccaaacgcaacacaactaacaacgagatggagcttcaagcagtcctagaggctctgctatggtctaacaaaaaccctggccgaccaatgacaatctatctggactcaacctacgttaagaatggttgtgagtcctgggtttggggctgggaacgtaaaggctggaagaaggcagatggtgatacacctctgaacttagaccagtggaagtggattatcgacgaactcaagaagtatcgtctaaaccacaatgagatcccaactttcgttaaggttaagggccactctggtgtagaaggtaatgaggccgcagataacctgctaaatgttcgaatgaccgaactggaaatggaggatatgtgatgttggagaatttacgccgcctagttagtgaaatgaaatatgaagtgctcctgatggagcctggtgtggatcgagtagtaatgaaattgcgaatcgcacgtatggaagcccaaatctttgaagcggagtggaaagctctgaggggtggagatgaattataatggcaccagatttgagggatttgttcccaaacgtaccacagtaccagctcgatctctacgccgcgtttctggaagcgtcaaaatcggggaaccctctacgcgtctaccgccaagaccgcaggcacgggaaatcctggatcctgagatggctgaaagagaacgagccgctcttaaagaaattgagcgaaagaaactctgtacagcaccggcatacaacaaaggtgggtaccagtacatcagctcagaagagcaggcaaaacatatcgggcggtaacagatacgagttcatcatcttcgatgatcttgtagatgaaaatgaaaaaacgcagttgctaaacgctaaaaattaacgtataatagattcataaattaacgaaaccaatcaaaacaaaggaaataaatcatggctaagcagacctctaaaaaagcagtagaaaccaaagttgcaacttttcccaaaactgaagcgaatcgcaaagcacgtctggagcgtcatctgcgtaaacatccggctgatactcaagcagcatcagctgtaggcaaaccggccccgcgtcgtaagaaaccggtaactaagggttctacctccggctacgtctccaagatcgtcggttggagtacgccggataaggcagacaccaaagaagtcctgcgcaagacccagggccgtttcggtagcgtcaagccgaatatcttcggttgcgaatacagccgtgagaatgttcgcgctctgtgctacggcgtaggtattaaatttacgggcaaggcaaataagccccgtaatcaaaaacgcaaaccagctaagaaggcataatgcgaaattttgtagctaaaaacgatttcaatcgtgcagcgacacataagtcggccctagattattctagggtcaactcccgggaactgatggattcgtgctacgaagaactcgaagattgggcggctgactggccgtcgatggaagaaaactgggatgtgtccgaagatatgactaagccaccaccggaggtggcttctaaatgtgataacacatctagaagcaactttaataacaaaggaaacaatatgcaaaacttacaagaccgctggatctctgtttgtgatatcgaatccctgggaactccgggagattgcaagagcacctttatcgctatgccgtttttcgctttcgtactgatgaaagatctatcgttagatccgtatatcgttctaggcactcctaacgttgcccagcagttggccctcggtgctaaagtctccgccggaactattgcattctggatgaatgaggctcgtgctggtagtgctccgtcgctgtccattattgaagctctgaacgctaaagacggtgagtctactgttctggtctgcaatccgactcatgaatcaccggtatccaagcatacgttcatggatctgatctgcccgttcgtagaggcaaaacaggtaatcgaaggcattatcgatgagcaaggtatcgacactcgttccctgcgccactacggtaatggcccgcagttcgatatgtctatttacgaaacagtggcggctcaggctaatgtcttctctccgtctgatcctgcaatcgttccgtggaaattctgggatatctccagcgcccgtaacccgcgtgactatttcgaggctctcggaggagactggaaagcattggtacgttgtgctgaaatctacgcacacgacgtaatcgagcgttacaacctgattcctgagggggtctatccgtcgaaacatgatccggtgtttgacgccctggtagaagcgtactgtattaaaactatcgaatcgaaattgaaaatttgatttgactaaatggctgatttcttgatatactattattcttgaatcaaagagtaaaggagaaataatgaaagcagccattctaatgatctccatcctcactagcttccatgcgcaggcaaagattgatgcgcatgagatagagtgcatagctaaaaatgcgtactttgaagctagaggggaaggagttaaaggaatgaccgcagttgcacaagtaacgaaaaatcgtgttaactatggaaaattcccgtccacatattgtaaagtagtctatcaaccagggcagttcagctgggttggtaagaagaaacataaactcgatcgtaaagacgaagagtggaaacaagctaaagagatagctagactagtttactatatggatcttccagtagatccgacgaagggagctttgtactttcacagtaaagatactaaaccttactggacaaaagacaaggacttcaaaagaacaagtaaaattggcaaccatgtgttctataaattaaaatctcagttgcctaatgcttaaaaagttgatataatagtttcataaattagagagaacagcgggccgattacgctgaacttgttttaatgtaagactcgtcgagcattaagattcgttgaatagaatcggtgagttaagagtgatcacctgacaaagttcgtgcatccagtccggataccggccagggagcgtattggacgggcgtaggttagagatctcttgtaaaactcgggagctgtcccggagtctcgtaaactcttctcctgatttatagaaaatggtggaatctcactttaacctgttcgaccagaatgaagcatggtgcgtggcctgccggagaggtggggagctgagcctgagaaactcgtatagctcccacactgacctgcctaggccgcaaattggagacatgtgtatagcgtctggtgctagttattgccaccatagtggggtgtcgaaatcacttatgtagcaacactgccgtcctaagttaattggtagacttctgcacgcgggggagctatccccagatgcggaaagtgcaggttcgaatcctgcggtcggaaagagttagataactgatggttctattggcgaaccactcgctgctggagtacctcctcccctcccagggaaagacttgggagactcgcgggaacgaaacagctaaaccacgaaggactaggatgctgatttggctacccttacctgatatatgcccaaatcaaaagaagaactttaggcggtagcctagatagtgaagtttttcctccacgaggagtcgtttttcaagctatcaaaacttcacatcgaaaattcggaggtgtgattttaacagaccgcgtggaggatggctctccagaagtgccaaagttagtccttaaatacggcatgatgatcgtatgaactttggcgaggtgggatctcgcctgattctaagtgggaaagagtattcggatagccgttcgtacaaacggtggctccgagacctaagtgggacacggttgagtatacagctccgcggaagctcgtataccctgtgtaagcatgtgacaatggagtcatgaccggagtcaccccgagcgaaagcataaccactatttatagcacccttacacaggctagaagtgcccctgtttggagtgtgccctcgcctagaccgcattagccgacggatatccgttggccgggatctggcaaggtatattagtcaccttgactataactggaagatacgtctgacagaagggccgcatagcttaaacaggggaaggcccctacctactggcggtatctttaagagctttcacgagagttcttaaagatactaattgacgaggtctatatgaacgatttaagtatgttaactaagatacgctctgatattgagtctatggtctctcgtcgtagtgagctgactaaggctaaacagatcatcagcggtggtacacagaaacgcttcacattgcaagccggggatattaagtttgacctatgtggcagccagactcgagattacactttcgaaatgaaaccgtgttacgatatggtgaagctggggcttatcaaagctctagacaaacaaatagatcagtgtacggacgcaatcaaaaccctaaacgtccagttcgccgctgaatgcgatcgtctcaaaaactctatcaaggtataataatggtggcatctgtgcatactcctccgtatgaacgtccagcacctaatctgacacctgaacagaaacagctaatcgccaggcgcactctagagtttaaagagtcgctgcataagagcgttggccggtattctgaacaggttcatgatctggttgtcaaaacacttaaactttattaatgcttccttagctcagaggatagagcaacggtcttctaaaccgtgggtcacaggttcgaatcctgtagggagtaccactttaatcgctgcccacacgtaatattcagtgggcagcggcagaaagcccgcagggcgcagattaagattgattctacaaaatttttcttgcaattttgtcagaaattttgtataatagttctttaaatcaaagagaaagtagaggataaaatggcttacaaaattgaatatttaaagaaaggggttctcactgagctggttatcgatgcgaacatggctcgcaacgagggcactaaatccgtattctataaagacggtagcgtagctcgtatgatcaataccgaagacattcaggatctgtacgtgatctccgacgaagaagcaggtttcgtaaaagatcctgagccggctgaagatactccaactgaagacactccggtagcggataccactactgaagaacctccggtggaaggcactccggaagatgaagcagcagtataaataactaagggctagaaatagcccttctccttgggccggtagcttaaagttaaagcagtggcctcataagccaacgagtgggagttagagtctcccctggcccaccaatttcataacaggagactaaaatgtctactaaaaacgcaatcgtatccttcgtggatgacagtggtatcgtcctagagtccacagtaacagatatcagtcctaaacgtctgctccatcgtgatggggatatcctggaaatccttaataaacggtctgagactatgctggtcattcctgtaaatcgtcttctctcgattaaaatcgtgtgggaggactaatggacgctcaattacaaacccagtactatatgcttttaggcatgttagaggatgctggcccgacagtcagagggcactatgagcgtcacaaagcagcttttgaagccctactaaaagaagttaatgagaacgaaggtggtaaaggctctgactcctatgccgcattcattatcgcgctacaagttttcctaatcaatcaactcaagtaataggctaaatatgctgaatattaaacgtaaaggttttttctacaagtggcttaatttctcttccgcgtctttcacctaccggctgaacgacaacagagtcaccttgtgtagcctattttggcactcagtgtggtatttcctgctacagattggcgtaactgctattgctgtactcttctccttggggatgggaagtatcttatctacgttcttgggtctcacctttgagctgggaattactccctggtacatgcttgtagggttaagtctagcaggactatctacgataatagcgattctgctggcgatagctgggatcggctgggcctgcgctaagataggggatcggatccaggaatggaacgcgagtaaatcctttgaaagggcacagaaagagtataatgctcgcgatgaagagctccgcttcggtaatatctaccagaagatgcgaatctataagaaggacaaactttgtccgctcattcgtgtagaccacggcgagtagtttgcatagtgacctgcgttgccgggtcactaccaaaatacttgaaaaattcaattgctaaatgctctagaatttgagataatatttatattgaaagggaatagccaagtggttacggcatcggcctttgactccgagatcggtaggttcaactcctccttcccttgccaaatttaaaatgctcctgtcgtctaagctggttaggacaccactctttcacagtgggaacacgggttcgaaccccgttgggagtaccaaatttactgaaaaattcagttgctaaatgcttaacaaattgatataatatttatatagtttgttaaggaattaatctgatactgagccggaaccaaaaaacacgcttgcagcttgctcggtcattgtactgagggttcgcaagaatagcggagtggctctccgaaacgcctggtaagcgtagagtgtatcgtatttgtgaagagcgggtatcctcgcatggctaattgcttagggtattacgcgcaccggattaattgccttaacaaattattgggggatgggtctgctgggagtggacaccgcacttgcaatgcgggaatcagaacggttcaaatccgttatcctccaccaaacaaatggggatgtggcgaaattggcagccgcgctagatttaggttctagtggtgaaatatccgtgtgggttcgaccccctccatccctaccaaataacgggagttcgtgatgaacatagaaatcatgcagttggatcgtaagaaaaatgagttccgtaaggtccatacctttccgagtaaagaagctctagagtttcatattaagtgtatgggactggtgctaccggaaagcgagatcttcgacttagcatgtgctaacggagttctgtacgtctgggaaatcacgtatcatgctgatcctgatgaactccgtaaagaagtagagcaaatactaactggagagtagtgctcatgggagcaagctgacttgaaatcagtcgccatcggaaacggtgagggttcgattcctttattctccgccaaacaacaggaaagctggtgaaatggtagccacgcatcactgctaatgatgagtccgcaagggcatgaaggttcaagtccttcgctttccgccaaacaatggcccgacgagtaacggttgcccctttctagagaattttctgggcgaaaatccagaagtaatcaactagttgtaagtgggaagagtttcttagggccgccaaatttgagtatcattggtattgtaggagtcgtacgctgggactacgtagtacactgagtgtcctatgcccagtacgcggtctaaccaaatacggtgtgcgatgtgcattttatttgtggaggtctgatcagccctcgatggtactcaaatttgggatattatcataactggataatgacctcgattgtggatcgagtctatcttggttcgaatccaagatatccctccagattactgcaccattagtttaatggatagaatatagagctacgaactctatggttgaggttcgattcctcgatggtgtaccataaaatctctgctgagagtgacgaggttagccctctgggtcacatccctcttctagccgcctcccataaatgcaagcatttatgttctggccctagacacaacgttgtactcagcagagtgcgcataattggggtatagctcagtaggtagagcggaggtctctgaagccttaggtcacaagttcgattcttgttgcccctgccaattgcaccgtagaggagaggccgtcctcgccagtctcataagctggagatcgcaagttcgaatcttgccggagcatccaattctaggagaagatgatgagaatctctttcacagaacgagtactaggtactggagtaatgctaatcacttcctgggatggagatagctggtgtaacgtgacaggcttacgtaagtcagaacaaacacccgagaatatcgctaaaatcaagaaacgaatggcagaagctgctagtcgtcctggagcacctcgtaatggtaaacgttgaggtaactatgactcgctatcaaggtatgctaattaatacccacacaaaagagattgtattcttggcaccggcttttcacgacacctacaatgaagccgaggaagacgctaggatcgctaaaatacacccggacgaggaaatctgcgtccgtcagcaagaacaataatgcttcaatagctcagttggtagagcaaacgaccgataatcgttaggtcactggttcgagtccagttcggagtaccaatttttgccccctataggatatttcatcggaatatcctccagcgatggtacgtagtatagtacttacctgttaagcctagacaaagttactttgtgggcaaaataggtatagataggagaagaatagtagcgtaagattcttgaggcaaattctttaggtcagttggcagagatggtttatgcactcgcttcatacgtgagactacagtggttcgagtccactattgaccaccaaatacttgcttagctcaatcgggagagcatcgtctttacacggcgagggtagctggttcgaaaccagcagcaagtaccaatttacagtgagacttcggtcagggccgtcaaacgtccgagaagctgtaatttctgatggggatgatcccagattcgtaagaataccccatccttaccttcacaggttttagcgcaatgctgagtaaaagtctagggaatcgactccatgccatccaggagtataaatggcgaatctagggacttagacggtcattgccctagcgcgttgatgccgtgccggaatcggtatacggggcttcctaaagggcgtaagcagaacatccactctgcataagatacccgaaaacgaggctgagataaatggtctatcttgtgaaggttcgatcccttccggggttagcgtgtagtggggctaatcaatccccacgttaaacgttcaagttaagatttgttagttgcagaagcggggaggctatcaaccttcagtccctgaaagctctcagcgcttggaactctttcgtccgtaaggaatagatcccctttcgctttgactcgcaaacttcaaaccaagtgtaactaacagctgcaagcctggattaccgctctccggagcggataattcaaaaatttatttgctaaacgctcttatttactgtataatagttacataaattaatgagaggagttagcaaatggaaaagattactgcaactggtattgaatctgcactggtggttgattgggccggatgggacggagatcacgaatggatggtcttttatagctgtacgcttcagccagaactatggactaggcttaccgatgagcatgctatgccctacggtatcatagatgtagaaattgagattaacaaactggttggcaccattatggtgcatcgagccgaaggcgatcacaaggaaatcttccgtaaaagcattaaactggtagtttctaccggtgactttatttaacaattaactgaggaaatactatgtacacacgtcctactaatggaaattctgctgtagtccgcctgatgattgttcaggacaacctgtccaacaacatcgagtctctagaccgtcgtattgaggagtatcgcactgagatgctgtctctgatgcgcgaacgtgaggccaagatcgaagagcagctagaggtttgcgaagctatcgaccgcctggttgacggaaccgcagtatttatggcggaagctcccgcagagcctaccttcaccccggtagcaccagctgacatgcagtatgctattctgcctttccatctggaagaagaagatggcgaaggcccgtcgctggaagacgttgttcgcttcctgcttgcctccggtttcccgaacggaggtcgttaatggattttatcgttgtttgcggagcgaatactgactgcttcgagctgttaaacgacgccctagacaaggttgatgaacacatgcaggaaggcagaactcctacattcattgacctgtctcaaggtaaaacttacttctacccgtctcttgacgtagagcctacagttctacctatcttcatgcactccctctcgtgggacgaagaagacgattaatgaaaaaattcatttgcctagtactctgaaatttagtataatatttatattgaaacgaaagaggaaaagcaaatggaagatgaagttaaattgtacatttgttcagattgcggtgagttaagtgaatgtcattgggagtgtccgacttgtgagtcagacgacttaatggaatacgaaccggaataacaaattaagcggctatggtgttcagcggtcaacataccggcctgtcacgtcggagccacgggttcgaatcccgttagccgcgccaaattctctctgggctaagagatatgaccgtctagaggtatcctcggacacaagggtcagccctagacacaataagttagcaaatatctcgtttcgacgaggtatttgaagaggttcttacgagagcttcttcaaatacgggggaatattctgtaagtggtagcagagcggtctgtaaaatcgttgccattgcggctcgggtggttcgactccatcttcccccaccaattttgacaatcaaagagatccgggttcgaatcccgcgtacgcaacaggcggcataaaggccttgtagcttaaatggacaaaagcgctttggttgttaaatctatttgggagagaagcagtaagtggtataggcggtcgcctgttaagcgaatgacagtgagttcgaatctcacctctcccgccaattttactgaaaaggttatttaattaacctaggcacgcgtaggaatactgccaccgcaattgaaagatgtggcccggtttggagaggtatgccgctacacgctacggtgctaactccgtctaggtgaaaatcctagtcagtaatacagtattagggtaagaacagcgacttaggttgcagtgaagcgttagcaatcggtggaactccggtgcgccctaattaaacaatgcatcattggccgagtgactaggcagaggcttgcaaaccctcgaagcatggttaaaatccatgatggtgctccaattaccagaggtactcatgattaagtataaggcgtttgtaacaagagagtcccaaacaggagattcaagtattaaattcgaaggtacaacgttacatgatacatttgaagccgctttaacagaggcggaaacccatatagtatcgaaaagctgctatgctcatgtatgggaagtaaacactattttagatcgctagctcaatggttagagcactcgccttttaagcgataggttccgggttcgagtcccgggcggtctaccacattatgggacttcagctaatcggctaagcatcatagatagcggggaatcccggtctgtgaggggagtaacacgtagtctcccaagttccaccaaacatcagagcagcgtgatgtactgcaaagacccaaagaccggggtggacttcttgattcaaaggagcaggcacgaattacgcaactgtagctcagcgaggtgagagcactggtttgaaagtccaggggtcgttcgttcaaatcgaaccggttgcaccaaattaatagtatcaatagcttatggagctgaatagtcctaaagctcacggtaaagcagctggccgttagtgagcagacttggaaacgtctggtgatctctaggttagccggtgcgttggttcgaatccaactgatacttccaaatcccaagtagcccgttacaggctctctgattaaactcagacaagcaggtaatgggtgacaggataaaataagccactgatttccggccgtttgacgtggtaaagtcctccagtcaagaagaatttctgcgtgcagaaatcactgaaagcaatggggcagacaacttgagatagattgcctataacgtctcaagtagtggcattgacttagggtggcattttcccgaaagttcgaaaaattcatttgctaaacgctcttatttataatataatatttatataaattaatgagagggcttacctaatgaatcaaaaaattcttatgcgatataatccacgagctttatggttccgctgggaagtgattgtatcgtaccagatacgagtgcgtaacggtgatcctgagaacaacattatcgtgctggagacattctctaatagagatgcggcagttaagttcctgaacaccatcgacaatactttaatcaaggtatattaataatggatatctttactactcctgctatcaacttggtcggtgtcggcctattccaggcaacggtctatcgtattgatgatagcactgacgttgtaacgttcattgtaccggagttcttccttgagaagttctttgaagagtttgagcaattccgtgaagagcatgatgcttactccaatatggaagatctagcagcgatgttcccaactgtatacggctacatctttgaaggcaatgatctgcttttggataagtcagagctggtggaactcaactggggcattagctttgaagtgggctctccgttcccgcggtatttccaaggtctggagattcgataatgggcggctactctaatttcatcgagaattacattaattccgtagattcctggaatcaggaaactctagtggtagtgcttaaagagagattcaatatctctactctggaagccctagaggctatagaagcctacttggataacgattaacacccacttggtccaatctggtagaggcatgaggcttaagacttcagggttcccggttcgagtccgggagtgggtaccaaattatggggcacgactgatggggaagtcggcctggctagggttgagatacggttcgattccgtaggcaaatggtctcgtaattccgcacaggtgatggaaggttcgagtccttctcccataactatacaaattccccttagctcagtctggcagagcgggcgctttgggagcgtcaggtcaagtgttcaaatcacttaggggagaccaatcttgcctcaatagctcagccgggagagcaaccgccttgtaagcggtaggtcgtgggttcgattcctacttggggcaccattttcgctggttatggttacagtgtgattaagtttacattgtcattaaattccggttcaattccggaagccagcaaccctaaaaggtaagcatatgaggattctttccatagaagatgttatgtgtgacagatgctacggctccatattctctggtggctgtagttgtaagtaatctatctaatgaccttctgtagatgtaaaataaatatgaagggtcgatgctcgcggctaccgacagatcgtaatgatcaagtagtagtctgaggtgaagagactagcccattaattccgcgactaactgtacgggttacagcgtccgccttccaagcggtactgagtggggttcgattccccctagtcgctccaaattcggtaaaatgaccgtcccctgacgttacggggaattaaacaaggaactgggagccggtaaggccattttatcgatactacttgagaggtacaacaatgaaagcctttgatgcagaactagtgttctcactcttagctgagatggaagcctgcgtagatcgtgtacgtgcgctgcgtcttagtatgtttagctcttaaattatttgctgttctagagccaatgtagtccggggtgccgacgtctgcctggaactcgactctaggatagtcaggacgtcacttaacagccagagatggcaaactataggagaagcaaatgttaacagttaaggtaatgtcacctaacggtggcgaagaaattcacgatggttcaagcgttgggtttaatcctaagcagaagagcatctctattgctggtcttgatcagcatatcttcctgaaagaagatgaggtagcctacgttatgaatcagaatggtaagacagtatccgtctaccacggtagctaattttattccccgagtgttactggacagcacgccggtctccaaaaccgtgcagtaggagttcgagtctcctggggtttgccagtttaaaggagtgatcatgatcacttatagcaccaattttatggggccagtctccaacaattggtacatccgtatgggtattccgtatacggaagtaaccgagccgaatcgttttgcagatggcgggcaactaactcgaaaagtatttgccaaacgatatgccggtggtcgcattgacgttcgtggtactgatgactatttcggccaggaaatcggtgttccgattatggaggccgagtcttggaatgaactacagcagtttctctggacattctcgtccgataaggttctgacattggagcaaattgtacaggctctagaggacgagacaggttttcgcattgtttggtttaaggagccagcatgtacatagatcgtaaccaactgttcaagttcctagagcttgaccttcgttggcctctatcagtcaatccgggacgagctactggcaagacgttcgaggctattaatacagcctatgagtttgcagtatttaaaggtattcaagcagtatacgtagcatccggtgtccgtgaaatggcacgcctagagaagaagtataacgagcttcaacctcatgttaaaattacaacgtatagcatgttagaaccctaccgtataggtcgtcgattcagctgcatcatgtttgacgaacctagcttggctattaagtacggcgtaaacgcttacgtggttcgtatagccagagaaaaccagtgtcctgtaattatttttggagagtaaagtggaacaagaatttcaagtcttcgtagacgcctctaagagagttttattcatacaagccaccgatgaaggtcatggcctgcaactttccttcgactctctggagcaaataaaccagattgttctgagggcacaaaaatcattagaaaagaacactgaggcacctcctgacctctaaacaaaagccccttgtctctttgagcgaggggctttttctttataaattcacttgcaaaaatgccaattttgccgtataatagatttataaattgatgagagagatttccaagatgaaagtaacaatggaaaacacagaagagtttattgctatctgtactgcgtatgcagatacgcttccacctgagggtatggacgaccatacaatgcagttagtggcggatatctacagattagcagagttagctaaagaacaacacaacaggctggtgtacgtcaaagaacgcctcgaaatgatggataaggagtaatcaatgaacgaactcaatgaactgaatgaattgcattacgctgaacgtgctattgacgagctggatttcgctggtggttattatacccgacacgtcaacgcaatgaccgccgaagggcttaatagtaagtctgcgattgctgcggagttagcagttcgggactttgttattgatagtctccagaagactattagcaacttgagcgaaaacaacaaagctgctctggaagccctggataagctgtccaaccatcttctcgcattggggatcaaataatgaataagcaatctctacgtggtatccgagtatttcgctctagccttgttgattccttctatatctggggcaaggcaactcgacgcactgttgagcaagcactagacggtacgttctatgattggcgagaaaaagagcgaaaccccgtattcagccgcccagggctgtatcacgatcgtgtatctaagacagcgtggtacgaaattgaagtaactcctggcgttatcagagcgttctatacggattgggagcacgaaaagtgggtttgggacaatcagattgctcccggtgatcgtattatgaactatgccgaatataaagaaatgaagcgcatgttcgagctatacgatgttcctaggctgtctcgcccggctatcttcattgctagccaagagtactggcatactgttcgtatgaagcgtgattttaacaagcatcacctgcgctatgagaaagaacacggcacactgcgggaacgtgtggccaagagaaaagccgaactgcgtgaaaaacgcctggagaagaaatatggtgaaagctagtctcttacgcttcactcctggagttggcctccagtataaaattgttgggggccacaagttccaatacttcaccccgggtaagctctacttcgtcgagctacacgatagtagagcaggctacaaattacgaagcgacgcaaatgagggcatttgggtgagcttcacacaagttaaacgctggtttacagttgaaggatataatgattatgagtaaagttgtatatttcgtaaaatgctgccgtgacaccttagagttggtcgaccacaaaatgcagacctgtaaatgtggtgcttcctccatagatggtgtagccggagcctacgtccgttttctaggcgacaaaagtaacttcatgcgtctgaatgagttccagctcgaagttgaaaagaatcgaccggctctagaggcagaggctgagcgtctcaaagatttcgatgggaacatcgtagcatataacatggtaagcggtaaagacttctggtctgaactggctgaaaaacttaacatgccgcggcaggcggctaaggctttatatcatggcttcaactactcgccacgttggaattaagttttccaacagtaacagaacttacgtttaccgagtacctagttattggaaatactccccggagataggtgatgtagtagtcataccggggaatgttatgtttaataatcctcgtcgcgccaaagtagtagaagtgcatggtatgtatggtaagccagagtataaagaaagaaagaacatctcttatgtagagctgcatgattacttaccgaaggaggaacgtaatggacgataaaattgctcgggaagcaatagaacttgttcgtaaacgactggaagaacgtaatgtcgaggtgccgaaaataatgcttattggctacggcggcatcggcggatttcccagcttcactgacctagaaagaatggaacgagagtcccaagccagctttctagagttagaatcctacgctcgggaaatggaacacgaacatccgattggtaataactttatgccacgtagctcccgccaggaggttactcacggcaagactaattcctggcccactccgaaacgcagaggtagaaaatgattactacaggatttggatattctcacgaagaattgtgcaaaatggttgaaagcgctccgtttattaagaagttagtcgaagagcaacgaccggtgtgtttacatgctgcctgtaccaagtgtcacggtactggtgttgataaaaacggcaagatgtgcgtacatgcactgtcctgcccgtgtcctaaatgtagctggagctgttaatatggatctaggatattgtgtagtacatgaattcatggagcaggggttgccggatcgtatttgcgttgtaacctctcgtaatctggaagcagcccaatcactagtggagcgactctctggctactatcgtgaccatgaacgttaccagcagaaagtatttgacctgacaaaactgtaccatcaaaaagctctggatatgccaactccacagctggacgatctcaagcagtttagtcctgaggcctggtatagcgtcaaagacgccagtccggtagactataccgtacaggtattctcgcattatggtactcgtcactggataaaccgtaaatgggattccatggttgactaccttaattcggaactcgaaaagggggctgccgaatacaaaagaaagcgtgccgaagccaaagtattgaaaaattcagttgcattgtaggcgatttttcgatataatagttacataaattgatagggagagcttcatgagcaaactaagtattgaaagcattatccgcccgttaatgcacgggtatgtacaaggttcctgtgtaagcgaaactgaagctctcaatgttatcgaagaagagctggtggctaacgggtataatcttcacgaaggtgttatcgaggatctcttctggcagaccgcggaagatatggagatctttcggtgcgtgaattgcgggtggtggtgtcctgcttttgaaagggcggagaaccaaatagaggaaatctgccgggactgcgagccagacctcgaaggtgaagtggatgaacaagataacgaaggtgaagactatgagtaagcgttcaattgcagcaatcatcgcattcagtatgatgtactctggagtatctctggccgcagagcgtaacaaagtagagatctctgataacgggcgggttcgtgtaacaacgaatgggattactaaaggagctggtaagtttcgtaaatctgaaacccgttttggggaaactaagatctacacgaacaaaacctatggcaagcccgctgtgacactggaccgctatggtcgtcaggtagaagacgaggatgatagcgatgagtaatcttcaccccaaacttcaagagaccctagactggattaatgaagagtgtgcttttgaggaagctccttactgtgtctgggcaagagctggtgcagccccctcggagtggtgtactgtgtttgataatcgctaccggataacagttgaactaagcctaaaagaggacaaagtatacgctaaagcctctatgaccgcgctaggattatccgggttcgtggagatgcaagagctctgtatgcctaacactcaccttcgggttcagatcgagcagttggcaacaattcgtttgatgttgccggaagataacatcaatgaccatttccataaggttattgaaaatgaatacaaactacggcggcaacgtcgcaaagcacgacgggaagtagagaagactcggatgatgtgtaacatgaatccacacgtataacggtagggccgttataggagtcaccttcctgttcctatccggagtcgtgcccgcgatgaacgaagtggtgagttggtcgccgaccactaactaattcaaagacaaactgaggaagcaaaatgtttactttatttatactggcagtatcggcgtggatggcagttggtatcaatcatggtctggactctgctaagctgctgtcagctaaagccttcgagttcctggctaagtttgccacccgtaaagatatcgaggccatcatcgcaaaaggcggtgccaaagatgcatcgagtgtcctgaagtccttcgataagatcctagagcttcgtaacggtaagcatgccgcagagctacgctgtatgagtcgcaagaccataggcagactgtgtaaggccatcttcatcgtacagggggcgttgaaaggcccattcgctaagtataaaccggatagtatcaagcgagccaagatctttaacgattactgtgtggaacaccacccgttaaatcgctaactacccaagcccagcatcgaaaggtgttgggcttttcttttttaaaaattacttgcgctctcgcttaaaatgttgtataatagttttataaatgagaggagaactaacatgcgtattatctctaaattaaaggacgtatatgatctacaaggtactatgtatgatgcagaaagagcatggtatcgcgaagaagtgaaagaggttgtaaacgtatccgctgatttcgaacaaattattttctacgctgaaatcttgcgtaaccgcacttcctctggatatgggggtgttaatatgggaactctggaagttcgtccagtattgatttgtggtacactacgctggctgtacggttatcacactggcctaggagcagatgctgtgcatatccagactttcgatccagttaaggtaaaagaggtactggaagaacaaggatactacctgcggatgggttgggacatgaatacgattgagaagatcgacgctcatgttcgtaacgcaaccgccacggcttctgcgttcctggagactttcaacaagcccattgcgatggcatgggatgccgcaaagtctaagacagacccaacggttaatattaccgttaaaactgatttcaacttccatgcggaagatttcccgtggcaggaaattgatccaaacctgtatcgctggcaccagaccttagaatcatatatcttcggtgtgctgggtcaaggggaaccaaagaccgagtctacgtctgatcgtgatcggttaattgctaagggcttcgatgctaaagtttctttcaggaatatggagcggtaagactggagcaattctgtttctggcaattgcagcaggcaccttcgggggtgcctactacattaccaataagctaacagatatgtccagttctttacagtccctatcaaaccgtaatgaacaactagaaaagacagttggtaatctccaaacagagattcgaaatcgagatcgcaataccactacctacataaccaacctagctaagaaccaggaagatctggatgggcgtattaataaactggatgcagctagagccaaagagggcgtggttgccgctaaacctaaacttgctaccaaagtggcaaaagacaaagttaatgagttccaggagagactttcatgcgtaactggaaatatggactcctgctctcggctgcaattatcgcatccgggtgtgcagaacggccagacccaagtagcacagtaacgggggttgagccacagcacctaccgtggccagcaagcctacagacttgcccgtttaattttgagttcataaacgaagaagggaaagtgtatgttcgcataccttaccaagattggatcacgatgggcaagtgtaatgagcaggtctacacctacattgccaatctgactgcattaacgtgtacctatcgcgtgtcacttaatgaatatcgttgcaaaccgttcaataaggaaacaaaatgaaatacgttttaggattaattggtgatgccggcgcaggcaaagacacctttgctgacatggctaaggtttgggcctgggaagtcctcggtccggagtattctatcagtaaatttagttttgcagctccagtttacgaactcgcggctgtaatccttggagtcacgccagaaaagctggcagagcgcaggacaaaagagattaagcagtggttttgggtgactcaggaagcccttgagcgtactgctaacgtctggaagcgttttgggatcgacaagtacgccgatttctcttacgtttggcctcagtttgaagcgtcagcactatatccgttgattgctaagactgccccagacttttatcaaggtcgtgagaccccgttgtacccattatacacgtcccctcgtaaaatgctagaatttgttggcacggagttaggacgcgctctggtggatgagaacttgtggttgaatattgtggtagatcgtatcactgctaccaaagctgacattagtattatttctgatgttcgttttgataatgaagccgcgctagttcggaacttccccggtgcgcagaactctagtattcttaaagttcacgcccctaacaacatccacgctatccaatctactcatgcctccgctagaggtgttgcacctgagtttattgatgatgtggtaacaaataactttgacggactggaaaatttccgtaaaaatgtaaacgcattttgcgacgagaggattctctttatataaggtaacaatatgggacaaattaataacgtagagcagaaaggtggcaataagactcctaattacttcgcctccctagtggcgactaaggcagagtataatatttaccactatcatctcgatgggccgatagtcgatgtagactactaccgcgacctatcagtcactctggcaactatgcaagagggagacactcttaatctttatattaatagtcccggcgggtacgttgatacagcagttcagttgtgcaaccttatcatgaactgccagggaaccgtaattgggcacctggttgggccatcagcatcagctgcctgctcaatcttcctagcatgtcatggttggctggtgcacccttacgttatgttgatgggtcatacctatcgtggtgcgcactatggtaagggcaaaaatgagatccagcactatgctgatcagtttaactcgttcttcgaggatatgatgctagacctgtactacccgttcttctctctagaagagatcactgagatgatagaaggtggtaaggatatttggctgacctccaaagaaatcaacgaacgtgtagatcgtatggctgcgcaccgagaacaagaagctcgtaaggctgcgggtcaataaaactaagtaggccagggctttttagctctggcctttttctttatctaaattctacttgacatttgtctcaaagtatgatccgagcaataactataaatttttattgcaacttcctccaataaagggtataattataaatggttagaggaggttcttaatggataaatttatacagttgatatcgctgttactacaagaagcgaaggatccagcttctcttcttaaacgtttgctaaccttactggttggtctagttatctacctctttatagctaatacgagtgaagtcatgtcgtacttaaagactttctctacttcggcggtcctacaagatgttaaagtacaacgcacactagagtttccaaatgtggcacgagagaaggcaatgatcctattctcacagactcgagctgatgccgtcttcgtggttaaatacaagcctgaggctataaatgattaccaaactatcatagcttgggaaagcaacgttcagttggataaatccgatgtatccgacaaagcagttgacaagacgtctatgctctaccgagcgcatttagacggtctgaactttgcaattgatgcaagggaaaaacgaggtttatcgaagtggtctggaacgggtttgccaccgttcaagagtgcaaatttcgagtatgtgtacacgtgcccatattttaaccttaataacatctactctggatatgttgccgtcgcgtgggagaagtatcccctacaagacgaagacatgggtatgtttaatgactatatggcaaaaatctgcgcatcgccgcagagatcattagggagatcaatatgagtttcagatttggaaaccgtagtctccagcagctcgatactgtagatcctaaactcaaggctctagctattcgtgctctggaactctcaccgcacgattttaccatcattcagggcaaacgtaccgtacaacagagtgcccagaacattgctaatggtacttcatttttgaaagaccctagcaagtctaaacacgttacgggcaaagccattgacttcgccccatatatcaatggtaagattgactggaatgacttagaagcattctgggctatcgtcggtgcatttaaaaaggccgcaaatgagatgaacattgccgttcgtttcggagcggattggaataactctggcgactaccgtgatgaaatccaacgtggtacttatgacggcggacacgtagagttactgtaacattaaggggagcttttaggctccccttctttgtatggagcaagcaaataggaggtggttatgtttgcaagtttagtaactataatgctactcaagatatttcaattcggtatagtattcttttccgcaacatggattttaatacgcggtgccatttattttcgcagaacacgcttagctagggttgctagatggcttctacacttcgtatttggtattttcggtgtgttccgctgcccatgtgagaaaaagaggggaacagggttctgctggctctggttaggtctagagaattgcttctcattgtccttagcgctactatttggtatgatagtcctagctataacgctagcatttataccactaatgtacgcaggtggggtgacgtctatcctaacccttacttctcctgtgttgatgtactcagtataccccattacactatttttcgtaagacgaggcaagcactgcaattaatgaaaaattcaattgactttccgctcgatttagcgtataatatatttataaattcgagagaggagatacaaaatgtcagacagattctacactcaaatgtgcgagcattttaaagtatctccctacgagttaaatatagctttgtgggatcgtgaatcaccggagtttaagaaaattgctaagaaatcggagggtgttatgtccaacggtaagaaaatgacccgaattgaccttaacaatgcgctaaccaaactgctcggcgttaacattgagggccagaagctctctatgccaactttgactactattttggagaaagtcaaggctggagatgttaaaaaagtagcagtgcctgagggtcgtctgaaaaagccgtatcaggaggctataattgaggctttcggagaaaagctagacctggataccgcaaccgtaaaaacaatgaaagcactactggagagtattaataatgtctaagaaaattgtattccttaaaggttctagctgcgttccttgcaagcaatttgaaccagtttttgataaactcaccgctgagttcaacctgcccgttgaaaaacgcacggacgacgtagattccctacgtaagttcggccttcgcactgtacccgcagtagtcctggtggatgtggagaacggacgtgaagaagcacatcacattcttagtggtgccacgcttcgctctgcagtagttagtaaagctatccaagactttatcgactacgtagaagaataatacctaaccccggctcaatacgagaccggggttttttattatttgctaaacgcttaaaattttgatataatatttatattgaattgagagaggattacacaatgactaagcaagcatatcttatcctgaataatggttttgcagttggtactaccttcgttgatctggggtatacaaaagaagagtggcaagctctcgatgctgcccagaagaatcagcttgttaatgaagccgcctgggagtatgcagaggcttacgtggaggcggtagatgacgagttagttattgtcgtttcccttggtgccgtaggttgtgatgctcatgtacatacagacttccagagcgaagaagagtgggatgagttagatctaacccaccaaaatgctctgattaacgaagcgttctgggaagttgtagactgctacgttgcgttctgtaaggacgatgatgaagctaacacctgcactaactatggctatgaacacgacgatgtggagtgtgcataatgcaaaaattctctagagactggtcttctgatatggcccgtaaaaatcgagctgctgcctactataataagaaaatacagctcgataagttaataaagggtatcacctacaatgtagagaggggcttttctggtataaaagtagatgctaggagcctggactactcgtgcatactgtgggctaagcagaatgggtacgcgtttaaacgaataggcaatgagatactgattgcctgggaacccgaaggtctagtgcaatacgtaatatacgacccttatcgaggggaatatgtaagagatcacaggcagcagcccacagacttctcgctgccaaaacgttactatttagagacgaggtattaatgaacgtacatgaaacagttaccgtgcccgacaatgctaatatcttcttcattggggatattcatggcgagtacgacatgatgatgggtgccctgaaacttgcagggtacgaggaaggtcgtgactacgtgttctgtgtaggggatcttatcgaccgtggcccgaaaaacttacaggttctagcgaagttcctgtacaacccgaaattccgctctgttcgcgggaatcatgacgaattcatgatccaagacgactacgctaactggatgtataatggcggtagctggactatcacggaaggtttcgatacggataccatgaaaggtatcgcggaagacatggacagcaagatgccatacattatgaccgttgaacaccgtggcaagcgttacggggtagttcatgctggtatccctctgcgctaccaggctcaaggtatgggtgtaactgtaccggtgtgggacgacattgtgcacgaacacgaatctacaccagatttacgtcgtttgggtgtactgctgtgggatcgtgatgtaatccaagaagttgggtttaatctgtatcgtagtggggagaagcatccgtacttcgatcgctacgccagcttctccgaggagtgcgcagtcgatgtgcctgaaatcgtgggagtagactacacgtttcatggccatactggtgtcccgttccccattcgctggaagaaccgtgtctatttggacacaggtggcacctttaacggtcgtatgaccgtggcgtcgccggttctaggtcaattatacacattcaccacagatcgtgatgatccttgtggttccgctgacattatttaataggtgaaacatgaaacttttcaaagatctggaagaaggtgaagtattcgtagttgctggtggcttcgagttgcagaaatgtgtagcaatgctggacaacggaaactccgtgtttacagacgacgcgaatatctcggttactattgccccggacactgaaacctggaaacccaaagaattctgggagatccataaagatcgtccattggacgacctactggacgacattctattcacagcctgaggtaaatcatgaaactcgaatctcgttatattgtatttaagcagtcagatgccgctaagtatctaaccagcactgctattcgggagataaacgacagcttatccctgatctataaaggccgagaggctgacggcaaggtaggattcccgaactatatcgttctagaagaggattggccggagtatcctatcgctaaagaagctctagaaggacgtatcgtgctggaagagtttaataaaagggcggagaagaaacgtggggctaaggctgcagaagatcactataagcagcaccagtctacggaactactccggggtatttctccattctgtgcagggtggaacgactacatgagaagactggtaatcgaggagtaaccatgtatactggaatgggaaatgatatggccaagatgtttattggcctcttaatcctggctgcgttagtcggtgcagcaattgtaggcggaatctgggcgctcgtagcatttgtattttaaattcagttgcttccgagccaatttttctgtataatagctgtataaattgatgaggaagcaaatatgaaaaacgcaatgatcgaactgaatgcaaatatggaatcccttcgccacgcagttaacaccgcccgtgcttcttttaacctgctgatgcgcgatgagagcataccgctgtccgctcgtgtcaaggcatttgaggagttcgccgatgagcttcttcatatgggagactatctcagcgactccccgtttaatgaagatcgccgggattatcaacacgcctactgcaatcgtggggaaatagtttacctgaccgatgttctggagagtgtgctggagtatgcgaactctttcatgcgaactcctgacgaatgggaagacgcttcgaacgactatgttctagacgagattcaaaagaactggccagaaattaagaaactggtagaagaacacattcattctgaagtttacgcttaccggatcgattggtaaaaagtttaaaaattcacttgctaagtagttagaatttcggtataatatacgcataaattaacgaaacggacggaaatcattatggctaagaacactatctcttacaccactggtaaaactgctgacgaacaagctaacaccctgaccaaagacgaaatggttgcggtactggtaatcctgctggatatgagtgggttcgaaggtcagcttgctaaactgtctctcccggcacttcgtgctctgtacgaaggcactaacaaaaatgctgcggcctacaacctcgctaaaaatgaagcgcgctgggccaaggaacaccagcaggttgctgaacgtcgtgctgagagtttcgaacgtgacctgaaacgtgaaaaggcgaagaaaaagtaatggtactagaaattctctccgggctgttaatagcagccctagtaactggaacgggtctggcggtttgggtatcgatactgcgggaaaataaccggagaatgagattaaccaacaacgggctacacgagaagttgatggatcaagtacaagatgctgatgaattctctgctgcggctgagcgacttttagttcgcctggcgaagattgaagagattatcggccaagattccacaatgtcgaagacaaccaaaatgcggttgatcacggagattaagaaatgatctcaccaattgtagcggcactttacttagtagttgttggctacctgtccgggaagtaccacggcttcggtttaaaaggtactattaaggcagcaatgctggttcctctgtatcctctggcaattctgttaaccgggtactacgcctgtgttttaagaatctttggccggggcaaagttaactacgataactgcacagcgttgctggacgatattgaaaacaccattaagaaggaagaaaaatgaaccttaactccaaagaacgccaagtgttagtagatgctctgcgtcaggtagtagaccacgatttgctttgtgatgaagatatcgttgagtcaattgccctaatcgccaagattgagcttgctgagaaggactcatggcgtcctttgagtgagcttcctcctctaggcttggcaatcgtagtacaacgtgctgatggcgctccgtttaacactgtgatggttcgcagagatctggcaaagtcctactccccggatatcatcacgttgcacactaagatcaccaacgaacctttcgagtttaatactcgtcactattactggagactgaccaatgcttaatcagcttcgaatctataatttcctggattgcaacttccaggtctggcgagaactccccagccgtttcctaggctgggcattattcctctccatggtggtttcagtcttccataacgtcccagcgacgttctatatggaagcaattcaaccagtcactgtattcatttacgagatgtacctcgtagcaattaacgggtggaaagatggccgcatcaactgaagttttaaatcaatattttaatcgtgagcaccaagagttcagcgatctgttcatccagatgttcgtcaacgctaataacgctctggactatcgtttcttcaacgagttccatgagaccacgttcagccatcaggatattaactccgctctgaaagaactcattggctctaaagtgataccattccgacagactgcaaatgcagagacactagagcttagtgtcgtttggggcttgttcaagaaagcatatgagtttggcaagtatcagaacgcacgtcactggatttacgaggtgtatctaaacactgaagtcattctacctcgccaaatgatgttgggctggattgcaaaacaacgtcctgaacgcaatgcaaaatcattcgcaccaattaacgacgggaacttataccatgcaagtgaaaaatttgatgctccaaaatccgtggcttgatcgcctggcgcaagtagagaagatgcttctggttgaaggtctgaccgaagaagacatttgtctgaactccttctacaactgcaaaactcatgtaatgcaggcgctagacgaagaacgtgttgaactgagtaagtttatggtgaacatcgccacagctcaagttcactggcaaacccagggcttatccgccgacgatatcctgaaacataccctgaacgctattgcagagtatgggaaagctcgtggtgaagctctcttggcttcaaagaaagagtttgataaatcggagtcaatgctgaaaatggcaatcgatatccatatggaaggcatcgacggtactattcactaagagggtgacaaatggctaagaaacgcgttgtggtaaacttcctagaggaagattctggggactgcgagtacggttgctggaatactggctacggtgttgaggttatggtggatggcaaatgcgtccaccgtcaggaagcctgggcaagctgcactaacaacagcagcgtagactttgacgtattggcccatgttcttcagggcatcaagacgaaagaaggctacccggtcaaagcagatcacatcgactttggagatccttccgattatccagaggagtttctggatctgttcacctaacaaagaggccagggctaattgctctggcctttcgtggtttaaggatattaaatgaaatctatagacaactatttgcgcggggaaaaccctgtagatcaagcagcagttactgttgaaaaggtcaggaaagagtgttttatacttactcaacgtggaggtggtaatcggcctaatcgagtgtatcttaattggactcagtccaaagacctgtatgagaggttgaaaagggaatttgagtagtggaagaattaagacagaagataaatcaagagctagtatgggaagctaaatccttccccattaatcaattccttaaacgtgacggttcgatcaaccataataagataaagcaacttaggccagatttcaggcaagatgctaagaacctcatctttattaaccgtgctctagacgctcatggagtattcttcggatatgagaagctagtgttccacagcctaaatcagctggtggaaatatggtgtcccgaccatcaagattactttatgcaaactgctagaagccaccttgagggtaacggttgccaaaaatgcaggcaccgcatggttaccagagttacggattatggaagttacaccgtgccagcatactatcacaaattttcaattgacggagactccattatttggtataataactccagtaaattgataaaacctttggaggttaaagatgaaatacgattccctgaataatccgagcaccaactatctgactgaccagtcagtttctgagatcaagttccatccgaactactccccggactctagcaagccgagtgtagcagctatctccttccgcttccgcaatctgcgctttacgttcgttggagaggaagacaagatgatctctataattgacaaggttaaagcagtaagcgagctgtccggtagcgataccgttaagttcgaagcattaacctcgttgctgctgactagtggggctaccgttggtaagttcgaacttattcagccgcatgtttctgcactgaccaataccagaaacttctgggatcaagccaatgtcgagagtctcataaaatgggatagtgctactgagttctacaacaagtaagagggctaggatatgttattctgtacagttgattttgaagaagctaacgaaacgtatattgtctacggtatgtctgaaagcaaagtacgtatcctctggaatcagttccaattagaagtaccggacgacatatccaagactccgaaagacttctttcatttaatagatattaaggcagtaaaagctcgtaagaagctaactccttatgtgttcccaggtgcagtattcgtccatgagttaacagcgtatactaacgtggttctcaaaaagtctcgacagcacccaggctatctcacgatgttgacgtacaaagttggagctattcacgacggtgagctggtggttcgggtagatgcacgcttgcagcaagaagtcgaagaaatgattaggcagtgccaaaataaagcagaattaaaacaaagagcacgcctattcgacatggcagcacctagcgaggcagttgccgcatatcacggcttttataaagaaatagctgaatccgatgaagatttctttatgtaagaggataacacaatgaacgagaaatatgaagtatggactccggttggggagaattgtagctatcttcttcgtaccctgtgtactcgggaagatggcacatctttctcagaatacttgagtgaatgccacgctaaggcccagcaggacaacccgctatttaagatcagaggagaggatatcctcaaagttaacggcgtgccgtacacgccagtggacagtttcgccgctcttcaggtgtttaaggaacacagagagcgggagcaccggaggatgattgaacgcttgacaggtagagagccgttttcacatcctaggtggaatgaggagacataatgagcagagttgaaaagctacaacatatctataacctggttaagaaagctgaccaaaagaaactctcagagctgagtgaagaagagtatcaagcagtcctcttctgttgttcggcaatgccagcgaagctcgatggagtactggcaaagtcagacatccacaacggtaaggaaacaactttccagccgccgtataaatggctagcgtccaacatccagcaaatggtgggtaaagttacgggattctcaaatagaaagacgcctaacatctttattgacattactcctagaacccctgagtttacgaaggactggagagacgcattggattcatttccgtcatggaaagtcttttataaacctgacgatgaaacgtatgcacatcttccctttttgaagcacccaggctacacagttgaagacccaagttctggggttaatttcaaagacttcaagtgtactgacgagaacattgcttacggactcatgagaacatccgtgaggattgcaatggatcatgaactagacaaacaagatctagcagtcattgcactttgtaaggatcgttatattaaagttaagaggatcgcagagaaactttccgtactttcttgctttgaaacaatccgagattgtgaaccagagggtgaatatcctaaaggttccctctactggaaagatgttaaacatctaggactatcagaagaagccgtattcctaggcttagtagttacgggaagattcctgaggctacaggagaaataatttgtacttcgtctatattttcgctaatgaagatcagcggcttatccctaggcggaccaaggtgggttactcgcgtgacccattttatcgtctaaattcgcttcaacttcacagactacctcatcgtggattacaggatatcgttattcattctgtctatataatagatgacgccagcgagttctcagcaaagttgctagagaaggccgcgcataaaaagtttaagcctatgcgagttaatttcggtgacaagtttgatgggcatacagaatggtttgatgttgaacctcacgttattgagaagttcttcctgtcagttggggcaaaacaagtacctattgacaaactcatagcccaagaacaaaagattcgtaaatccacgaaaaagtagttgaaaaaaaataaaacgcatgttataataatctcatagattacatacaatttgaaaaatttatgaagctaggcggattatgaaccagaatcctggactcggggcgaccaagcctacgatgtcacagagactttctggtctcacataatccgcatgcctagcgcttcgtaatctcacatgaacgatggagaacctataactataataaaactaaggagaaaatagggaaacgtgggaatgaactcacgattactcacgtttaaaagtaggtaatccttacacaccatcgtttcgcggctctagggttgaactctagtcatctttctatacagggcggttctttctacacacctaaggggagggcggttattttccaaccctaaaaatccataaccattttctggaattgcacaaattttcacaccgtctcacatatcccacgaagccctcacacatctcacgcaatccctctagtacgcccaagttctaagcgtcactccacaaagtcctcacgaagtccccacacaaacccacgtcattcacccaagttctaagcgtcaaacattatcggatttgcacattgctcctagatattaaagaagccaagcatttctagatttctagcaattgtcggatttgcacatgacaaaacgattgtcggaaatgcacattaactctgaatccaaaaacacggtcgaatttgcacatataaaaactactatcggatttgcacatactcaaacgcttgtcggaaatgcacattattactgaagtacggtagctgtggatcaaaatcttcgattctgcgaatataaaatatctcctgaaatttcaagcctttttacacggtgtgtcaccgtgagttcgcttgttttttagagccacaggaattttttctgtgtcaacccctttacccccatttaactcataatattttcccttatctgccccctgcccccatcatccccgaattgccccaattaaccccctttatccctcccccaaaaatacccatcggatttgcacatactcacccaaactcgcgcccaccgctgcgctgcagcaaactgcgcgccttccccctaaaaattattatcggttttgcacatattagtatcggatttgcacatgcccctagggcggtaaaaataaattttcatcggatttgcacataccctagcagcctcacgtaatattttgcttgacacattgtcggattcgcacataagcgcaggaagggccgggggtcggaaatgcaaatacgaatgagagtgattcgcatttgagaagttgaatgcgaatgataattattctcgtttgataagctgaatacgaatgagaatgattcgcattccggaaaggaagtgagaaacactatcatttaagttatcaccagagttatacaaaattgtaaattagcttgacgcttgagaaggtcgcagacgagttatccacagacttattaacaggttatcctactggttatttatacagtagttcccggcaacactttctgctaatgtcgattttaagcgccctaggaagcgttctaagcgatttaaatttcgtgggggtattttagtaaggggtcacgcggtggaggagggcggcgacactcttacatatcgccgtggaaacactcaggccaaaagtgatttacaactgaaactattagcagggtaatgattgcgctaataactatcatgcccaaaaacattaggccgctcattattggcagctctgcaaaaagactagggtcataataataaccatgatgacacgaaaaccccactcaccgccgccaccgtcaccgcctttcttctgaccgtcacgaataacgttcaattggtaagtattgtggctacggcttttgggactaccaccgcgtaaagttcgcattaaaaagcctccttattaaacaaggtatcatagagggcatagccgcaaaaagcgataaaacccaaaaccatcatagaaccaactaaaccgagcgccaacattgaaaaagccatttttcgatctcctatatgggtgagtcataataataacataactctagcgggaagcaactactatttttagtagtagacaagcaaatccgatttccctatagtttgatctcccggcaagcatagaaaaattttgcttgctaaaaagcggagaatcttgctataatagattctcagccaaacaaaggagtaacagaagatgttacagaaatttacccccgttgcaaatttgcctatggttcgcggtggcgctcgcaacctgttagatggtagtaaatgcgcctctatcggtcatattttaggcgtttaccgctcgaatatggaaagtagaaccagccgcgcttttgaacatagccgcgactacgttctagccaaccccggcgctgcgattgttatttttcacgacgatcaatatttagttgacagccagcctattgatctgatagtatctaccactacggacgcttatttgtataaggcatccgaaggtaagcaagcgtcaagacgtttttgctaccatgaaagcgaactgctcgctttcaccgatgcccgcgcatggattaaaaacctgtgcgatcatctggaactaccaccagcacgaatttctagcgaaatgatgatttttgtgcttgacaaagacggaagcatcctgctaccatgtgacccttacgatattgatatcgaagaaggggcaaggactggaaattatcgttatgatggcgagctagaagaagtcgccccggctgttactgaaaatgtagttaaccctaataattttgagactggagcattacaaatgaatactatcaaatctaccgctaccgctatcgttgccgctaacaaaaacgctgctgtaaacgctgctaaactggaagccggttctatcgtcctgaaaaaagtttccggcatcgccgccagcaaagcgccgtttatggttcgcggttatgttgacacggcggtaggtcgtgtggtaattgctaacctgctgaatttcgcggttagccagtatgcgccgaacaaccgcaaagcggtgattgcggctgacgctgcaatgcaagccgccatgttggaactggtacaaagtttcaatgtaggcgaaatgattgacgaagtattgaaaggcgtcaatctttctagcctgattgaaagcgacgtagcagaataattatatctagcgcctctataatggggcgctataataattaccctgctaactaactggagacaaaaaatggaacggctaaccgctactttcgaaggcgaaaagatgacgatcgctaatgtatggcagcgcctgcgccagaatggcgatcgtggcaattttgctattttcatcgagccaaaaaacttggataatctggctcgccagattgaccgccgggactgctacccggatacagatgatatgctgggaatccccttgcggattatcggggtatatggttacggctttgatatctgtattggtgatagtagctttgaaatcgactgcgaatccggcgctaccgaaatcgaagtattcctaattaatttaggctcgctaacgtttttggatacgccaccagcggaaccggaaccggaaaagctggaggtgaaaacgtccgttattgttagttcgctaactatggatgagctggcggatatcgtgtcaacgtatgacgaaattcacgccgacgcgattaaagagctaaacaaccggcttgatactttccgcgataagctgtaaaatccctcccggcggctaaatctctagccgcccccattattggagaaaaccatgtttcatgtgaaatcttgcgtaccgggcattaattatacggtagaagcggaagaaggtttatatttagagggcgggcgaattgagtctcaggaagtcgccgccgtcctcaaatgtgacactaacgtttgcggtacgtcctggactgaccttcattttttaggtcgtggaattgacgtcgattccctttcctgggaaaaggcttgcgaacatgctgaaagtatgctaaacgaggatgattgggatgatgacgacagcgacgaaaaatacgcgaatgctggggtagaagggtcgttttatatgtactggcccggtcattcctgcaacctcgttaatggtggttcgcccctccattccgttctagagcgggcaatctatttgggctatatccagatagttgacggaaaggcagtcattaatctgcgcgaattgaaaacgtttatctatatcccggatgctgaaactatccttcatattgaggaaggtttaaaatccggttggaaggttagcggggtcgtatacctatgatttacatctatgtcaacaaatattttctggcgcactataaaacgatggaatctgtgatccagtacgttagccgccagaatgcaaggcatattgatgaagtggctaccctcaaaatcggattgcggggcgatgctatcaatattagctggcctttgctaattctgatttgccgtgatcttgtagcgggtaaacctgtttccgtttccgcgctgggggaatcttacccgctttccgatgatctggatctctatgatctgctaaccaaatataaaactgaacgcctgttttatcgtggcgggtcggtatgctctagcggcgaaacaatagaaacggtattccgctaatagttagctaactaatgataagcctgcttataatgagcgggctttttatttttctgcgcccctctagcaatggcctttaatatggcctctaaatttaaagcgcttagaatgcgttatagagcgttctagaggcatattagagtattaccgctgccggaatatactgtataaatatacagtaggataacctgtggataagtctggggatatctcgttataccgcttctcagcttgtcaagtcaaaacgtgctgtggataacttgcccggaaaacaggagtgcagcataaaataaacgcaagcattattttgcgttttcataaaaataatcgtttgtctagtggataactaattaaatttcatttttgctttttgtataagttattaacagggtctaaaacgccctagaatcgattctaagcggttttagataaaagccgatgaatcataccgggtatgaggcgatctcctcacctcgtgtgaaatagattttgtcaaccccttttgaagaaaaatagtaaaaatattcgttgactttctttaaatattgtgtcacgtgcgcgcgcttcctatctatcgtgggcctgttttctctttacgcttctttacaattctccgcttgcggctaaacgccattgtgttattatttatctcgtagggcggcaagacgaaacggaaacacgaaagcgtttcactagtagttaggtatctggaggcggcaacattgccccgactaccgcgaaaggaaaggccgaaacgatatagtctgcccccgccctacactgctctttaaaaatttgggattctataaaaagcgttatcggctttctttacttaataagggaaccgaaaacatgttaaaagaaaacgtaatgagtagcgagattgtaaacgagtttacagtagcagacgccgaacattttatagagacttatttaaatgtgtacgacgtagatttagccttcattcataaagacggccaaatc

SEQ ID NO: 2 - DNA, 45423 bp, Klebsiella pneumoniae phage Kle_F17/19
tgaggtaacggcggagtttgccgaacgattctcttcagccggatagcacaaagcgccctttacgggtgcttttttttggcctatgatcttgagtcgataagaggaggaaatttatatgacaactaacgtttcaaaagagtttgcgaactgcttgcaaaagctgttccctggcgagatcttaacgctgattgacattgacgccacgaagttcggcgggcagatctatcgattccacaacgagaatatcgcctactcaaccgaagaacttttggcagcggttaacggtggaacgctgacaccgaaggcgatcacgtttcgcggtgagcaatacggcccgcgcccgttcgggataggcgggattgcaatgtcaagtgacggcacggtagaaaagccaacgctgacggttagcaatattgatgcgcaagcgagtgctcttattcgcgcctacaacggcctcatgcaagcgaaagttacggtatgggttttggttaaggatttgctgaaagaagatggtagcgtagctgatggcgattttcggcggttcgtttactatatcgaaagacctaaacaggttgacccgcagaaggccacgtttgagctaacatccgtcttcgatatggatggattgatgatcccggcccgacagactcaaaccgtttgttattgggcgcaacgcggctggtacaaaaccgggaagggatgcggctacaacgggcaaaacggatactttgacaaattaggaaatagggtagatgatcccagccaggatgtttgcggtggccttgtttcgtcgtgcagattgcgattcggcaatgaggctttagattttggcggttgcgctactgcaaccttgaaaagcggacggtaatatgttaactccaaaaatcaaaatgcagatcatgcagcatgccaaggaagtttaccctcatgagtgcgcagggctggtgacgcagaaatcacgcgtgcagaaatatcacagactcgataacgtttcgccagatcctgagaacgaatcaatgccggacgaaacacagtacgcgctggcgtcactggaaggagagccgatcgccttcgttcactcccacactggcgacggggcaaccaccgttccgagcgccacagatttatgcttctgtgatgaatctggcttgtcgtgggttatcgtgtcgattccagaaggtgatatgcggattatcgaaccgaagcgcaggccgctgattggtcgcccctgggctttgggcgcgtatgattgctacggcctggtgatggattttcacaagcgccacggcgtcacgctgactgaccgccgcctaccgttcgaatggtggaagccggaatacaaagaggatctttaccgcgactactggcgcgaagatggattcatcgaaaacactggcgatcccgaagtcggagatatgattatcttccagcttcaggcggaaaagtggaaccacgctgggatttacgttggaaacaataatatccttcaccacgccttcggcaagctatcacgccgcgatatctattccggatggtatgagcagcacaaggttttaatttgcagacataaggatcttaaacatggcatcacatacaaagacgattaaactttccggctcccttgggcgtcggttcggtgtcttccataacctagcggtcgattcagttggcgaatgcatccgggcgctatcctaccaggtggaagggtttaaagccttcatgcagagcaaggttggttctaacatgcgctttggtatcgtggcggacggaaaaccaatcagcacgaatgactttgcaacgttcgccgtagctaaggagatccgaatcatccccatcccgaaggccagaaagaacggcggattgttgcagatcgttatcggcgctgcgattatggttgcagccttcttcactggcggtgcatcactggcggcaatgggggcgttttcatcggcggcgtttatggctggtggcgcaatggtcttgggtggcgtaatgcagatgattgcaccgcagatgggcggcaacatgcgagcgagcgagtcgccggaaaacaaaccatcgtatgctttcggcgggcctattaacaccacggcagcgggttatccaatccaattgccatacggttacagattggctggaggcgcattgttcggttcaggatcttacgccgaagacaacaactaattaagcgattcgctttttggcctgggggcatagcctccgggctttttgtcgtgtacaattgcaaaactattaacaggaggctaaacgatgactaatatcaaggcccgcaagggcggttccagcaagacgcgaaccccggtagaaatgcctgataacctgatctcgaaagacaagattaagttattgttggccgtttctgacggtgaagtagtggatgacttcagcctgaagcagttgcatttcggaggcgttccggttcagaacgaagacggtagttataactatgagggcgttattgcagaatttcgacccggcacgcaaacgcagagttacatccagggcttcagtgaatcaagcgctgaattccaggttgctcgcgacgtcactcacaacacgccgtacacgcttaccgtatcgaacaaaaatctgtctgctattcgctttcgcctgttatggccgcgcgtgctgactcaaaaagataacggcgatatggtcggatcggttgttgagtacaagatcgagatggcggtagacggcgcaagttatcagacctacctaactggcaagattgacggcaagaacacgactggcggctacgatcgcagcatccgcgtcaacttgccgcaagacttcacgtcgcaggttcttatccgcgtaagccgaattacgccggacgctgacggggttaaggtggttgatgccttccaggttcagtcctacgctgaggtgatcgatgcaaaattccgctatcctctgacggcaatgctttacgtcgagtttgatagtgatctgttccagaaccagatcccgacaatctcgctcaagaagaagtggaaaattattcaggttccaagtaactacgacccaattaaccggacatactccggaacgtggaacggaacttttaaatgggcgtggagcaacaacccggcctgggttctttacgacctgatcatgaatcagcgttatgggttagaccagcgcgagttaggaatcccggtagacaagtggtctctctatgaggtgtcgcaatactgtgatgagcttgtgccggacaatcgcggagggatggagccgcgttacctgatggatatgatcgtgcagtcgcaggtcgaggcgttccagttagtaagggatgtttgttccgcgttccgtggaatgacgttctacaacggtgaaagcctttcgattatcgtcgataagccgcgcgatccggtgtacctgtttacggctgataacgtcgttgatggcgtgttcgttcggacgttcccaagcgaaaaaacgatgtatacatcgtgcaacgttatgttcgacgacgaagagaaccagtatgaacaggacgttgaaccagtgtttaaccctgacgctgccatgcggttcggtcacaacccaacaagcatcacagcgatcggttgcaccagacggacagaagctaaccgccgcgggcgctggattctgcaaacgaacctaagcgcaaccaccgtttcgttttctactggtctggaaggtatgatcccttcgtgcggcgatgttatttacgttgcagatccgcactggcaatcagccttcaacctggtgttgtcaggtcgcattatggaagtaacgggaacgcaggttttcctagcttttcgctgcgacgcgaaggcgggcgatacgctgattctgaacactgacgacggcaagccattacgccgaaccattgccagcgtatcagcagacggaaaaaccctgacgctaaacgtgggatataacttcgacgttgcgccggacagcgtattcctgatcgaaagtgatcagcttgtagcggaacagtatgtagtaacccggattgaaaagggtagtgatgacgacgaattcactttcgcgatcacggctacgcagtacaacccgaacaagtatgacgcgatcgacaacggagtaattaccgacgaccgcccaacttcggttgttgaccctgattcaatgggcgctccgaaagacttaacaattagttcgttttctcgcattgtccaggggatgagcgtagaaacaatggttattggatggtctgctgtgcagtatgcaaaactgtacgaggtgcaatggcgtaaggatggcgggaactggaacaacgttccgcgaacggcgacaacgcaggttgatatcgaggggatttacgctggcgagtatcaggcccgcgtaaggtgcattagcggcgggaatgtagcgtctccgtggtctgcgttggcaaccgccacactgaccgggaaagtcggagcgccaaaaggcccgattaacctttttgcgtcggacgatgagatctttggcattcgcgttaagtgggccatgccggaaggtgcggaagacacggcatacattgagctttaccagtctcaaagcggaaccgatcaggatgcaagcctgcttactctgattccttatccggcggctgaatactggcactcaattttgcccgctggctatgtgaactggtacaaggcgagaagtgtagacaggatcggaaacgtttcaccttggactgattacgctcgcggcatgtcatctactgacgttaacgccatcacggatgtgatcctggatgagatcctcgacagcgacgcaatgaaagaacttcaggagagtgcgcaggatagcgcggcaaaactcaatgaatacgcgaacagtatcattcaaaacgcattagcgaatgatggcgatgttagaataatgagaaaggagaatggcaagaggaaagctgaaattaaacacgcagaagttctcatagcaaatgagacggaagccagggtgcagcaggttaaccagatctcggcagagttcaacgaaaatctcaatgctggattaactcaagttaacgaggcactagccaatgaaactgaggctagggttacgtcggaagaggcactttcagcaaggatcggagagaactccgcggcgatagatcagaaacttgactcatgggctgacgttaatggcgtcggttccatgtacacgatgaagttgggattgaagtacaacggccaggaatacaattccgggatggccctacagcttaccgcgcaagggaacaacgttgtttcgcaagttctgtttattgctgatagattcgctatcatccgaaatgctgagtctggagcgtacactttgccgtttgttgtgcagaatgaccaggttttcatgaataacgcgcttattcaggatggttcgattaccaacgcgaagatcggaaacatcattcagtctaacaactacatcgccgggcagcaagggtggatggttaacaagaatggtagttctgagttcagcgaggtaacagtaagagggacaatctacgcaactgatggttggtttaagggtactgtctatgccgagcacattgaaggtgacgtgatgattgcggagtcaaatacgataccattcaaggattattcgaatattgatagtggtgattatgagatacttaaaatcaatggtgaaaacttcgataggagcatcgacacaaacctgattgtccaagttaactctacattcaggaacacatttagagttattatccagacaccagggaaagaagagatcgagttctatagtatagatactggcaacgacggcggaacaagatcctatgcactccgaggcttcgatgttccggcagcaggaaaggggcaacagaacaggatcatagttagagtcacgcagagtggtaggggtagcagtgtaaaaacatttactccgtgggttgagcgaaataatgggtctacagggacagaaagttcagatggaacaatatctaacattggttttataagagagaggacatacatatcagcatacagaaaaggaaatagaatagtagtctagtgacaaaagcgcccggttgggcgctttcttttaatatgaataatagatgtaaaaaaggggccgaatggcccccttttttcttttgttgccttgatattcttgatatgtcatatcgcaacaatgtcgattttgttttcggtaactactattgttttggactgcgccaaccgttgcaggccgtagttggtatgaatttttgcgtattgcattatttgatcccaatgattagataaagtttattagcacgccatcgaaatcgacaactgcgccatcatacgcgcgcatagaaataactgccgttgccgctccaggaggaacgatacctcttataaagctcccataaacagcccaatctgcaacgttaaccggaaggttagctgtagggccagcaatctgtgttccgttttggctgtagaatgagaggtttatatttcccgccgcattcccatttgcgccgatgtttacaacccttgcccaaaaagaggtcatataatactgtcctggttttacgttaaactgctgagttgcaaaaatgctcaacccggcgattgaagtcatcctcatgccgtatgacccagccttcgcatactccgcagacacaacggcagtttgtgatgcagaaccagcgttattaattgaccatccggcattattaccctgctcaaaacctgaattataaacaggtgacaaagatcggtgtacaggaatatttccggctcctgatgctatgtcgctagtacaagcattgcacattacagatccaggcccctcaacaaatgcccgaaccatatccgatgagttctgttcgaatttgtatgaattaccaggaaatttaaccacattaaatattatgaatgcgtttattccgacataaattataggctgtgtttgtgctaatggctgattaatggtcagtgtgctttcagaaagaataaatcttgcaccagtgccagtacaggtaacgtaaggataccacaccgactgcccagggttttctatgttacccattccagacattgatactgtagccccatttccggtgatctgaactcttgtattaagagttgatgtgccgtcaaggccaagattgaaagcgtcacatgcaattatgatcggtgcgccgcttgcgtctgcaaccatcccaccattaaacgttatgctctcgcctgaatctaaaagaccagccggagcatagaatatcgaagttattcccttagtaataacgcactcattgaacttgtacctccaggtgctgtttgcacaagaaactacataatcgaagtcatggaatgagcaatgttctatgcagcactgtccattataatattgcgcgccaccgggcctgccgataagtaatccatttttaccagcaactttagcaccaaagcattcaatcccggacagcttgttgactgtgttgcggtacagtggggctggatatgttgctgatgaatagacgttaatggcgtaagtggaggttacaccagaaaaatctattgacgcgcaacctacatggcagactaaggatataaatcccaagtcaatttccagcatttccgaaccagtgtatttatactgtccagcagagataactaaacccaaacgttttggtttggcatatgaaatcgccttattgatggcgttggttatatcgccatccgatggaataccgaaccatgcaacgtttactgcaccatcaaaaatccgcttccagcgctttccaccagcagtgacaatgcagatacctccgtcatcaacggatgtactgtcagtttgatcatactcaaaatagccagttattggcttgtagcctgaggcatattttatgacttctatcttttggtttggtaatgtcggctctattgtccgtagggtcgcaatgtcactacagcgccctatgtttttagcaccatttggctgagccaatacaaccagcgttgactgttcgccggaatcatcaatgatctgctgaatttcatcgcgagttgcttcagcgtcatctgctgcctgctgacttgatgccgctgcctgctgacttgctgccgctgcctgctgacttgctgatgctgactgctggcttgctgatgctgactgctggcttgctgatgctgactgctggcttgcggcagcttcctcttttgcctgttctaccaattcttgcaaagtggataaatcaaagtccttaaagaaatctaccgcctcagcaatgacggtctcctgcgattggtagtaacgcagagtttccgcaacatcctgcgccaggccgtcaacggtcagggagtcgcttaacaggatcgcgtaatcactggacggaacagccgcgccgtttgtggagatagcattgatttgcgtatcacttacaaccttgttaactaccgccatttgaatcggggacgataaaaacaggatcgtagcgcctgggcgaattagcgttagcgctgattgccagttagttccagtcccggtgacgatgccgtttgcgtccatcgcggctttgccttgtctgtatagtgccattttaatacctcttttggttggttgagtaacgaagagataatatcatcaatgaagcaataaaaaaaggagccttgcggcccctttagttgtcaaatcagaacgggatatcatcatcgaaatccatgccatgattaccaccaccgctttgcggcttcggttgttgttgttgcggcttaggttgctgaggctggccccacccaccctgctgattaccgccgccctgcgctggttcgcgctggctgaattccagttgcggcataatcatttcattgtggctgtaagttgcgccgttgtgctcgcggtttacgatctgaagtgtccggcaggttacgctaataaccttgtcgacctgcaacgcttcatcgtaccacgcaatcatgctttccttcgcgaagaagacggcgcgatagttggtgtatacagtttcgtcctggccgtcgcgattgcggatcttcattcgctccgacaggtctaccgcatacatcttccacggcccgttattgttgctgccctcttttacgtaaggcgcttttcggattacccctgttacaacatgcatttttattcctatggggcggtttcccgcccggttaaattagttgaaagatgagatatcttgtgcttcaggttcaggctttgattctacctgttccggctcacgtttcgcaacctcttgcggctttccggggttaaatccgttcgctggagtcacttttagttgcgcctggcgcttcgtgatatcgtcttccgtaaccttccattccgcaggcgtcaaagtctgtttagccagcttgtaaatctcgcggagagattcgaggtcttcgcacgcatcaatgcgttttttgaagtcttgcggcttcatcttcgcgatctctgcgtcatcgtcggcctgcttgatgcccagcgcggcggctaacgcataacggcgagcgtatgaggtggtcgatccatacgcttgctcaacggttttgctgatcggcatgttgaactgaaacgccatccactcgccagattcgtgcagaaacatcgtttcaaggtgcataaccttgtcggtgctggtatccatcattgattgaatgaccataattttgttcttttccagcgatggcccgatggcgtcaaggatatcgccgagattggcgtaggtgttccccaggtggctattcttcccgcttttcttcgcggcgacgaaaccagacttcgctttgattaacgcaacggcgatatctttgaaactttcagaagtacgcatgacaaactttccttttcctgattggtggagcgcactatatcacaagtgcgccgtagtgtttagctatttgtgccgtatacttccgggaacatgtatttcacgaattgcggcgtgggcaacttcacttccgctgcgttcgattcgtatgacggccaaacgtcatgcttgacgcattcggcgtactggtgaatcacgctttgatactgcttccgcccgatctcaatctgctgatccgttaacgtgaacgcgagaggcgcgaacggtgattttttctcctgcgtcagcaggcggacaacaaccgggcgtttttcgttgtacgtcttaacgaacagatcgcgctgcaatgccattttgagatagtagcccaggttgaacgccagccgcccgaagtcatccggtttagaggtctgagtagttttgtagtcggtaatcaccaccacttcgaaaacttcatccgggttaaacccccactctttgatgagttcgggatccgacactacgtcaacatgatcgagtcgaacctttaccgggacgccgaaaatttcgccgaagattgacaactcgcgttgcgctgtatccgattcaatgcacgccgcgtgccgtgggttagctagcatcacgtttcgcatttgcacgaccgcatcgaaatcagaatacttgaccagctttcggcctgagttgatggcggtgctttcgtcgcagagttcgatcgcccaccacacgttaacatcaatcccggcgcggtatgccatttccagcaattcggggtagtccttgctggacgtcccaatcaggccacacgctttcaatttcgcagacaatgccgacttcgaagtaatcagatccttaacgtcgcccggcgcggtcgctcgcaggtactcgccattaaattttgacgtctcaagcatgcaggtatgggagcaagtgccgaatgaaagcgcagctgtttcttctcgctccttgtatttccagtgggccggggatgttgcgtaaatctcgccgaggcttgagccgctaacgtatctggcgcaccaggagttaggatcgtggtaatcatcgttggataattccgcgttggtgtatgccttaaaaattgcttcagccattggtatcactccgtttgtggtttcgttgcgttaagtatacgcatcacgattccaggtgcaaggcaaaaagtgctattcgcaactggtcaaaaaatgagcgaaatttacgtaagatttagtaagatgcatcttacgtgattttctttatatatttcatgcggttaaccaaaatcggtaagatggtaagatgcctataggtaaatattcacaggaaaattgggccgaaatcgcaaagcaaaatacatatacccggagaaaatcttacctacacagataaatagagagagtaataataataatatatttagatatcatatacttatacatatactatggttcgatattggttaaaaaatgagcgaaatttacgtaagatgcatcttacctaatcttacttaaagtggtcgaccagttggtaagttgctgaatttttggcataaaaaaaggtaagactgaaattatcaatcttacctaaaatttgttcacgctttaatcagagaagtgatgatgccacgtagagctttcgttcgaatccgcctttgaagttgtcattgttcggcgcgatcactttcatatcgcgttcttcagccgcagccagcatatcccgatctttcctgctgcatacgactcgcaattctcgcttgccttcgccgccgccctttcctttatatcgataggccacgatctcaacgttggacgggatgatgcaggcccacacgtcacacttaaacgagcttgcgatattgaagtgcatcgcctccacccaagagcgagccaggtaaattggcccgttaccgccttcgctctgattggttacgatcaccgatccgaaagtcaggtcgccagctaacatcttctcccggccctcctcatcaatgaataagatgttgcagtattcatcgtccggcccgtcttcatgcaccagttgcatcgggagcgcatgaaataactcttgcctgccgttctcgtgggtttttacgccaacccggtatgattcaacgaactcattttcaatgccctcatagagcgttacaggcgtgctatcgacggcctccgttctgttcattatcgccacgacgcgatcgtgatctgccatcttgccatagtcgtacccgttatcgcgagtaacctgcttgttgcgcttaactacgtactcttgcggaactttgcccaggtatcgcccaagaatgttgatgcactcgctatacggctcgccgctcaacttcattaaccatccgatccccttatcggcaccgcagccgctgcaatatgccccgccgtcgccgcgattctctaatttgtcagtccagcggaagcgatccttgccgccgcagttagggcagtcctggtgtttgccgttgaaatagcgggagtggatgccgcaaatattttgtagcgcctcgcgccacatccccggcatgtatggcaaaacctctttttcgtcgtaaaaatccacgtcataacctccaaataaaaaacgcccacgcgagaatcataacccggtaggcgtttagtagttagtcaaattgtgctatcggactactcgaagcatttcgcggcggtcgcagcggcgcgtaatcggcttgccgttgctgtcgaatctgagatccgggcggcaaaatgaagccctgaagcctttgcagtcattgcggcggtaactcttatgcaccaggtaagcaccatcggctgaaatcatgccgcgcttaatccactgctgaacaacctggatgctaacgcccaactcctttgctgttttggcgatgccgccgtatgcctcgatcaccaattccatccgggtagtcaggcccgcccttacttcatccttcaggacgtaatagcctgtcggcttcttgcggggcttcttatctttcccgcgtgacgtcccgttatttccgttcaaagttctattgtctacctttgccatttgttccataatttaacctcacagcactttttgttaaacacgataaaatttgctctgtattatacacgcaaccatgcgaatgacaaatcaggattaaccatgctgacaattgaacaacaaattgaagcctacgcagacaagatcccatctattcaaaagcggttcacggtcgggaatatcgttccgtacccttaccaggctgtcgcgtacatcgagacggcgaagcggatcgcgaactatgagcatccgttctacattaaggcgtccgtatccgcaggcaagacaatcatgatcgccatgctcgcggcgcagtgcaaaacaatgaacttgcctatgatggttctcgccaggcaggccgagatcgtgaagcaggattccgaagaaatcagcaatcttgacgtgccaaactccgtttattgcgccgggttaggtacaaaagcggcttacttcccgatcgtcgtcggctccgaagggacggtggttaatggcctgtttaagatgcttggcgattacgtgccttcagtcctggcaatcgacgaatgccatcaggttgactggcaagacctcgcagaagcgatcgcgaacgatgaatccttcgagtacatgagccgcgcaaaggataagccgtatcgcgtgaatggtgagatcgtcgatgccgattacccgtacaacgaaaagttcgaaacggttgaatttggcggcgggcgcactcaatacaccatcgtaatcatcgagttaatgcgccgttgccttcagaagacggggcgagaacttcgcatcgttggctacactggttcggagtttcgcggcgtcattccgatcttacaggaagacaagagccagccgggtttctggcgcgagcagatcacggacattaacacaaactaccttgtcgaattcggttcggtggttccgacaatctttggtgacactgaggccgacggcctgggctatgacctgtcagagtttcacggctccagtcaggacggcacgcaggattttagcgcggaagacttgcgcaagatggagaagaaaatccacgattccggcgaaatgactaagctgattatgcagaaggtggtcgagcgtgcgcagacgcgaaacggcgtgcttatcacttgcgcgggccagcgccactgtaaggaggcggcgagctacttaccgccggacgccacatacgcgatcatcacagagaagacgaactcgaagaaacgcggtgaaattctggacaaggctaaccggggcgagattaagtacatcttccaggtgatggccctaacgactggcgttaacgttccgttttgggatttttctgtaatccttcgcaagatcggttctctgacgttgctgattcagcttttggggcgaggaatgcgattgctcaaagactggcagaaggaagcgccctattcgtgggtcaaagaagatcaccttgtctgggattttgccggaacaatggatgacctgggccagttatatttcgatccgattctggagcaggcgcagtatcagaagcgcaagagcagcaagaacggcccgaaaatttgcccggtctgcaaaggcgaaaatagcgagtacgcccgccgatgcatccacaaggatagtaacggcaatcggtgcgagtatttttggacgtcccagcgctgcgaagaccaaaaggatccgaggacggggaaaatcaaggtcaaggggtgccacgctgaaaacgacattgtggcgcgccagtgcagatgttgtggcgtgcagttaaaggatcctaacgacaacctcaccgggaagcattacacgcaaaatgactggtatcaggttgtaggctttgatttgggattgacacgaaaccaaagcgggatcatctttaattacgtgttgctcaaccatgacggcgagcgtttcacggccagggaaaaattcttccctgagtcagaaagtgcgatttgcggtaagttgtggcgacaaaaggcagtcttccagcatgttgatgatgctgtaatgcgtggcaagttgggcggcatgaaaaacgcccgcaagatcctggagtatgcggaatacttccgcgcgccgaagcgcgtaacgcatcgcatcaacggcaagaaggaggacattatttcccgcaaagactttggaggcgaggagtgattacagacaaaggggattacctcgaatattacggcgggcctgtaaaggcttgcccgcttgaaaaaatcgaccagatgaacagcgtttcgtggctgcgttacgaataccccgattatttgttctggcatacggtcaacgagggcagcaagcacaaggcgagcgccgttgctgatcatcaaatgggattgcttaaaggcgtaagcgatatccttgtgctgattgggcttggcggcaaatacccgttcgccgccattgagctaaagcgccagggtaaatcccaggcatcgccagtgagcaaggagcaacgggaattcctagcagccgtccgaagccgtggcgggttcgctgccgtggcctatggattcgagcagtttaagatcgcgtttgaagacgcaatcaaatagcactttttgttaaaactacccggcgagagtcgggtaatattctcccatcgaaacgaatcacggagtattgaaagatgaaaaagatgcttgctttaattgttctgtctcttggtcttattggttgtagcgaaaaaccaaaaacatatatttgcggtaacgaagcatttgaggttacgagcgattatatgaaggtagttagcggcaagagcgctggcgttttaattgatggcattggtgaaaaccagtataagctgtttacgccacttggaacggctttttatactgttaacaaaaacaccattgatattaaagttagcgtttatcagaacactctaacttgcgaggttaagtaataatggcaaaagacacccaagacaaagacactcacgacgcgttcacgacgtttgaacagcttgaacgtgaaacgttcataggcaacgcgcttgccactggcggacactatcaggccgtcaagcctgataaattctaccaggtaacgggcaaccgctacgcaggtagcaagacgccggatatcgtccgcgacaagtggtcaaccgatcgcagccttattgcttacatggaggagcgttacgggccatacgacctggacgccgccgccgaggaaagcaacaccgtctgcccgaagttttacgacgaaaagacagattgcctcaagcgctggtgggggaaaaataagcacgtatggcttaacccgccttactcgttcccggatcccttcgtgctgaaagccattgagcagatggagcatgacaaccagatcgatattttgcttcccggcgacaactctacggcgtggttccgtgacgcacagaaggcagcggctgagatcatttggattgtcgcagacgtcacggaggatgaagacggaacgcaactaagccgatccggtcgcctggccttcattaacggcctaagcgggaaaccagtcgacaacaacaacaagggcagcgtgattttcatcatgcgcaaactcaagccgggcgaggagcaaaaaacgttctacgttccggtaagcgaaatctgcccgtcattaactaaaaagcgcatgcgcaaacgtgggatctgaaaaatggaacagcttgaatctttcaccgaatacctccacatcgtcgttaccctgctggacaagtacggcttcatcggtacgaatgaagaaaagatggccttcgccgatagcatcgacggcacttatcaagagttcatggataacggcacgccgcttgctgactggcccgcgattattgagcgcgaattgctggattttcgggcgcgtgaaggtgcggaatatttcgcgaaacagcactaattgctaaacaatgcccgccgcgagcgggtattattgttccatcaaccaatcaggagctaacgccatgaacaccaaaaccatcgccgacaccatcaagatcgcacaagttaagccgcacatcgtatcgcgtcacctgattaacctttctcgcctgtgcatggccgactacgtggcgaagcctttagagaatggccttgatggtgaaatcggcgcgatctacttccgagcagcccacggcatcgaagaaattcacatgtacgagcagatggctgaatgcttctgcatttacggtgacgaataatgattgtcgaatctggccgcgctgccgtatggcagcatgccaaagaggctggcattagtgacgatatcgtgatgatcgcaaagtatttcgatatcaaggatatctccattgtgttcaatgggaaatttacctatcttcatgagcgcccactgaagcgcacgcgtatagcggtcgcgacgcgagcggaggccgacgcgctgaagatgttcattaatgagtcgaagcagacaaaaaaatattacaagtagcgggagtgttgagaatgcgatatattgcgatcctgattacggcggtggttctcactatcgcaatctttaactacgcaattcaattgggataaattatgcaacctaaaatcacagacgaagaatttttagccgcccgcgaggaaggcaagacctaccgcgagatcgccgaagagttcggtatgaacgttcgcagtgtggagcgtcgcggcgttcgacttgcccgccaggggcatttgcacggtaacaaccacgtagcgaagcacatcccggacgggttcggggtcaaaggcacatcgaccatgattcgcgcgaacggtgacgaagttgtgcgctgggtcaagtcagaagtagaccgcgaccgcatgatcgctttgatggagtcggcgcaagcatcattctgcgaagacctcccgcgtgccgaaccgcagccgaaggatgaatcgaagtttttcattgaggatcagcttgccctgtatccaatcttcgacttgcacatcggcgcgatggcccacaagcacgaatgcggcgagaactacgacaccagcacagccgaaagggttcttaaccgcttctttgactattccgtttcggttgccccgcagtcgaaaaaggcggtcttgctggtcggcggtgacttccttcacagtgacggcctggacgcagtaaccccggcaagtggtcacgttctcgaccaggacagccgttacgcgaaacttgtctatgttgctattcgctccttgcgccgcgccgtgtcgctactgctgagcaatcacgcggaagtagaggtgcatgtgatcgaggggaaccacgatcaggctggtatgatctggttgcgtgcagcgctcgcagcgttctatgaaaatgagccgcgcgtattcgttgacgtcagccccgccattctgcatcgtaccgtgtggggcaaaacgatgctgggctacactcacggacacacgatgaagaaggcagaaacgcgcctcgccgcaatggctactgacttccgcaaggagttcgggcaatgcgactacatctacacgcattcgggccattggcatcaccagaccgtaacggagcactcgttagggattgacgaagtacacggccagttaggggccaaagacgcttacgccgctcgcggtggttggcgctcctatcgccaggcggcggttattctgtacagcaaagaatatggcgaagtaggccgttttatctaccgtccgaacatgtaaccacaacggccccgcgaggggccaacaaggaaaaccgatgaataaaaatatctgcattttcgatctcgatggcacgttgtccaatggcaaccaccgtttgcacctgctgccgaaaaaagatctccatctaaccgaaagctggacggcgttcaacatggcggctggcggagacgctcccattacggacacgatccgggtaatggaggccatgcgcgcagcaggatttatcgtcatcatcctgaccgggcgaagtgatgaggcacgcgagatcactgaaaaatggctggtgcaaaatggcgcatcgcattacgatctgctgattatgcgctccgcctccgacaaccggaaagatacggttatcaaagaggaaattttacgcaagattggcattgagcgaatcgttgcggcgtgggatgatagcccctctgttattgcgcacttccgtagcctgggtatcacaacttaccaggtctgcgactacggcgaaaaccttcacgaccatctgaaatctcatggagtagacaaatgaaaaatgtaatcatcctcaacggcgcgcctggcatcggaaaagacaccatcgcggaaatcatcgcccgcaaatgggagtatcggatccttagtttcaaatcaccgatgtttgacattgcgcgcgccatcctgggtgcaaccgactttgcccgcttcgccgtccgttaccatgaccgcagccggaaagaagtcaagtgcgactttttaggcgaccgctcgccgcgtgaattcctgattcacattagcgaaaatttcttaaagccgacgttaggcaaaaagcagttcggcaagttgctttgcgattccgtcgtgaattcgcctgttagttgcgtcgtcagcgacggcggatttgacgaagaagtagagcacatcgcggcgcatgatagcgtaaacgtctttgtcgttcgcctgcatcgtgacggcatgactttcgagggtgatagccgcaagcatattagccgcccggatctgatttgcgaaacttatcacgaattcgattttgatatgacgactggcgagccggaagacgacgcgcaaaaaatccttgatatggtttttgaagggtattaatattctctacgtaatgcctttattatcaccgccttaaactttgggagccttaacaggttcccttttttttgttctttatttggcccaacgcatatatcatcgcattatcacttaactaacagaggttgcatatcatgcgggaattcatcaacgcggcaaccaatggtagcggcggtgttaccctcgcaggctctgcgaccgggcaacttatcattgcggccattggtttatttttcatgattctattcggctccttcggcgcgtggttgcgctggcgagattcaaaggcgcttcgtgacgcgctggaagccggggatatcaaaacggcggtgaagatcaggagtaaataacatggggattaaaacgcgggttacattcgcggcggcgatggcgatcgctgtcgccttcctcccgaaagtggaggacacgaaatacaaggtttataccgatatcgctggcgtttcgacggtctgcgaaggcatcacagggccggacgtaatcaaggggaaaacctatacccgatcagagtgcgatgctcttttggcgaagcatatccaggtcgcaaagcgtgtggttgacagcaaaatcaaagtggccgtgcctgacaccttcagggcgtcgatgtacagcttcactttcaacgctggcggcggcgcatactccggcagcactatgctgaagttgacgaaccaggggcgactatatgaggcgtgcgaacagctttaccgctggacgtactatcgcaacccgaaaacgggcaagatggaaaaatcgcgcggcctgcataatcgccgggatcaggaattcaatctgtgcattaaggatctaaaatgagtagcttaaattttcagagagcactagcaatcggcttcatcgtgtgggcggttgcactggtttccggttgcgcttccagcgtcccgatcctttccgatctggttggcagcaagccagatatgacggcgcaagtcggcgcggagaacgtgaaacaggcggttggcgtgacgaacaaaacggacacgtcgagcaagcaggaaaccaccttcaaagaatcggcagttggcaaagttgacacttccaacaaaaaggcggtgacgacctccagcattcacgcgaaccagattacggcggagaagatcgagatccgaaacgacgatagcggaagcatgattccgtggctgattggtgccgttggggccatcatgttcgcggtcagtgttttcggtatgtggcgggagcgaaaaaataaaggggcgtaagcccctttttttatatgtaccgtttgacgtgcagcagtgccacgccatcttcatcattcaacccatgttcaaccgtgtttgtcgcggccatgccttgatatagcaaggtgagcgcggcacgcaagtaattttctggtgtaatctgattcacgcaaacaagcctgtgaacctccgttatcagatcttccacctgctttcccgaatagggcttcatcgtcgagttgttcaatatgcatcatctcccacatgtatttgttgtccatcccttcaaacgaccggaagttaaatccaatctccctgttatccgggccagtgcaccacaccacgccgttttttccgtccaggtatccattcgtatagcttcgcgctaaaaactccttagaaaccattgaggcgaacatacggtgtgacacgttggcagcttttgccatgcgtggcgattcgcggtgcatgtaaacgaacttcgcaaaatcttgccgggtaaattcccggcgagactcgcagaatcggtaaatgtcaaggataaacattcattacctcatgaacgaaggattaacgaacacttccgaatcaatcacgcagacgaaacctaattcctccatcttcggcagcaatttttccttaatccttttgctcaccccggactgacctttgaagatcttcagattgcggcacgcgttatagatgctctgaacagtcatcacgcctttcgactgcttgcaacggctggcgataacatcgtaaacggctttaatctccgctccctcaccagcaaagccggacgaatcagccgacgacaaatacgttttgctcaactcatgaaacatgatgatggcttcgtcgatcgtttcggttccgatcttcttcgagcgcttgccgcaaggttgccagttgcggatagtgtggattaccgacgccaggcgcatcacctgtttatcgaatttacccatcgcaccgcgcagcatcgtatgcgaatacttgccgccgtcgcctaattccggctccaactcctggcgtgccttgttcagtcgacgcatgcctgcttcagtcacttccagcttgatatacgattccgtcataatgtcatgaatgaggcggaagtaatcagcgcgtaaagacttgtcgatcggctcataggtcgaattcccgttttcgtcaatgaacacacgctcacctaaccgcgtttgctcgcgaaccaaaaggaaacgctctgatacaccgatgccgcgagaaccagcttgcatgatggcgtcgatggtttcatcctgggcgattacgcaaatgcagcccagcgcgaagaatgacatattgttactgacgtctgaacgtgcgatcgatacgtggcccttatcccacgccttcaacaccagttcgctgttcgtcttcttgccgccgtcgttgccatacgtaatccctaacaggctgttaacagccgtcgcttcatccgagataaccgcaaagttgccttgccgattgttgattttcgccaggccttccggcgtggtatcggaaaccgggaacgtcagatcgcacatgctttctaacttctcctcaagttcgtctttgtcttcgaacagtttcgccatatcggactgtgacaactcgcctttcatggcctgcttgttggcggacaacttcgccatgattttcttgcgctccttcttgcgcgcctcgttgattcgctcaacctctgcgacgatcggatcgatagccagcgagttaatcgcagacttaccagcggaaggcggctgcgacgtaatgacataaagcgtcgtcggttgctcgctgccgtggtactccacccaaaagcggcccatcatagcggcggacacggttccgaggaaatgcatataagcagatgattccgggaattgaacggagcgagcggcattgagcgccagcttgccgacgatatcgtaatcatttgcgatcgagatcgtgggatacttgtccgcgtttacgtcgatatctttcggcttgggccagaaagaaacggagtcgcgatacccgttcgcacggatggcgacgcgcaaggggctgatgccttccttctctgcgatggcaataatatcttgtggggacacgcggtcatttaaaaacatgccttgctcctgattggttaatcgtgcggctaatgataagccgtcttaaatccgagaaccagtgataatctaaacgcacttgcaaaatgtgcttaggttatcggggcgcatggccccgaatccgttacagatatttcgcctcaaacgtggtgccgtccgacacgctgaaaccgacttcctcacggtagagcgtccagcggcaaccgtcacggtcgaagatatacccggcgacgccgcccagcgcgcgaccgctttccacctggtagcgcttgccgacggtgaaggattttttcaccgggtttttgtggtcgaggccaacgcatttcagggttttcgttttcagttcgataaacgtcgccaccaccacaccgccaacgccagcaatgaacaactcaccgctaatgccgaccgataagtaaacgcgcttcttcttcagttcggcgctgttataaaccatcattgacacgtttccttcgtcgtcaatgcgagcggaataaaggttgttttcctggatattaacagcgcggcttgacgtgcatttaattttgattgactttgccattttatttcctttctcccatttcgatataaaggtttaccagatccagaaagtcggatttgtttctgcaactcaatttaaagccaacgtgcgattcgatcttattttgcatcgccgccatcgttgcgccgccgttattcatcttcagaacttcgcggcatacgtcagcaagttttttagatatcatttattcgcggcctctattttttcgcttacaaaatatctcttcttatcactagcccagcgcttaaatctaatatcaagcattgttttgtgatgcatgacgtcaacaatcccacaaacccacaaaatcatgattgaaaagataagagcgccaggcccatacatagaaaatagtgcggccattactgcgatcgtgaatttcattctggttttctccgttgcgtttcgatggaataactatacccgcaattaagcgggtacgtttagcaattcgtgctattcattaaaattcgcctggaacacggcgcgagcgaatccgcgcggagtgatagagcgaagcatctttgttctttcagactttccgccaaggaatttccacgcccaaaagaagtcgacgccatccacaccttccggcggtaataaccgtttaggctccacgaatccgttaccgtgccagatgcacgttttcttcgtgtaattgtcgcagtgcggcattttggggtgccaaacttcctcatgcggcgcaacatacccgccgaagtcgcgagggtggaaataaaaatccggcttgcgccaaagcgtcgataatttgccaaccgggttttctaccatccaggggcaattatattggtcgcccaattgctcgatcatcttcgcatcataggcggctgatagaacatcctgagtctgccgggcgtgcttcaccccgctatgcgctaacagcgtgcaggaggggaaagcgaaaataatgtcaggatccgggatgcccaggatggtgcgcttcacgtcgaaatctttgtcaatccagatgttgacatactggattttggaatgctccatgcggattttgtactcgccgtggtcgcctgaatcggcattgaagcaatacacatcgcaaccgcgaattgcccacggcagacccataatcccggaaccgtcaaacatgcaccacacaacgcgctttttcattgtcacctcaccagaaagggatacattcgcggcaaccttcttcaccagtgcagccgcaactattttcttcttgctctaccagatcgtcacgcatcggcagactttcggcatccatatccatactgcccagggcatccgatagcgtcatttcctgattggctactttgatgcaatattcgcggttaagccccgcgtcctgggcggttaaaaaacggtgccagtaggcatcgtcgtaattatcagccattgcttttcctccggttaaattctcgcaaactggcggcgcatgacttctaacaggttttaccccatccacgtttaagatccgccgccttgaccttatagacagccccgcatcctggagcctcgcagcacttcatgctttccttagccccgacgtgatcctgtcgaccagtcagggagctatcgccgggattcttcgtcgaccagaacagttgcactgtcgcgacctcctcgccgaactcattaacgaacatgtaaatgcccttgcttttcagaagcacggtgcaatgatgcatttcgccgttgacttccaggtatgcgcgatcgccctctttcattaacttctccacgactcaatgaacatgctgttcgattccagcgtcagcgtgacgccagccagggaggacggaatcaggcgagcgcccggaaccgtgccgacaacatagcgaccattcgatttggtgatcgtgatctgagtaaagccggattcgcggttcatgcggagaacaacgcgaccatgtgcatgcaggacggccaggatcgaatcagtctttggatggttcatgttttatttcctcgttttgttggtgtgggggtagtatgccaccatcccccagcgatgttttagcaattcgtgctattcgggaacgcaggtcgtgtaatcctggcagatgtagattgtgcgctgaacgcgcggcgcggcatcctccaggctctgaatctcgatatcgttgctgtcgacccggcagggtgttgcaccgaaaatgaaataccccgtctgcttatcgttgatttgccacgccttgctttcgggaaccgtgttgacgacaaacggcgtgtcgtcggctttgaagtcttcagggtgacgcccgtaatagtggatgattgcagcagttgccgccgccttaaacttttccgtcacgtcacgggcggcgatatcacgcgagatctggatatcaacatccggcgcgccgaccatgtaggtaaagctgttcgaaatagtaactgttctcatttagattttaacctcgtttcggtggatgaagtcggcgacgtaatggcactcgatcgggttttcgatcccggcgagaacctcacagcgcccgttgttcagtaactggcaatgctggcaatcagcgccgccaacattagctatgacgatggcgatcccgttgcatgaaccgccaaacattctgtgcgggaaggggtaagcctcgcaggtgcaaaccacctccccggccctacgcttcctcatgcgctacccttctcgatgcagaatacccaatcgcgagcatcaaccgcaaagcgcgccacgccgtcaagcacgatgaaaacggtctgcccgttaacgccgcgccctttctcgatatcggtgacggtgcggaacaggtcgcttgcgccatattgaattttgtcaccgattacgacttcagcaaatgctttcatgttgattctcctgattggtttcgatggggtaatgataccaggcctcgcggcccggcgtttagcaattcgtgcttatttgatggattttttagcggctttgtatgcttctttgaacgccggaacatcttcgatctcaatccagaagccggagccatagccatcatcatagcatgggcattggtcgcagtagtcgccccaggttttctcttcgccgccgacagagaagccagttttcatttcgcagagcgcttcttcaacggcttcgattttatctgcatcggaatccatgattacgaaattccatttgccattgtattcgttgccgagtttgattgattcgcgcttcagtttcatgatgtttgcctctttgtctgcttcgtttcgatgaagtaataatagcaaaagcccacgcggggcttttaacaaaaagtgctatttacaccaggcggaaccgaacttcatcaccgtaaatcacggcctgaatgtgatcgccaaatgtgactgtcgcgcgtgtggtttcgtactccgtatgaccgtatcgccatatcccgcgctggggataatgcgccgcgctttcgtgccaaatgttcatagtgaactcgccagactccccgaactccaggcactgtccgaccatcgctttgacggcctggtagtcatgccaacgttgagtagcacgatcggtaactcgctccacatgcttgcgctctttgtgctccgccgctgcctcaaccacggcccacttactacgggtaaagattctcattgttggtactcatttttgatttgggtgcggaaccgcgaagcatctttttctgcccgttcagcgctgtgatatcgtttcagaaacagggtatcaatccccatgcaaagcgtttcgtcggcgctgtatttccccacaagccaaagactgaccgtctgcctgtccagaccgtagttcttgtcgaaatgaaggcgcacattcacagggacgtatccatgcgtcgaaatgaaggcatcaacgcgatcgaatagctcttgcttgttcattattattcctcaacggtgaatttaatcggttctttcccggtcgccttacgatgggagttgaccagcgtttcgaacttgccgatgcactcgcgcaaatcagtgaacgtgcaagcgctacccatgacacgccacgattcaacatttttaccgcgaacgtagttgatacagtgcttcatgattttgtactggtcgccatcctttttggtgtacacgtgcataaccgcgatcctggttgatttgtaggcgaatgctttggtgatgtatttgcaagtttgcatcgtgttcgctcctgactggttgatgtgatgactatacccgatcccactagccagattttgattatttgtgctattcctccgattgtcgattattccgttattctggaatagtggaataggctgtgattgcatctgattacttggtgattacttttgacgattcatgattgattgtgattgtttaattctgtgcaatgggatgatggagtgtttcatgatgtgcaacgaattgcatagatgcatgcaccatgcacataatttgccgttcgaatcctgccaaaacggttgccagttcacagtaaccgaatgcagttcacgcgcagttcagtaagataatcttacgtaaaaattgatcaaaaagtgcgcagcataagcatataaatatacatataagtatatgataaacttaatattattattattattctattattctctctatctgaatattccagttcacgtaagatagtaaggaggtgtgctaccattttcgcttgcgacagcctccaaaaaattcagtgttccgcgccgccagggtatataggagggtatgggtatatcttactttcttacgcaaactgacttgacgccttgatacgcctggcttagtatgcttactgaattggaacggaagtggagagaattacagtgaacaagagcgaaattgaacagaaatgggagtttgaacaggtcgatcccgaagtgtggatggcatatccgccggataaacaggccgcttatcatgcctggctcgcgggcgatatcttcggcgatgatgacgaaccggagccggaacgcaagcgcaggcaggtcggcggtaagcgagaggggccaaccatccagaacctgattgaacaggattttattgatggcggtctgtccggcaaggtcgacgtggaggagttcagcgccacgcatggtaagcagatcacatcgcacgccatccggtcggcactgaagcatcacatcggcaaacatggcaagttcagaaccaggacgcgcgacggcgtgatcgaatactggattgaacgcgatgaatcagcggttagcagtgatgagcaaatccaggcagaccgggaacgggttatcggaatcattcacgcgctgaccggaaacgatcttgatgtgcgtatcgaaccaatcacggcccgcgtgcttatccaggccatcaaactgaacgtcaaagcaggagatatcaaatgaacagcgccaggattatcagcaaagccgcgaaggcagagcaaaagagggcgattgagatcgtggaaagcacaatcccgaaccatccgctgtacagaaagtgcaaggcggagctaatcgccagaatcaaaggggaaaaggtatgaaactctatcaagaccagtccgacccggcagcaccaatgcgcctgggaagcgagacagtaagcgagcaggacgttatcgacgcagtaatcaatgcatcacaggccgaaaattcaagcgccttagaatgcgttacaggtgacttgcttttgatggcatttcttccagattctgagtcatacatccagcacatcaccgaaacgggccaggcagtgacgcgaccggagttcgtcggagcgcctaacgtgcttcagatgatgcttgacagcgctgacaatatcgttagcctggactatccgcaatagcactttttgctaaaacttccgcaaggccatttgatatagtggccttattgaagcgaaccaatcaggagctaaacaaatgagcattccgtatccgaccaaagacaaatcattcctgggcctggccgttgttgagattgtcaacccggatgtgaccaactccccggatgagcctacccttgcacagtgggccggattcaaagcaggtgatcgactgaccgtcgaatgctatagcgacggcgagaagtgggcgcaggtagggcgcgactaccacaccgaagaattcggccaggtgctccctaccgactggttcaaacttaacgaaggcgaatataaggtgatcgaagaatgaacaacaaaatttgggtgctgacctacactatcgggactaacgaggggcgcaaatcgcgccgcctcacctgcgacacgaaagcgcaggcggaaatgcagcagcgcgtgctgggcggcgaagtggtcgaacacatccgccagccggaatccttccaggtcaattggcctgagaagatggatgttgacgccgtgctgcatgaaatgcgccatgtgcaaaacaaccctgcggcgtggaaagatctgtacctttgcgacttcgacgctgagtcagtgcgggatcccttccgccttgtccggcaggcgcacgcggaatggtcggatcgccagttcggcaacgttggcccggtcggcccgctgaaacacctggcaaaagaggccaacgaagccgctgaagcgccggatgacattagcgagttcgccgatatcatcatgttggtgtgggacgcaacccgccgcgccggaatcaccgatgagcagttggcagtggcggtggcggaaaagctggagcggaacaaacgccgccagtggggagccgttaaagatggcgagccgtgccaccacttgaaaaattaacgaattcgtataccatttaaagcgcctgtaagcgattctggcgggcgcaaatttcattaaggtgattctatgtctgagttctcaaaagtcgaagatatgccgattggcgcaacggtaacgggtatccagatgagtgagtcggtcgatacgattactccgctggccttccctgttacacaggtcgaaaccgatagcaaaaaaggtttcatcttcatctacaaaaatttcaacgcccctctgcgtgtcgagatcttcattgctcgcggaacctgggtcgaatgggagaaggcttaaatgttcggactgaatgaggcgcagtacaacgctgtgaaacgcgtggcgaaacgaatgacagcggaaaccaaagacgccatacggaaagacaaaaagacttacgatcaggtcgccgcccggatgatcgataagcattgggccgaagtcaacacgctgttaactcgcggtcagtttatctggctggctggctatctcgaaggccgtttcggtcgccgtgatggcgagtatgagtaaaaagtaagcgaacgaatcaaccgcctattgacgcattcacgctgaccgggtatcgttaaaagcgtagacacaagaggcggtaagcatccgcaagtctcgccccgcttaggggcttttattaaggggtggtgatgcagtacaaggtcatactcacagcaaggaaaatgggcggcttttgcaagtcctgcattcaagagttcagcatgacgattgaagcgaacgataccgccgacgcggtggagaaagcaaaaaagcaatccggcgtcaatctggatacgcataaaatcaacataaactacataagggaagttaatcaatgttaactctgattattgcattgttaatgctgttcataggctatcacgttggcgcggctcatcttgtcgagcgcctttcaaagcgggtacatgaaggcacatttgcggccatgatgtacaataagaaaacggcgcgttgggaaaaaattggcgatccggatggtatcgcaaaacgaattgcgttttcgccgctcccgtatgttgactgtgagccttttgcatcacttcagaaaacactgaagcggcgtaacaaactgatatgaacataacccgcttcggcgggtttttttgttcctgcaatctggtatactcgcaactcaacacagaaggaggattcacaatgtctgaagaacgcaaaaagcgcgtaacgaaatcgcacttcgaaggcaatttcaaggcgctatacgaaaaagattttggcgtagtgctggggcgaaccgccgaaatgacgccacaacaattcttcgagatcgcgaagggttatttccagtgggccgaagaaaacgccatcaaggctgcggaaaccgccactttccagggtgacgttaacgagtggggcgtgaacaagccgcgcattttcacgatcacagggttaagcctgttttgcggcgtgaaccagtcaacgcttgtacgctatcgccacgaccctaactatgccccggtcatggagtttatcgactctgtaatctatgagcagaaattccagcttgctgccgtcggcatgattaacgcttctttcgtcggtaaggaaatggggatcgacaagcccgccgttctcaacattgacgccgtagctggcaacaagaacgagatcaccgatgaagtattggagaaggctgttagtaacattctggacaagatttaagggccgatcatgaatgacgaaatgattatttgggaagacctgaagccagccgacaagctggcaatcaaggcattgagcacgcgcaacttttcgctgttcctgaagatctggtttcagattatccagggcgaaaagctgatgtggaactggcaccactcctacttttgccacacggtagatgaaatcatcgccggaaagcgcaaaagcacgatcgtaaacgttgcgcctggttcgacgaaaaccgaagcgttttctatccacctcgcgccgtatgcgtatctaaagtgccggaaggttcgcaaccttcagatctcgcagggtgacgcgctttcaaaaggcaactccgaccgcgtaatcaagatcttttcatcgggcgaatggcaggagctatggccctcgaaattcgggcggaagcagatcgacgaattccaggttttggatgacaacgatcgcgtaaggctggaaatggtgtcccgttcgtctggtggtcagatcgtcggtaagcgtggcgggtacatgacgccgggtttcagcggcctaatcgcgctggatgatatcgacaagcctgatgatatgttctcaaaggtgaagcgcgaaaagagccacatcttgctgaagaacaccattcgatcccgtcgtgcgaagaagaaaaaaggcgacgaaacgccgatcctgtccgtacagcagcggctgcatgcacaggattccacttggttcatgatgagcggcgggatggctatcgaattcgatcgcattgttattccggcgatggtaacgagggaatacggcgaatcgctccctgactggttgcgtcctgagttcgaacgcgacgtgttatccgggccgtcggtggtcattgatggtgtcgaatactggtcattttgggaggacaacgaatcgatcgagaacctggttgccttgcgcgatgccgatctttatacgttcctttcgcagtatcagcaggagccaatcgcattgggcggcaacgtgtttaagtcggaatggtggcgctattacggagacagcgaaaaggctcatgaaccgcgcccggacaagttcgaatacaccttcattacggcggatacggcgcagaagacgaacgaactaaacgactattccgtgctgtgctattggggcaagtaccgcgatcgcgtctacttcattgatggaatccgtggcaagtgggaagcgccggatttgcgcgtgcaggctgaggcattcattaagcagtgctggcgcagaaacaaggaatgcgggaatctgcgaaagatttacatcgaagacaaggcgagcggtacgggtctaatccaggatctgaccaaagcagttaatggcatgggtgagatcgtcccggttcagcgcgacaaagataaggttactcgcgctatggatgcgcagccaattatcaagggtggccgcgtcgtactgccggacagccatccgttcattgcggagcttgtggcggaaatgagcgcgtttacctatgacgattcacatccccacgatgacatttgcgataacgtatttgacgccgcgaacctggaaatgaacctgagcgatgacccggtagagcgaatgaaacgcctggcaggattgaaaaagctgggccgctaatacataatgtgggcttaacggcccacaaatcacacaaggttaaaatatgaaagctatcaagatggatgattacaatcagatcttcaatggtggcgcggggtacgcgtcaaccatggcgatgattgcaagcacgttcgggaatatgtcgcaaatcgaggagttctatcacaataacggactggcgaaaaagattgttgacgtgatcccggaagaaatggtgtcgcccggcttccagcttaacggcgtttccgataacacgaaattccagtcagagtgggacggccttaaacttgagccgcaaatcaccgatgccctctgctgggcgcgcctctatggtggctcttacgtcctggcgatggtcaacgatggccgcatgctgacttcagcagcgaagcgcggtaagccgctggaatcaattgtcgtttacgactacgattcggtgtccgtagcggaggaggagaagaacccgcgaagcccacgattcggcaagcctaaaatgtacaccgtcaagccgttaaacggcggtagcgactttaacgtgcattatacccgcatgcactacatcgacggcgatcgtgtaaccaacaaggtgcgaaaactcaacaatggcgctggcggcacggtgctgaacaagtcgatgattgaagcgattcttgattacgattattcggagtacctagcaacgcagttgctgaagcgtaagcagcagggcgtatggaaggccaaaggtcttgctctaatctgcgacgacagggagggcgaatacgcggcccgattgcgcatggcccaggttgacgcgaattccggcgttggaaacacgatcggcattgatgctgacgacgaagaatacaccgtgattaactctgatatttcaggtatcccggaattcttgtccgccaaaatggatcggattgtcgcgctgtcaggtattcacgaaattgtgctgaagaacaaaaacacaggcggcgttagcgccagtcaaaacactgcgttgcagacgttttataagctggtggagcggaagcgcaacgacgactacaaaccgctgttagaattcctgctacaattcatcgtcactgaggaagacttcagcgtagagttcgaaccgctgtcactgccgaccgattcagagaaggctgatatctttaagaaaaacgccgaagcggtacaaggactggttacggatcaggtcattgacgcgaacgaagcgcgcgataccctatccgcgatggttcccgaactgaaactaaagggcaatgcgccggaacagaagcagctaccggatcgcacgtcagggtcaggcaagaccaaaacgcaaagcacgcagatcctgaacaacaccgaggagggcggcgatgaaagttaacggcagaattccaaactggcgttatcctgaagcaagcgagcgggatttatcccgcttgatgcaggatgcgacaaccgatctggtcgtggaaatgcgggatcggttagaccgcctgaagtttgacgccacggctgaggagattaacgaggcggaagacgatatcaacgaggcggcgattgcgttctttgccaccgtcattgcggcgctggcctccattggtctgactatctaccacttcaactctaagcagtggctggttgttgcgctggcggctggcgggcgcgataatgaagcggtcatgatgctgaaggagttcggcgctggtggttatgagcagtggtatcagcaggagctgaagaaatggcaggataccgcaaaggcatcaatccagaagttaacagccgatatcgtcgctgactggacaacgaaagttcgaaccgccaacaacatcggaaagactcgcgaacagatcgatgaaatcatcgagggccgatacgccatttatggcagttggtcgcgcaaccgggcgagcggcattatcggaagttttaacagtatgttgatgatgcagcgactaaaagatgctaaagtatcgcattacttttggttcggcatgatggacgaccgcgaacgcgaaagccacatcaaactagaaggtaagcgacgccgcgtaaacggtgacggcatttttcccggcgaggagtacggttgccgttgctgggcggttcccgattttaataacgtagaggaattacaatgaaaagagttcaaagattcgacacggtaaaggtgaaggcccgctttgatgaaaacggctttctggtcgataccccgatcgttgcgcgtatcggcgcacagacctaccagaccccgaacggcccacgcgttgagtttcgcccgcgctccgaggtgttcgacgctgaatcgctggcgtcataccagggcaagccaattactttgggtcacaagatggtcaacgccaaaaacgcgaaagggctggtggtcggatcctgttccggcgctggcaaagaggacgggatcggggttctggttccggtgatgatttacgacggggagtcgatcgagcaggcgaaacggcgcgtagcggctgaattgtccgttgggtacacctcgatcgatatcgacaaaaaaggctggggcaacaacgcgacgggcgaatactacttcgatgaagacctaccggaaaacttcgaagaactgaaaaacgattccgtctctgattgggttcgctttgatgccgtgcaaacgaagattcgcgttaaccatgtggcgcttgtttttcgcgggcgtgctggaattgcgaaattaaatcttgatagtgagcaagaatttccctatgatgacgactcaaaccacaaaggagctaaaacgatgattatcaaaattgacggcgtagacgtcgaagtagccgataacgtaggcgcacatattgctaaactggacgcacagatcgcaaccgcaaccagtcaggtaacgagcatcaccgcagagcgcgacgcacttcaaaccaaagttgatggcattgaagatgaagtggcggcacgcgttgccaaaatcaaagccgacgaagacgcgaagcagaaagttgtcgcgatcgtctccgccgctggcatcaagtgcgacggcctggacgttaaagcgatgaaagttgcttacatcaaagaagcggatggccgcgacttgtcagataaagaagattcgtatatcgacgcttcttttgactttatctccaattctgataagatggcaagcaatcgctctaaagtcttcgccaaaaaagaagatggtgagcaggaagacaagaagggcgcgccgaaactcgatggttctaacatcatcgacccgcaagccaaattccgtaactaataccgtgcggccttcgggccgctacaaaatccaaaacaaggagatttaatcatggcaattcctgcaacctatacccgcaagcgcgatatttgcattccgggccagatcgcggatacttcgctgtacaacatcgacggcacctgtgccgctgcaaatgacattttaactggcgttatggtcgcgttgtccggtggcgttgttgatggtcataaagtggtcgataccgctaaaactgctgatgctgttctggtgggcgtaaccactcattcccattaccaatcgccggaattcaaatacgacaaattttccgccgtaaacgttatgacgcatggccgcgtgtggtgtcgcgctgcatctaccgtaaccgcagctgattgtgcttttaagtcgctggtaactttcggtgatgatggcaccgtggcaaaaggcgatgctggcctaatcaaaaccgggtacactcacactggcgagtggttcaaaaacaaagatggcgtagtcctggtgaaaatccagttaacgcaggacgcaaccgcgccagccgctcccgctactggtggggcgtaaaaataggggcttcggcccctttttcatacgttaaaaaaacctttgaccgtttagcaattcgtgctattcttcatctcgttaagtcaaaacacacaaacaggagtttcaaatgactatgaaattagatgcattcgagcagaatgcaatcaaggttgcaatgcagggtatgggcatcgacgctaacaaactcgacgcgcacggcatctggactgttaatcagcttacccaactcttaaatcgccagtacgagcaggcttacccgcaaactggtgtgctggagcttttcccggttactaccgaactgagtccggtaacgaaaaactttgaatggctggagtttgacggcgtgacctctgcgaaaatcatcgcggattataccgacgacctgccgaccgttgaagcgatggcgtccgagaaaactggcaaagtgttccgcctgggtaacgcgtggtttatctccatcgacgaaatcaaagcaggtcaggcgctgggttccagcctgagcgaccgcaaagcgtctcttgcacgcgaaggccatgaaaccttggttaacgatctggtgttcaaaggttccgcgccgcataacatcgttagcgtgttcgatcagccgaacattaaccgtctgacctccgcaagctggactacgccggaaatcgcattcagcgagttgcaggatctgatcgagacgatcgaagatgtaacgctgggtcgtcaccatgttaccaacatcgtgatcccgccgtccaagcgccgcctgctgacgcagaaaatgccggacgttacggaaagttacctcgcgtggttcaaagagaactacccgaatgtcaccatcaccgcgattgcggaactggaagatatcgacggcgcaggtactaaaggcgtgctggcatacgaaaaagatccgatgaatatgtcgatcgagatcccggaacgcttcaacatgctgccgatgcagccgaaagacctgcatttcaaagtgccttgcacctccaaatgcactggcctcatcgtgtatcgtccgctgactatcgccattctttctggcgtgtaattagaatggcccttcggggccattttttattgttgcgaaaaacctttcattgtgtttaaatcaaacctcaccaaaacaggagagatataaaatggctaaaaaagatactgctaccatcgaaccggaaatcaacgaaaagccggaaagctcacagccggaaaccgcgaagccggatatggtaacaatgcaaaacgtgggcgcttgcctgattaagttcgacggcaagaaagtgctacctggcgaaaccttcgagatcgaagagtctcaagttgaccgcttccgccacgatatcttcaaaggccgcgtagaattccacgatgatattcgccgcactcgcgactacatcgcagcagtgaaagcgaaagcaaaaaccatcgtgcagcctacgagcgccgaataaaaaaaaccaacaagggcgctacggcgtcctttttcatatctggagaaagaccatgaattatacgattcaagatgtgatcgacaaaatgcgcagcctcgcgccgccgcttaaagaagtacctgaagaactgttaaccgcgtgggtggtgctggctgaggaattcgtatgcgcgtctaagttcggcgattcgatcattacagcgttggcgctgatgaccatgcacctgatgtttctggacggcgcaatgaagcaggaaggcgaaagcgtggaatcatactcgcagcgtgtagcgtcattcaccctgaccggggagttttcccaaacattcgatcgcgtgtcttcggctagtgacaacgaaatgctatcaactccgtggggtaaaatgtactggcgcatgctgaaaatgcgcggcggcggcttcggccttatgaccgctggcggtcttcatcgttgcggcatgggaggttaaacgccatgaactacaaagcaattcaggcccgcgctagtgcgggcatcaagtttttcagcgatgccgatggcgtattcaacaagtacaccaaaggcgctggcggcggcatcgaccccgaaaccggggaggatatcatccctggcgaggttgtgaccacaatcaaaggtgcggtgcgggatatcaaagatcgcgatatcaacggcgaaactatcctcgctggcgacaagcgcggcttcttcactcacgacgtaccaatcatggaaggtgacgaaatcgacgtcgacggggagcgctatcgcgtggttaatgcccgcccggtgaaaccaacgggaaccgtcgttgcttaccgtccagttttgcgaagggtggcgacttatggctaattacaccatccgtgaattcacaggtgcgattgatgcatggtgcaaggcggctggtgatgctctggaggacgttgtacgcatgacgtgcgaggatatcctaaaggatcttgtgatgcgttctcctgtcgatacaggccgcttccgtggcaactggcaaatcacttttaaccgtgcgccactttacgccatcaacgcatacgaccagaccgggacgaagactatccagaacggcaacgcgaacattgctctgttcgcaaagggagccgggattacttcgatctggttcagtaacatgctaatctatgctaacgcgctcgaatacggacactcaaaacaagcgccgaatggcgttatgggagtcgtggcgatccgattgggcgtatacgtgactgaagcaatcaaacgagcgagggcgaaaaatgcactatgatattgcgttaaaatgcaaggcagcggcggctaaatttgccgacgataacgggattatgatcgcgagcgataacgttgactttaagccgccagccaacggcggtacgtatcttaaattttcatacgtcgaagcggattcccgatctgttagcctctccagggagtgtcgcgtttacctggggctggttcaggtcgatgtgatctttaaacccggaactggcaccgacgccgcaaggttaatcgcgcaaaacgttgcaaaatatttccctgaaggtaaaatcttaggcgatgacaaattagatctttacgtaagtgagtgggctgaggttcatggggttcagaaggcgcaaaccgggtggttcttccccgttcgctttacggtaagatgtgaacaaacggaggcgaacgggtatgcacttacctaacgggagtaagatttttattgaatcaagcagcagttaatgaaatgcgggttgtgacgctacgcatgtctgtacgaggcgcttataatttcttgccatccagctaactagcgggcataatggcgttgttaaactttcatcaaaacaggagtattcaacatgcatttaccaaacggtgcaaaggtcttctttgagaagactcgcggtgcggaggttccgttcaccgcaatgaccaacgacgcgaaaaacccaaaaatcacggtggcagatggtgcgctcgatgttaacgatatcgtaatctttaccgattgcacttggggcgactttgttaacaaggtggctcgcgtaaaagcagtggcggcaggcgtggcaacacttgaggagttcgacacctccgacaccaacaagtatccaaccgccgcaaccggaagcgtgagcgtcgtcactgactggatcgaattgccatgtattcaggatttgggcaaggatggtaacgagcagcagtattataactatcagtgcctgagcgacgaacgcgagcagtccgaaccgacctacaaatctgcggtaacgctcaactaccaatttgcgcatgagtacgacaacgcgatctacccggttctgcgtgctgccgatgcgagcaaagaggcgaaagcgatgtacatgtatgtaccgcgagcggcggaagttcgttattggtctggaacggcttccttcgatgatatcccgtcgacggctgtcaacgaaatggaaaccgtgaccctcaacgttgcacttaaaggttcgcacgttttcttacctgtaaccaacgcttaattaactggcggggcttgtgcctcgcctttttttgtgcataatagcaatcaacacataccaatcaggggattacaaaatggctaagtttaaaattcgtattggcggcgaacttccttccttcaaacttccggtaactttcacttgcccagatggcaaagatgcaaccatcaacatgacggtaaaacatcactccaccgatgaaatgaaggagttttatgaaagcgaagaaaaagcgccgaaaggtaacgtggattttatccgcttcatggccgaaggctgggatctggacgaagaatttacggatgaaaacatttcctggctgtgctcgcattacccttcctttgtcatggcattgcctcaaacgtacatggccgcgcttgccgggcatcgtgcaaaagtctaaggcgggctgtttatctaacgcttcagcctgagttaaccgatcgccagcttgctgagtatggtttacggcgatcggattacgaggctgatttagaggaaattttcttcgatgagcagaccgcccaaagttggcaactattccaggccatgcaaacgcagtggcgcattggggtgaatggcccaacgggtctggactataatacattgcctatgttctttgaattgtataaaatcgacaatcgagaagcggcattgcttgacttgcagatcctggagggtgaatacctcaaacagatctacaaaaaatcacaataagcgcctacgggcgcttttttcatatgggggctaacatggctgataaagtagctggcttgacgttcggcgttgacgtgtcgcaggttgacagagcggtgcgatcacttgccgaactgaagaaccaaagccaacaaaccggggcgggtcttcagtcgctggcggacgctgagaagcgcgcgacggcgcagactgaggaaatgaaccgcgcgttgcagaaccaaaagaaaaccacggaaaaggccaagaccaatttcaacaacatcgcgggcgctatcgatcccacgatcgcgaaaatggctaacttgcgcaaggcttcggaagaactggataaggcctggcaattgggccttgtgccggataaggaattcttccgcctgggcgctgtcattgagtcgaccaccaacaagctgagacagcagcagttggcgctaaccgaagaaggccgcgcggcaatcgctgaggctgaggcgaagcagaaggcgaccaacgccgggcgtgattttgtcgccagcctgaaacagcaagcagactcgatcggcaagactcgcgccgaactgctggaaatgaaggcggcacaattaggcgtttcaactgaagcggcaccgttcattaacgccctgaagcagcaagagcaggcgttaaagaaacagcagaatgctatggggcttgctggaatttcagcaggtcaatataaaatggcgatgcgccaacttccggcgcagatcactgacgtagtaacgtctcttgcttccgggatgccagtatggttagtggctattcagcaaggcgggcaaatcaaggatagcttcggcggtatcgggaacacgttcaaagttttgttgagctacattaacccgctaacagttggcgcggttgctttgggtgttgctcttgcggctatcgccaaagctgggtacgactcttggaagtcacagcgagatctagcgaatgcattggtgctgactggtggctatgctgcaacaactaccgggcaaatcacctccctaaccgatgaaatcaacaaaacatcatcggctactgtcggaagcattcaggcgatcgcaacgtcactggctcaatctggaaaatacagcatcaaccagattaaggcgatcacgaagactacggcggaatgggcggcgcagaccggggagagcgaaaagaccattactggttactttgactctatcgcaaaggatccggttaaggggcttgccgaactgaatgatcagtttaatttcctgaaggaaggtcagttaacctacatcgaatctttgcgaaaaaccaaaggtgagacggcagcagcggaagcggcaacgaaactctttgctgacacgatggataaacgactgaaggatatcgcagacagcgcaacgccgcttgaaacgatgtggactgacattaaaaagtgggccgcagactcctggaagtgggtaggtgatcacaccgtcgtcgcgctaaaccttattgttgatacggtttcatctatcatcaacgttatcaggaaactgattgcggatggcgacgccatgatcgcacaattcatcgttgatgctggtcggcagttgcagaaagtgcctggcatgggtgactttgggaatgattttcttgcacagcaagaacagctaatcaaggattccaaagccaaatcagcggaactggcgaagaccattgcggagcagcaggcaaggatcgcgaaaggcgaaatgggttacatcgacgccgcaaagaacaggacgttatctggtggttacagcagtgcaacaaaggataaggtaagtcaggaagaaaaggatatcctgaagaaccgaaacgccaggaaggagcaggtagacgccggggttaagatcgatgaacagtatcagtccgaattgctgtcattgcaggcgcagttgaaagtattgcagcagcacaaagggcttgacgacaaaatcagccagcagcgcaaagactactttaccactgtagcaaaattccaggtgctggaagaagcgagcgctaagcgtaagctgacgcagagcgaaaagcagatgttagccaataagcaaagtatcatcgctatggcggagcagaaggcgatcgtgggcgatcagattgtcaggcagcagcgcctgaacgccttgcttgacaagtcggtgaagtatcagaaccagatggctgagaagacaaaagcgcttcaggataccgccgggatggggagtaagcagcaggagcgctacagggcaaacgcgcagatggcggctgattggaagaataatggaggctcgcttgacgatgcccaatttaagcagatgcaggcagcaagcgaacagttttacgcgcagcaggatgcgcagatgcttgattggaaagcgggattcacgcatgcctgggctgacattggcaacgaagttaatgacgtgtactccaatatcgggagcatcacccaaaacgcatttaacggaatggctacggtgctaactgattttgtcatgacgggtaaggccagcttcagcgacttcgccagaagcgtgattagtgacattaccagcatgctaatcaaaatggcgctattcaatgcaatgtctgctgcgttcggaggtggcggtacgttcagcttcgccagcatgttcagcaaagggttcgccaacggtggctatactggcaacggtggcaagtacgaaccaaaaggcgttgtgcatggcggcgaattcgtctttactaaagaggcgaccagccgattagggccggaaaatttatacaggctgatgcgtggctatgcttccggcggtctggtaggctctaacggttcgtcaggatctggcgtgacgaatggcgggaatgttgcggcatcggcagcaatggtgtttagcatgggtgacgtgaacatcacgatgggttccggtcaggatagcaagggcttagagcagggagtaaggcagatcgtgaatgatatgttcacggaggcattgagccaaaacggacgcattgcgaaatacgtaaacgagaaaacgaggggttaacagtggattctttttcatggtgtactcaaattcaaggaggggcggcgaaagtcgccgtttccaacaacgttcgagcggttagtttcggcaatggatatatccagactgcatcgagtggcatcaacacaaagcgccggacggttccgatcgtttatggcgggagggattgggaggcggtttatgatttttgccagaatcacgtgacgaagccatttatctggaaggcaccagatggaagaatgggtatattcgtcgtaacggcagactctgttaatctcgcgccgatgggtggtggggtgtatgaggtaacggcggagtttgccgaacgattctcttcagccggatagcacaaagcgccctttacgggtgctttttttt

SEQ ID NO: 3 - DNA, 169725 bp, Klebsiella pneumoniae phage Kle_F58/19
gacgcagtcacttgcgctaagcgtacaggtcctatccttccagctgaagacttctttgggtacactgttattctgaagaaatacaacgactctattaaagcagtccagcatactattaaagaaggctctagtatgcagatcttcggcgaattcgctggttctggtatccagaaaggtgttgattacggcgagaaagatttctatgtcttcgatattctggttaaaaccgataaaggtactcatcagtttgtagatgattttatgatggaacagatgtgttgcacctttggtttcaaggttgcacctctgatgggccgtggttcatttgatgaactggctaaactgcctaatgaattccagattaacgttaatcgatacaatgaagccgcagaagtagacctcagaaatgctaatacccgcgtctggccggctgaagaagccaccgataatacggctgaaggctatgtactgaaacctaattatccacggttcctggctaacggcagtcgtgtggctatcaagtgtaagaactctaagttcagcgaaaaagctaaatctgataagcctattaagcctaaagctgttctgagtgaaattgaccaagaggttctgcagaaattctctgaatatgttactattcagcgtgtgaataacgttattagtaagattggtcaggttggacctaaagactttggaaaggtcatgggcttaacggttcaagatatccttgttgaagctgggcgtgaaggtcttgaagtcattcaagcagaacagcctgacattgtgaagaaagaaatcactaagttagtacaagatgtgcttcgtccggcgtggattgaattggtttcgaactaaaaaaataagggaggcctttcggcctcccatttttgttatttgcccgcgtaaaaggaaatataaattccacgagcttcataataagaatctacaaatccgcattctttagtccatttgattatttctttagcaatattatcttgatctatattatatagcgtagattcataaacaacctgatttggtgaaattgctcctttagaacttgaattccacgcggattttagttgggttatttgagaatttacaatatttttatcagaaaattctttagatgactgcaaaaataccgggataaaagtctctgtatttgaatctacattagttttaatagtcaaataaaacgcctcatctttttctggaagaatagttaacggcttaggatttttactaaaaaatccagttaaatttaaagaattaggaaacaccggaatggtggtagatactgcaaaatcagaaaatcctgatttcatcatccaaacagctatctcagccaatccatctgaagtacttacagaacccatgcttcctacagagccgtaaacctggctaagctctaggcattccggtaaaatagcttggttagtcattttttctattaaaagatataattgacctgtaggactatacatataattttgatttaccgtatcgccgtcaagaatgctggcaaaaatatcttttgcagtttccatatgagctattttagtttcaaatgatttgatcatgtcattaaaaacttctttatttgtagcaatagtaatagccaagttatttctcctgttgttttaaaggaaaggacccgaaggtcctatttctcgagcggtggtaacttaacgccgaggtaagaacttaatagactttgtccagccattttatccatatcagaaccatcaatgattcgagcttctttttcatctttagcaactgtataagggttagcagacaaagcataacgtgcgagtaaagaaatagtgggctgcagagattcagggtcaacaattatcttgaattcacccatttggtctacatcatcttctacatcatcttctacatccataccttctgtatacggtgcataatacaatgatgcaaccgtctgaccttcacccatatctgcattaacaccaacaataacatattctgccggtgtattcatatccgcataaagaggcagtccattcttcagatagccataggctaactcatcaccgtcatctcgtttttcaacccagccagaagcagctaaaatagccgcgcaacgcgaactagctacagcatacgtcccggcaaatgaagtattacgttggacggcagaattcatctcgcacatgtagtaatacagcgtacgtgcacgatcctgggcgttatcatatttaggatctgtaagatctaaaacacctttagacgacacgccgtgaactttaaaccggcttgaaaccgtaactaacgattgcaaaacatctttgttcacttcttcagccatctggatgccgagcatatcatctaagaaatcaggggcatcaaatccattagcttcaaggtcttgtgctaattcaaccgtgacagaagttttcagcttacgagatttaactgtagtttgccatttatcaacacggaacccagcttcagctatttcagggtgcccggcctcaaattgagtagtaggagcagcgtcagaaaccatacggatagttccggcagctatagcttcggaaatcaactctccgtggtctgtttcggtagatccagcaaaaggattatcttcaagagctttaaagataacagattcatgtttgaataagtcacctttattatacggtgcctggccgacgaattcttgcgtgctggctctagattcaggacccataagaccaccaaatgtagcaccaccgacataagtcatattatcttcaggattcagaactctgataccatataacgcggcaaccggttgacgggtacgttgttctgctacgagagccttaaagattcttttattggttgctttagtataagacaacaagtcaggtctcccgacctgatttgaagatgttaaagtactttcatggattaagcgatctattttcatttcggctcacttaaaatagcaggaagtttaacgccaagaagaatagacatattgctttgacctgccattttatccatgtctgttgcatcgataatacgggcttctttttcatctttagccacagtatatgggttagcagacaaagcatatctaatcattaaggaaatagtaggctgtaaagaatcaggatcaacaactactttaaaggcgccgacgtgttcgtcattatcaagatcaagaccttctgtataaggagcatagaacagagaaccgacgatttcattaccgccgaaggattctttaacgcctacagtaacataatctaatgggctgttaacgtcaacaaacacaggaagaccgttagcaaggaatccatacgctgattcagccaggtattcgtcatcttcagggcgatgttttaaccagccagacccagccagaacagcagcggcacgagcactagcaacgacaaaagttcctgaataggatgtttctcgctgaatataactgttcatttcacaaactaactggtaaagtttacgggctgcttcaggagcattatcgtaagataaatctacagtaccattatctgaaacacctgcaactttataacgacgggaaacagtaatcagagattgaaggacatctttattgacttcatctgccatcgttgtagcaagaagatcttcaaggaacatattagcatcaaagccgttagcttctaggtcttgagccagctcaacagtcagagatgttttaagcttacgggttttaactctagcattccatttattaacagtgaaattagcagaactaatttcgacactattggattcgaatttagcagtttcagcagcttcagaataaaggcgaagctttaaagtaactaaggcttcagcaagtgcctcggcaatcgtagcgccgtctagggctccagtttctaaggccttataaaccacattgttatacttataaagtgcccctgccgtcacagagtgtgtagcagaagtgatttcttcgatactatcccggtcagatgcagaagcagccccaccgtaagtagcacctgtgttaaaagagagttcatcgtctggagtttcgtatttaacaccatacagagcagcaacaggctgagtagtacgttgttcggcgacgatgtcagtgtaaaccaatttagtagtagcgcgggtcaatgcaacgagattcgggcgaccatattggatgctattcgttacagttgattcgcgcagaagttcgttaattttagccattgcgctttcctcgggttgatataattctatttataaaaggtctacaaacaaaaatgggagagcttttgctctcccataaaggtttttaaaaattacagacctttcacccacacacgtctaaagtaggcgtttttaccaacggagttaacgatggacggcataccggactgaatacgtgcaccaggagcttgcagtttagaatcagcgaacgggttaataccaataccgtaacgggttttgaagcccatgaccggctggaagttcttcggatcggaaccacgcagcggggtcagcgcaacgtacggagcatagtagataccagcgtccatttcgttggtgcctttataaccgatggtgaagtagtcctgcggagcatactggtcgatgtagacgcggtatttgccaccaagaacacctgcaaacacggatttagtggtgtcaacgttatagcctttacccagaccttgtgcagcataggtgataccagaatcagtagcagccagtgcgtttacaacgttacgggaagcgataatgaagttaccttcaccgcgaccggtctgacgagcgatttcagcggattctttgtcaatctggaacaggagagctttaaagctttcaccggcccaacgagcaccgcgaatatcaatcggatcctggaagtcgaatacaccagctttagaaccaacggtctgggtcataccggttttaccaggctgagcagagtagttgatccagtcaacaacttcacggttgatttccagcataatttcagtagccagaatagaactcagttcggcatctgcatccataccatgaacagcgcgcagatcctgtgccagttcgatggaatactgagctttcaactgacgagatttagcttcgataacttgcttatcgatacggaagcccatttcgttccacggattgtcttgggaaccattgaagccttcctgcagttcagcgatagaagtagccataccttcagccagttcagccagtttaccagcttcaaccagaccaataacaactgcatccagtgcagcggcgtcggcagcagcacccggggttacggtttcaacagcctggaagtaagcaacaccggtttcctggaatacgtgcttaacaatggtaccttcagtgatttcagtgcttgctttaatatcagcgaatgcacggccctgagcggtagcaccctgaccagagaacattgcatccggagcaaacatcgggtggaaagcttctttagcacccggtgcaatcggatctttaccgtatactgcgcgcagtgcaaatacctggccagtagggctggacatcggttgaacaccgcagatatcaaaagcaatcagattaggaatagcacgacgaaccatacccattacagccgggccgatctgagtgatagcaccagaggtctggcctgctgcgatattctgagcatcgtaaccatggtcaccgccaatttctgcttcgctcaggaaagaaccaaaagcttcagcaattttttcgtcacggaaagccggttcagtctgaatagctgcttcctggttttccataatttttgcaatcagcgcttttttaccggcacctacgatttccggcaggccttcaccttcaagcagcggagtccatttttctacgagtttgttagttttcatgtgttaataacctttttaaattaagaaatacgagccgctgaagcgacatatgcagccatcaggccaccggattgtttaggagtttcttcaggttctacagcttcgacaacgaagttaagaccgtcagcatctttgtcaatagtatttatatcagcactttcgacaattgctgaagcttcagcggaggactgtaccatttctacgatggcggccagtttagtaccgaatgcatcagaatattccaaaccttcagtcagagaagcaactttctctttctgggattcagttaagtcgcgggtagcttcgttcatagcagtttcgcgttgaacgtaattgatataagcatcgcgcatatttacttcttcaaacagacgtgcagtttcagctttctgttcagcaatttcttcttgcatttctgcaacaacatctactgcatcttcaggaaccacgacgttgtgttcaacgaacagatctttaagaccgacaaccatggattcgaacagatcagctttaataccgcgatctacagccagtttgttttcggcgagccactctttagcgacatggtcgaggaatttagcggcagattcagcgatttttttctcggctttttcttcagctttttctttttcttcttcgactttttcttcggccagagtagcgattttttcgatatgagattcagccagctttacagcggcggtttttacagcggattcgtaaacagtttcaaacttagccttaacgtcggcagacagttcaactgattcgaaaatgctgtccagttcgactttagcatcaatagtctgagcttcagtcatcagaagttctttaagcattttggtgtcctgttgttaagttacattattatttataatgcttttctaggctctcgattagagcggtaaatgctttatcagcattcatcttggcagttgcttctaccgtttctgtagactcacgaatttctcgtggtgctacaaacgcatctggagctgagggaccccaaactgcatcaacaccgaccgtcaggcggaaaccttcttgtacgatgttatatcctttacctgaatcttttaatgtgcccaggcctctactagacactccaggaatccatcctgctcggatgttagcagccaatttatcacctggaccgtggtcaccttcgatgatacgtgcacgtccataaacatcattacccttccaccacatatcttcaataatgatagcagcttgcattgggtcgacgtttgcacgaggaggatgatttagttctcctaacgactgtttcgttttaacttgttcggttatataccggtcaatggcacgctctaggacagctttaggatataaccgtttattacggtttaccacttcggcctgaaggaaaataccttcaatatacagtcctggctttaaaccgctcgacccgtctgaaggttgttcaaacgattcgagcataggttttccatcaatgatttcacccggtattccccagttctcaattaagagttgttcattgatgtttgacattagctcagtcctagtgcttgacgacgtttaatagctttcttttgtttgcgaataccacgagttaccgcgctcggattcgcccgtttggatttgaccgcttttcgagcaatttgacggcgcttcgatttagaaagaccagtagtttgataagcattacgctcacgagttttgcggtctttaaggcgggtcacttcaccgcgactgttgacatgcttaacgataaattcattaagctgcatatcttcattgagagaacccattgcgatagccagttcagtttcactagccatcatgttctctacaattgtatttataccgtctttatcgaatgctttagacaaatggtcaatgcggccctgagcttcttgaattttgctttcaacgttctcgataactaattcgaaattttcagggagtatcaacattattcgtcgtcctcatcgtcgtcgccttcatcagcgtcatcctcttcatcttcttcaggttcttcgccttcgatcatgactgagcttgcaatttcaactttaagctcttcaattaaactagacgtccgttcttgcagcagcggagccacggctttacgtactgcaacgaggtcgttagccttaattgcttccattaattcttccatttagaagtcctctacattttctgggtcttggaaacgagcctctttagactctaattcaatttgcttagcttcctgatcaatctgttcatcagacatctgcaagaaagttttcatggcgtattggtgagaaatatatttaccgataaaaggttcagccatggttagcatattgattctacgttccatcacttctgcatccttcatttcggtgaagtatgaatcctgatggaacacaactttaatattatttatctctttattccactcatcttctgtaataactttcttcaaaagaagattggttttcagaggatcaagcagaatttcttcaaacttatgctgaagatctcggataaatttacggaaatctagttcttcacgagtgataccagttcctgcatcaaactgtacaccgccttgattattaaggtctggcatacgagataatggaacacgaagagccatataaagagctgcacggaaataacgtacgtcatccatgtcactcatattagtggcacctggcagagtatcaacttctgttactgctttaccgtcacgtcgttgtaaccagtagtcttctgtcatggacatattatgctgctggtttttaatcttaccggtcgaagcgtcatacacaacacgatttttcattgtgttcataatgtgctgcatatgtgctgctgccttacgcgaaggcatattcccagtatcaatatagaagatacgacggtcgggagcacgcgtgatgcggtaaattactagggcatcttctaagagtttaagctggttcgcgggtttaacggccctgtgaagataaccgataatgcttttaccgcagcaatcaactaagcctgagtgggcatatgtaatagctgaacgcggaattttaatcttagtattagcagaataaactttgttatttacgcagtagctttcgttagccgtgtcgtagataaaatattctttgtagcctttaatgactttaacaccggcttcttcttttgtgattagttcacgcacatattgaaggttgcgtggatctaaacgacgaagctctttaataccatctttaggacgcttaagatcgataattttgtggaagaagatacgcgagtctacataccaacgacggaaatggtcattaccttttctttcgaagtcaagacaattcaaaacttctttgaattcgtccatcattcgttcttttatatttgtactaaagtcggtggagtcgaggtttaaagatactacatcatggccatcttcataaacgatggaatctgaaataatatcctggactgcgttatctacttcatagttgttcatcaaactacggtacgtgtcaataagctcacgagtagttttcatgcccggtaactgcgtaccaaacatcatctggttaaacgcgttatacggggcatcatgctcattagtttctacttcatgagctccatcatcgaatttgggtccggtaatggattctaaatcgtctctaatttgtcgtttatattcgaactcgtcgtgtttttcccacggagcgaataaacttagaatgttgaaattcattacgttctccaataatgatatcttactactatttatatcgctatttcgtaaggtcatttgcttaagaaatagcgatagggtccgaagaccctaccagaattacaaccaccagtcaagggcaaaggtagcttcaaatgtttcaacttcgttattgctatcccaatcgagggcaacttcaccaacgttggtcggccaaaggcctgtgatttcgtgttcattagtgatagttttaccatcacgggagaattgacgaactttagcagttttcttatattcagcaggggtagaaccagtaatttcgttaccctgggaatgagccatagcagcccaagcaataatagctttacgaacttcatgagcgtcatcgttataaattgtgatagtccaatcgtcgaatgtacggtcaccagcaatgttaattttgcggttcatatatccaacaggaactttttctacaatacctgcaggcatagtagcagctttacactggaagctgaagttacggccgaggaatgggatttcaacttcaaacaggttaggacgagcgaagtcaccggattcgaaagcacgtgtgatgtctgttaattccatcagtaatctctcttatttatatcgcctggaagtgtctgtggacgccagggataatttaaattgtggttaatataatttattatcctctaaaaggattcgcctggcggcgtcctacggatttattgctaaacctgaagccatagtataatgacttcaggtctttccggtaggtgattatattattgttgaacaccaatcagttcatcaaagtctgcaccggttgcagtagcaacaaagttcagagtaatgaagttgatagaacgtgccggcttgatgtagaacgatgcaacgaattcgttacggtcgatgaccgccggggtgttgttagtagtatcacagacaacacggaaatcgtacataccaccaagagctttaatgttcgctaagtattgtcctgtttccatacggaacgaggaacgagtgaagttatcgttcaactcgaagatttgatacttagaactatcaccgatattggttttcagcatgttaaacagacgacgaacgttaatacggtcgaacggagttggaaccgtagtagcggttttatcgccaaacagaacaaaaccttcacctgtaccttgaccaataacagggttaatacctacttgatacatgcgatcacgagccgcttgacgaggttcaatagccagtttgatgcagttcagaatctgcccacgacgatatccagccggagacatccaaggctgagaagttgtatcagtacgagcacacagacctgcaatatcagctgctaaaggaacccaacggttaacgtcattatatttatcatactgatatttatagttaccgtcaattgctgcgtatgtggtattaatgttcatattcgaatcggtataagaaccatcaccttcgcgccaatcgatcaggttgtcaacagcacgcgtcagcggaacattaacaagcgcagaacgaggcggagaaataagaactaagcagtcttgacgttcatcaccgatagaaacaacatgtttctggacggtagaagaaacttcagcttcttcaccggcacatgcaccggcaataagcaggttaacacgcagtgcttcacgatcaccaaacagatcccaacctttcatcagatccgcagcagtaacagattcgttagcagaggcaccaccatctaactgaataataccactgaaatctttaggccagccttctgaagtagcaaaaatgaatttagaagaaccgttggcgaaataatcatccataaagatgttattgccatacatatctttatcaccacgcttagtagacaggataatattttctactaaggtcccatcgcggcgaacgataatagcgtattgatctgctgtctgagggccatagccaaatacagatttagcagttgacgcacgagtaccaccgcctggatacatgtctaacagagaaactgcgttagcatcataggtagctttagaaacaatttctacttccagagtagaaccgatttcgccagggtaaacagccacaaccccaggcataccatatttagataatgcagtttggaaggtggtattagtaattttttcatcggccgtttctgtatcagtcaacagaataccggaatcaacaacaattttgcctagggtaataccaccggaaacaccagcagaagttcctgcgatttcaacagtccaagaactgcctaaatctggatactggttgatggatttcgcataagcgataatttttgcagtcgggataaagaccgaaaggattttaccgtcggtgtctactttagtaatacgaccttctgtttcaacatccgtggtcagatattttacagtcagtttatcacctacagagtagttagtacctgcggtatcgatggtccattcaatgttaccagcgatagggcttgcgtttttagcagcctcttcatttacaacacgtacggtacgaagatcattaccatactgaaggaagttcataccggacataaagtagtcagcggtctggttgttcggcccaccaaacaggtcaacaagttctacttcattagtaacctgagtaacttgatatgccggaccccattgaaacttaccaactaatgcagcacgacctgtagcgttaatgactaccgtgctttggacgctcgtttctttgagctcaattccaggagagattaaagccatgattattcctcaaagtgatttgctttatcttatttatacgaatgaaattccaggctccgcaggagcgtaggaagcactattatcggcagcatctacaaataccacaggcgcgtattcatcgttcatatcttccaattcacgactaaagacttctgatgcaagacgcatgtcgtctttatctgcgtaatcggcgaatttctgttgggttgaaagccaagcaaaaattactagactcataaccaggtcatcatggaagccttcttcagcagcccaagaaagtttcttctggctaaacgtacggaattccataatggtaggtttatggtggagaattaatttatctttctcgataaggtcttttaaagtagagcaaccgacagctttagtcttagtagtttgcttcatacctaaatcaaccattgaatcgcaaataacgttttcgtattcaagatccatataaagagattttgcaacagaaacaccagtcgaatttaactcgatatatacaggggcttcgttgtattccattaagtatttatgcactatatccggaaggattaagtgagaaatctcattagaatgtaatacagcaacctgttcccaagtatcagacgttacatcgataatatgcattgcatgataatcttggccacgaccttcagaacagtctaaagcagcaatatatttatggccttcttcaggctgcttaaagcgataaaaatagttctgttcggggatggtctctatccattccataatagccaatttcataccactgattaacgttcctgacgttccttggaattcagccatatgttcctgccggaattgttctaaagatgacccagaaattgtctgacggctccactgccacccgtcatcaaatctatcattatcgtcgtacagccgttctttaacagagttccaaatagccgtgtacggttcaaaaccagatttaccttctacagcggcagtccatatatcatagaagtggtttaacccattaggagtcgttgtgataataatcttagaacgacgcccggatgaaataaccggctgaatagcaagccaagcatcaatgaagttcgggataaacgcacattcatcaatatagatcatagcgaaggagttaccacggacggcgtcagggctacttgcgtaggcgccaatggaactaccgttgtctaactcaatgttgcccttgttccactcagagatccctggttgcaaaaaatcaggcagaagttcaatagcttgtttagtacggtctaatacttcttcagacatactacgtttatgcgctagaacacctacagctttatctttgttaaagcaaacaaaatgggcaaggaaaatagctacaacagttgtcttacctagctgacgagacaggttacatgtcgtcatacgcttggaggacataattttcagcatatcacgctggtaatcacggagttggaccttaatggtcccgtagtcaatgtgcgtgattgcgcaataagtttctgcaaagtagacaatgtcgtctcggcatttcttccattcttgaaccatttctttagtccaagctgttttaatgttggcccttttaaggttaggaagagcattatagcgagtacgcttattgttcttatctttaaatgttctaaaattagttggatcttcgccttgaagacgaatcttgactattccgttaatgcgaagataatcatcaaaagactcaggataccatttctggtcccactgagactggaagaacttaatcccgtcttctgttttaaccgccatagaactaggcggcttgattacaatattatcgccaacgttcaacggatggtcatcgttaaagacgttaactggctgttccattaattaacctgcttttctaatcgctcttgggcttcataactatcaccaatctcatccatcaaatcagatggagagcccatgaaaatggtagcattctctatgttagttgtctggtgcccgccttggcctttggtatcaactttctcgtcggtgatttctttcatttctttatgaagtttaagaatttccttgttcgtggtagtcatctgtcccattagggtagcaaatacttccatatggcgaggggagtcagcgtttttagctgtttcaaggaaaatcttacctgcatccattagcatttgctgttggaagtgaaggtttttacgaactacagaatagtcatcttcaaggtcaggtttacggtcgttagggttagatttaacttcaacgagttctaatttctcgtatacaggaatttcttcaccttcaattcctggtaagctgtcgatatccaacagcttgctcatgtctagtacttcactcatttcgcctcactccaggaggaatatctcctgacggaataggaatattttgagaataactttgttcatatgaaccatcccaatcagcagattctatatgcctcgggactgcctctgtatcaacagattcaaaattatcattgtgggaaagttctttactgtttgcaaagaagtccaaataaatcgttttgatctcaccttcaagttcagcaacaggtggataaagccagccgtttacttcaaacataatagaccactcaaggcgacgtcttgaattagcatcaccatcaacctgttcatccggtgcaatactttgaagaactatttttatatcacgcttgaattgaatattgttaggataaagctcgtgaatagttgtgttaaagtgtggctgaaaatacggtaaaatttgctcgacaatctgatacatatcatcttgatgacgagtatagattccaagctcaaaaatcatcttaataggagtagggttatactgcgagactggagttttaccatcttgagtttgggacttagtcctattaagctgagaagttttaaaagctgcattgtattgcatatctactaaatgaagattcattcttggaagaatagtttctatttttgctttatcttcagtagattgaacagcagtccacttattaagctgcatcataaatttttctttagaagcatacgaaatagggactttaatatatttaaggccctggtcattgcgtagacgtgctacttggacatgggaaaacaaatcacccatcaacacaatatatctacgcaaggacgaattgtaccagtgtccgaacattttatctccagagcccggggtttccgggcatatagattatttaatcatctaagaacccatcgtcaaaaggagaagatttaggtggaacataatttcctcttccgttattgacatagaaaggttctacacactgagcggcttcagcattaatctcttctgtttcggcgtactgatcaatgttgatatcagccaacccatcaatatttttaataggctgaagatcgagttcactgaattcaggaatattgataccttcattacgttgtagttcaggttgcatttcttcgccggagtagatatatttctgagcggtgattttacgcattgcattctggcctacctgataaaacgggtcgtaaggttctacccaagttatttcaaataggctattatccatcggaaaataaaccagatcaccggcaataggctctttaccatctgcttgatgtttaaacaagcgaggattaatagtgaatgttacttcgtcattaacagacataccgaacttactaaagaacgtattgtcacctgaatacccgtcaaagctattcaaataagcagcaaacatccaagccttattaaagcttgattgaggatcttcgccaaacagaagatctggattgttaaactggcgcggtagataataaagctctattccacgcatctgaatactttcggctaccaatacatcagctagagtttgggtattagtatacttgttgaagttaacaaacgggtttagaatttcagtttcgttggtcttagcatagcccgaacggtcttcgagcttagcaaataaattcgaattataagtgctcattatttaccccaacagaataggacacccaggatcgagtaaatcaagctcggcacgtaaggcttctttttctatacgagcttcttcgattaaacgggtcccgtcaacagttacaccacccggaagcatcatgccttggtgtttggctaaaatctgaccattaagttctttagctaaacaatgggcgtaatctttaacccagcgattattataagccccttcgcgattctggttctgttcgcctgcaaattggcctgtgattcctttattaggattatcatatatttcgccaacaccccactgattagcattattagaaccagcataaccatatccggcggtgttacctacagcccaatctacgttattataagatttagtccatacctcacaaacgattatatcacccttcataaagttacccattactttgagcatctctgtatcagagttataccaataatctggaagtggggacaaaaggtcctgcatcatcgaccagtaagtcatcagctgggtaaaataccctaggtcagctccgaacgcattcggcccgtagcccttttgacaagatgaacccataccgccattgataccggccatacccataagaaagtctgtaaaccaaggataagtggcatttccgtccattgaagtaatcgagccgacattagtacgtactatctgggaaatagcaaatacattacgcccgtggagattaaacactccgttcaagaactgggcattatcagattcttctgtaccaatatagaaaacttggtagcctttgttcagaccactaaaatgatattcaccgtagagctctagagcgcgctggatacaatcatatatttggtcagtagttaattcaacatttacgataggtgcgcccatgcggcgcaggataaggtccttcagttcagaaggattattagcaacttgtccggccataaaaagtcctcatataataatcatatttatacaaaaatgggagaccgaagtctccccaattttagcgcgctggaactacagtaatatcaacagcttctacatttaaaacaacttgagaagccggttcaacagctttaatggctaggcgtaaacgatctgtttcttctaactgtccaagccaatcaaagctaacaacttggtaacctgctacagttttaacacctacagagttaacatccacagaaacgtcatcaacataaaatacaaggtcgagaactttatccgcgttggctttttcgataataaccgaaactgtcacctcataaattgccgagtcgtcgataacgatttcgctattaataacacgaatctgattattaaaatcagccgctacaaggccaggaccaaacgaatactcaaaaccatcaccggttaaatctgcggagaggtctttacctttaaatgaacccatcgccatggattttttgatccactggtcttttgtacggaggtaagcattcaggtcagttacatcctgaacgtaatccgaatttatctttttaacagactctaaaagaccgctcttttcaatagggtctgacgcaccggaattaccgtaaacggttttacgaacttcgtttaattggcctgtaatacctgtcgtgccattaccaatttcagcctgaatatcttgaatagatgattgctgctgagactggacgccgataacttcagtcaaacgaccttcaatagaactaggatcaggagtttcgccaggaccaacaatacctgttttctgctgaatccaagctatctggccgcgtataccagaagatgcatcggctccaacaacagtcttaatttcatcttgggtagtttgaatcgcatctaatctacctaaaatgctgtacggttctgaagctgatccgatttttacttcaatgacacctactttatcaacaagaccggttccagctgaattaacggtagcatcaatagatagagtacgtgattctaaattagatactctagacgaaattgtttcagggcctgtcaaattaatagcagcagaaatgttttcttgagtggttttaagaagaccaatatcgttgtcagccttaattattctgctgtaaatggaagtcccatttgctaaggttttaggtcctatttcagtacgaatttcatcaattctgcctgataaagcacctacatcagaatcgataaaattgtcttcaagatcttggacacgggaggtaactttagaaatggcatcactgttagaaatgatgcgaagcttcatacctgagccagaagaatctaaatcaacttcaccgtttttattctggcctggataagaaccgatttcatctttgatgaatacaatatccccacggacggtacggtgcttaggatctaagttcggattatacactccgacttcagccgttaattttgtcgaagattcttttaaagaagctatttcttgctcgtgaagatctaattcagaattaaccagtataatgtcatcgcggtttttataaacctgagttactgcatcaacatcagtggtaatttctaaagattctttaatagattcgatttcagtactgttcttgtttaattgttctacagttcgttctacttcatggtgaacaaattcgatattctcttggagttgaacgccgacacggttaagttcaccttcatgcccgtagcgagtagaagccgcattaagcggctctccatttttaatccaagcaattcgtttttgagaatcacctggctcattagttacattgggtaaagtttcaagctttataatcattaagaatccttaatttgtgcagaatgtggccagtacgtctacatgtaaaactcgatgccattggacaggctcgatgtcaactctagctcctggaggaatgttaccaaaccggaaattgcctgatgcataagcctcaaatctccaggaatgaccatttcttccgcctttagtgttttctatatttaatgtaagcattccgccttgtctttgcccattaacacgaacatagaactgaaaaatacggttatcgccgccatcattgtttctttcaaatttacaaacaacgttcatacctacagtagaagagccttcccacggactttggcaataccaagtatctggactaccgttgtttcctctgtagttaaggtaggtagctccttcaatgcgggtaatattgccaaccatcctgtctttaggcataaaacggttgtcagcttcgcctttagaccaagcgtttacatccccagcacctaaagtaatatttccagataaaggcttaccgtttacggtagttgttttaaacacgacggcatcagctcttgcggcagtttggcctgattgaggaacaccagatagctggactaacccgtattgggatgtagaaccgcgaagggccatcaattgcttaggagaaactgctacagaatctaaagtcccggcagctgtttcagatgcagtagataaacgaataacaccaaatttagattcagatgctttagcaccaagtaatcctttaggtgatatagcgtatccatctcctaaagtcccttgctgaatttgagcgctagtagctaacttaaccaaccctaagttagtttctgtcgccatcgtatatgaaggaggattagctgaatgagcggcaatcatttctacaacgcgcttaggagttactgccgtagtatcatcagtaccagcttgtcccatatcataagtggcacgcttaatagtaccaaatctagtttcatttgctgttacattggtaaaaacatggcctaatgcagaaggaactatagcggcgttgccaatagcgaatccggctgcttcagcattagtagcatatctggttaggccttttactgtggtagttgcatctggcctagtgattacagattttaatgtggctgggctaacaaatttatttgttattacccctgcatctacttcaggttgagtagctatagccacaatacccggagttgtaggggacgctataggtaaacctacgtcagtacgtgaccagtcgccaagcattgataatgctttttggacattatccacagaatccggccaatctgtattagccggtttaaaaattgtgtattcagctaagtcactatagtgctgaaagttattttgagccattacgagatcctctggaaataatggaagcgaacagtattactagcaaatactttatcttcgctacctaaaaagttccaacttccgtaaccttgtttagcaggagataaaacactaaagttaactgtaataccattccacttaaaagtttcgaaagtatggttctggtaatccagatgggtaatatctacaatattctgggtagaagcagagctgaaaactgctgcaatagcaaccttatcggcaaccatagactcgagaatgttaagccatttagcagcagctatctcggcagtatcgccatttttaagtttaacagcaacacctagaatctcaacgatttcttcagtcacttcatcttctaatttgacaacaccggaaaaaacagcccgagtaatttgttgagttttccctggactaataccagttacattagtaacaacttctccaacatcgctagggatcatcgcgcggacatcatctattgctgcctggatattaggataccaaacgccgtgctgcgtttgttttagtgtgaccgatccataaggaacttgatcacctacagaaatatcttcagggtctaattcaaactgaagatagttagcttctcttgaagggaccttagccccttcacgtgttttagtaaaaatcattatgccacccttatccagcgatagacagttatagaaggttgaacgttagagataggtaagccaccagcgcctttgtttacggtgatagaaccttctctatatttagaatagccaggaccagatgcatcggggtctaattggcatccgccgataactacagtaccattatcatcggcaataagaactgtgtcatcagatttgagttccgggatgttatttgccgaaatctggaattcaacctcaccgaccgtgcctccagcactatgagacggcctacctgaactgtctaggtcattattatttaatgcaaagtaaggattgcttatatcagaagcccaccctacagaagcatagccttcaccgtacatcttccatacacccatgcccatataatctcgcgggttagctgggttatgggcattttcataaatggttccgataggataaatcgaatcaaaaaatgccgagatgttagataaccgaacatcatattcattaggcacatcaactttagttttttggcctggattctgataatcagtgtattcgatcctgtttttaaggtccactgtttcttcggcattcatataaatcaaatctgtctcagaaacaatatcatccagggtcatcgtggttccgatattgttattataccaacggacagtaatgatatcttggtcttcaaatggctggccgaatctgatagccgaaactttaataccatcagtatcaaatactaatcgataatcggagtccgattggacccaagtcccaccattagctatacagtcctgtgaagattcgcctggagcaccttcacaaataaattctgggagcccgccggttcctgcttcaacctggcttttaccgtttatttctacctcaatagaattagggttcatgtctataccaggaagaacacctaaatccgcagtgctaaattcacgcagagtgctcaaatcggctacaattttagaaccttggactgttttctcagaggtaagtttgttactgcgaaccataacagtaactttgttgtatgagctacgccatacacctataccgtctaaaaaggtttcgaaaataattacatcgccggctgaacaaggtttaactaaccgaacatcttttttattcaaggccacgatagtgttgtttgggccaggagaaccgtaatcagcattatctggatcaaatccattttcaccataaaacagcatattaccgcgacgatagacgttcagagattctggattatattcttcgccatcaaaaatatctaagaagtcagtttggccttcagtagcaataattgatttcttagaaactgtagctaaatccccgttggtaaatttctcaatggttttgccgttgatgtattcccatttacctggagagcaataaactagctcaagatcttggtagttagtattaaaggtcttaggagatgcagagcctttaatagtatcgcctgaggcaggaactacggtcactggagataaacgccaggtagaccatacatcacgtagacgaataacattattgtaatctgaaggtttacctttaggaagataaactgttacgcgagccgcctgggtattaatcgcgaagctatcacccattttaggatcaagtcgaatagaagtagccgcggaatgggttttccaagcacctgcggcatgaggataagtaccatcacctaattgatagtacagttcgtcaaagttgttattaatcttttggccaccaaggcggagataatctccgctgccgtcgtcgactacttgaccaatatttaaattctgtttcattattagattcctgtaccgacggtctgagtaccgatagatttaattgccagtctagcgttagagtaatttgatttcacgttaagaactactttaccggcagcattaaccgagtaagtagcggtataaaagaattcatcttcacctttttcaccacccattctgataacgccgtattcagtagagtagactttgttcatgcttgaatcaatcaataaaagggtttcagaagactgtctgatatttttaccaggtggtgttgccacataactcattagaagcttaatagagttaaattctctaactccagcaataacaatatcagttcctgtttggtttattgtaaatgttctttctaaaggaacgtgctgctgaccaaacatacttgtaatagagtaatcccaaatacaattacctgaagtgtctttagaaatacaccagacttcaactttagtatatggagaaactatttcaattttattaccgccagaagcgaatccgtcgttagcactagttgttattgtaagactggtttctgccgaaatagaaccgtcagcatttacaaaatagatagcttcaccaacattgcctttagctatagtaacattaagcttttgcccattagtagaaatgtcgtggcaagttcctaaagctatttcactgctgctataatcagcaaatgatagtttttggtaatatccggtagcatggattatctgagtgttttcgccatcggcagccgcaaaaagccgttggtcgccgaaagcattaaaaatatctgagaagttgttgtttattttaacgccgccatcgtataagatatcgcctgtcgaggcgttaccgatctcacctacgtttacgacttcttttggaatttgtgtgaatgccatagtgtacctcaatatgtttatagagatatttataaacaaaaatgggagccgtaaggctccctagttaaaattcaaagataatactaacttcttcagtttggtccattgacctaatgattggctgtctattttccatataaatgatttcaccggactgcctaaacaaacttaccgccggataagaacctttagtggccttaacattagggtcattaggattctttttagcttctaacggattactaattacagaaagctgtctgaatccagtattaccaggtaaactaaactctggaaagtagaccgagtccaagaaagctttaaaccgcacacagttggctttaagacggtaaatgatgtcatagtcatattgttgccagctgatgttattatgataaccccagcgttgtgggtctgccgctacttcatctggccatggaactacaatatattcattagtgcagcggttaatagaaacgtctggtggaatctcataaaggtattcccagtaatacccgtcaccggtatctatgataccgttagtactaccactagtatctgctcggcctctaggaacaatagtagaagttcttgaaggagaccacttaccgcctaatgctagacattcatctttattagacaactgtcctatagaacacataccttcttttggaacatcaacgcatttataaattagccagccagccccaacttcagttttattatacggagcattattagtaacgacaatatcaccaattaaaaatgttgaattgtcgtatcttgaatcaccccaatcgcgtctaggaataacgcaatctaacatgctcgggtcaactttaacagcgcccatcatgttagtccagatatcaacaaccccgtcagtactatctgaaggatacggaggagcaaatcctggctcgttttcgttagcagaccatggtgtcgatttgccgaagcttatatacattgtattttgttctgggccatctccgacggatttatagaacgtgtacattttttcagtacgaaatttagaagtaataacagcgcgataaatcgcgctgttcactgatgctagtgcagatgtagacatttattcacctatattgacctgggttggattctcaggatcacgaggatttcctacattatctttaagtcgttcatcgactaactctctgtaacaagagaatgttacagcagactggtcaaataaaggactataccgcttacgtctttcacaagggccttggccttggaattgattattattcgctgtgatatattcttgagtacacggataaggaacacctttgtttacattgtcgtagtaaatagggtcaccccatgcatctcgttctatagtcccacttggcgaaattgcggcgtttctatcaggataatatgaaggaatacctgagtcccacttatagttcttgagcttatttatggtagtttcaacgtgtttcatactaagaccagcattaataaacatcgtgatcaaagtaataccgatgaagccgaaaccgaccggatgaacaaatctcagtacgtcatctctatatctagaagtaggaagagaagaacgaattttcatcacataataagaacgagaacggttaatatattctatggtattagaaataagttctttgccacgaacaccaacaacaatcataccttcaaaagacgttttttcagatttaacttcttggccagcaacaaatcgccctatcatattatgaatggtcatacgccacagaagcttaccgtcaacgtattcacgctcaatatatgtcacattacaacgacctgatgcagtataaatcgttcttcccacaatatcatcattgatattatcagaatcgacaactatatcatactcggtgctgtttttagattcgatatcaatttcaacatcttcattatacaagagcttgaacaagaactgatagctttcttcaataccttttgtggcatagaagtcatttttacgagcttcgaagaaacgaacaacagcgtctctttggtcctttgacagatagatattacgtttgtaaacttcagaccataagtattcccaagcgtgttcttcacgtgggtacttatcacgaacaagattcagaagcttattgtaatgagtcccgtatcctttagaaatgaactgtaagtaataattactaaacgcctcaaagttagtatcttgaagcaaataactatctggcatcattttacgaactaatggaaccaaatccgggtcttggagaccagtatcttctgttggggtccaaggaacttcatgttcttggttttgtaaaaatgctttgaacatcacatttttaggtttccaagtaatcttaacataatctctgactctgtattcaaattcatagaaccctactaaagtaccatcaggctcgtgaaacataatgccggaggcatattttaagaagctatcgaactctacgttaggacaatagaatacgccatcgcctttattccaaacttcatgcaaaatacggttaggatctgttaaagacagagtatcaacatacttggtataagtagtgttgttataaactactaaagcatggtccgtagtagtaacccagtttctatttccagaacgggccatccagttaaaccaaggctcagcataaaatttcatacgaccaggtttaaactcttcccaaccgctattttcgttagatcggaagctcatcatatgccaatgtttatcatgatggtactgatacggagcataatatgaaacggcttctgtaaccttggcagggtattctaaaggaagttcattatcaggaacatattcgttataccgaaagtcgccagagctaaagaacaattcttggccatcagtggacatactagaccatccgtgctcaattctcctgcgttcttctgctgtagtaccaaaaactcttctaaagcttaaagtatcttcgtcaaatacgtaaacgcctttaattgcttcagcttctttagatacacattcaacatttttagggtcagacgcatcaagccctgcggattttaaacggcccgtaataagagcaaagattttgccgccgatagaatccattttataacaaacagctaggtcatcacctgttattctgattatttcaggctcaaataacttttcaccgaatgtaggggacccaataacggtatcaactggagcttgttttaatttaattctgcgaactgtgtctctggctacaccgtataagtgggtattgttaacggtcatagcttctactctagtattaagaataccaggccatgaagcatatacaccgaagatttctacttcgaaccctaaattaagttggtcacctaacttagtaaatgttacttcgttattgctaaaacgaacttcatctgaggaccaacgaacatcgctggatttacggccatagaacactttattatagccaaggacgtatgtagtagtgttggattgataaacacatgtccttgaaacaggattgcctacgcgatcactaaagaggcgtacgtagtgccagttttggcctttatcattagatactttgaccatgtgctggaaacgttcaaacagatagaggttgccatctatttcagcaagcatggttctgtctttatctacacagaccgcttctataggcccttgaatttcatggaattcatcttcacctaaaacatattgcttaacttgtgaaagatctttatattcggtagagataggaaagttttcagccatcaacgatgcctgtaatgcagccgtattgaagttaacatagttcatacggttcttaatgaacttttcttcaatgaacggcttcataagcatgaattggctcatctgttcaaatgtgtaggcgttaacttcaaatgtttcaaagtcttcagtttctacccattccgaaggttcgaatcccgccgcggtcgattgtacacgcattctgtacgtggtcataggacgcaagttatttgcgaaccattcgttttcagtggtataacctagtgcacgccaacttaaagaagaacggacgagaagtcgaccggtttcatcttcgatttcggctatttctacaaaatagtagaagtttgcaccaacgtcatcccaacgaatattgacctggttcgctgatagcttgtgaatacgtaagctggtgacgaaaggtgcaattgtcattgagtaattggctccaatgtgatagttgtgtattgagggcgtaaatcgttctcaaatacgataagtgttccatctcttgagaaaataatatcttgacttggcgtagaataaagctcgatgctttggtcttcaaaacgatctgaagttacaccaagagccccaaggttccaatagatgttatcacccggataagagatttcgccgactttataaaatttagtacgaccttctatagtaggtttattaaaatcggatcctgtgtacggtgtaatatgtgtattctcaacaatatcaccgtctcggaaaggccctataacaatattcccttttcctaactcatcaggatctgccccgtaaattctaacaggataactcaactcagaattagctacaaaagaaatgtcgcttgactctaaagtccgcttattaatttgattatagtatttaataccgctttcaggcagaacaaaataattaacaacttcacgaaccagctgaattgtggcagatgaacctaaaatactaatatttgcatcatctacataagtcagcattttagacttactaaatgagctattgaagatttctacttcatcgcgataataagtatctatctggtctaaaattttattttcaagccactgttcagactcttgcagtttattcatagcataagaaactttaacattttgcttaatgaacaaataatctggagacataatagctggagtaataggagctaatgtaaacggctctaagtaagctttaatatcttctctctggacagcagtcagttgtagacctgattttggtttaattgcaatgaatgcataacctggtttatcttggtctgtaaagcattgaacagcctgaacaatattaccaaaacgagaactaacaaaactttcataatctgacttagaaacgcagcgcatttgggattcacgtttcagagtagcaagttctcgaatacgttcaattccttcagggtcgccgccaccatcagcgccaacataatcaggagaattggtccagttttcaatgattttattaacagtaacatattgaagtgtgtcggcataactaaaatctacggcaccgttagcaacatcaccctctgtacgaatatactcaatggttattgtgtcattgcgagtaggttttagaccaccaataaagttagcttctaaaacaccgccagctacagaagtagtttctacaccttcgccaaagaagaattctgtatcaccatcaacagtttcacgcatatagtaaatgtgcgaagtgcttccggtatgaaccattgatctatcggtccagttggtccattcagagccgttaaccattaagcgaacttctgatctgtcaatattagggtcacggactaaaataggaactttagggtcgtactgaattatctggcgaataattcgaccttgagcgagctcaacaacaggccaataatgtttatcatcatcacgtgttacaactacatctttggttgtgacaaaagaatatgggtctgctgatgtatcccgagcataggctaaaaacttggtcccacgcggaatactaagtgatactgggtttaagtcatgggtaacatctagcataatagaggttttagccgcggatttagaagcaggaagataaccgttttcttgggctgcctggactaccgagctacgaagattagcagttccgataaaactttcgtatattgctgtgttagcaaattgttgaatataaagagtgttataagctaaaagatctagcagtacgttgattcgagaaccagagaaatcataatcttgaaactctgattgacttgaaagccaatcaataagattatttttaatttcgtcaaaggttgcgccaacgaatgcttctgggatagcgtttgcagtacgcgtcaactggtaatttactggttctgtagtagccattagtgaataaacaccttatgtgaagattgagcgatggtgtcaccgcatgaaattggatcggccatttgggctgctttcttcccgttaacgaatactttactcgttctagcctctacgacgcctccatgtgtatcatgaggcttcgttgtttttgtgtgtggagtaataggatcgccgtcacgaacgacagctataccaccggtgaatactttagattgtgtagcatttacatcggtcggcggataagcatcatgtcccgttgtaatgcaatgattgtatgctaagcctgccattagttctccgcatatacgtaatcacgtagcttattggcccaaatgctccagttgcctttaactgtttgtgagtagacctttgttagctttttctcttctacagagccacttcctggttcgccagggaccgctggagtagttacggtatagattaactcaacagtgtatgtaaatgtcttagaaagcgccgtaggggccctgaataagtatagatctgcttcttttgggggaggtaactcctcccacgctgaagcagatttcaaatcatcaccttcacggtataaaagcgcattagcaccaaaagtgaaaattgagttatagttacctgtataatggttttgagctacagtgataccggttactggttggtagttagtaatagtaattgacttaagagtttcatctgcctccaaagaggcagtgaagtattggtctacaaagtcaccttctataagttcttctaatgtcgtatttattggaattatctgagccataattaacctacatcaattctgctaccgtcgatagtgtactgaccttgagcaatagaactcatcgaagccattgtctcagtccatgacccgcctacattccaagaaaccgttcctgcaacgacataagacatgttcccgccaatattcatttcatggttcccatccactttagtggtagcattgccatttacttggatatccgcatttccttcaacgataacttttacattacctttgactacaacagtcccgtcgccttcaatggttttagtttcatttccacgaatataaagagtattatcaccatcaatttgctgacgacgattgtacatattataataaacttcgtttccaccaacgttaactttcttatcaccggaaatcatgatattcccatcaccttgggtcatatcataaagatcggcaactgttttacgcgtacggcgcccgtcaggagcaacttcttcataacaaccagttggatggaccagtctatatcgttcgtaaccgggtgtgtcgtcaaattcttgaacatggcccgcttcagattcatacgtatgaacgtaaggatattcgcctttataagaactttcaggttctttgaatagaatccttgaatctgtaggagtccatggagcgcttggatcgtcagaatcatcggaaaccggagcagcaaatagttgaatcattcttcctttagcctgttctggaggaacaggaacaccgtatgactctaaattaccggttaagataatcatagatacacgagaagcacggcctttagtctgattaaaccaaacagaattgcgggcttctttaaatgccgtttgccagtcttcaacaagcattgcgtctagcatacctttgaattttgctaatccgtggatgcccatttggaaagccatattctctaaagccatctgacgagacctgtttactttagcataaacaggacccactctaggattgctttttacatctcgttgaacatctgctaaatctttctggaataatttagaggcttcttccatttcgatgatgcctggatttccagatacttgacgccctacttgattgctcagaattttattaatctgggccatatcgcgtattttctgcatcacaatcaagtggccgatacctacggtagggtacccttcagaatcccagtatacttttaaacgaaggccttcatcacgacgaagcatgttttcaatagtgaaattagggttgtcgtctggtttagcatcaccaatatccgttgaatcaggattaatacctacatcaaggttagcatcttgaataacgttagtattgctgctataacctatttcagatccttgagtcaacgggttcgtgtctgaacctaagtatctagggaactgtcctgtagggtcactgaacccttctgtatagttaggcttctttgtattgtgggctgatataacatgagtaacaatgcctgaggttttatatttgtcgagccattgtccaaaaactgtactaccttcaacaatacctgtaattgaatgacctactccactgataccagcagagttagtaggctgccctacagtcatccatggtaaatcagcggtagaaataccagaaataggcccttgtgttttttggaacggatgaaggcctacaacacgtactcttactcgaccttgttttaacgggtccatcctatcttctacgacgccagtgaaccagcgtacttcatcattcatttcaatcattctggggccttttccatttcacgaactatatctgataaaaaagattcgatatcaacagggttaatgattttgatctgccggagtttttcattttctaatatagcgtcttcatagacatcgactgcagctaatgcaccgcgatattggatgtgtgaatccttagtataatcgttctcacctttggcccaccagatgcctgaattaggaggaacctgctctacgttgtaatatctgttcccgtcggcatcaacatgatacaagatctggttcccgccaactttggagtatttctgctcagctgcttggtaacaagcatcctgtgatttaatccatccatgataagggtcgtagattccgttacacattaagagaatccaatagaactgcgtgttaccgtacagttcataagacagttcttctggacgtggagcaccttgaatgtaatagttctgaagcaagtagttaaccgctacacggtcaaaatattcttggtaatcacgatagatattagctgtcgggattatcttcttgctgccagtcgttttagcttggtactcaattgggttgaaaaacgaaaagagcatagtgtatccttttataaatattggtatatgtatttataaggaggcttcattatggcctacagcggcaaatttatgccagagaatatctctaagtacaaaggagatgtaagaaaaattacgtaccgttcctcatgggagcagtatctaatgagatggcttgaccgtcatcctgaggttgttcagtggaatagtgaagaagttgtaattccgtatttttgcaatgctgaaggaaagaaacgacgctacttcatggacttttgggttagatttaaagatgggcaacagttcttttttgaagtgaaacctgccaaagaaactgttcctccacctaaacctgccaagcttactacagcggctaagaaacgctacataaatgaagtgtatacttattcggtgaatcaggataaatggaaagcagctcaagcaactgctgacaaaatgggtatacaatttagactaattactgagcattccctcaagaaacttggatggaaaggctaatggctataatcgaattcttgggtgaagctgctccggcgcttaatcgtgcaccaagtaaatcagagcagcaatgggttaagattggtgttgaatacgctaaagcaaaggccaaaggaatcacagctaaagaattcgctgaagaacgtggattcaactatgcttcgttcactaaagccatgtcacgataccaatcacgtatcaaaacggctgtaaaggtcgaaaagttagaaaagaaaccacaaaataaactgactaaagctgaacgtcagttaattatgattaacagtttccgtagttctcttcgtgaaaaaataaagaatgaaggagccgctgttaacaacaaatcagctagatggttcaatgataccattaagaaaaatgttcgtgggcaccaagtatctaagcctgaaccaggcaaattatacgcatacatctatgatgctaagcataaagatacattaccatattgggacaaatatccattgattattaatcttggatttggtcaaaacggaagttctagggttatgtacgggcttaatcttcactacatacctcctaaagcgcgccaggctttcctagaagaactcctgaagcaatacgcgaacacgcctgtaatatccaataagactaaacttaaaatcaactggagccaagtgaagggctttaaaggcgctgaccagatgattaaatcctatttgcctgggcatataaaaggttcgatgatagagattaagcctgcagattgggctaatgtagtaatgttgccacttcagcagtttatgtcgaaaggcaaacgttattctgctacttcagtatggaaatcataatgcacgattcaaatggtgggttatttaaccaaactaacacaacaaactttctattagaggttcctgatgggaacctcactaaagcatttaaactgaatcttcaagtagctgctgttccagggattcacatccctgttactgaagttgctggtcagcctcaaggcatgcatcgggcttctttaagttctgccacaatagagttcgaagccgttccatgtcggtttttagtcgacgagaaacttgaatcatggcttcagtgctatcagtggatgctggcctgcaataactatttgaatagagataagtctgggtggaaccgcaaagatggtttcccggatgcggttttaatgcatattctagataacgataaacgtgatatagtattgactatcagatatgtaggagcttggatctccgaccttagtgaaatcgaatactcgctggccgaagattctgacgttcccatgtattgcactgctacattgaactataagtacatcgaagttgaacaaaacggcgtaattatagagggacgtgctcctattaatgaggcacgcgaagttcaattacaggcccaaatcaggggtcgtcatccgagtatgcaatgaaacaagtttatcttttaggtaaaaaacgcagtggtaaggataccaccgctgactatattatggataactacaacgccattaaggcccaattggcttggccaatcaaagaagctttgcatcacgcgttttctgttgtacaataccagtattctgatattcagaaattaactcgagaccagctcgaaggtattgattacgaccgtgaacagaatttgttccttgataagaagcgtgtgttagctattatgctcgccgcaatgggttggttatctgaacagaagtatttgtgcgctacctacaacggcttacctatgaactcggtagaaagagaagttgaacgcgtcataaatactattgaagatgattggtccgttagacgtctgatgcagacccttggaacggatatcgtggtcaattgctttgataagatgtattgggttaaatggtgtatggatgaatatgctgataactttgagaagtataattacttcatagttccagatgttcgtcaagaacatgaaatttcttttgccagggcgatgggtgctacaattatccatgtagttcgcccttctaaagaaactaaaacagattctcacatcaccgagcgtggccttccggttatgcctggtgatattgtaataacaaacgacggttctattgaagaactttacaaaaaaattgataaggtattaaaatgagtcaagatattattaacacactgaaagtacgcgtattcgacctgtctgaagttgctcaggcttcccaagaccgcattaaagaactggaagctgttctaggtgaactggttcagattctgcctgtaaaagctaaagaagacggttcttttgaactggccgctatcgtagaagcagctaagtcggttgttctggccggtgatgctgattctacggacgaagttgaaggctaagtgaagtttaaagactttactacaggtctttatgttgcagctaaattttctgaatcgactttagatgaaattgaggatcttcagcgagagttgaagattcctaatccagttcctcgtcataaaatccattcaacgatatgctactctcgcgttgatgtaccttatgtatgctcgaccgggagttttgaggttgctacttcaggagaacttgaaacctgggatactcaagacggtaaggtcctagttcttaagttagactctgactatctaaagttccgacataactatgctcgtgcgttaggtgctactcatgatttccctgactacacaccgcatatcaccctaagctataacattggacctgctcactataaaggtgaagtcaagtgtcatgtagttctagaccgtgaatacaaagaacctcttcaactagactgggctgacgatttaaaataaggagacttcggtctccttttttgcattcttagtttacatcattaaatagtcgtggtactatactctcataccaacttactcaggaaaccaaaatgaaaacgtatcaagaatttattacagaagctaaaagacaagatgatgaactgccggttgttacaaaaacctttgacgggacgctggcggattttaaaaaaatccctagtgataaacttgctaaattgaccggtagcatcggtgaaaaagatggcaaggttacattttctgcccgtggttctgccaacctcaaaaaacttttgaaagctgctggtgctgaataaattgttttacattagggtgtagtcgtgatactatacccttataccaacttactcaggaaaccaaaatgaaaacttacaaagaatttttaaatgaatctaaagatgctttcaaccatgtagaactgagcatcactgattcccagtacggtactcacaaactgttccgggcagtccatattaaaggttctcacgaaaacgctatggttgacctcggtgaagcaaaagacctagaaactctgttgagtcatgttaaatcatgggatgctaaagagttccgtatcaatggtaaatctgttaaaatgagcgaaatcaaaaaacatttaaagtaagtgtttacaatggtaatgggtcgtgataagatgaccctatcaaaacaaaagtgaaggagattaaaatgaaacgcgcagaaatcattcgtaatgtggcagtaacagtagctcttggagccacttcggtttctatgtttgccggttttatggcaggtctgttaactactaccgaaaacgccgtatccgcaggactggcattatcaatgattattgtagctttcctaatgaacgaaatctctaagtgaggaaaatatgtacggattattcatgtggtcacggtactacccttgtggcgggacaggtgattttgtagattgttacggctctatcgaagaagctattgaagccgctcgtgaacgtaaagactttaacgacaagtatgaagtcattagttcttcattttgttctataacaagcgggtacaccaatgacttataagctgtatggtgattttgaagcgaacccacacactcgtcagaattgccgtatcatgtgtggttcttttgaaacataccaagacgctgtagaatatgctcgtgatgtgagttattcgtttatcgaaatcacagaccagtatggtaacttggtagatgcttctggctgttctatgaacactggtaataactttgccctgtttgctgggtctgattactacccatcaggcggttaccatgacctgcgagctaaatctcagaacatcgatgaactgcgagaccttatgaaaaagcagtttaatggtcgttttgactggtggcatattgccaaccttaatactcatacaattgtagagagtggttatcgttaatgaaagtattcgctaaagtaactcatcctgataacggctatagtcacgattctgacctggctcataatctgattgctgtggctccatttggtgctgaatcgtttcatgaagtcaaaaagatttctatcggccgttcgagcacaaaggtccagctggttgatgataaatatcattataacagtgtgaacttcactttctatgttatggaagataatgctccggttgagtatgatattttcaagaatgaacagatggctggttgtattgaatatgattatattatgatcagtcggtaatatgctttaagttttaagtgatattattactacatggagacaacagtctcaaaatgacctcaagagaagcctgcggcttaagctctaaaacatggtgaggtctctattcagaaagttcttgacacgataatttggtgggaagtgtacagccttcctggattattcgagtgaagccctcatgatgggtggagagcttttgaaatagatgcgggttaacttcagttggtagaatgacgggttcatatcccgttacgcgatggttcgagtccatcacccgcctccaaataaatgggatactagctcagttggttagagcaccggacttttaatccgggtgtccgaagttcaagtcttcggtgtcccaccaattagggtcgttggctgagaggggaagcgacggactgttaatccgtgtcttaggacaaggcaggttcgatacctgcacggcccgccaaataagaagcgagaccaaactggtaatgaccttgttgaggcctagcccagatggatgcgcactcgcaagtcgtcgaactgacgttccctgttatacaggtccctcattgaaaccaggtatatcacagcttgaagcggcgcaggaaggtactagttatctttcgaggtatagcgattcagttaatgtgttaacaaagattatatctgtgcacgggtatatgagagccattaactcttattcagctttccggaaagcttgagagattcgtatccctacgaaagataattacatggcagtctaaagagatgagttagaggtccataagtaaatgccgggaccgaaggcactgttcccgcatagcgtgctatgtgttctacaataacctacttgtttgtactctttagacccggataatgacgcttcggcagaacatcgcacggggcttttggccctatagctcaattggttagagcacccggctcataactggtaggttccccgttcaagtcggggtggggtcaccaaaacgtaaggggtggaaatatcttcaggacctccaatatcggaccctgcggcccaccccaaggtatacggttcgaggacgtattgacttttctccaggatgacacgtttggcgacgggcctgttgaggaaacgatttattggccttataaaaatgtagttcaacgccaattgttctataacctggttcgagtccaggcgttccgtccaaacaactactgagaaaaatattatgaccatcctaaatgaagatttacttccttgcccattctgtggcgaaaaattcccttttacatatatcagttatagttgttgcgtgttagaatgcaaatgcggtgtcggaatcgcaggttgtaaaggcgctgttaccgtggtttatgactcaatggaagctgttcctgaagaattgaagccttacgctgaaccagccaaaggtcttgctttcagagataatgatagaatcatcgagtgggaagaacacggtaaagtcggtctttcttgtatgcgtgccttcgagcatgctggtctaactaaaatttggaacaacagatgcaacaagattacgtaatttttaatccagaattcggctcatatgcttcattcgttaataacgaatgctgggaaggatgggaattcacagggctccttgaagcagatggttttacggatatagcacgggcccaacagcgaattgacgggcttattcttccgtcgggtcgtgtactaattgaagctgctgaagaacttaaaggatgcgttgttcgtcgacgctcaatcatgattcacgtagaggatatctaatggacattggctcagggtcaagttaccctagttgcgctttaagcaactttgctccgcatgcttttgaaatagacggcgttaaatgtgcttcaatggaaggcttcttacagtcgttgaagttcagctctcctgaaatggctgcacacgtttgcactttagttggtaaagccgctaagttcaaaggtaaaaagaagaaatggttccgtacgcaaactctgtactggaaaggcgtccctatgcaacgccagagtgacgcctatacgcttcttattgaaagggcatataatcaattggccactaatgaaggattcaagagagctatcctggccacacagaacgctacgttaactcactctatgggacgttctaaaaagaacgaaactgttctgactgaacaagagttctgcagcaatctttatcgagtacgtgaaatgcttaaggcctcgaaatgatagaattaatgaaagaccgacacggcgttccggtttttgaaggcgattatatcctgtttggtgtaaaaacctccggtatgggccaagatggtgaaatgcgtgttggtaaagtaactggcaaaacaggtcatgggattggtgtggattcttattcgtatcgattcaaggtatctcataaaattgttaaggtagatccacattttgctttagcttttatagagtatactaaataaacttattaaatgttcagtggtgaaatcggtagacacgaccggaatagcttgtttgaaaccctggctggtacccaggtgagtaaggcggaaaccggtgcaacagacctaagcgtacaggttcaagtcctgtctgaataatattccgcgcatagctcagttggatagagcaacggccttctaagccgtgggtcgaaggttcgaatccttctgcgtggaccatttcgcatcgatggtggaactggaatacacaggagacttaaaatctcccgccgaaaggattgagggttcgaatccctctcgatgcaccaaattaagacctgggaatagttgcttaaacttagtgaggaattcaacggcagatacgtctgtgcaactatataaggttcgaatccttatttccgtcttatgccggtttagctcagtaggtagagcaactcacttgtaatgagaaggtcggcggttcgattccgtcaaccggcaccaaatttatgaggaaaatatgaaagtttacgtcgtagtagaagaaaattggaacgtcgagtacgggtataatgagtattctctgcctaggaaaatctttgcatctgaagaagctgcaaacgaatacgctgacgaaatgtccaagatgaattcttacagcacttatgttgttgaagattacatcgtagaataaagaatgacgtcctagaattgcactcgaagacgtaaggctggcgaatgacctctgggtgcatctaagtcatcagagccgagaagaaagggttctcagaaatgggaaccctttcgggcatttagattaccagagtttacacactcaaaagaacgtggtatagtactcctacacaaacatgaggtgattatgaaattttttaaagatgttgaagatggtgagaaatttgtcctgcctaacggccaacaactgattcgtgtttccgaatcaagcgagtataacgctgtagatactattgattcatctaaaggcttctttattgctgatgacgatgagacgtatactgttgacgaattctgggctgatgttgaggaagtagaatgaaaattaccaaaacgtttgaaactaattctgatcgcgaaatcccgctgggttgctgtctgattcactggaaagaaggcggtcattctgaggcggtgatcggtcaattagaagatggttctagatactttgcttgtgcaaactggactgcacagcatgaaactcgtcacgaattgataggtcttattaagtacaattcagacaaaattgaacgaattgaatttatcaaggtgaaatcatgagacgtaatattactgccggatacccggcatctctcttcgagagtcaagattttattgacttcaacgaagttactgaatttctaccagcttcttgggtcctgcaatacgctgacctgattgtaagaaactgctcagaaccattataccaaagcgcctggaaagcatgtatggaaggcttccgtgctggtgctgcttcctgggaagatgaagatggatacagtgattctctgttcgacgtttacgtcgatgaagcaattacagaagctcttgaagaattcatctacaaaaatatgaaataaggtgtttacttttctagagggtgtgttactatagacacatcacacaattgaggaaaacataatgcattatcctgataaagctaaagtaatagcgcaaaagtattttaaatctttcggtggtctgtttggcaaaacacctaatttcccgtcaggtgaagagcgtttgtcagctttacgttcattagaaaatatcattttgtctaacatgcagacttatggcggcgatccggcgttgtctcgtattattgaaacggctgctatagatttcatggaagaagttatcgaaacttataagtagtttacaatgtgtaggagacgtgttatgatctcctacacaaacgggataatttaaattttacacactgaaaggaaaaatacaatggctattctgaaaaaactggttgcattcatccgtgctaaactgggtaccttcgttgctaaaaatactactattgaagatcagtatactgaagcggcgaacatcctgattgacaaaatcacgcagcttcgtgtttctcacgttaagtctgtcaatgaagagaaacgtatcttggctctggccatcgaaaaagataaactggctgaatcgaaagaaaaagaaattcgtcatctgatcgctgaaaacatgaacgtggaaacacacgctaagcttggtcttctgtatcgtcgaactgccgcggctctgcgtgaaaaagctgctggttatgcaggtatgcgtgaagaaatcgaacgtaaagtggttgagttggacgatgctcgtctggacctggctgttaagctcgaatacatccgtgaaactcgctctgctgatctgggtattagctctgccgacgatgtgatcgaactggcttctctggctaaagtatctgtagacgacatcactatgaaggttgaaacctttaacggtgctgaacctggtactaccactaccactgcagatgttcaggaatacctggcttctctgaagtaatacgaagagggcttcggccctcttattgggataatagaggataatactatgtctaaaagattacaaattgaagcactgcgtgaaaaaggtatccctgaaaaagctattgagcttttgatgccatatatgcaagaccgttgctctgaaaataattttttgcagacccgtggaagccataacaacaaaaaatacccacctgccattatgctggccaatactaagtttataaaagaaatcgtgctggaattcagccgtcgttataatggtgctcgtatcgaaggaatctatcaacttgaatacagcatctatgcgtacaatgatgataacgagcattggttctcttacgcgagagataaggcaaagtttttatctaaaaacgaggaccattttgcttttgtcttagaatttatgattgaaggtaaaatttacacggtttctttactttgcaaccctaactatactaatatgcctggtcaagggtactatagtgaacggcgcacttatcttagaactgtactgtcaccagagatctatgagttcagccagaaccctgaattagcaaagcagatcgttgtagaagagttcatcaaacattgtcgcgaggcagtaaatggctagatactatcgcaataaacctattgactggggttatgcaaaggtaattacagacaagttcttgtttatactggcaggacttactataggtctgtgtgctatgatattttatccggcattcatgattaatgaaatgggtgctgagatgtctgtactatcaattttgatcatgatagtaactgggttttcttttggattccctattatggctataccgtatgtcattaatggctgtgctaaattaaaacatatgtataattaccgaaaagaagtaaaagagtgggaaaaagaaaatgcagaacagcgccttcttgattccagagatgaaaccttagctttcttaaaagagatgagaaaatgagcaaatatacttttgaagtagaagttcctgaatacaatgattataaatcagcacgagaagctattaaggctgtcatgggcctgactgaacagatcgaagctgaagcagaagctatcgctgacgaaatgggtgtatcgtttagtacaggtgaatatggtaacggccgcacatattaccctaaaggtactagtgctgaagactacggtcttgaatactattgccagtgctatgatgtagaccttgacgaaaacggtaaaacggttgaaggcgcatgggtatcatctagcgagatgtgctaatgaaacgaaaccctaaagaagaaatggctaaagcggttgaagctattaatgatgcaattcgtcatgctaaagttgtagcggatcgctataatactgaattcactattagtcctgcttacggtatgggcggtacgtattattcgccaggtttcttgaagaaagaacttgagcagtacactgaacaaggtaatccttcatgggctaccgttaatcaacacgagtactacacgagccttgaaacaggtggttgggtctcttcatctatggaatgttaaaaatgagtaatgtaaccgaattagaagcagctgcaaaagaactggctatcctgtttaacaaagtttctaaaatggctagtgaacagaacttcggtattgaatttgatacctacgacggttctattcgtttcgaagattggctgaacagttcttgttatggcgaagaagctggtcgtgaatttaatgtagaagcagacggtcgtatttggcatcctagctcttgttaaggaaataaaatgattaaagcagaatttaagcttggaacagtcagagatgtcgagcaagccatttctaaactgatctctgaaggtgaagctatggcagaagcaacaggcgagcatttctgcgctggcaatgaagagtatatccctgtaacctctgatgagtgggaaagctataagtggattcgtgatgataactatctgcctcacgaccaaggcttctggtattccagctctatgcagaactgctagtttacattcactaagggtcatgttatagtgacccttacaggaggaacaatgattttcgttaaaaattgctatttgcattaccaaaatgaacatggcttcgaaagcacttgttctgtagcatatttgcatacgctgtacacccagtacactgttcgtgaaaaggtaaaagcagccgcatatctttatgaaaagcttatccaagaattagatggattcacttatcctgacgctaaattcattgacggtgaatgggtcatagtagaaaaccaacaaatcactgaagctgaaattaaccacttcatcaaatattgcagaggtaaacatgtctcgtgaattagattgtgctgtacgtaccgctaaagctgctctggaaaatctgttccagatctgtgcagaagaaaatgaaaatgtcgatatgtacttcgatgaccgcagtggcctgaatattcatagcgctgttcaagtaggctacaataatatttcaattcttggctggcagagtagctcacttgactgttgattactgtatcgaaactcaaaccccgttccataagaacgggtatatccattggtgggaaagagataagcaccacaacctatgtctgaatctgttcaattgggcgggagagatttcaagagtcttagaacacatgtctataggtcgtaaactactatacgatatgcatgaccagtatcctgttctgcgtggtacatctcttcccgttctaatctacgactttgaagaacagcgttggaaagagcctccttcagagcatgaaatgtttttagctttatgccgtaaaagagtttacaaccgtcttaaaaatcagtataatgctcctataaatcaactgaggaaaatattatgatgttagttatcggttctcgcgctcttgaacaccacggtcttatcagttatgatgacatcaaaaacagtgattgggacttcatcgcagaccaaggctcttggaatgcctttaaggcccaaatgctaggtattgaagtagcagtcaataatcctgatgtacgtgcattcaaatgcatgcataatggccgtgaaactcactttgaagcatatatcgttcctgctatgactcgtattcgtcaggaagttaataagatgctaccagaggatttcacttcgagctttgaactcttacgttattctgaagagttcctcactaaggacaatatttctggattttactgggctactccagaaatgtgcttggctatcaaaatgagccatcgtttcaaaaagaataacccgttcttccgtaaaacaatgcatcatattcgcttcttgcgtaacaaaggggttaaactctctttggctatggaagagattatgctccttcgtcagaaagaaactctgaactacaaccatcctaagctggatgtgaacaaaagtacgttctttaatgataccatttatacacttgaccatgacagcatccaccgggccgtagctctggcagatcgccctgcttatactttctatatgaaagatggctctgaagtcatgacctctcgcgagaagttcgagtcgctgcctgaggctattaaactggccggtgtttatgaagaaacttgtgtcttggctttagaacgttctcaggtccctaataacttccagaatgtaagttctgagcatagctttatgatggcccttgagaaagtatgtacatcgattacttcaggctggttccgtgaatacgcctgggaaaactaccataaggtagtccatatgtacaaaactcttggcgtgaacgattacattgatcgtttccacaaaaatgagcatctgattctgccgtttcagaaatgatgcataaaccgtttacagaaaagacctgtcctaagtgtgggcaggtcattagaatcaggtacaatagcaacaattactttgtagaacatgttcgctattgtcaagccaataaaatttcatggcagaacccgagatgattactaaagcattgtatttgtactggactaatagcagttacctgtggaaatcagaaaaattctttttctatcttttagttatggcagctactgctataggctttggcgctctgacgtttaacgggcttttaatctttgttccagctttattgccaccacaggtctggtttatcagtcttgttgtagtagcatgtactgtattttattgggtactacgctggtgccaccatcttaagctggaagctagcggtaaaatgactagcaaaggcactcgtcttgataaagaagatctcaagactcgtaagaaggaactagagctcaaagaacgtgaactagcccttcgtgaacgtgaacttgcattaggctttgcaaaaaatctgcgatgagtgtttacaacggtgtaggaacgtgttactatgctcctacaccaacacggagatttatatgaaaactttagttgaacttacccttgcttcgttgattatagttgcgtcatttgctttactggcttgtgataacaaatcagaggctactgaaagggtcgaagccgaaaagactgaactaggtattacaactacaggtcatgtaggtgtcagactgaacgacgctcagtgtatgaatacctccaacggtcaaatcgaagtttgcttaattgctattgagtgagaaaaatattatgaaaggtccaaaaacttttaacagccaagccaaagatgtaggtcgtttcgctatgggtatttccaacgaagtgatcaaaaatgaaacaatgttctttaactgtggtgtgtattttgcctggaaacatggtggtcctatcactcgtagctttattgaaaaccttccggaagattggcagtgcggacctgatttagtaattgacactcgtatccacatgctgatgcctggctggtatcctgctattcctggttatcaccatgatgacgtaccgcgtcctgatattccagccggtcaacatttcttaactgcaggtcaacctgattatgacaaccctcgctatcatgccgagcatattattggtcttgctaatgccgatatttgtcccactcattttgctactggtgtggctgagttttctgaagttcctgaaggtgctacggtctacaaacactggcacccagaagtcttacgtcacatcgaagaaggtacacttaagcaatgggaagctccggaccgtacacttcttcaattcaaccatgatacctggcacactggttctaaagcggttcttaacggctggcgctggttcggacgagtctcccgtaatactgaccgcgttaagaaaattacgaacgaaatccgagtgaatgctcaagtttatcttgagttccctatggaaggttggtaatgacgatctaccgtaatgagtatgataaggacgctatcggcgtccatcatattgacccaagaatgtctggagtcttgaaagaaatggctgaatacaaagaatacagtgtagaatatcctcagacctttgtagataattgctctaaagtcggtagttttatcggaccatttttaagtgtatctttatttctctattgtaattggaccgtttttacagcactggaactggttttgctttctgttttagtgagtattatgggtactcttagtgggttgctgattggtggcgctataggatgtggtattgctggaaccatcgctgggttcagaaaaaccaagcacgacaaacttaagcttgaacaagctaaagaagccgagtttgctaactggattaaaggatgtagaaaatgaatactatgagtagtcgccgcccgcgtacttggattgatgatgacgaggcaggattcaagattggctttgctcttatttttattaacttaggagccttaggcatatacggtctttttagtccgattcctcttgtatgggtatttgttacagcggtaagtatcttagggttcccgttagccgctgctattcttgcatacattttcacggttgttcttgataagttgtacgcgaaaaaatacaagaaatatgttgaagaacgtaactttatgaactgggtcaaagggtgtagaaaatgagtgctccaacttatttgatttcatttaaagatggccaggaaacaggtcgaatctactttaactggggatgggttcctccttctcaccaggaagtcgggattaaaattctgaagttcattgaactcggcattcaacctggtcaattagcaagtggtaaactcggtgaatgggataccattaaagaaaagtattatggtactctgaaacgtgaaaagtactatgatatgtatgatgtgctgaaggctattcactatggcctttatgatggcgaatttaacgtgaaggattattttgaatgaaagaattaagcgccgggattatgttctttactaaagaccatcgtctgttcatggctcgtatgaccggcattaagcgtaaagacggaagttctcgatgggacattcctaaaggtcacgtagaacctggcgaatctcctaaagaagcagcaatccgtgaatgccaagaagaaactggattcacagactttaatcctgaagccctgtatgacctaggcgagcacgagtacgctcggaataagaatattcatctgttcagttatatgcgcttcgtggacatcgagagcttcaggaattgcaaatgcactgcttatcacacatttgaagatgggtcttctatcccagaaatggacgcattcgcatgtattaaacctgagatgtggaaacacgtaatggggccgtctctttaccgtattatgcagaagttgtatcctaatgtagcatgataaatactcctatcaatagataggaggtccatatggacatttttgacatgcttcgtatcgatgaaggatacgacaacaaaatttataaagacaccgaaggttattacacaatcggtattggccagctcttaaccaagagccctgatattaatgttgctaaagctgagcttgataaactaattggccgtaagtgtaatggcgttatcactcgtgctgaagcagagaaattgttcttgaattcagtagacaaagccacccgtggtatcttaggcaacccagttctgaagcctgtatatgaatctcttgatgctgtacgtaaagcggcattaattaatatggtcttccaaatgggcgttactggcgtcgcagggttcactaattctttgcgtatgcttaaagagaaacgttgggacgatgcagcagttaatttagccaaatctaggtggtataatcagactcctaacagagctaaacgggttatttcaacatttaaaacaggaacttggaaagcatatgaaaacctataaagaatttatcgccgaagcgcgagtaagtgcgggtaaattagaagccgccgtaaataaaaaggcccattcatttcatgatttgtccgataaagaccgtaagaaacttgtaagcctttatattgacaaagagcgtattctcgctctccctggcgctaatgaaggtaaacaggccaagcctttgaattctgtcgaaaagaaaattgataactttgcttctaagttcggtatgtctatggatgaccttcagcaagcggctatcgaagcagctaaagcaattaaagataaataacaatttacttttacgatagctgtgatatagtagccttacaaacttaatggaggctactatgacacgtattaacttaactcttgtttctgaactcgctgatcaacacctgatggctgagtatcgtgaactgccacgcatcttcggtgctgttcgtgatcgtctggctaaaggacaaggcttcagcgatattcctaaagacttccgcctcggcgcaggtcatgttaagttcttctacgataaaattggctttttgttcttgcggcaaagtgctattattgaggaacttttaaagcgtgggttccagattcaaaatacccatgttgacgtatcggatatcccgctatgtttaatgaacgattttactccatctgaagctgacatcgctttaagtcaggctcgtttggatgaaaaaattgcacaaaggcctcagtggtataaacattatggtaaggctatttacaactaagataagctttgatagaatgcttctatcaacttaaaggatataaaatgaaaacatataaagaatttatcgctgaagcttctgcaggaaaggtcaatggcattaataaagatgagtggacctaccgaggaaacggctttgaccctaaaacagctcctattgaacgatatttagctacaaaggcttctgactttaaaagcttcgcttgggaaggacttcgctggcgtaacgatctaaatattgaagccgatgggcttaaatttggtcatattcaagatgttgttgctagtaacttagacgcagcttttgttaaagccgatgcagaccttcgccgctggaatttaaaactattctctaaacagaaaggcccggtgtttgtgcctaaagctggtaaatgggttattgacaataaattggctaaggctgtcaactttgccggtcttgaattcgctaaacacaaatcagcatggaaaggtcttgatgcaatggctttccgtaaagaattcgccgaggttatggctaaaggcgggtttaaggcagatctagataccactaaaggtacgtttaaagacgctaatattcagtacgcttacgccgttgctaatgcagcccgtaattaaaactaattcctaataaagctaaacacgacctctcctcatgaacgtcgagtcctctgagtgaagtagctttccctacctgtaataaggtcgagcgcaagtgcggtaaggggtttacatagtgtgtcgatggattaaacatgtgccaaggaatggccccatttaatttaaatcttaccatcctaggatataaaatgaaaacttataaagaatttatcactgaagcaaaagccccaaaggtttttcaaattgctgctcaaggaaatttagacaaagactacgtcgatgctattgtgtcaagcatcactaaacagggtgtagaagtagatttcgttgattttactaaaggcaacacttttaatatcgttgtatctaaaggctctttggctaaaattaaaaaggcttggggtgttatgggtgcattcgaattggatgatgctgaacttcaagctaaaggtcgaatgaatacaattaaaggtcgcggttctctataagtttagtgtagagaattccctttgccaaggactaccacgtgccaaggaatggccctactaactgagaactatcatgaaatacttaacagctcaatatctatctctgatgcatgctttcacagaccacgcaatcagtcgtttagataacccagaaaattcatggcaataccctattgctatgcttcaagaatatggaacactacgtcttgatggcggccgtcaaaccggtaaaactgaagcagtctctcaattcgctgcagactggctttcagaaggcaattctgttgtagtcattgcaaacaaaagcgaatacgctcgagatacttgcggccgtattaaacgccgctggaaagttttagaaaatattgataaagaaaatggcgtactgatgcatgatactgtgcgcagtttcctcagtggaaaggataaattccgaggcatttcgcttaaacggacgctctttataattgaagaaccgattcgtattccagagatgtataagttctacgaagcttgggacgatctttctgtttgctactctgtgaataagcctaagttacctttatttttcgttttggggattcagtaatgacacgttatatgtttatggatggtccttttgaacggtctgttattcagacagaagcgactattgaagagctgaattctaagccagatattgaaatcatatttgttactggaccactttgcggtctagtatctcgtagtctaatttgttatgatcgtaaacttattcgagctatcgaattgtttaaaaagcctatcaagttggacatttctatccgctacacaaccggtggttaacatggcccaggttattattaaaggttcagataaggctattaaagccttttgtgattggttcagtaacagtggtgagcaagggctgatggaagcctgggccgacgactggaacccagaaaccatgaagtatgaagaagcaacttcataccttggcactcgtggctactcgtcagaaatcgagctagtcgaatatgacaaagaaacagatgaggaaattccttatggccgttgaagatatcaaaggttacaaaccgcacaccgccgaaaagattaaccgtgttaatgccatcaaagatgctgaagtccgtcttggactgatcttctcagccatcgaagacgaactgcgtgaacatgtcccaggtcaaagcgatgaagattttaattctgatgttcaggcattaagtgctatagtgctcgctaaagaccgtcttaaagaagcttctatgtgggcctgccgagcagtgttccggcctgaagaaaagtactagtttacaagtgtgtaggagtatggtatagtacttctacaccaaaatgagaggaaattaaaatgactaacatcgatccaagcgttttcatccgccgcaataagctccgtcgtgtctttgaaactactttcagagatatcaattcttctgtgaagtctgccgctatcaaggctggaattcaacctttctttattaaatactctaatcatctgttggaccgtgctatccaacgcgatatcgatgaagagtatgtattcgccctcttcaaaaagattcatcttcatgtcgaagaaatctctgaattcttgaaattaccacctctgcctatagatactgctgatatcgatcctgatgtggtgtatcgtcctcagagacttgaagtcactgatggcaccttatggctgggtatgactgtggataaccatattccgggtcgttggtattcattgaaatgccgaatggcttttgttaacaataaacgtttaaaaggtaagatcagcacatacgttattaatctctaagaggaaaacatgaaaaaagcgatcctggcacttacgatgctttctgcatctgtttttagtgctgagcaccacttcagtaatgtccaactcgataatttgcaatacgcatatcaattcggggaacaattcactaaggacggtaagtataaaactcacgagactcgtgataagaaaggacttggctacattatggccgcgatagcttggcaagagtcaagtgcaggcattaacaccagtaagaataaaaagaaacaccacgcttatggagtgtttcaaaattacttgcctacagttcgcaatagggctgcacaaaataactggaatctttctgatacagaaatcaaaagaatgctatcagatcgtaagcattcggcccgctgggcgtatattgaactgagttattggttaaatatacataacggagatattcgtaaagctatctctagttataatgcggggtggaacgtaaaggcaggtaataaatacgcctcccaagttatagaaaaggcgaattatcttaaatcgcatgaaattttaattagtggatagatatgaaaaaattatctttagcccttggattatttttatgtttgggagttaatgcggcacagagtaccgataaatccagcatcctcgaatacgcacacaggaccgctaaagactactgttcgccaaaaaacgttcaatgcataacagagttctctaaccaagttattatggcttttaaagacggtcagatggactcgagatctcgattccgtgaaaattcactttcggatcgttatgaaaataggctgcttacaacagaatgtattccatctgatgtcaagtataaagaaatatgcgaatctatggtcgaccgtttagtcgactcatacaatagaggtttaaactctaaatgataacccaatacatctccggcaatatcgtagaactgtttaaaaccggacatcatatcgctcatggctgtaattgccatcaccaaatgggcgctggtatagctggtcagcttgctaaagcctttccacaagtattagaagcagatatccgtgaaactgagcttggcgaccgagaaaaactcggccttattactatagcagcaaatagccagcctgaaggaacggcgtattgcttcaatttatatacccaatataatcctggtcggaatctagattacggggccttgttgaacgccttcagacgtcttgatatgatatctgaaaatcaaatcactcctataacagtttatattcctcggattggagccggtatagctggtggtgattgggctaaaatcgaaacaatcattaatatgttcaccgaccatactaacattattgtagtagattgggacggttcataatgaatatgtctgaagaagaaaaagagtacatcatcgaaaacattgatgaactaatccgtttagctaaaaaggttggtactgcttctatcatcggtacagatgatgcatatatccaatctattatagctttagataaacaacgtctcaaagttctaagtatggatggtatcgaaggtgactactgaacaaaaggccgagatgtttcgtttaatcgatgagtacgttggtgctcattatgcttatacctggctagcagatggcggttgggactgcgacgatgaaggtcttgaccacgctaatgcctggaacgctttgaaggattttattaatgggctgtgaattaaaagaaatgattcaatcttattcttctttggcgtctgatatcgcttttgaagaaagtaagtggaataaatcccgtcggtacattaaagaacttcaagaagaatacgaaactctttggcagcaaatagaaaataaaatcgaagaacttcgaggtaactaatggctcaattgtactttaactacgcagcaatgaacgctggtaagtccgccaacctgttgacggccgctcataactacaaagaacgtggtatgggtacattgattcttaagcctggcgttgatggccgtgattcatctactgaagttgtatctagactcgggcttagacaggaagcgaatatcattactcctgatatggatatattcgaattctttaaatgggcccaaacgcagcgtgacatccactgtgtatttgtcgatgaagctcagttcttaactcggaaccacgtgatagaactgtgtaaaattgtcgaccaatacaacgttcctgtgatggcctacggtcttcgtacagatttccaaggtaatctatttgaaggatcagaaactttattagcagttgccgataagttggtagaacttaaaggtgtttgtcattgcggccgtaaagcaacaatggtagcgcgtattgatgaagaaggtaatgctgtactagaaggtgataaaattgaattaggtggtgaagacagatatgtttctctttgccgtaaacattggttcgaattagtgactggaaaataatatgaaatgggtgatgtgttatcgttgtctacacgtctatgattataataaggcgcctaggactgctaccaagcgtcttaaaaataaagaacctgaatgtcctaaatgtaaatgcaaattaatttttagctgataaatacgtttgtaaactaactcaagaggtgtatatgaagcatttgtcacataagcaactccgtaatttgaccatttctcaattagacgaaattcgacttgaggttgggcacattatttcagcacttcgtatttcttataaagaaactgattctgaagcggactataagcgtatgcgttctcacgaaagatacctcgatgcagttaaggctgttttacagcataaacgtaatactggtcaaaaataggaggccttatggcctttaaacactcactttgtctcgccgtaggtgtttgtattagtcttcctgttgtagcaacagaaacagaaggatttaatcaatacgtcgaaggcgctttaaaggtttacgcccagttccaaaaaccaagtaaacaagagtcagagcagttctatagctttattaaaaacaaatggtcaactgtaaattgtactagtgaatgctctgttaccggcatcaacgccggactacagtatgcatcccaaatgaggataccattagaccatgaagttcaatgagtttgtaacatctggtaaacctaatgaagcagataattacttaggtcttttaatggcttctgcgtcatattttcactcggcgcattttgaaaccaaaagttactcgcgacacaaagcatacgataacttttttagcaacattccggcgttaacagattcttttggagaaacttggttaggattttctggaaagaaatactctcctgctattcctgctcaaaaagatctcccgcctgatacaataaagatgcttgatcacatcataagtgagtcagaacgcatctatgaaactatgcctggggccattaaaaacatcattgatgagattaatgctctttgctatcaaactaagtacttgctttcattagattaaatcttaaggggaccttttggtccctttaggggtttacaacggtagtaggacatgttagtatatccttacacaaacatgaggagaacattatgattaacacattccgtaagtatggcgaaatgaaagtgcttcacaaagacggcagaccgttgactcttaactttaagttcatcccagctatcgatgaagtcggaatctgcactgaagatttttcagttaatgcaccgttcggattctgggtagaaacttctgcttcggtagaagagaaaactactcttattgaagatgagttcagttggctagccgatggtgaagctgaacgtgttgctaaagaaatcgctgagtgggtgctctaatgaatatggtaaattcctctgtaaaagcagaattgattaaagctattttcgagcttaaagaagcttccgttctgtttggcgccgcagttactcctacgttcccagctttagagcataaagctcgtatggaaaaactccagaaacgcgttaacgaaattgtggaaaggctatgaccgcagatcaaaaacctttcgattatcttaaggagctgtaatggctgttgggtttgctaaagacggagctgagcaagatgaaatcgctgcagttgtacaggctgcagtagttcacgctcgctcccagttcaataccgaacgtgaatcgctttatcgttgctttgagtgtgaagaacctattcctgaagcaagacgccaggcaattaaaggtgtgatatactgcatcagttgtcaggagatgaacgatgaaatttttaaacgtgagccacgtaactgctggcataggagcatgagatgagcattggtaaacttgaagttggtgatttgattttaacccgtacttacacaggcgatgaatctgtagaaatctgccagtaccgcggcgctacaggaagtttaatgtatatggtatatcaccctgaagctatcttgaaatataaccaagaaaggtttgttaaagatattgatagccaaccggctagcgtccgtatcgttcgtataagcaatgctgatgcttgggctgaagtaatgactcgtgcagcagagagtttacaagctggtaatgttgtggtatagtattcctacacaaacaaacgaggaaaacaaaatgagtttactaaagtggcagtacccgacatcatcttctgcggctaaaattgaatcagactttccgggtgaatctgacacagatctgtcggtattttctacttctaattttgcagaacttaactgcgacgtcattcatgtaaagcagttcgatgaaaaggaagatgaacttaactgggtcacctttactaaagctgaagctcaggcacttgttgcttatctgaattctgttattccaactatggacaaataaaaatgattatcaaacgtgattcttggcactgcaaaatggtcacttatctgctagattctcctaagagagatctctgtggatatattcgtcaagtgctctggcttctgatggtgtttattgctttagcagtatgtttacttctgtggttcggatccactggtgtaagtatttttactgcacttggtattagctcggcaggttttatgcttctgccagcaacattaaccggtctgatggcgattgtgttaattcttggacttctcggcagtcctatctactgtatttggtatctttacgaaaaacgtagatctcgtaaagaagctgaagaatacgaagctcgtgcaaatggtacgtatgttaaacctgagccaggcttcattaagtcagcgtataaatcattcaaagacaaaacctgtcattttatcgagttcaaatgaaaaacgatcaaacttatgtagtaaacggccgagaggccgtttttaatgcaacagtgattaaaagtggcatgttctatttccctgttgctgtatttgtctttaaagacaacggtgaagtgcttaaggtaattcataatactatttcttctaaacagtatgttgaagacaataagccatttgatttagacatttattttgacggccaatggcactcaggtaaacgaggttaacatgtttattaaacaggtcaaatctaaagtccagctgaaagatactttggctcgacaggattctcttgagattcttactgaagacggatataaaatcgaatgtaacgtcgactttatctcttttaacaaccacggtacttataatattttcttggctgaatctggtgcccggtatttagttaaaggaagtaatactgggtactgtgaaattgctatctttacagaatataaagtatcttgccaccgtgtaaaatgcatgcatcggacggctcgtaacttccgtgaagaggtaagtcctaaagaccttagctacaatcgtgcaggtgaacagtatgtctgggtcccagttgaagtaggtgattcattcgttaaatccggcaacacgtttgaaatcttggcgattagttctaatggtcagatgatgtttgcaagcaccccggatggcggcggtctgcagattaactttactgacaagtgtatgattgaaaaattccatggtcttcgttggggtacaccagattgaaaacaattgtaaaagcttacttcgggtctcatctttatgggacctctactccggaaagtgatgtagatttcaaagaaatcttcgttccgcaccctcgtgatattctaatgggacgagcaccgaatcactttaacatgaacaccaacaacacggctactaagaacactcatgatgatgttgaccatgagctttatagtctgaaatacttctttgaactggctcagtctggtgaaaccgtagcacttgatatgctgcatactccatcagacctggttgttaaatctgatttgcctgacgtctggaaattcatccaagacaaccgttctcggttctatactacagacatgaagtcatatttagggtatgtccgtaagcaagccgctaagtatggtgtaaagggttctcgattagctgaactgaaacgcgtattagaagttctgaaccaatatccagaatggaaatatgctgatcggcctaaagataaggctcacaaccaacgctggaaattaggcgaaattgaacaccttctgcctacgtcagaattcttgttctggaaagacgacgttgactctaaaatgggtgttcagcgtttctataacgttctaggccgtaagttccaagatacaatcactgttgctgaaattaagtacagtctgactaagcttgaagccgaatacggtgaacgtgctcgcaaagcagaagctaacgaaggtgttgactggaaagctttgagccacgcatatcgtgctggcatccagttacgtgaaatttaccacactggtgatttagtattccctctatcatcagccgatttgattaaagaaatcaaagctggtaagttgccgttcaaatatgttcaagaacttctggaagacactgttgaccaggttgaacgcctagcagcacttgctcataagaacggaatgcgcgataaggtcgacatgactttctggaacgacttcgtcgagaaagtttatcttgagaaccaccggtcttattatcggtagtttacaaagcttttaacgtgtggtagtataatcttctaccacacaagaggaaatattatgagcttattttggattgctatgcctatcgtgattgctattgtgtactttgtaattgggttcatcattgctaacaagatggttgctactgactacatcgaatcttctttagggtggtggacatacgttttgttctggcttcctattgttatctggtctggcttctaccacgctataggttggctaatttctattccagctaatttagctgagagccaaatcaaaaagcatctgccgaaaggtcctcgtcctttcaaataaaagtaaggctccttcgggagcctttttcgtttaggtgtttacatcctcgtatgtcatgatattatagacctacacaactaaagaggtaaatatgaaagctattatgtggtgctttggccctaataagattataggcgagctccaggaacaacgtgaggtagaaggtgtaatgtattacacttttcttgaaaaagccacagggcgttgctacacactccaggaatcagactttacatttatgtgaggaacttatgaaattatatgtatatgaaatcaaattcaaacgttttgttgacggtatatgggacggtaagttccgtaaagtttctatcagcgccgaaaatgaataccaagcggtgttccaacacggccagacaaatggggtatctttcccagagcagaatgttgaagatgttatcgtgatggtctcaggcggtcagcattcttactttgaagtaggccatacctacgaaactctagaaggcaagaaagttaaaatcgtcgagttcaaagaccttaacgttaaagaaaagcgtactggctatgaaaccgtgatggacgaaaatggccaccaccgctactctcgtcgagatcgtggtcgttgcaccggctctaagacttcggatccaggaaatattcagctcggtgtattctggctccgtatggacattgacgacccttattgctacatcatggaaaggaaatcataatgggtgctttactgtttttgatgctttggatgtctgtaggtttttgtatcaaccataagaccgattgggctatgccagggcgtaacgaccagaaagtaagaaatttctttttggcgttaaacatgttcacctggcctttatggcttatctggtacttagcagtggagttctactaatgagctgtgtaagaaaataccttaaaatccgagatgtctttgcatctgatatgattgcagctgcaatcaagtatgcgcaggctgttaatctagcctatcactatgtaagaatgtatccacgcgatatgatcgagtttagaaaaagacatatcttgaagtctaaagcaattcatcttattattatggatcacgaccagctccaagcagcaaaagacttcaaagaagctttagatgagctggatttagagattgaagctaaaatgaatgaagcacaacttaaggctcataaatccgtttggttcacaggagaccggtaatgtttactgccctaggtttagctttggcaagtttgctcttgattgtgtggatggccgccggttgtgtaatagcaataatgacgtccggctgggtcaaaggtcggactgcggtagagcgtctacctaagaacattttgattatcctattttggcctctttggtgctggtatatcttaacggtggtgtaaattatggaactcattttaagtttgtttgcagctgttgtactatgttcggtgtacttatggtatgggtttaatttgtatactaaaatctggtgggcctatggtaagcaaagccaacttaagtcaattttaacctttatcggtgctgtcttaggctggccactaattttcttaaagtgcctaacaaagtagtttacaacggtgtaggatggtgatactatcatcttacaccaacacggagatgcaaaaatgttaaacgaaatcgtaatgaaaattattgaagaaaatcgtaaagcccgccaagcacaccgagcgaaagttgaaaaacgcgccgaggagttgaatgctggatggtgcaagtcccgtttcggacgtgacggattcgataaagtagttgcacctacctggggagtcgatgaccgtccgcatgctccatttgacggatatctgtgggaaaacgaactgggtgaagtagaagcttatcatgccgggagttatctcccgtatgtgactgaatttgataacatggataaacctgagtatacaggtgaccacgggtggtggaagcttcgtttaacacaagaaatgtacgacgagatcttcgatatctacggtatccagttccaaattccttataagcgttggactatggaacagacgaataccgaagttctaatggttaaagtacgagctcaccaggacatcctgaaagcgatccaggaatactctaatgaatggtttaataacctattcgagagtatgaaacaggacaaaggcgaagctcctgtgggcaaacaggtagtaaaaggacgtgttgtatctgttaagaccttcgaagattactacggtattgtttctaagatgactgtagtgttagaaaacaaatctactgtctacggaacattgccgaaatgtgtaccactggaccatcgtggtgaaatcgaattcagtgctacattcgaacaggctcgtgatgataaaactcacgcattcttcaaacgaccaactaaggttaaaatctgatggaaatagtagtatgggttctggtagccttcattggctaccttgttattggtaatattgttgcctgctgcatgaagttacgtggtcacataatgactgatacagagcatgatttcaagatagtgatagtcctttggcctgctcctgttattttgggtattttggaatcactcattaaaatcgtaacatttccgttcggcggcatctatcgtttcatggacaaacgcaaatgattattttatctaattggatcgaaaacactgctttataccctccagcatatatctacgccggaatgcctaaaggaaagcaagagaagcaggctgaagccatttgtgaagaactgtataagtttaattggggtaaagacaaaaacgtcctaggcgacctcagggagctctggcgccatactagtgttttgtgttctactaattacgacggctctgttaagcgtgaagtaattgaaaaatacttcgatgatttacaagctgctcttattgaagctaataataagcttcagtcagtgttcaagaagcaccaagatctgaacaagtggtacaatgaccatcttaaagtgtctaatgcacaaattaaagaaggcatttacacctacaaacgggcccaacttcgtatcattgacaacagtcgttttgctttaaaataaatgtcggtataatggtttcatggatgattccattacactctatactcggtgaaaaaagaatggaaattttatttggtttactttgtgtattctgctatgttatgataggaatcctgatgacgaagttgttcatcgctacatcaggttggactggtatctatgatgatgtcactgaagatactgagatagcatggattgtaactttctggcctatctttgctgtgttctgggtagtagctattgcaggactgccactgttagctttaattcgtaaaatattctgaggaaaatatcatgacccgtaacgaagttattgaaaaagcacacgaaatttttatcaacgttcaggctgaaacggtagggttccctaaggcaaaacgtattgatgttctgtctaatcgtctgaagaacgagttcggcgactttgcccatctgtttaactcgagccagagtcggaagtggcgcgtaggatttactcatcattacgacttcaaagagttcttcaagcactctcgtattctgtctaacatcatggctcgtaaacgtcttggcaatttcggtactatttcaagcactcgccaacgttatgacctgcgagcagcagaataccgtggtgctgaagcagttatcactaaccaggttgagtacttcatcagccaattcaactacgaccgtgatctgattctgaagcttgcttctaaaactatgcgtgatactaaccgtcaccaactgtttgatcatcagaaaactattcaggtgattgctaagcgtccttctatgcttggcttgaatgactcagtctgtattgatattcgcatcaacaaagacaaagctcacaaagatttccgcacgctgaccgtagttaaagagcaggtagcacaggctctgtgcatcattcctagtgtttcttcctatgaatggaaaccttctaacggccatagcggtactttcatagtatttctgaaagatgctaaaccggctaaagagtttgatgaagaaattctaaacggtaaagtttcctcgtcaaaatatacggtagataaatcctttatcgctacagttgctcagcctgaactagttccagctgaacgccactttgcttcccagatgcttgaagtagtgtctaagctcgatgacatggttaaagaagcagaacagaaggtggaagatgcccgtaagcatcttgccaacattacaacccagcgtggtaagttgttgcgcgcaattcaggcactgaaatgagaaaccaactgaaagccgacctcgtcacggatgacgaggattttgaaattgactggaaagcagccttagaaatggttgatagacgtgaagctgcagcaaaacaagttgtcccttgtgaaaaatgtagtagtatgcaagtacaattagttgactggggagctctgatattctgaaaatgaaatgtcggacttgcaaacataagtttgagagaacattgaaatgattggtattaaattagacacccacgcagtacgacagatgttcccagaaggcactgaagcttattctcagttacagcaatctgttatttcgaacattgctaaagaccttatcgttaaagataccggtaataaagttcgtaaattgattcaagatgaaatcaatgcttacggcttagttgttcctgatgtatcgatggctgttcagaaagaagtcgaatcatggttcagtcgtcgtggttatggcgctccttatgaaacctctagcgttcttaaagatcgtcttcgcgaagctgctaaccgcgaagcacgtaatgctattgactctgttattagcgatgtaattaagaatgctaccgataaagcaatgaaagatatagaatacaaaatcgaatctgctactcgtcgtatcgagaaaatcattgttgaccgtgttaatcagaactttgcttcaatcatcgaccaggctgtggcagagaaaatcaagaaagctttcccggggttataatggctaaacgtaaaaaatatctggaagaagccgaacgagctattgttcgcttattaattgcttatgctgaagagcatgatcagattcctgatttcagcgtattaaaatctgcattgactaagagttataataaaattcattcagatatccatgacgaaattcgtcgtagtttgaaaattaaaacggccccgatacaatcgtattgggatgccatcgccagtccaacgtataaagacgcttacaaagaattcatgattcagattaaggaacaaaagagtgtacacagtattcggttatgattcaactatccatcgctgcccgtattgcgacaatgcgaaacgcttccttacaatcaaaaaggagccattcgaattcatcaatgtcatgccttcaaaaggtgtttttgatgatgctgttattgatgacctacttaaacgccttaatcgcgagagcaaagtaggattaacgatgcctcagatttttgcacctgatggcacccacattggcggttttgacgaattacgagcaacttataaatgactatagctttcttaattcttattgcttattttgttatcggtattggctacggtaaaactctagttgctttagatgacggctctgctcaaggcacagttttcatcgcaggcttcttgttctggccaattgctttaatcatcgcttctttctggaacttcaaatgaatgatcaagaatttgccgaatttaaagatacagtagattttcttaactgtgaactggaaaccgaccataaggtcggtacagtgttcttccattgtccacagtctattaaagatgaagccaaacaatggggttggtctgatactgtagttcgtgaagaactttgtgaatttatggaatcactatgaaagatgaaataattgttttaaatgaagccaaaggtgaagaagaccttgaatggatgctttatgaattcatggtcaagcgcgctaacgaattcggtattgactgggccgtccaaaacgcgtacggcgaaaatgaagttgttattaaaggcgtagcttacacagttcagtggcaatacgtgggtcttgaaccggccgactataaagaaatccgtaataaagaaacaggtgaagtagactacgatcctgttggcccttggtcatgggattacggtggtcctgacttcgaagtttctagctattggcgtgatgagtaagccatggtacagcgctcgttgggaaactctcgagcctgaagaaccagtttacactgagtctgaatcatgttatgatgaacctacggtaaatgatctacttaatttagaggattactatgatcggtaaatgttttgaaatcgttaaagaagataatgaagacggttacggcacttcagtgttgtttccacaactgaaggtaggcgtcaaattcaaggttctgcgtactggtgttgaagaatcatgccatcgccacggcattaaaagtattattttagaagacggaaccgttattgatatcgattctgtagattcatctttctggtgtctttgggccccggcctctatggacgaaattaaagaaatcccgccagaatcttttggttctatagctagcacgggttattttgacgggcaatatctgcctgaccgcttaaaacgtattgaaagggaatttgacgctgagtataactattacgatgcaaatgtaattaaagctactattgaacacattaagtggcttgaagctcagttgagattttctgacagaccgttctaataataaatagattcatctacttatgaggtgaatctatgttattggctggtaaactctacaaagaagaaaaacaaagactatttgatttgcagggcggtgtatgtcctttatgtaaacgccctctagataatgacgtacaagccaatcacttggaccatgaccatgctttatcaggtcctcaagctggtaaggtgcgtgcattgctctgcaatctatgtaacgtaaccgaaggtatgatgaaacacaagttcaaccgttcaggtctgaaaggtcaagatatcgactacctcgtttggttgaaagaattagtggcgtaccttgaaaaagattattctggagctaacatccatccagagtacgtgacagacaagtcaaaagaatttggacgtctcagtaaagaagagatggttcaagaaatgcttaaatccggatttgtatataatgaatccgacactaaaacgaagcttcaagcttcattcaaaaaacaactgaggaaatcgttaaaatgattatgaattttgtgaacgactttatcgactgcggcaacgaaactgttctgcgtttcggatctgaattcccggcgatgttttccttcctgttctttttctcttactaaggattaaaatgactaagttccgtgatatcgtagcaaacatcatcgtttatatcgttctgttcttctctgctctggttgctgtcccagccattgcactgattatggttgctgaagctattcgtggtactaagcaaccgagtgtttctaaagaagaaatgactcgtcgaatggaagagcttactaataagattgaaaagcttttcaaatgaatatagaaatctacggcatcccagaacaagtctggagatgtcctggatgtcattggataagaggccttttggcctctttgaacgttgaatatacttttatcgacgtaataagtcctggtgacaccgggcctgtttatgataaaccactcatcgaatctctggctaaacgtctaggccaattcccttcactctctatcaagtatcctgttatctttgtagataatcagttcatcccatctcttaaagatttccagcagttccttattgacaaaggctatgaccgagatttaattgaagattagggtgtttacaacgccctttagtcatgttagtatactcctacactaacaacggagatacaatgtgaaaactgtcgaaatccaacgctacatcatcgctcacgtgcaagttgaagtttcaaacgggtatactcaggaccttatgatcgaggttcgcggttctacaatcttcttccgtagtcgtgggaagtgtactgagtgcaataccatggctgctaaagctcatgttggcaaagcggttaatgccgcggttgctttgattcctgagatgtgcgattacgagcgaaacgagatcggccgtttagtattggggttaatctaatgattaaaattgaagatgcaaaggtaattattcgttgtatcgttgatgaatgcaaagttatgttcatcactatgtggcccactgctgaactaggccgaatccaaattgaattctctattggttcaaaagtatgctttattaaaaagggccaatatgtccatcttcaaactaagcttaaagaaataggcgttaaaataactgaagatgaagcttcgcatatggctagtgtaattaatcaacatttggaaatattttaatgggtaaaacataccgtcgtaaagatatcaaatcgaaaggtgaatactggtcagggcgccgtcaggctccagcaggcatctctgatgaaactgtggcagataacatcttccactctgataagcctaagcggtggaaaggcctggactctgcagttaaagaagaccagaatcgctatgcacgggccgacaaacgtcgtctgaaactgaactacgattctgaaactgaactcaaaactgacaaatactacgctcgcaaagccaacgaaggctggcaacactcgtagtttaagaactcgtgaggaccgagtataatgtcctccttggataataaaatttaaaccacagaggaaaatactatgtctcaggctatcaaaaatgttctgcactcttttgcttttgataaagtcgaagaaatccgtaagaagggtggcattgttactcctgaaattttagatcagtgggaaaaagaccttcacggaactatggttgaaaatgagcagaaaattggtaaagctcgtattcgtgaactactagtctgctatatcattggcgaatttgatattgatgcttttggtgtccagtcagttgtcaagaaagctggtgaaatttctgacacgtgtattcgcaaaatgaagaaccaacgtaagaaaggtttcaacgatctcaagatcgttaaggcggcaaaatgattgttcttaatcaacgcggctgtccggaacgtgtacggttccgtatcttagaattaagaaacggtgaaccttatttagttgatagctatgcttctatcataatggttcaacgatacgttaacctgcgtacatctggtctcacgcacaaagaacacgggcttccggttaagtgccgattctatatgatgagcgcggaccaccctgattattcaggtcttagtaaaagctggaccgaaggtcttacacttaaagagcttgaggattatcttaatgactgaagaacaactagctgaactcggataccttggttatctgataagtcataccgaagatgggcgtgttttgattgaaggttcatctaatggcatcgactggctcattgaagaagactttggtatctggtgggtatacgaatacacttcttcagatacatttatttctgttgatgctttcggtgtatttgaacaagctcatgaatgtgcaaggaatctaagatgaacagtttaactttggttgctaacgatgaacactttattgtagatagttacatgggtgtgtattatttaccatttggtgatgcatcagatgattcgtataatttccacgtgtctgaactgaaccacagccattatcaacgcttatataaaggcttggtacaataccaacaagaaaatggaataactgtttctttaaactatgaacagttccttagtatctgtgaacgtttatttgaaatgtactgcattcttcgtaagctgtaatatgctttaagcggcctcttcgcatccgatgatataatgaatatatactatttgatgaggccaattatgagtgattacgtaaataataaggagttgtacaaagctatttgtgcttggaaggaacaggttagaaatgctcccgaagggactaaagtgcgacaaaatgacacgattggccttgccatcatgttaatagccgaaggtctgagcaaacgattcaacttctcaggatatacccagtcttggaagcaagaaatgattgcagacggtattgaagccgcaattaaaggtcttcataacttcgacgaaaccaaatacaccaaccctcatgcatatattacaatggcttgttttaatgccttcgtccagcgtatcaaaaaagaacgcaaagaaatggcaaagaaatatagctacttcgttcacaacgtttatgatagtcgtgacgatgatatggttgcgttagcagatgaaacctttattcaggacatctacgataaaatgacgcaatacgaaacctcagcatacaaggcacctgggtctgataaaaagagtgaaccggtagttgaaggtaatgaattggaatttttatatgaggctcaagattaacctcgaaggatttttagaagaagtgcaagaccaagacgctatcccttatttgcttaaaatgtatctaagggaaatacttgatcttgatattcacattgaccctaagaacccacatgatacggatatcacatccgaaactgctttaattaaccatcagtataatgtttctgatgacggatttagcgtagtaattgattacactcctaaggtataaaatgactgagattgaaattgatagcatcactgaagcactagaaaaacgtggtgtcgagaaggacgaaattgaagaagcgattctggctcagcaagctattgctgaaagtcaggcgcaggcaaaagctaataaaatttaccgtaagaaccgtcgtgaacttgaccgtttgtataaacactctcaagtagctgttcttgacaataacttcccggcgtataagtatgctatcgagaaatctcgtgacatcttacgccaaccatacactgatgagattattctgaccggttggcagacctctcgcaaaatcatttgggacattatcaatgatcaagcaggtaaaatttaaacgtttgcaggttaacgcaggattcactctgtctatagctaatggtactatggctgttaaaatatcagaaacgcactatcgcattctaggctcaacaggggtgattattaaagctgatccgaaagagcttgtatgggctgacactgcacaaattaaaaggtggtataaatggtaagagaacctgattcttctgaatactttatccatgatgtagacgatcttggcgttgtagaaaccaaggtcggcttcggtgaaggttgatggaagctgtaatagctaaagcgaatacattatctcgtaacgggactatgtattctcctgaagctttggaaaaagcaattgattatgcaaagattagtagcgataagaatgaaatgattaaccggttcaatttcgcttacgataaagctaaagctgaaggtactatcacgtatcgcaaaatttaaagggcttcggcccttttctgctataagagtggatgataaaatatctttatttaaagaggtgactatgaaaattatccattcgggtgattggcatttaggtgtaaaggcggacgacccttgggttcaagaaatccaattaaaaggtatcagagaccacatccaatacgctaaagataatggtattgatacgattattcagtacggtgatatttttgatgtgcgtaaagccattactcacaagtgtatggagtttgcacgacagattgctaatgagcttgaacatgccgggattaacctgattactattgttggcaatcatgatatgcactacaagaacactctgacccctaatgcggcaacagaagttcttggtaagtataagcatattactgttattgaaaagcctacgactttagacttcgatggaactctgattgatttgattccttggatgtgtgatgaaaacacaactcaaatcatggatcacatcaagcagtctagcgctgaatactgtattggtcactgggagctgaatggcttctatttttataaaggtatgaaatctcatgggctcgaaccagatttcctcaaaaagtacaaacaggtatggtctgggcacttccacacaatatcaagcgcagcaaacgttaagtacatcggaacgccttggacgcttacagcgggtgacgagaacgacccgcgaggattctgggttcaagacactcgactacaaacatttgatttcatccctaacgaaacaacatggcacaggaaaatcttctaccctgtaacagggccatttgatgcaaacgaatttaaaaatctagcagttcgtgtagttattaccaaggtagacgataaactaccagccttcgaaagtgagcttgaaaaagtagttcatgaacttcgcacagtttctaaagtagataactctgttgacactgaagaagccgacgaagaagtagtgggtctgcttgatatgatgatggaatatattgaagctcttcctgacgcgacatctcaagatgacatcgatgctcttaaagcattagctactcaactgtatatcgaggccagtgcatgaaagtttttaagttaaatagggtcaagtaccaaaacattatgtcggtaggtcaggcacctatcgatatccaacttgataaatgccataagacgttaattacaggtaaaaatggcggcggtaagagtactatgctcgaagctatcacatttgctttattcggcaaacctttccgtgatatcaagaaaggccagttaattaattccttcaataagaagaatctggttgttgaactctggatggagtacgacgggcacacattccacatcaagcgtggtcagaagcctaatatttttgaaattgtacgagacggtgataaacttgacgaaagcgcaagttcaaaggatttccaggcttacttcgaagagcttattggaatgacgtattcatcttttaagcaaattgtcgtacttggaactgccggctatactccattcatgggcctatcaactccagctcgaagaaaactagttgaagaccttcttgaagtatctctactagcagatatggataagctaaataagagttcaatccgcgagattaactcccaggtccaagttatagatgcccgccaagaaggcatcattcaacagattaaaatctataacgataacgtagaacgccaaaagaaactatctggcgaaaatgtggctcgtttgcaatctatgtatgatgaccttgcttctgaagctcgtcaaattaaatctgatatcgaaaaatctgctacacgtttagctgatatcgttttagatgaagaccctagaattgcactgcaagagattcagcagaaaactgtaaccttacagagtaagataaattctttcaataaagttgttaaaatgtatcatgctggtggcgaatgtcctagctgtatgagcttactgcacccaggcgatcctgttgtcactaagatagcggataaggttcatgaatgtgaatcatcagccgctagtttagaagcccacagagagtcactggagacgctagtcgccgaatacgaagctaatgttaggacccagcaatctctcgcccaagatattcgctctaagaaacaggcgttagtatccacagttgagaaagctaagaaggttaaagcggctctagaccaagcggcttcagaattcattgaccacgcttcagagattgctttgctccaagtagaactagataaaatggtggccgagaaaactaagttagtgatggaaaaataccaccgtggtattctcactgacatgttgaaagatactggcattaaaggcgctattatcaagaaatatatcccactgtttaataaacatattaatcagtatttgaaagtaatggaagctgattactcattcacactgaatgaagagttctctgagactattaagtctagaggtcgagaagaatttagttatgcgtcatttagtcaaggtgaaaaagcacgtattgatatagccttgttgtttacctggcgtgatattgctgaaaaggtatccggtgttaaaataaactgtcttttcctcgatgagatttttgactctgccgcggatgctgaaggtataaaatctataggttctatactgaatactatggctgatgttaatgtgtttattatcagtcaccgagaccatgacccacaagcttacggccagcatctacagatgaagaaagttggtcgttttacggtgatggaatgaatgaagatgcaattacaggccagtggttgctggcctggcctgaagtgaaacgcttcaggcttacaaacgcgttcagtggcgatgaacatatcgttactgaacaaattattaaaaatgcttttaaagaaacagattataatcgcatcatgtcaaacagacattctgcatggttccttgaagactacattgattaattgataaggtataagtatactatgaacattctgaccgctaaagatatccaagttaaaaacgtgcgtactgattcgaaccctaacaatcagaacaaaattcgtaagtcttgggtgttggctcttgatgaaggcactcaagccgctattaaagctaaaatcaaaacccctggagctcgctgggcgtattatgaagctcttgattctgctgtaaacgacaaatggttagaattgatgcgagctcattatgctgattctattaaagccggtgctaagatcgttgtagatcgtaatggcggcgaacgtcttgaagacgactactgcgtagatgcagatgaacagttggttattgctggccggttcgtcgctgaagaagttatcgctgaattctctaaataatgatttgaaaggtataaaatgaaattttctaaagaaaccatcgcagtactgaaaaactttgcaaccattaactctggtatcgttctgactccaggtaattttgttatgactcgttctattatcggaacgacttatgcagacacaacaattcaagatgaaattgataatgagctggcaatctatgacctgtctggctttttaagtgttctgtctctggtcggcgatgatgctgaagtaactcttcaagacgatggtcaaattgcaatcaagagcaatcgttctgcggtgtactgggccggtgctgataaatctaccatcgtcgtaccgaaagccaaagcaagcttcccagtagcaaacgttatcactgaactgaaggctgaagatcttcaacagctgaaacgtgttgcaagcggtctgaacctcgacacgattaccatttctaataaatctggtgaaatcgttatgcaggcttataacaaaactgaagatccggaactggttaaacctaaatacgcattaactcttggtgagtatgatggtactgaaaacttcaactttgttattaaccgcgataacttgaaaatggttcctgccgattacaaactgatgctatggggtaaaaacctggctgatggcaagaaacaaaccgctgctaaatttgaaagtgataactacacttacatcacagcaatggaaatcgattcgactttcgactttcaataaaatttaaatgggagctacggctccctctgaactgaggaacttatcatgctgactatcaataaaaatgaatttatgttcgagcaaaaataccgcccaagtacaatcgaagagtgtattcttcctgctcgtgataaagaaatatttcaggctcttattaagaaagggcagattcctcacttaatcctccaaagtaattcacctggaacaggcaaaactactgtagctaaagcattatgccatgacgttaatgctgagatgctgtttgtcaacggttctgattgtaaaatcgattttgttcgtaatgaattaacccgatttgcaagttctatctctatggaaggtcgtcagaaaatcattgttattgatgaattcgaccgtagtggcttggctgaagcccagcgtcacttacgttctttcatggaacaatattcaagtaactgttcaatcattattactgctaacgaccttgacggtattatcaagccacttcagtctcgctgtcgtgtaatcacatttggttctccttctgaagaagacgccaaatcgatgcaacgcgaaatgttgaaacggtgtattgccatttgtgaaaatgaaggtattgttgttgaagaccgtaaagttctggccgcattagtatctaagaactttcctgaacttcgcaagacaattaatatgctagaccattatagttctaaaggaattattgatgcaggcatcttaaacattgtcactaaagcctctgattcaatcgaagcagttatcgaagcacttcgaaataaagatatcaaatctcttcgtgctttggccccgaaatatgcagccgattatgcgtggttcgtagataagctatcgtctgaactgtatactatggtcacagggccaagtatcgtacgtatgtatgaaattattggtgaaaacaaccaatacaaaggtgtggcggcttcaactgaactccatatcgtgtatatgttcattcaattggctatcgagatgcagtggaaatgagtttattcttttttgatgatgaaccagaatataatgagcaccaaatcgcatggctgactaaggattggtcggcagttcaaaagtgtgcagaccagtttaaagaatcagctgaaagcgagttcttcagattgatcggtgcaattaatgataccaagcaacaaatcaacgtgtctcaaatggactacagcaagttcatgattgaaaatgctttaagtcaacacgctgattgtatgccagccgtttatgtaatgaacttggtgggacaaggtctctcagaccaagcacactttaactatatgaagcacgctgttcctaaaggcaagcggtatggtaagtgggctaaactaactgaagattcaaataaaatgcttgttcttaaagtaattcaagcattttataaaatcaacgtctatgatgctgatatgtatcataagacaatgttcgctaaaggtaatctgctttctttcttaaaacaggctaaagctcttgttacagatgaaatggttaagagtattactaaaaatgtgaaagaacaaaaacaactcaaaaaacaagcattggaatggtagatatgattgaaattacattaaaacagcctgaggattttctgaaggtcaaagaaaccttgacccgtatgggtattgctaataatcgtgataaggtattatatcagagctgtcacattcttcagaagcaaggcctgtactacatcgttcattttaaagaaatgctgaaactggacggtcgacctgtagttatttctgaagaagatgaacaacgccgtgactcaattgcttggctgcttgaagactggggtctgattgagattgctccaggtcaacgtactttcatgcacgagatgactaataacttccgtgttataggatttaagcagaaacctgaatggactctgaaatctaagtatactatcggtagttaaagatttaagcaatgggacttcggtcccattttggagtataattcattcaccaaataaaagacaattactcgtctaaggaaccaaatgaaagaattctttttaactatcgaacagcttggcaatacacttcacgaacgatatattgattcaaacggggttgaacagacccgtcagattgattataaaccaaccctgttctatcattctgatactgaatcaaagtacaaagatatctacggaatgagttgtaagaaaaaacacttcttagatatgaaggaagctcgtaactggattcgtagaatggaagacatcgggcaagatgcaatgggtatggatgacttcaagctggcttatttgtctgataggtatccaggtgaaattcattttgacccgacattagttcgccagtgtaactatgacatcgaagtaacagctccagaatttccgaagcctaatgaagccaagtatcctatcgatgcattaactcattatgatagtattgctgataagttctatgtgtttgaccttttgaattctccttacggcaaagtaactaagtgggacccggtattagccggtaaatcagaagctgaaggtggtgatgaaatccctcaggatattcttgaccgagttgtttatctgccgtttgataacgaacaagaacttcttctagaatacctgaatctgtgggaacaacagacccctgttattctaactggttggaacgtagaaggtttcgataacccgtacgtttataatcgactgaagaacgtgtttggtgaaaagaccgctaggcgtttaagccctcttcgtaaagtcaatagcaaaattattgcagataactttggtgatgaacgtgaaattatcacaccgatgggtatcagtgttcttgattacttggacttatacaagaaatttagttttacgaaccagccttcgtatcgtcttgactatgtagctgagtatgaacttggcgtgggtaaattagaatatgacggccctatcaacaaacttcgtgaaactaaccaccaacggtatatttcgtataacattattgacgttgctcgagtacagcaaatcgataaaaaacgccagttcttgaacctgtcgttagatatggcttattatgctaaaatgcagatgcagtctgtattcagccctattaagacatgggacgcaatcatctttaactcgttgaagaacgacaataaagtcattcctcagaaaaagcaccacattccacagagttacccaggtgcattcgttaaagagcctactcctaatagttataagtacgttgtatctgctgacctgacgtctctgtatccgagtattattcgacaagttaacataagcccggaaactttgcatagccagtttgctgtccgcccacttcatgaatacattgctggtactgctccaaaaccgagtgaagaatactcttgttctcctaatggctggatgtatcgtaaagatattgatggcgttgtacctgttgaaattaagaaagtattcgaccagcgtaaacttcataaaggatttatgttggcggctcaacgtaatgcagaactgattaaagaagcattgcataatccaaagagctctgataagccagaaccgactcttgattatcgtttcgatatggaagaaagtattaagtcacaactccatgatttgaacgaagctattttgttgagtatgcttgataaagcaactcgtactgaaatcgccggtatgactgcccaaatctgtcgcaagttacttatcaactcactttatggtgcacttggtaacgtacacttccgttattacgaccttcgtaacgcaacggcaatcactttgttcggtcagttggctctgcagtggattgaacgccgcgtgaacgagttctacaatgaaacactgaagactgataaccataagtatgttatctatggcgatacagactcaatctacatgtgtgtcgactcattaattgaaaaggttggtgagaacaagttccgtgacacgaaccatttagtagatttcttagataagtttgctaaagaaaaattagaaccagccatcggtcgcgcattccaagatatgtgcgactatatgaacaacaaagaacagttaatgtttatggaccgagaagctattgcttgccccccattaggttctaaaggtcttggtggtttctggactggtaagaagcgatatgctttgaacgtgtatgacatggaagggactcgctatgcagaccctaaaatgaaaattatgggccttgaaactcaaaagtcaagtactcctaaagcatgccagaaagcattgaaagaatgcattcgccgtatgcttcaagaaggtgaaagctcattacaggactactataaagagttcgaacaagaattccgtaaacagcattatatgactattgctggtgttagttctgctaacaatattgctcagtacgatgataatggttatcctgggtacaaatgtccgttccatattcgtggaatcttggcttataaccgtgctactaaaaacatcccagatgcagaagctattgttgaagggtctaaagtaatggtactaccgttacgagaccagaacccgtttggtgacaaatgcatagcgtggccatcaggtactcagatcgatcctagcattcgtgaagatgtattaaagtaccttgactactcaacgctgttccaaaaaacgtttgttaaaccgctgtctggctttacatcagcggctagaatcaactatgagcacgttgcaaccttagatgatatcttaggatggtagtatagattaggccgcgaagccctggaatcactctagggcttcgctttaattattatgtagtacaatagctcgtcaacaaatgaggaacacgaaatgcgtaaagtagttatcttgggtgccggtcttgggtcgcgtttatacccgattacaaacgaaatccctaaagtgcttgtaaactacaaacagcacacagtttttaaacacttggttgatttgtataaaaatcaatcagatgaaattattctagtaattcacagtaaatttgacgggctagttcgtgggtacatgaagtctgttggaataagtgatattaccattcgtcatgttgatgtatctataggttctgcacacgctattgattgtattgctgacgatattgtaggccacaatgtactgttcaactggtgtgatattattccggaaggccatgaagtagaatggggtaatgattattgctatacctttggtacagattgtcgtttcacttttgaagaaccgtatttccgtgaaatcgggaccggtggtggcgtggttggcatgtatcaagtggctcaatggcctgggttctattcttttgaaggtaactatggcgaagaccttgccgacaatttagattcaatggtttatatgacagagcgtaagctagagtcagttgttgacattggtgataagcttaaattagccgaagcgcataaaacctcagagattaatcgtgactttaacaagctcaaattcacagaacaacatgctattaaaatcccgactaatgaatatggtaaagaaattcaagctaaagagcttaaatggtataattctgtacaatcgaaatttattcctactattgtagatttcacgccaggtgaatttattaagatggaacgaatctacggtaaaaccatggcagatgcataccctctgatgaccaaagatactcaagtattaatggtatataaagtaatcagtgctctgaaatcattaggtggcgatatctatattcctaccgacgagcaatggtatgcagatgttaagaaagaagttcttgataaggtattaatccgcaacgattctattaaagaacttttagaaggttttgcacctgaaggcattacccatgttaatgattttaaagtaggtaaaccagaaaaactgctggaacacgcattagaaattctatccgagcataaagaaccgtaccagttgatccacggcgacccgcattactctaacattatgtttaccgattcgggtgatattaaaattatagacccgcgtggatattttggtagtagtcagtacggccctaccatttatgatgaagctaaagtgctttattctattgatgggtacgataaatttaacgcagatccactatggggtggtatttctcggaatggcactagaatgtttgtcgatattgagaaagtaatgccgttagatgatatccctatgtgtacctttaagcataaactgtggattgccgttatttggatggctttagcagggtatttcaagaacaaccctcttaaggctattggcgcttattatcatggattgtatcagctgagtgtgctgcttaaaaggcggcctcgcaaacgtaaacttatcgatggcaccgaagtgactgaagttaaagacccaatcacagcttctattattaccagatgccctgataaatgggaattgcttgataaagaaactggtgtaaaatataaaccaattggcggtaacatcgcccaccagtgggaacgaatcggatgattctagttcgtaatgacactgttaaggaggctaaaatgcctcctcctaaatcttttaaagatacatttctttataaagtattgacctgtgaatcgtctttaactataaggcctattaatgaatctctgcttcgacatcgacaacacaatcacggtatggaatagcaatcgtgattatctgaactttgcaccagaccatgaaatggtggccataattaacaagctctatgatgaaggccacgagataaccttgtacactgctcgcggtatgacctcttgcggcccaggtcgaattctgactgaagttgtccctgctcttgtcaagaaccttgaaaaaattggtctgaagtatcataatctgcttacacataaaccaagttatgattttattattgacgaccgagcaattcgacctgatcactttaagaaaatgattcaagatggcacatttgaaacatacaaggcatatcatccatgagtcctaaaattattgttgtagacggccctgataattcagggaagtcaacttttattaagattcttgttagaaatcctaaggtaaaattaatcgactttcctaaatcaattaatggcaagtgtatttcattaggtacagataatgataaagccctggtcgaaaccttatacaagttcttagatcctaattttgtctatattcttgaccgtgggtaccctagtaatatagtttactctggattccttcgcggtgaaatagacccgtatgagcaccttaaagattggattcgattcaaacaagaatttgacatagttgaagtcattttgtcacgcaacaaacttgacgaagacttcgaagatgatttgattaaactgactaaagatgagtttaatctgactattaccgagtacgagcgcaactttgaaaacgtgtttaagcttttagagcatgatggctttaatcgggttttgagatataatgaagacgaagctaaacgccttttcgcatatattgaaaataaaacaggtattaaatgaacgtatttaaggtcggaactaattttgacttggctcttctggataaaatcgttgagctgaatgctaagaatccgcaatcactgattaatgaagtttacggtagtacacgagcgatggcctttgtcgctgctcgaccggacttccgtttgcctgatgttaaagatgaccagctggaaacttacgttcgacgttgtaacgaacttggtatttgctttaactacactttaaacagtataaacccagggaccaaacgtgaactgacggaatggaagaaacaggccattcaagattatgtacagtacttatcttctatcggcgtttggcgtatcactatcgctaacccggttgtaatggaaattgttcgtgaagtaaataaagaaattgaaattgaggtatcgactattctgcatgttgatgctgttactcaaataaaatatcttcatgatcagtacaacattaagaaagtgtgctgtgggattcataagaaccgatcagttaacttcttgaaacaagccgcagcattttgtaatgaaaatggaattatttttgaagtcctagttaacgagttctgttctaacgctggtaagggttacacgactcattgtagttatcgtgattcttgctatattttccacagtaccgacattactgcagaagatgcaaaatcgctcgacggttatccaatgcagcactgcattaaagctcgagacaccgaccctttcaactggctacgcacgcgttttgtgcgtccacaagacctaaaactgtatcgtgatattggcatcacacagttcaaagtctctggtcgaaccggctcaaccgaatacatcatgaaggttcttgaagcgtattcttcagagcaatttgaaggtaaccttcttgagttgtggaaacctctcgaaactatctacaacggtgaatctgacgctaactatagccatactgtgaatatcgagacttctttgttagatggcttccttgaaaaacgttggttcaaacatcctacatttgactgtgctaatgaagtctgcggtagtacttgtacttactgcgaacgttactacaagcgccagttatctaaaaatgaccggcctctggattctatccagatcgtagatgttactagttcagacgacgaactccggtacccggaataagcaaagaggcttcggcctctcttagtatataataggattttttaatgtcagagcacaacagaatgaaacatattacatacccgtctttccaaaccgcattcaaagtactgaacgaagagattctcaacaatccgcagttcgttattgattctcgtattggccgttgtaatgaaatcggttctgcttcaatagaagtaaccgatccgacaacgttcaaattcaccgattctcgtatcaaccgtattgattatgattatgctgaagcattctggcaattcatgatttcaggtggtactgatgccgctgaagcatttaaggaatacccgaatgttgctaagttcattgataagccgaaaagtgacgtactgcctgcaaacttcaacactttctatggacctcgaattgctgcccaactccctgcattgcttaaagagcttaaagaaaagattaactcgcgacgtgttgtgttccaaattctccaagagcaagaccaagcacttctagattcagatgaatcacttgaatacccgtgtacagattctataacgtactatatccgtgaaggcaagctttatgcccatacacatatgcgttcacagaactgtgccgtagtgatgcagctagacttttatctgcaaggcaagttaatgcaatacattgcagatcaatgtggtgtacaattaggtacgtattctcacactatggtttcagcccacgtctttgaacgtgattttgattatgtaagaggttttatctgatggcttatttccgtgtaccaatctttagtatgcgaagctatgaaactggtgaatatgccgtccttaaggacggcaacttccagttacatttgaatcgtgctagtgctggtgatatcatagcggttccacgtaacgcaagtgatatcgaagaatgtaaagaactattcccagagtttgaatttgttcctctttggtacaaggaaaatgcttatgaaactcgtaaacatttctgggaagaaaatcagtttatagttgattctttagttgattactacgactgctcttatctagtgacagatattactggatatcaaggcaaccatgaggtattctttgtttttaatataactaaagaccctgagaatcctcggtattatattgacgagtttattgacattgacgttgaatctgtgaacaagtctgtagcaacatttgttcttaatcacggtcagaaagacgttctcgtagcggctggtgcaattccaggccgtatctttgtagatcaacgagtcgtcaacccaagtgtcatggaacgttatacagaaggtctggaaccaatccatcttgatggtatttttcatccattccgtatcagtgacccttgttatcgttttgaccaagttgttgaatgcgccattgcagcagatcgtgtcttgtatatcacagacccgaacatgagtttcaaacgtgaacagtatcctaaagaagccaagatccacgttctgcgcctaaccaagaaagaatactaccaagtactaatgggtcagccaactatccagtactttgaagaccctgaaaaagtgttccatcctgggctagcagagttcatctattttaatgctaaaataaattctccacataatattcctgcttataagcaagtagtaattaatggataaatttattttgtaaactgcgagatttacattagtgtaggaatatgttaaagtgttcctacacttggagaagaactatgaaacttaaattaattctcgtactatctgcactaactcttgccggatgcgctcaatctgaaggaaatacatatcgtcgtctttctgatgttggtacaatctttaccgtcggcggcaacaatataggctatgtccgtaatgcaaacgctaaccaagttcactactctagtgaccatgaagaagcaatggctatgaagcgagaacacgacgctaagatggatgctcttatcgctgaagagcaggcaaagcaaacaagtttaaagatttaattgctgttaaaccttgagtgtataattactctactttataaacaacgtgagaaaaatataatggtgactaatggtaaatattaatgtccgatttgaaatctcgcctgattaaagcttctaccactaaactgactgctgatctgactaaatcaaaactgttcaatggtcgtgatgaagttatgacgcgaattcctatgctgaatattgcattaggtggtggattgaatacaggtttacaaagtggcttaacaatctttgcagcaccatccaaacactttaaaactctgtttggtctgactatggtcgctgcatatatgaagaaatacccagatgcaatttgtctgttctacgacagtgaatttggtgcttctgaaagttatttccgttcaatgggtgttgacttagaacgtgttgtccatactccaattcagtcagttgaacaactgaaaatcgatatggtcaaccagctggaagctatcgaacgcggtgacaaggtaattatgttcatcgactctatcggtaacacagcttctaagaaagaaacgcaagatgctttagacgagaaacaggttggtgacatgactcgtgctaagtctcttaaatctctgttccgtatcgttacaccttatttgacgattaaagatatcccttgtgtggctattaaccatacagccatggaaatcggcagtatgtatcctaaagaagttatgggtggtggtaccggcattctttattctgcaagtaccgtattctttatctctaaacgtcaggttaaagaaggtaccgaactgactgggtatgattttactcttaaagctgagaaatctcgtacggttcaagagaaatccacgttcccaatcactgttaacttcaagggcggtattgatccgttctctggactgcttgaactggccacggaaatcggcttcgttgttaaacctaaagcaggctggtattctcgtgctttcttagacgaagaaactggtgaaatggttcaagaagaaaagtcatggcgtgctaaagccacagatgatgtagaattctggggtcctttgtttaaacataagccgttccgtgatgcaatcgaaaccaagtataaactcggtgctatttcttctattaaagaagttgacgatgctgttgctgacctgttatcttctaagccatcaactaaaattccggtaaaagccaaatcggctcctagtgctgctgatattgaagacgaactggacaatttcgctgatgaagaataacttcgaagacctggacctagagtctctggccgaagaaattgaagctgaagctccccttgaagaaggggagtttgaacgctctcaaagaatcttcgacaaatcccatagtataatcaaagaagcaatggctagtgttatccaagaaatggtgataagattagacggtgaagaacacatcgtttatatccatgaactggacattaagccaaacggtgaagtggctgttaaattcagtacaccctccgataaagacattttatatcctcatgtcatgcagtgtcttaaaagacaatttgagatggtgcctaaaaagcgaggattatttttctaagaggtcaatgtggtagaaacaattttagctaatttgcttttcaatcaggctttctttaataaagtatggccttacatgaaagatgaatattttgaacgaggcccagctcttactttgtttaagactattcatcaccatgttaatgaatataatggaatcccgtcgaagactgcggttaatatcgcattagacaagtcttctatgtcggatgtggaattgagcggtgctaaagaacttctatctaaacttacagatgctcctgaagatttaaactggctcaccaaagagacagaaaaatatgtccaagagaaggcaatgtataatgctacttctcgaattattgaaatccaggctaatgaccaattagacgaaaagcaacgtgataaacgtctacctggtttaggtgccattccagacatcatgcgtgaagccttatcagtatctttcgatagttatataggccatgactggatggatgactatgaagctcgttggttggcttaccaaaacaaatctcgtaagattccatttaaactaggtatcctgaacaaaattaccaaaggcggcgttgaaaccggcacattgaatattttgatggcaggggtcaacgtaggtaagtctctgggtctttgttcattggctgcagattatcttcagactggtcataatgtcctctatatctccatggagatggccgaagaagtatgtgctaagcgtatcgatgctaaccttcttgatgtatcacttgatgatattgatgatggctgtgtatcatactctgaatataaagccaagatggaaagatggcgcaagacctccactctaggtcgtttgattataaagcaataccctactggtggtgcttcagccaataacttccgcgcgttattaaatgaactaaaacttaaaaagaacttcgtcccgtcaatcatcattatcgactacttaggtatctgtgcttcttgccgtattaagcagtacactgaaaacagctataccttagttaaagcaattgctgaagaacttcgtggtttagcagttgaaaccgagacagctgtatggtcagcggctcagaccggtcgtagtgcttgggactcgagtgatgttgacatgtcagatgttgcagaatcagcaggcctacctgctacagcagactttatgctggccgtaatagaaacgcctgaactagccgagatgaagcagcagttaatcaaacagattaaatctcgatatggtgataaaaacatctacaacaagttcaatatgggcgttcgcaaaggcaaccaacggtgggtagaaattgcccaagaaggtggaccgtctcacactaatacccagcgtgaagcccaaggcactcagatgaagcaagccgctgataatcaacagaaaaaggtttcatcacgagctgaccttgataagctggcagaagatttaaaattctagtttacatctgtgtaggagcatgatactatgcttctacacaaacaaagtgaggaactgacatgaactacgaaaattcttatcctgggtataaagatgctactttagtaaaaggcgggctttataatgttagtgaacaagaaaaagttcctaacaagtacttagaaaagtataatggaagagctttcattaatttaggtctttcggctaatggctttgtgttagatgggtacgagtgtgcttaccattatcttacccgcccagaaattcgctatggttttcctgttaaagtaaaagggaaatctaaagctatgtttgttcggcagctgtataaacacgagaactctgaagttcgtgtctgtggatggtgggcagaccaggacggaaatcttgatcgggactctcgtgcagacgtacctcttttagatgtagaataagcttaattttgaggaaaatataatgaaaaaagctattctttctttaattttcgctctttctgcctgtggcgcacagccggcctttgccaaagctgactacagttctccgctgtgcgagttagccatatcttctgatatgtctatactgcgtagtgttgttatcaaagagcttgaaaaagccgctgataaaacccagtcagaagaaatcaaaaacatcaatgataaagatgtagttgtagctgcaactaatcttttctgtgaaaaccttagtgctaaagaaactttagagtggattggattatgagaacttatattatgtaccacaaggcttgcatttccggcctctacactaagcgtcctattatgtccgacgcccactgggaagtagtattagaccgtggaagcacagataaagttgtctttggacacgttctgtctgagacatttgtctgtaaacctacaaagcgacaactgagaaaaatgcgccgggctttccgaaaggactgggaacagataattcaacgacaggaatttgagaattcttgggaaggaatccactgcgatgttatcgggctataaataaaacatttaaggagaagctacatgactttacttgaaaagtacaaacaatctttatccgaaatggtcgccggcgattctggcggctctccaaccaacatcgcttctggtacaacaactggtgatgttgtcaataagggtcctgaaaccctttctcagaaaaagcgcaaagataagccttctgaagagtgatataataactccaagaggtaaatatgaactttgttgactatgaatttgcagaacgggccttgtccgttctgcctcgttatcgaaaactcccaggaagtacttttaaattaaactgtcgttgtcctatatgcggggactctcagaagaaccagatgaaagctcgtttttggtgctttgaagtagagggtggactacgtgtaggatgtttcaactgtgactacaaggcttggttcaccaagttccttaaagaaaccgatgaaacgctgtaccgtgaattcttgctagaacgtcgtaaagagaacatgtatagggagaaaaaggccccatctatctctgaaaagattaaagcaaagatgcctgtgattgagaagttgccaaattgtgagcgtctggatagattaccagccgatcaccctattgttaagtatgttaaggcccgctgcattcctgaagtaaactggaaacgtttgtggtttactatggaatggcctgctcttgttaactcagttaatccaggaacatataaagatgaaaagagcgaaccgcgcttggttatcccaatcttcaacgcccgaggagacatcgagtcatttcaaggacgtgcacttcgaaaagatgctccgcagaaatacatcacaatcaaagcacacgaacacgcaactaaaatctacgggctcgatacagtggatgagtcaagactcgtatgggtcatggaaggaccaatagattcattgtttgtacctaatgctattgctattacaggcgggtctatggatttgaacacggttccttttaaagagaaccgtgcttggattatggataatgaacctcggcatgaggataccattaaacgtatgaagaggctggtagaagctggcgaacgtgtcgtgttctgggacaatgctccttgggcatcgaaagatatcaatgatatgattaaggacgaaggggctacacctgaagaaatactcaaatatctgaatacaaatattgcttctgggctaatggctaaaatgaggttaagtcgttatgctaaagtttaatctataactccggcaaaaaagtttataatactcagcacgtgctccacagtaacttgtgggagtatcactccttgggtagctgctatagggaccaccaaaaggttccaaaggaatacatctaaggcaaggaagcccaccatcaattccttgtcccttttatccgataatttccaaatttgataaaatattgttcccatgttatcctcctgttaaaggtatttatagatgaatagtacacaacacgctcttcttaaactatcagaagagtgcaatgaagtagctcagatttgctctaagattatgcagttcggcttcgactccgaatacgctggtaagatcaacagagaacgtctaactgaagaactcaatgatattctaggggctgttctaaatttgaccttataaactgatttcaggttcgcagaagatgcgtatgctacgagggctaaggtagataaaatggctcggtacagagaaatctctaaaaacttagggttcgtccaataaacagtttacagcgttgtaagagcatgatatagtgctcttacacactaaaggagaagaccctcatgaaaattatcttaactgcagttgctttaaccattgcttctttttcggcccaagctgcttcgtttgattgctctaaagcttcgtctaaagtagaaaagctgatttgttcagatgcgttagtttctgagctggattcgagccttaacaaggtttatagtgcaatcgaagaaaagcctgttgaagaccagcgggcttggattaaaaacgtcagaaacaagcaaactagcgtggaaagcctagcttacgtgtatgcttctcgtatagaagaacttgccaataatgaaaagtacgcatacactgtagtggataaagggcctaaagagccaatgaaagaagttaaagaaactaagcctaagcctaaatccgtagtagacaacttaaaagaacacgtcgccaaaggctatattgaagctgacgggtacagagtttctttagaagttttagaaaagtttggcgatgcttatttagatcgttgtaatgtaaaaatggcttatgatcaatttaaacagatcgaatccgaagtttaccgcacagctcctatgaaagacgttgcttggcttgatgcaaacaaagaagctgttgtaataggtttagcaaacatgaatacagtgtttgttgaaaccccagaagatgaggcttctttccgagatgcatgtaaagttctctttaagaaagaagaagcccgcttagaaaaagaaaaagaaaaccgacgtttgactgaagaattcttcgaaaacgctaaaaagaagtgagtatataattactctacttaccacctgaactgaaaggaataaaatggcacattttaatgaatgttctcaactgattgacggcgttgataaagcccaagaagcttactatgacttgtgggttaataaacaagacgctttgcaggttatgcttgatatgcaaaaatctctccaggtccgtctggctattgataaacctgagcataatgctcatcctgatgcattagcacgtgctggtgatgtagttgattggcttcgcaatcagaaagactacattgacgacgagttccgtgaacttctgacttcattaggtggcatgtctaacggcgagaaagatgcctctgcagtctggaaaccttggaaagcccaacacgctgaacgccgcaatactttgattagcgaactgtctcctgaagaccagctcgaaattaaattcgaaatgatcgatattctgcatttcgttctgaacatgttccagggtcttggtatgacagccgaagagatcttcaaactgtactatctgaaaaatgctgaaaactttgcccgtcaagaccgtggttactaatggctacaagaatatctaaacgccgtttaaaaattaaaagaaaacaaaaagaaagggccttagtaaaggtccttcaagaagaaattactagtgaaatcgaccgagaaatcttaaaagcattagaccctttaaatgaggtgttccgtattgcggttgatgaagccaatgaagagctcctaaaagagttagtaaaaatgagttataaatattcctgatattaatcacaaaggggtataatatggcttatgtaaatattaaaacttttgatcataccgtagccggtggcgaagttaaaggcactgaggtatcggttgattttaaggtatactctaactcgcaccgcattgctgatgctcggtatcaaactttcccgtctgaaaagcctgcttattctacagtaattgatgatgcagcggcttgggctactgccaatgcgaaaatgtttgaagccgtcccggctgatagcgaggtataatattaaggactcctttgggagtccttttttgcttttagtagagtagagtaggtagaatacttaaaacaacatgaggaaatgaagatggttaaaagtacaccttggcgtaaacctattggtgactgcggcggtattaatgaactaattgagcgtcattacaccttgctccaagataaagagactttttgttattttgatgttattacccatgcttaccgtgaagtaggtacatctcttgaagcttctattgaggtccagtactggatgttctctaaaggcaagcgtaaaataacccatatttcgtttgagaccttccgtgtatgaattggattaaacgattcttagattggtcgctggccaacgagaaatctcccggccaagatttctgggatgctttagaaaaggaccgcaataataaagcaaagaagaaacttatcaagtacagtgaaaaggaagttattgagctagctaacaaatatctggtgattccatgattaattggttaaagaatttatttgcaccacggcctgttcctgaaaagcagtcattcattaaagagcgtgaatatgtttatgtcggtgatggcatgatggaagaggttattaaagaccctcgtaccccattacaaaaagctttagatgaagtgactattgctaatgcctctagatttaagaaaatccaagaagaggcggaacaggccagaaagcgtaaccggaatagtatggttcctttgtccggttctagttctagctctaattatacttcacactacgttgattctaccccaataattgctgctacagcggcagcagtttatgcaggaagttcatatgactctggtagcagttactccagttgtgactcaggctcagttagttgcgactaagcctaattatcaacatgcgatctacaaaagcccacttaagaaaaccaactttgacgcctggtatagaggtatcagagcaatgatttttctgcttaagacaggaccagctataatggctgcgaatgataagtggtttgaagaaaacaaaatgactgaaggagcctattgtggcagacgcaaaaatctataaagtcggtattagcttagctgataaaaacgggcgtatcgaagatacaattcgaacctttgatgtggcattatacaatatgtatgctgacgacgcccgggtcttaattgacaaattcctagatacaagctgggtgcctagtattgaatatgggcaagttcgtatcatgcacatgtatgatgtgaattatcgtaaaggtagcccatggggtgttcacgatgcccttattaagaacggctacaaacatatcagctgctttgaaactcctttcgattacgatagcagtggtactattgagaaaggaaataagattattcttaatacagcacctgatgatatccggtattttgctaagatgatctggaactatatgaatgctgatgccgcagattgggtaccatctactaactttaaacgcgctgtagaagattatgaaaaagctgcagctatccgtcaacaccaaaagttagagtggtttatgtaatggatctgtttgaaatgttcgaggccgccgctcaagagcctgtatcgccgcctgaggacttgattaacgaacttgatatgatcatacagaaacacggaataaatctccctcccgccgccttgttagagctggcttcttactatcgtgaccctccgccatgggcaccttggaaataaatccaaaggtgtttacatcctctttttaacgtgttagtatctcttacatcaaacaggagatactaacatgcttatccaaatcagttcagaagaaaaagttcgtcaaagtatccagtctaaagtggaagaaaaattcacagatatggagcaaagtactctatggctttgtttgaatgataagaatgaagaacttattcattccaggctgaacccaatagtacgtaagcacatgtctactacggtaccatcagaattgtatcgtggggtgacactgattgaagcgcaaaaactttataatctagctgaaggtgatacatttactttaggacgtgttacaagcttcagctctgatttctccactgccaagcagtttgcttctaaatggcattatgattcaaatctgattctgagcatacagaactgcccatgggcatacaattatcaagaagatatcactaatattcttcttggtgctcctgatgaagagtacatgggtttagtgtcatctgaagatcaacgcgaggataagttagatatggttcaaggtgagtgtgaatttatgcttccgaaggaagcagcgtaccgcatattacgtattgaagataaatctaatccagatacaaacacgacggattatactattcttcatctggaacttatcagctggtaatagcctgaagccaaagatccatgaaaccattataccgttaaaaattctaaagcaaataagggttcaaaatgcacaagttcttaatcatgttgtggtacactatagatggcgaacactaccattccagcttcgaaaaaactatcttcgggcctgacgtccactgggtagcccagcagtacgcggaagtcaacgataactgcctttgcaaaatcttcttaggcgaccgcaaaatcgctgagcacggaagccataaacaatagtttacagtggtgtaggagcgtgttactatacttctacaccaacattgaggataaaatcatgcaagcatctaaccaagttatcgtagcgcgtcgtctggctaaaacttttgaatctgctaagtccagaggcaaagaattcaaccttacctttgaatacctggctaatgtattagcccagacccactgtgcttactctggtgaagagttctcaggcacaggtctcgattcactaactcttgaaagatgggataacgacaaaggctacgtcattggcaatgttattccggttaaagagaaatataactctgctagaggcaattattctatagagaaaatggaagctcttgctaaagaaaaatcggcccgcatagtccgtggttctgataataaacctgtaacattggctgaagaaactcaggctaaaattaagctcatcgagaaaaatatcgaaggcataaaaagccgccgcgaaggtcgtattgcagctctgaattctattctctctgaagaatatcttgacgaagcccgtaaggtaatgatagcttctctcaaagccagaattgaaggcgctaaggctgagattggccggcaagaagctaccctgttcgctcttcgtgacggccgtagcattaagaacgctactaagctctctaaagcagaggaacaggttcgtaactatggtataattgttgaagctctgaaacgtttagagaaacgcagctggttagacaccaagaaattggaaaaagggctgccgctgaattgttcgctgcttcaattaattaaaggtaaaatgtaatgatgatgcactatggatatgctctggtgtataaggacaaacacgggttcgtgcttccttatgaatacaacgggtgctgcaatgtcttctctgatgaagcagcggccaaaaatgaatataatggtttaaagtcttacttggaacatagattcgatactcgaattaaatctgtcgagagacgagtagttccacgtaagtggtggttccctaaagtgcttatcgtgacaacgcattatagtgacgatgaacttaaatctctccgccaaatcattagcacactgcatgttaaaagggtaaaattagcttgattacgttcgatatgctgaacaaaggtcagaagaaagcctttgactatattatcagtcgtattaaagccggtaaaggaaaccatatcacattgaatggcccggctggaactggtaaaaccacgatgaccaaattcatcgttgattacttgatttctcaaggagtatctggtgtcgttttagctgctcctacgcatgccgctaaaaaagtattaagcaaactgtctggtattgaagctcggacaatccacagcttgctgaaaattaacccgacgacatatgaagactctgtaacatttgagcaaaaaggcgatgtagacgtatctgaactacgggtggttgtttgcgacgaagcctctatgtacgaccgaaaactgttccagattctgatggccaccattccacggtattgtcttgtcattgctataggcgataaagctcagattcgtcctgtagaacctggctctactgtaccagctctgtcgccgttcttcagtcataaagattttgaccagcttgaacttgatgaagtgatgcgtagtaatgctcctatcattaaagtagctacagatatccgtaatggtaaatggatctacgaccaccaacttgacggtcatggtgttcacggattcacatcaacaacagctcttaaagatttcatgatgaagtattttgaaatcgttaaagaccctgaagatatgttcgaaaacaagatgtttgctttcacgaacaaatctgttgataaattgaactctattattaggcgacgaattcttgagacagaagatgcttttatcacaggtgaagttatagtaatgcaagagcctttgatcaaagagcttgaattcgaaggtaagaggtttaacgaccttaaatttaataacgggcaatacgttcgtattgtaagtgctgaatacgtttcatcttttgtttcttgtaaaggtgtctctggtgagtatatgcttcggcactggcgattaacggtagaaacttacgacaaagatgaagattatcatctagaaactataaacgttatctctgatgaacaagaacaaaataagttccagttctttttggctaaggctgctgatacatatcgcaactggcagaaaggcggtaaagctccatggaaatctttctgggccgctaaacgcatgtttcataaagtaaaacctctgccggcttcaacattccataaagcccaagggttatcggttgatactagtttcttgtatacaccttgtattcatgtagctgatgctgaattagctcaacaattattgtacgtcggcactacccgcggacgccatgacgtttattatgtataggtgaactatgttacgtattaacgaatatacagctaacaaattaattgaatcttatgacactcgccatgaaaccatgatgcctcaccaagctgaagcaatgtataaggttcgttgcatttgtgaaaatgctattagtgttgcatcttatctaggagcaactccagacatcattattccgagcagtttagttgaagttatgtcaacaattgcttatgaaggtaaactatgattgatattattatcgactacgaaacatttgctacaacttctaactctgcggcaattgacctgagtttaattgcctataacccagaccctgaagtggttcaatctttagatgaacttatctctaaaggcctgcgggttaaattcgacttagcttcacagaaaggctccagattattctctaagtcaactttagcttggtggaaggaccaatctccagaagcccgtaagaacttagtcccatctgacgaagataaaacagttatcgaaggcattaagattgtcctggattggattcgagaacaaggtgtagatccatggaacagccagatgtggtgccgtggaatgagcttcgatattcctattttcacagatatgattcgtgaattgtatcgtgctgaaggcattcctgaaaatgaaatcgatacctttaagcttgagcctgtcaagttctggaatcagcgtgatatccgtactgctattgaagcatacagtatggtacgcggtatgaccacaactcctctgcctaacggtactctaaaaggcttcgtagctcatgatagtcttcatgattgtgctaaagatattttgatgttaaaatatgcacaacgttatgctcttggtctggatgaatgtccgtctgaagctgaagccgatccactctcattgagtaaaacacgatgaacgattttgagtatgatgaaaactttgaagaatggtttaactcgaccattcttcctgaaatctccccaagtatggttttagttgttaaagcaactatggcgaaaggttgggaagctggttataattttggtgttgacactggtattgaactttctaactatattcgctaaggaaataaaatgatccctattgactctctggtctatgtatccactcagtcccgttcccgtatcgccggtaaatccggtgttgtagatgccgttcgttttggttggcttggcgaaattaaagaatatgttatttcttctaacggggtcgttggctatgtatcacctgagtatgtatcagtgattgggactgtggctctcgatgatgatgactacgatatccgtgacgatcttctttgtaaagcagttcaaatcgaaaccccattcatccgcgtatgcggatgggttacagaccagtgggttgaagatggcgttgaactgctgaacgtagttcacgacggtgattactctgtggttccgcgttccgctgtgaaaaatattatcaccaagtagtttacatccaggtaggagcatgatactatgttcctaccaaccaaatgagaggaaaacaaaatgaaagttgtagctaccacaaattcaaatcgcgatgaattcgaagatgttctgtttaacccggacttagtagttgtgcagaaagatatcactgaatttttatgctgtacccaaatcgtgtatgtctataacaaattaggcgatactcttccggcttatgctattttccgcgaaatctctgaaaacggcactgattactggaaagaaacttacgaggcctaaaatgcaaaacaaatattttcgcttcatcagcgatgaagctcgctctcaatttgaaagcaattattcttcaaacatcgaaattggtaatgctatccagaatagcatttttaaagttattcaactcgccattgacgatgtcgtaggtatccagtttaaaagcggcgctactcttacatatagtaaagattgtctctacataacccgcgacgagatggaagaattctttgtagaagtctccggctttgacggcgattcagggctgtgggaagaaggtgaaacctatatctgcatcaatttaaaaggtttggttcaacgtagtaagttgaatattcgctttgctgaactagttcatttaaatcggtttacggttttagctgttgaccatagcctcggcagccctcgtgttaaaaccgttcaagttggtaaccagaaaatcaatctggcattagttcaagccgaacgttcgttctttagacgttggacggacttcaaaaacaacatcaacggtgatatggttgttcaagaacatctcgatgaacttgacgaacttgatgaagctcaagatgctgagaacgacgtcggtactgttgatccggttccactagtccttgaaggacctgcaaagattgaactaatcgtgtcagatgaaaaatctcgtctagcagcaattaaatttttagaaggaactcgttttgccaacgtataactataaatgtgaagcttgtgacgaagagcgcgagtatattcgtaaaatctctgaacgggatgagccggtagagtgcccgttcctgaattgcgaaggtaaaatgcaccgtactgtgtctgctcctgcagttcactatgacggtcttaaatcaggtgattattaatgagaatgcgggcacgtcgtatctatatggtatcgggtgaaatgcgtgaatttaaacttaatgatggccgcactgtgattgtgagaaaacgatgactacttcagaatcatttggcatacatccatattacaagcatttgactcacatctttactcttaaaggtcttggtcgaatacggatgatgtatcgtccaactaaattgacagctaaacaaaagcgagaagctaagaaagaattcaggtcctttcttaagactggtttacacgctgcaaagtgggtagaaaaatctaaataagggcttcggcccttttgctttttagaatacgggataatatagcttccactacaagaggaaatgaaatgattaagaacgaaattaaagtactttcagacgttgaacacatcaagaaacgtagtgggatgtatattggtagctcagcgcatgaagcccatgaacgctttctgttcggtaaacatatgtctctgatgtatgttcctggcgtagttaagctgattgatgaaatcattgacaactctgttgatgaagccattcgtacaagctttaagttcgccaacaaaattgatgttgaaatccgtggaaataaagttatcgttgaagataacggccgaggtattcctcaagatttagttgaaacccagacgggtgaacagatcccaggtccggtagctgcatggactattccaaaggccggtggtaactttggcgatgacgctgagcgtaagacaggcggcatgaatggtgtcggtagtagtttaaccaatatcttctctgttttgttcgaaggcgccacctgcgatggaaagaatgagattaccgtacagtgttctaacggtatggattctataagctggaataaagttccggcctctaagaaagaacatgtcaaagctaaaacaggaacagtggtttcatttattccagattttgttaactttgaagtaagtaatctgactgatgtttatagtgaaatcactttagaccgtcttcaaactctggctgtaatttatccagatgttaaatttactttcaacggtaaaaaggttgatggtaactttaaaaagtttgctaagcagttcggtgagagtgtagtaattcaagaaacagataatgtttctatggcatttactaattcagatgatgggttccgacatctgacttatgtgaacaacatacatactaagaacggcgggcatcatattgaatgtgtattcgatgatatctgtgaacaccttctaccaggcattaagcgtaagtataaaggtattgaagtaactaaggcacgtgttaaagaatgcatgactatggttatgttcattcgtgacatgtctaatatgcgattcgattctcagactaaagagcgtttgacctctcctttcggtgagattcgtagccatattaaaattgatgctaaaaagatcgcacaaagcattttgaaagatgaaggccttattatgcctatcgtcgaagctgctttggctcgaaaattagctgctgaaaaagccgccgaaactaaagctaataaaaaagccgctaaagctaaagtagaaaagcatatcaaggctaacatgtacggcaaagatgccgacacaaccctgttcttaacagaaggtgattctgcaattggttatttgattgaagttcgtaatcgagaacttcacggcggatatccattacgtggtaagtttatgaatacctggggtatgtctgctgtagatatcctaaagaacaaagaagcctttgatatctgtgcaatcaccggattaactataggtgaatctgctgaaggtatgaactacagaaatatcgctatcatgactgatgctgacgttgacggcacaggttcaatctatccttcactgctggctttcttcactcagtggccggaactatttgagcaaggtcgaattcgttttgtaaaaactcctgttattatcgcccaagtcggtaaaaaacaagaatggttctatgatctgccttcttacgataaggctaaaggatctctcggtaagcatagtattcgatatattaaagggcttggttctttgacccgcgacgagtatgaacgagttatccaagaacctgtttatgatgtagttaaactgcctgaaaattggaaagatctttttgaaatgctgatgggtaaagatgcatctaagcgtaaagcttggatggccgcttaattaaagggcttcggccctttggaggttatatgaatctgataatgcaaaataatgcgatagttaatttagataacatagccagaatggtaatggaatctgcaattaggcgtgttgaatttatagactcagacgccgccaggaagaaaaagtacctgttggctgtaagaaacatagccgataagattaaaatgaaccaggagttaaccagcgttgaacgtgactcaactttaaagctcatttacatgcaccgcgaagaaaagagtacaatgggcgaaatgagacgaatcttggctgagcttggttctacccgtgaatatgttcaacataaggtggtccagaatgcgaagctatagagttaacttgaacttgttagataatgctatattcaaagagtatagaatgattcaacggttctttgatatcgacgaagctgagttgtttaaagaacgcttcacagatattcgtattaaaataacccacgactgtgcatcggccgaagagctcttagagtgcgccgaattgattaaattacataatgattaaggtaaaaaatgaaactcgtaactgatgacgaaattgtaatgggctctggcggtaaatcaaccgacttcacgattaaagcctcagccaaagcgttccgtgttctgtcttctaacctgtacaaaaacaaaattcgtgcaattgttcgtgaactctctactaactgtatcgatgctcacttcctgaatggttgtactcgtccattcgaaattaaggttccttctcgtcttgacccacgatttgttattcgcgactttggccctggtcttgatgaagatggcatgattaacctgtacactactttcttcgaatccactaagaacaacagcaacgactttatcggtgctctgggtcttggctctaaatctccattaagctatactaacaccttcacggtagtatctaaccatggcggccgctctcgtggttatacagtaatgatggacggtgggctgcctaaaattcgtcctacctttgatgaagaaatggctgaaggcgaaacgactggtttggaaatcactgttccggttaaaacagatgatatcagctcatggcagcatgaaatcaagtacgttcttcgtcctatgggtgctggctctgtagttctgaaaggaacacagatggaagttgatttcttcccagaccaagatgtgcacacaacagatcagtacaatggctatgaaaataacggaatctatgcaatctatggtaagattgtatatcccttgaacgacgtccctggcgttaaagattcgtggcttcgtgctcggtattcacgagtgtatattaaattccctcttggtgaactagatattactccgtctcgtgaagagttatctcttgatccagacaccgtggcaaatatccaggcccgtgtctctaagattaaccaagagtatcttgaacgagatattcaagaactagatgaaatcactaacgagcgtgaactggttcgtaaatacaacgagttcggacgagccgagcgtaacatcattgctaactcaggtgttaaaactaagttaggttctattgcaactattctggccaagtatgatgtttccaagcttcgtgatatggcgatgaactctggcgcggtagcatacgacggcgatgacgttaaattgtatcgtttcaaaaacagctatggtggacgttctaagatttatgtagattcgatcttaggtgtggacaaaaagaaattgactgttcttattgatgacaagacttctaagcgtgtagcgactatccgggctcttaaacacgccggtaagattgcacgttatgattttgttctcgttatcaacccagaatcagaactgcaacaggccttcctgaaaaaagctattgaattaatggctgaagatgaagttgtcacgtataaagtatctgaatgtgaagaactgcgtgctaaactgcctaaagtagaaagcgatgagcccgcagagaaacgcccagcttcacctaatggtgcccgatatacgtactctgagaaaaatggttggactattgaaaatcttaagttaacctctagtgaactctctgaacttgaaggttttgtgttgcttcgcagccgagatgatttcatgacttttccggccaaatcatccattagctgctttagtgatacagatgctcgtatcttggctcaagaatgcggtatcacagagttcttggttatgcgtccatcaggctgccgtaaagcattatttgaaaatgaaaatcttgttgatatgatggcttatatcatcgagcgatacattgatgctattgatgaggtagattatgattgctatctcccaagaaatttccgtagcaaccgtgtgattaaccacattattaataagaagaaacttgggttccttctgacttactttgtagaaggtaatgctaactccgattcactgaatcgtctggttaatatgaaccagcgattgagcaacgttacggtgaaagataatactgatttagctttatgtaaccagatttataataaactggaagaaacatctacaaccgctttcactgttaaaatgaaagagttcaatgaaaaatacccggttattgatactgttctgaacgaatggcatctagaacaggcccaaattgacgatatcgttaaaatcatggctgctctcgaagcagccgaacaatccaaataatagaggtgaataatggctgtattatgtctgtccgagaatgaaaaagatgcaatcgtaaaaggttttaacggcggcctttacaataaaaaagaactggctgaaatttatggtgtttcgactgataccattcgccgagtctttaaagaacgtactatttctccaaatgaaccagctaaagtaaaagctccggctcctcgtgaattccagtgggccgcttcaagcaagttcatttctatcactgaaggacgtaccacttataacgcagacccaagtcatccagggtttaaatcggccctgatgaaactggctgaaggcaaaattgaagaagccatcgaccatatcaacatcgaacgcggtattgctaaatttgttaaaggcaacgtccgtatcgaagatggtgctctgtattataaggacatcgaactgaaatctggtctgactgaacgtattatttcagcgatggaaaatggtgaagacttcgaacgttatctgccgttcctggaaaacctgatgctgaacccgagccgtaaagctgtttatcgtctgtttgacttcttgaatgcaaacgacatcgaaattactggcgatggtcactttatcggttggaaggtggttcgttctaactactttgactgtgcatctggtaagtttgataacagccctggtaaagaagttaccatgcctcgtaaccaggttgatgaagacgacgaacgcacctgctctactggtctgcacgtttgctctaaatcttatatccgacacttcggtagtggttcagaccgtatcgtttcggttaaagtacatcctcgtgatgtagtaagtattcctgttgattacaacgatgctaaaatgcgtacctgtggctataaagtccttgaagacgtaactgctcgctggaacaacgaactgcgtggttaattaaatggggacttcggtcccctttcttataaggtgacttatgactggcttccaatctcgtgttgttgatgaattatctgaactgaatctgaaaattaatgctctgactgcattcactatcggcagtgtattcaaatcgcttgatcctgtagaccaaagtttgcttcttagacagcttgatacaatgtctgcgtaccgacacattctggaaaaacgtattcagaggttttaatgattaatccattcaatgccggcgatcgtaaaatcacggtctcaaatcttcgcggaaatcataaggcagctacagtctattgtctgaaattggtcaagcacccaggtgacgttcattacggctggcttgaatgtgatgaggttgttgaaggtgagtttcctttagtcggttctagtaccctagaatttgacgataggatttattatggtgaattacacatcagaggtttatatggtatcgatgagcttgacttggaaacaactactgaatctgaagccggttaatttaacggttgaaaatcttagtagagaaaacaaaattaaggccttgaaagaatcagaacactcgatgtccttagataactctcaagaacaacgctctgctttaaagcgatgtgtaatagcaacattaggcgagcaactgattgctcagtgtactaatggccgagtaatggatacagagtttaacaaagccaatccattcagttactccttcgatgttatctctgaagaaggtgtgcgcatcgaggtcaagacccaccagtctgattctcgatggatttcggtccacactaaaggaagacatccctacccttatgggtcagggattgacctagatgggttcctgacgtatcgattatctgacttaataatcatattagatgtagaagaagtgggcccaggcgtctacaggtttatccctcgcatgatgggtgaccaaactgcctttctgcctgggtctggtttagttcaagaaagcaagtatgaaaacggtggttggtacttacgctcttgctctgaagaagaataccagtttaaaaggtttacatactgattagactgtggtatagtactcctacaccaacaatgaggagtactaaaatgaaatctttaaatcaacgtttcggaaaagcaaaatatggttgttcagctgagcgtgagtatgtttactatcaacttcttgatgtagaagctcagctcaattctgcagctgtttctggaatagctccgatgtgtggagttgaagctctgcgtgaagttcgtgatggttatcgccgccaaattgaggaaatgtctaaatgaaacggtttggattgttcaatattgtaagaaacgagtgggtaactacttggtgcaccggaactttcgaaggttgggtagaacattatctcgactatgatgatgaccaaatcttcatggtgtctactgaacaggatgccttgcgagttctgtcaggtcgttctggctatctcacttatgctgagatggacttagagatttcccgcggtgaaataatcgtgaaagaaattcacctctaagtgtttacaacgtctgtaggtcatgatactatgttcctacagacaaaatggaggaaatcatgcttaagaacattttcaactttatcgttttagctatcgtactgatgataatgcctgtagtgttctttattgatgtgattatcataaatttaagacacttgttctgaggaaattattatgaacgcagtattaggctggattaacgcagcggcttttattattgggtgtaatgtaccgtattggctcggttggtctgatactatgttatggttctttaagccataacatctgggagctaggctcccatagaggataaaacttaaactttattgaggaaattgttatgactattgatacccgcaactacatgacccgtggcgatctgactaaagctggcgctgtaactgtggcccacgttaaaaacggccatactgtcggcttcgttatgcctgaagttctggctcaaccaggtttctactttatggttaaaggcgctggtgaaaatcgtacagtaggagcacggttctttgtaggcaaccaacgttctgcacaaggttttaatgctacgttgtcccatattcgtcaaggtcgtagccagctggctcgtactatggctagcaataatattgtttatgaagttttgtatctcccacaatctaaaatgaaacctttgactactggcttcaagaaaggtcaactggcactggcattcactcgtcagcacagttctgaaatgcagactttgtctgagctgaatcgtattctgaatgacaacttcaaattcattcttcagagctactaatgagtagttttatgaaaatgctctttgctatgggctacttgttagtcctagcattgatgatttgcgctgggacttatgttaccttgggtcttcccgttacaagcaaacttatatcgctgatgttgttctctgtaggctttatttgtttcgaaagaatttctaaaatgtgtggggtatacaagtgaatcggatatacgctttcttctggatgattatttgtatcgttctgtgtactatgttctttgctggtgcctgggttcctacatcggctcaagttgggttcatggtctgttggtttgttttagctgcggtaattgactttttaaaggcctgcaccgccaatgattaagaaaattttcttagccatccttgtgatggctattcttgctttagcgatgtactatggtatgatatatggagtagtttatattgtgcttttcatttctgatgttatagtgcatctcggttcatttatttggtaggtactatgtctgatatttttgatatgatgacaaaacaagctagtgatacttttaatgttcgcagcttagaaagtataattaataacgaagctcttgcgtatgccatgtatactgtagagagtcgtgcaattccaaatatgattgatggctttaaaccagttcaacgttttgttattgctcgtactttgatgcttggcaagagtgataaaaataaattccataaactggcctctattgcaggaggtgtagctgaccttggataccaccatggtgaaggttctgcacaagaaactggcgcactgatggctaacgactggaataacaatgttccgttcttagatggccaaggtaactttggttctcggttggttcaagaagctggcgcatctcgttatatctttgcacgtatttcagaaaatttctacaaactgtacaaggatacagaatatgccccagcccataaagacgatgagcatgtcccggtggcttattatctgcctattattcctactgttcttcttaatggtatttccggtattgcgactggttatgctactgatattctgcctcatagcatttcttctgttaagaaaagtgttatccaagctctcgaaggtaagaagatttctgagccgaaggtaagctttccgcagtttaaaggaaagataattcctgtggatggcgcatttgaacttcacggaatatacgaaatgaaatctcgtaatgtaatgtacatctctgagattccgtataagtatgatcgtgcatcttatgttaaaatcctcgatgctttagaagataaagggttcattacatacgatgatgactgtggcaagcacggttttgggtttaaagttaaattccgtaaggaatacaacttaggtgaaactgaagaagaacgacatgaaaaaatcatgaaggacttcaaattaatcgaacggcgttctcagaacatcacagttatcaatcatgccggtaagttgaaagaatacaaatgcgccgccgatttgattcgtgactttgtagaagttcgtaaggttttcgttcagaaacgtatcgaccttaaaatctgcgaaaccgaagaggctttcaaactggccttagctaaagcaaagttcattaagaaggttattgacggcgatatcactattgccggtaagaccagggccaaactgatcgaagaagttaaagaattcgacgaactagcagattatgctgaaaaacttgtaagcatgaacatttatcatattactagtgatgaagccaagaagctggccgaagaagctcgtactaagcgtgatgaacatgaatattggaaacagacagatgttcaaactgaatatcttaaagacctcgaggaaatcaaatgaaatttagcttagcttttattatcggtttagtagcttttctaattaacctgtcaggtggcatggtttggtctactgctgctttatggggaattgcattctggatcggcagtgcagttgttatctggattctagttgtcgttctgatagctattggtgccgcactggccaccagatgattactctacacgggctgttcgtaggaatggtcttttctttgatggtaatcactctgattatcgactataccttctaagtggtcgcctggacattctgttcaggcaccctttatgtttcatcatccttcctttaaaagtctccctaggcaattcctgccaactcaaaaaattttaaatccaccagtttactttgctttcaaaacaggttatagtactcctacaccaacaaaacgttgaatcggagaaataaaatgaaccttatcgaacgtgtagcttctaaagctttttaccgtaacggtgttttctatcattctattgaatcggctcaaggtttctttgaagctaaatgtaagcagcatggccttgaggattggcacttcatggttgtatcttctgcaaagaaaaataacatcgggtactgctcttattacaagcgccgcatcgctatccaagctcgctatttctacgcaatgtcaggagctcaagttgaagaaactatcctccatgaattagcgcatgcattgactaaaggtcataaccacgatgcagtgtggagagctaaagctattgaactaggtgacactcatgctcgtgctacaactcctattcgtactggtgtaggtttcatggaagaatggatgattaatcacggaacccggtatgaatccgagtttgatgcagtagcttttacttctaaagagctgcctcgttctaagaagccaagttctccaaactttggcggtgaaaagaaacgtattgctaagcccactcgcaaggcaatggctatctacaaccaagctaatgctcctacacgtagcgatttcatcgagcagttcatgagcctaggctacaagtttgactatgcagtagttcagtggaaactttgcaaaaagatgtaatttaaatgtttacttctgcttcaaacgtgttattatagaccctatcaaaacaaatgaatgaattggagaaataaaatgtctaaagttacttacatcatcaaagcttctaataacgttctgaacgaaaaaactgcatctattctgattcaggttatcaaaaacaactacatcacttctgcagaaattcgtgaccttctgtctgaaactatgaatgcttcttcagtgaactccaacattggtgttcttctgaagaaaggcttaatcgaaaaatctgaagatggtctgattgctaccggtgaagcaatggatatcatccaggctgcggcagttcttcacgctgaagaaaataagccagagcttctccagaaacgcagaactcgtagtgctcgtggtgttactgaagaaatgactaaactggctgaagaagttaaaactattctagaagcccgtgttgatctcaaaggtatcatcgaaaaccgtagcaacttagaagttcagttggttaagcgtactaacggcgttcgtcaaatcgaaatccgccgtgatggctcacttcgtgtctttggttataacatggccgagtctgagactaaagtgttcacttctttaagttcggatgttaaggtcaaagtgggtggcaaaaacacttacatcgatttcccaaacattacttctgaaatggcttcaatcatcgctaacgtattctgaggaaaatatgaaaaatctttatgaagtaaccttttatagaggcgacaatgttttacctaacacgtataatattccggcagaaaccgaacaggacgctttagttcggttaggacaaatatacggtgatgataaaacttatcgcgaagacctggaaataaaagtggttggcatccgtttagtaatgagaggttaatatgaacaagttagaaatcatcaacgaacttcgtaagtgtgctgagccaactcaagctggctgggatgtatggtaccacggtgcttatctaggtactattgttaaggttaaagccggacagtacggcattttacgcgatgatgatgcggcgccgttaggcttcagaactaacttcatggctgttatttcttcttatgttgaagccgctaataaggttcgtattgcagattacatcgaacttcaaacggctcaacctgttattcgccagattggtgaagcacctaagaaaagtatctggcagaagattaaaggattctttaaatgagtgaacaagaaattaatgatctgattatgggcctggtaaaggatattaataccagggccttgcataagtaccaagtaggcgactcttggacaatggctgactgtaaactaggtgcagaccacggtcgctatgtcattagagtccttaatgaaaaactaactaacgaggtcaaataaatgagagtactgatgacctctagtgaatactttaattcgctaaaccgttcagaaaaggctcaaattaaacggtttataactgagcttggatatgtcgaagcacatatgctgagcatgcacatccaagaatgcggccttgcaaaacggtttcacatttcaaccagatgcttaaaagaggttatcaaacattatgcttccatttaaagagcgcttagcattgttagacgaagccttagcgcgcgaaacccccgagagtctagctaaaaaattagcctcttatggtggcgaatatacagagcaaaatgttctggacgaagtacctgaaatctgttgggaaactgcgtattggtacgaagaccgtaagttccaacgtcgaattgtttcagcagctaatcgcttcaaattgaaagatggcggaactttagttattccgggtgcgcgccattattctaagtgtatggcagctgtgttggatgtggtttctgataaattagtttcagaccaagccgttggcgacgaccaaggcttcatcgatcaatacagcaactattggacccgcaaagaagctatgattattgccacatatgctaatcaggtgcgtattgagcgtggtggcccggctgatgaattatattcagaggacctttattaatgaataacttggtagcgaagaacgattttaacaagggtggtgctcataaggacaaaaagcgcgctgctaaagattccaaacgtaaacaaaagcataaaggtaaagataatgaatgtagcctatgaacaacttgaacgcgaacgtgaagcctatgacttgttcgaatacttgatgtcgagtatcgtagaagaattccgtgtagacatcaagttctacaggagcaacgaagctgaacctaaattcaagcttaatcatgcacttaaggccgacttcaatagtgaaccaggcggcgtcaggctaacccaggctcatcagacgtatacattacacggcccttctcgtagtgtagtttttgatgctctgaatcgtgtcaaaaagataagccaaaaacatgggtttacaatggtagtgtaacaaggtatagtattcgtataccaaaacaagtgagatgtctgttaaggaatagtatgtctaagtttgaaatagtttctgaaattattactatggcttcattactgattaaatttcaacgtgaagatattttagaaaatcgtgaagggtttattgcatttttaaatgagtgtgggattagaaatgagttcggaagggaattaaactcggctaatttccgtaagatggtagccgagttatctgatgacgaaaagaaacagctaattcaagaatttaacataggtcatgaaagcgtataccgccatatggaaatgtacgctaacaattaatcacttactccttcctaaagcccttacagcctgcccgcatattgatgacaaacgatcttccgttatagttggtcgtttgccataccaatacatctcaacagccccagaataaatgttatccaagttaaaatacggacaagaaaacatataagtcacttcttcaggcttcttcttagttggtaagaaaacaaattcattctcactccaatagtttcttccagacaagtgtgtagtatattccggcgttgttttatctataggatacccacctaagtttttaggatcgactgtgcttggtaattttccttcaaaagctattaaatctacgaaatagtttaagttctttggcctgaaattaaatactgcactaaaatcagcaccactagatacatgaactatttgaagttgctcgagcgctgtgttctcaaatttaagctcttgagtttttactagaatctcgttataactttcatacgaactctttacgtatgcgttagctatagtatcggatttataccaaataaaagtaagcccaaataagattacaactactataactctggaaaacaaaagctttccagaggcgttatctttaaacaagcggtcaagcaccccaaatagaatatccgatataggtatagatattttattggccatgtgccctcctcacttatatttatctcataagggacgctctttccttgcgtagttgtaatagctctgctttatctgcttcaatatcatcagacatgccaaattcaattttatcagataatgcatcgataataggcgttaattctcttatcttagcttctctttcaagtttttgagattcagcaatattagaatattccttaacaccgaacgggacccaatcatcggtttcccatttataatcgccatcaggtttaatcttaacgtttccggaagatttaaatttataataagcctctttagaagcattgtcatcaggtgttaaagtatacggaataatgatgtccccaaaggggacatccacatcgatataattctctaagtcatttgacctaggatttgttgctttatcaaaatttatcatgcaattcttttccaaatagttgtgcggtggacattattacggttagcagagaatccacatgccatccaagtaccagaaaggcgtttagcgccataatcccaatctccaccatcagaggttgtccaaaaaagattacttcctgctatagtttgccctggtgtaaattcctgggatccagtccagtcattgtgggtagctgccatgatatacataccgacatcaaatgcgcctcgactaatacgccaggcccaaatattatcattagataccttattagctacgccaggaacccagttattaatataaccttcgccacctacaccatatgaagtgaataggctagattgtaagttaccgtcagaatgcaacgtccaacgcgctccagggccagtaatttgtcctgtagcaccgtggaactgccattcactagaagcggctcttaatcccatcatggtattattgttacctggcatgtaaatcacaccacgctcgccaccattagaatgtcggaaccaaaggtgcgttgtaccattatctacggcaggggccttaacatctacagcaccaacaaaactaccaataccagtattactaacaacattaccgcctacttctaaattaccgttaagtgccgaatttccagtagcccaaagtgatgtagtttttaactgatcacctgtaattattaatctaccagttacagtaccacccgctaatggaagatacgtgtcgccagcattactaatagcagaagcaaccttatcatcaacatatttcttgttagtggcttggttatcctgcgtaggaacatcggcaatttgcacatacttggtagtaggaacaataataccaccattggcattaatagtgccattaacggttgctgcgctagaaacagttaactgtctattgatttgggcatttccataaacagtttggtttccgtcagcatctaccttgaatatgtcgttaaacgtgtcagcaggattaatttggttattggtattggttgatgccataacagtaaacgagttagatttactgatagccatttttcctggcataccgtttttcttgactaaaccaatatcagagtttttgccaagaacgatcattctgttatcttcggaaacgttcaagttacctgtgattttagagttctgggcctcgaactcaccaccgactgtcattttaccgttttggccaataactgtagcttggcttgcagtatcgccatttggtcttaaatgaagcgcctgacctgtttttgcgtagataacaaaggcaccagagtcgtttgaacgaatgcctggaccttgggaccactcaagcagaggctcattattgccatatgctttgactctaacttctgaattagatgaatcaccgtaaggacggaaagcgatatatttagcggctgcagtggctgtagtagaagctgacattattaatgcgccgatactaccgccttgggcttcacgaattatagcaccgttcttaaatgtaactattggatcattatttccaacgccgttaacaatcaatccaccaaaagtggcagtttggcctaaacctatgttaaaattatttcgggcatcattggcattattagctccggtaccgccagatgaaattggtaaaattccattactatttgaagtggttactacttcttcccatgccgtccagttagcagtagtcgcagcagtagttcctgtcccagcaccatatctaaagtaggttttgttgttacgtccagatataaatgtttgtttattgcggaaatctacacctgaggctgaccgcatcttttcaacaataacaaggaaatcacctttcaaattagttaaagctggtggcataccagttatattagtgccaggtccttgagctaaacacgaatatactctacgagttccgacatcattagagtctaagaaaagatcatcgatagataaagttcttccaccaatgtcttcagcaacttccgggccaagataaccactgtccatattaacacggacagatctaatactcgaagtatcaaatactaaattattatcagtttggttatctgcagttccccagcgaagagacccatctttagctaaaccaagtttacgctggagagtcggggaagtgccggttaatctgaatgcaatggatatattagcatcagaatttctctcaagaataagaggtgtgtgctggacacctttaatataaagagattgcgaattagtattagtgtcggccatatcgatggtaagacggccggtcatggtgtcgccagatttggaaactttactattagcatttccggttaaattgctcaggtaagtgtttaagtttccaccaaataaagtaccaccaacaatattaccatcggtttggaatgttactgctttagagttgtctgatgggaataatactaaatccccggtgtttctaatttgcgccaaatgacgcatattactgctattaccttcatagttccaaaaatcaaatccaccaggaacggtcgcagtattcgaactatggttaataaaatcagtacgacccgacccgttttctttgttccaagatatgtatgcaccttgagaaggtggaacgtacgctgcgccaccgccagataattttaaaactctgccactaatggtattagcagtaatatcaccactgattgtaacatcagcaccaaaagtgccgcctcgagatttagatacaaagtcgttatcgtcagctttcggtttatttgctgtaccgtaaagttcattgtagttaacatttccggccgcaagaccgctattattagctgcgtatacacgaactttagcactagcatagtcgatattaatagctgacatggtatcaccagcattcataaaaataccggagccgtgactataaaggccgttttgaacactaacaccagatttagtaacaccacgccagaaagtaccgccatacgcttttaaacgattcagcatatccgcattagagataatgtcgttgatggatttaccaccattaccacctaaaccgtaagcaccgttttctaaaaggttgccggtggattcgccgacgtcgcgaatagcagcagtacctagacctaagttattacgagcagcagcagaagaagtgccgccggtaccgcctctgctaataggcagaataccctcagtaccaccgtcaatgttaggcttgttaaattcatcatagatacgaatagctgtactctcaactaaaccttctggtgcagttgaagaagacgtgtaaccgttaggtagataatattcagtaccaccagcaatagataacagtgcaactttaacaccaccgatgaatgctcgttgcataaaccacagttctaaaccttcagaggtaagaattaaactataacgcaattggtttgtgtcatcaagtgctggattaccgagacgacggacctgtaggtggttacgaatgttactgcttgttaacgaagatggaaagtttctagcgctacaatcgataaagtcgatagagctgtaagtactgccataattaccgccattagtcaccattaattgcaggcggctctgggttgatccagggtcttttaaaagagcaactttagtccatctagtttcggaaccggtagttatagggaaattatcctgtttaagctggcgctcattttctacgtttgtcagaagagtaccggatttaccaggacctcttccatccatggcgtaataaacctgggaacttctaaacgtcagattactatcattagttagttgaagaacccctatgcctagctggtttttaatataaacgtcattagcgcctacacccatttcaagaacaaggtcacttccattactaattttaacggctttattagtagttaatcttaaatcgcctggaagagtagtattaccagagccatcaagtaaaactaattttctggcttctatatttccggccccacgctgtacgaattggatggtttcagcgccgtcgtcaatagtaccgatctcaagaataccactattacttgtaccgccagctccaatataccacccatcattagtggcgcatttacccacaatatgtcgtactatagccccacttaaatcagtggcttcgaaatttattgatttgttcggagcaacagtaacattatcttttaatgtagtagttccatcaacattgagagtaccggaagtatctaaaaatggaacacctttaccggcgagctgtactacggcgtcattttcatctttagtataaatggcaagagtacgcccatgggtattcaatgctatttcgccacgggctacgatatcggcactaggtgctttattctctgtactgcttcttttaaattgtatttgttttaagttctgatcggccatggctacctcttagtatgtgccaaaatctattgttactccgcgcgaaataacctgatctaatctaggagcatattcaccggatgtcgctttatttactataactacgccagcttcagctctaagtacgccagtcatagtatcaccagatttcaaaacccggctgtttgcattagtagtaacagtatttattcgcccgtcaacgtagtctttacgtgttaaatgttgagcagctgtaggagcattattagaactaagaatagaagctgttatacttcctttgtttatttgatccccggttctagcgttccaaactactgtccgttctttattatctagtgaagtacgaataccgacaccgtaccatgaaacaatgtcaatgttcgctgttgttagagatgctgagtctccattaccagaataaagcccatttaaactagtgccgccactgaaggtcgcagaaatttttacgccttctttaaattctacttctttttggaaagcttgagaacctgtgccaccactagccttggatacaaagtcgttatctgctgctttaggtttatcattagtcgtgtaaaccgtataaccacgataagtcaaagagtttccaaccgggaccaatggcatagtaccttggtgccaaatgggtaaacctccgactgtagaacctgcttttaaatcagccattttcgtccccttataatatgaacgtatttatacgaaaaaaggagcccgaaggctccttattgtagttccctgaattctttagcaatacgatcaccactactatcaactttagtgttgggcaattcgaacataacagcagcaggagttaaagaaccttctatagctttattcattcgaataccattaacaccaaattcagcttgagtagctgattgtataggcgccggttctacaatttgaaccatcatgacattttgaacagcactttcagcaacaaccgatgttctagggtacctagaaactacggcggtgaaaccgtctgcgccggatgggattcttaggtccttttcaaaacgaacccatttatcagcagaagccgcaggaacttccattgtaaatgaatctaacatagttgttccgttaaaccatcttacagacattctcgtagtgcaattagcagatattaaagcctgcgaagcgaaaagatctgcagtaaatctaacccacaagttttgttgtaaaccatagtcagataacttagaaataactacatcttgctgtgggtagcgtttatattcgtatctggttgtatcacgataagtttcattgttttcataagctctttcaggaataccgcatagccctacagtgcctacattatcgtaaactgccgcaagttttgctctagaatcttctttaacgattccgtcatttccagtaaaagcctcatacaaaattaatcctttgcctgcagcaaatatggcgcaataggcgctattaggatataattcagtataatgttgttcaggccaaacaactgatctctttgatttaaaccaggcggttacagaaggagaagttaaacatctctgattagtcattattgcgtacagacctgatgttttactattcatgtactcgacgaatgcggcgttagccgcagattcaccgtttgttaaatcaatagttttaggagtgccatcagctgccgaagtattaatctgccaaatgttgatgccccgtcctggggcacctccagtattgacttgattaattttgagcacatgcgtattaggaacagcattagcatgtgtggctataagttctatccaatatctcacttggttagattctgacaagatgttagtctcgacaaaatcttcaccaaatgcagccattaaattactcattaatcacctacccattcaaacttaacggtctgagaagtacgatcaggataaatcttgacagggccaatcttaatccattcttgaacttccaaggttttaacagaagtacttactgaactcggggcaccaatatctgcagctgtaggaggagtggcgctagtgtatacacgcatccagctatcaaacttcttaatatatgggttatataccctggtccacatagtatgagttcttgctttagctgtctgttcagcattagctggataaggagtccatgtctgataggtataatcagaagaaacacttatctgagctaaaagaccgccggcgcctgttttttcttcgtaacctgttacgatcatagaacctggaactaaaggattctcttctctgatcggaacaacataaccagggagtgtggctaacttagaagcattcttaatttctaccgtccaagaaccagctcttgtgtatttttcagagtttgctgtagtagagtgatcataccaaccttcagacccattaataacaagagcagaaccgttcatagttaagttgccggtcattgtatcgcccgctgacttaacgtacctagtatctaaaatgctatccttgtttttagttgttaaaataacataatttcctgagtcatcagtaacttgtgtctggctcgcatcatttgttaagatccttcctttcttagcaagattgccaaaagatgccacactgtcagaatcctggagttgaaccactgaagcatttttaacgcggtacacagtgtttgagttaacatctgaagcagaatgaattgtcccgttagaggttaatttattaggagcaacaaactccccattattaccaaaagaaaattctttaccttcatcaggcgatgaacctttaacacgcaccgtaagctgttgggaatttcctgtagctaccggtctagcgtaaattattccacgttcgtttccgtccgagtttaaaaatctgacgtgagaattagctgtaggcgaagtagcaaataccttaacctcaccaccagtaaaattaccaagtcctttaacgttgatatccgaattaaaatcagcggctttggcaagagttaatctgccgttaacggtttgatctatatcacgtctaataaactggtctgaagtaagaccatccaataaagtagagttatcagctttagcattcaaaggaagatagttagctaacgcctgtttcagcccattaggtgatactgcatagccattagattcttttgccacgccatcagaacctgtagtactgttaccagtccaagcaacggctccattagctacacgaactgtaccacgaacgttatcttgtgcacgccaatcagcagaagtctgaataacatacttcaaatggaccggagaaacggctacatcgttagtagtaccggtgttagtatcggcttgggttgcatatttaacaataccgtacgcggattcagtaccttttttagcttgaagctttttagacgtcagataatcaacatcatcggttcctgcattgacaagcgtctgagttgcaatcttaggcgtaccacgttgagtttctgttggaacctggatattaagcgtccatccgtcccacaagtttccggattgggccaacccagcttcgggtcttgttgtcatacgagcgggtctagaaaagtagtctttaatttttctagggttcactgcaacgttttctaaagatccaccgtcaaattcatcttggatggcataccgggtaataccagtttgtgtataacttgctgatctgccatttagtgttgctggagtaatagccacattacctaaagcactagacatagcgttagcttcagtttgagtagcgatacggattaaaccgatagcccctgtagtagaagttttagcatgcaatgtctctggagtaacaacagttggaatattaccggaagctggggtgccattacgaacctctgcttcggtagcaagccatacacagccggaggcaagttcagtagctttatattcacgtaaagttttaggagttactgctttagaaaaatcagtgtggtcgtacacaccagtaccgttagaagtacctgaatctgctctacttgttcctggggtaccacccttattaactagttggatgatgccatcaagagtagcagtagctttacgcgcattaagctttttaggagtaacgatcctatcatctacagtagagccattcgtttcagcttgggtagcaatttcagcaacaccacgcatggtttcagtagctaaacgatcgttcaacttcttggccgtaataatacgggtgtcttcagtagttgaattagcttcagcctgagtagcaatttcagcaataccagtacgggtctcagtagcagtacgctgggacaaagttaagggtgtaatagccctatcatcattatgaccgcttgttgcttgagcttgcgcggtagttgctaatgcaataacacccacacgggctttagtcgtatcgttggtagaatcaacgcgctctatactagagatgttttctgaaacaatccaatagttattaggagtagcttctacataggccaactctaagaccggagggtagtccgaagttccattgaatgataaactatcgacttgagtccacgcggcatcaggcggatactcagagcgtttagggaactgaagtaagccaaccgtagatgctatcttatctttagtgccaccggccggagcagggggagtaacaatgttgaccgtttgtcctttgcgcatgtagttcataccgattctaacagtatcacctggagcaacttgagtaggcaaagtaacagaaatagtttttacggtcgagttgttagcaccaaatatggtaatagcttcgttaggttgcatcactacatcgtcagtaacgactttgatacgttgctgcatgtcgatagaattgatgcgccataccttacgtgggccatcataaatcagaacaccgccgcctttaatcttagatgtccaggatgtagtcccttcagttccaatagaactatttgcatcataagtcttaatggtaaaatggtataacggtgttttgccttccggttcagcaaaggtaatcatatcaccatggttagcataacgaggaagacgaactgttaattttttattagaagtataataacgtacaatgtactcagaactctgtacttgagcgccaacatcagtagcagcatccagtgtggtagaatcaatagtacgagccaaagaactcagatctgaaagagttacgtaccacccggccgatgtaaacgtcagcagaagcatactataaggacgagtcagctgcagttcacgaaggatacttccttggaagatcattgctggagcagagtcttgtactttaattaatgcctggttgatacctggtttaccgccgatgtcccggataacaatcgtatcaccttgttctactttatttttaggaagaaccagtgtaaccggattaacacgagtatcgatgtttgcataggtccctggtaaaagttctgtggtccctgaactatactgctcccacttagggtcaacacgcatggaaatccactcaccttgtttaaaggtgtttactgccggcgggataggagccgcgatatctgcacgtgctacccagatacgtttttccatggtaaccgcaaagccttcaggatacccacgatcattggcccaaggctggattgtattttcttggataagatactcaacgttgaccccatcgctcatcacagtacgatcggctttagcgacgttgattactttgtttccaccagcatcaagcccgttggttgctctaaaatgctgtttgattaagtcgctcatttttattccttatggtattagaaatatgcttcattataggagtatttataatggatctgtccattactactgaataggttattcaatgtctctaaattttatgttcggtgaagaagaaaagaaagaaggtgtaactcttattgacttcagtcaaattattatggcggcagccttcaccgagttcggtgaaaaagcgggctttcctaaaatatctgaagctatgctacgacatcttgttttgaacagtatgcgtaaaaacatcaaacagtttaaaaaagaagggtacacccgccagattatctgtgtagataactctaagtctggttactggcgtcgtgacgcgagtcattattataagaaaaaccgttctaaagatcgtgaagaatctccatttgattgggaaggattattcacggcaatgcataaaatcatcgaagaacttgagaaaaacatgccttatattgttatgaatatcgataaggtagaagctgatgatcatattgctgtgttaacaaagttcttggctagccaaggtatcccagtccaaatcgtttcgtctgatggcgactttacacaacttcacaaatacccaaatgtgaagcagtggtctccaatgcagaaaaaatatgttaaaactaaatctggtgatgcgttacttgattgcgtgactaaagtcgttaaaggtgaccgtaaagataacgttgcttctattaaagtccgcggtaactactggcttactatggttgaaggtgaacgtactcctccaactaaagcatctgaaattgaacagatcgcgatgaattattatgaccacactaaattacaagaacttcttactgctgaacagtatgaacgttttaaagaaaaccaaatcttaatcgatatggatttcatccgtgatgacattgttgcttcaatcatggaacggtataataactacacagttccggctaaaggtaagatctatacttactttgttaagaatggtctgtccaagttaaccaacaacattgctgaattttattaaggtgaatataatggctaaacagaaaaaagaaaaagttgaatttgatgaagcccaagacggcgaaacactaaaaaatctaattaaagctgctagtgacgctaaactaaaggccgaaggttatatggacgaggtcgcaagcaaccgggctgaagcaaaagctattggcgtcgaggcaaaactgtttaaccagcttctgaccatgtatcataaaggcactcgtgaacgtttcgcaaccgaaaaagatgaagtggtagaaacctatgacagcctctttgattaataacgatgaagtcaagatcgatgacttaaattcacttcttgacaaacaagcaaatggcttcgctattgaagccatagttgctgaaggtgaactgggttatcttcaggctacttcacagtggctagaagaaaacagtattcctgaaggaaatttcaatagatttattccttctacaatagttgaaaaaattaaagctgaagctgttaatgaatacatgcttcgcccatctataagtaaaacacagaaaacaaatactttggatttcttattatgattcgtctccgcatgcctccaaataacgctcgttacgttaacggtaaaagtgtttatttattgtacttaatgcttaaaaatcacttcgctggacgatatgatgttatcaagtacaactgggttatgcgggtgtctgataaagcttatcaaaaacgacgtgacaagtatttctttgaaaagctcgcagagaaacacactcttaaagagcttacattaattttcatgagtaacttggttgccaaccaagatgcctggatcggcgacatcagtgatgctgacgccctcgtcttttaccgtgagtatatcggtaaacttaagatgattaaaaatacattcgaagatgatgtaaagaacatttactattttgctaagaaagttgaagttaacgcgctttctgaaatctttgagtataatgaaaaggtacaaacgagctatatctttaagctccttcagtcgaatgttatttcattcgagacgtttattatgcttgattcatttctagatataataaataaacatgattcagctaccaacaacttagtttgggaaacatattctactaagctcaaagcttatagaaaattgcttgaagtcgacggtaaagatgctaaaaagctctttattgatgctataaataaatgtaagatataataagtcttgtccaggtgcaaggactcatgttgcaccaccaatcgtaaataacccttaaattaaataaaaggtatattaaatgtttaagcgtaaaaaccctgcagaacttcaagctcaactggcttccctgaaaggtggttcttctttctcttccgacgataaaggtgaatggaaactgaagaccgataatgctggtaatggctccgcagtaattcgcttcctgcctggtaaaggtgatgaaggtcttccgttcgtcaaactgattaaccacggcttcaagaaaaatggtaaatggtacatcgaaaactgtacttctacccacggtgattatgattcttgcccagtttgccagcatctgagctctaatgattcatataacaccaaccaagaagaatataaactactgaaacgcaaatcttcttactgggctaacattctggttcttaaagaccctgcggctccagaaaacgaaggtaaagtatttaaatatcgtttcggtcagaaaatcatggacaaaatcaacgctgagatcgaagttgatactgatcttggtgaagttccggttgatgtgacttgtccgtttgaaggtaagcacttcagcctgaaagttaagaaagttggtggtttcactaactatgatgactgtaagtttggtgctcagtctgaaattcctaacatcaacgatgaagcttatcagaaagaactcgatgatggtatggtacagctgaatgacatcgtatctaaagacaaattcaagtcttacgatgtaaaccacgctaaatttgtacaaatcatgggtacttctctagttgccggtaatgcagcagcacaagctgataagctcagtgacgatcttgatgcttttgatgccgatatggcggcttttgactctaaacctaaacagcaatctgctccactgaacacatcgtcttctgatgacgacggtgatctggacgatctgttagctggcctgtaataatgattgggagacttcggtctcccattttggtatctaagggtttacatccactttagaacgagttactatatccctacaccaacaaatgggagaacaaaaatggctaagatttcattattcgttgttttgatcgataagtttgaagaagatattaaaacctacgggcttaatattgtcaatctatctcaggaatgtgccgggtatgatgctactatcgaaggtacctatccaatgattaataaattccttactgacctgtattgtcaaggaatggatgatgaaatgaaagctgaactttttacttatattgaggatttttaaatgaacgctttagttagtggcaagtactatcgtgtccgccctgcatttgaagacgactttacccgatattctaccagtaattatcggatggtcgacttaattaaagaacacggcggtagttttaccccgactcatacagctgtagaagatgggacccactatgtcagcgctgttgaaatggcagatggggcccgtttcgattctgatggtgactacgatgcgtactttgagctgcaagaaggcgagttcaagttctttgaagaagtcaccgatgccgaaaatgaagaccaggctgaaggtctgctttctatgaaaatcgaggtgaatcaccagaacttcgaagctatgattgatttaatcataaaaaatttcaagaaatagtttacatcggtgtagaggtatagtattatgcttctacaccaacaaggagaataaaatgaaacttcaacgtgaaagcattaaattaggttctgaatatcgtggtaagtggcagttctgcatttatgacaacaatcctgaagaactagagcgcgttgaagaagctctgtgcgaaatggatactgggttcactatgggcggtgaaacacgcacttggaacgactactgtgatggctgcccgagctatgaagatgggtttggttctggtttctggatcgatatcgacgatgttcctgctttcaaagcagcattcaaacttgctaaggctaagaaatgaagattttaactgattgggagtgtaaatattgtggtggcggtctatggcaattaggcggccgttgttttaaatgtggaatgaggcaaggctaatgttacaggtcgtattcgctacgtgcgcaaccaaaactgtagatggcaacactgaaattgcttttgggctgaatgacggattgccttggggtcgtatccaacaggatatggttaacttccggaatcgtactaaaaatactgtactgattatgggtgccaaaacctgggaatctctaaatgctcctttaccagggcgtcgttcggtagtagttattactccaggccgtggtaaacctaaaactaaaaatggtacttcgcctgatgaaattatgtatccggatgagtttatttccttcttagaaggcgagtgtattgttacttctacggccatctcagattatccatgggacactatagtggaccggaacacatacgatgtaagtattattggcggtaaaggtttaattgagcaggcggtccctcacgctgacctggttgtacatactagcattattaagaagcatcgtgtgaactcaaccgttcagatgactcaagagttcttgaatactcttccaatgactcgtatgcttaaagaatctcactggtaccaatgcgatgagatgactcaaattacagaatctgtgtatggcgaattccgtggctaaaatttttctagttaaagaagatgttatccagaaaaagtggcaacgctttaatatgaagcatggcatctataaacttaaagttcctctagaagagctttcttctgcatacggaacagaaacaactgttcggcaaatcaaaggggccccgtcatttaccctaacttacaaaattgagcagtggcttaaagcagtcgagcttaaaaacattgaagttaaaaacggtgaagcatctatctacggtagatttgttaaacacggctctaagctgtatttttctcctgaatacaagaaatcatatgaaactgactttatttgaagctgataaggtccaaaagaactgggccttggttattgaaggtaatgctcctactatagagcaatataaacgtaaccctgggaaatatagtacaattgagcacggtctttatcagcttgatttcgaactggaccctaatgctatttggagccgtgatgaaggtatccgtgtcaggctggcgcctaatgtagttatttctgttcttatgaataccagattcacactggtattaattgctaatggaattctgcagtataatgggtctaagttctctattaccggacggttcgttaaacagggctcagaaattaattttaaaccttgtgcagagtaaaataatgagcaataaacttaaagttaaagatgtgcctagtgcaatggcactttttatttgccgacagattcatcaaggaccgatgactcctcgtcagcataataaaggtgagcgttctctagggttcactaaaaaagcaaaacaaatgattaagttaggatacaagccaaaatatcctcgttatgaatcttcttattcctggatggactgatgaaacaataccaagatctaattaaacaaattcttactgacggttatgacactgatgaccgtactggcactggaacacgagccatcttcggcacgaagattcgatttgatttaacacgtgggttcccggcggtaacaactaaaaaactggcctggaaagcttgtatctccgagctgctttggttcttgtcaggctctactaatgttgacgatcttcgtgaacgtacccacggctctcgagacgaaggcaaaactatttgggacgagaactatgagactcaagcccgtcaactaggttatatggatggtgctttaggccccgtttatggcaagcaatggcgtgattttggcggtattgatcagttaaaattaatcattaaccgtattaaacagatgcctactgaccgacgtcagattgtttctgcatggaacccggctgaaatcgataagatggcacttccgccatgccactgtttttatcaattcaacgttcgtaatgggtacctcgacctacagtggtatcaacgttctgttgatgtgttcttaggtttgccatttaatattgcgtcgtacgccgctcttgtgcatattgtcgctaagatgtgtaatcttattccaggcgacctcgtgttctcaggcggcaatacacacatctatatgaaccatatggaacagtgcgaagaagttctgagacgtgaacctaaagaactttgtaacttagaaattacctggcctagtaattttgaaaactggccgacagaaactcaattggattgggtcactacatctatgtctcataaagacttcaaacttcatgggtacaaaagtcacccagctatcacagctaagatggcggtatgattaaacgaaaagacactaacaccggtgataagcttcaccaggttaaaatagaattagttaccgataaccactgggagaaaatccaagcagtggtttatcctaactactgtaacagaattcgtcttaagatgaatacccctaggcttggttttgaaattgataaagaccatgctcgttctttaatcgaagttctagaagctgcattaagggcctgcgaatgaatatccggtttgtacgcaatagacaccaatctaaaactatattaggagaaatgtttgatacgtctcctattgagctacctgagattaacgatactgtggtttttgataaaatagaacaaagagtcctgtctattactaaatcatacgaatggtctataggtaaagcacaattaatctgttggtttgaagttgatattacgtgaagatctgtcgtgtagtgaataaataccactccgacttcgatgtaaacattcatcgaggtactatgtggggaaatgatgtaggtaagaatgctggcagtcgtgaggctgccattcttgcatttaagaaagagttttataataaactcagaaccggagtaatcaaaaaggaacaccttgagactttacgtggaatgcgtctaggatgcacatgtaaacctaagccatgtcatggtgatataattgctcaggtggttaacaaattattcaaagacgaattcactatagaggatttatgaacgtagtaaaatccagcggctgcagccaagttttcactccaaataaaatcattcaggtccttacttgggctacagctaatacctcagtagacccgtacgaattgtacgatcgcattaagccttatattgttgacaacatgagtactcgcgatatccaaatcgcttgtgttaaagttgctgcgaacagtatctctattaatgaaccggactatcaatatgttgcttctaatttggctatgtttgcgctccgcaaagatgtatatggccagttcgagcctccggcgttcatcgatcatatcactcgtgttgttaacgaaggtctatatgacccggaaatcttgtctaagtactcagcagaagagatcggttatcttgacaccattatcgaccacggtcgagatttcgaactgacttatgcagggactatgcagcttaaagagaaatatctcgttaagaaccgtagtacaggtgtaatctatgaaacacctcagtttgcctttatgctgatcggtatgtgtctgcaccaagatgaacctacagatcgtttgctgcacgttgttcgtttctaccatgcagtaagtaagcgtcagatttctttgccaactccgattatggccggtgttcgtactccgacacggcagtttagttcttgcgtggttattgaagcaggcgattctttagatagcattaatgaagccgcgaactcaatcatcaagtatatttctaaacgcgctggtattggcatcaatggtggtatgattcgtgctgaaggttctaagatcggcgcaggcgaagtaaaacacaccggtgttattccgttctggaaacacttccagactgcagtgaaatcatgttctcagggtggcgttcgtggtggcgcggctactatgtactatccgatgtggcacttagaagttgaaaacctacttgttctgaagaacaataaaggtatcgatgagaaccgcattcgtcatatggactacggccttcagatcggtgacctgatgattgaacgccttttgaatgatgattatattactttgttcagcccagatgttgcaaatggcgaattgtacacttcttatttcggcgacccagaaaaattcaaacaggtatatgagtctctagaaaagaacccaacaattcgtaagaaacgcatcaaggctcttgaactatgggaactgttctttaccgaacgtgcaggtacagcaagaatttatccgttctttaacgataacgttaataaacacggcccatttattcctgagattgctcctgttaagcagagcaacctttgttgtgaaattgcattgcctactaaggacgttggtggcgatgacccagagatcgccttatgtaccctggccgcgtatgtattagataactttgactaccaggaccaggacgaagttaatactctggctgaaatcatggttcgtgcacttgacaatcttttagattaccagaactatccggttcagcaggccttgaaagctaaaaagcgccgtgcattaggtgttggcgtaactaactatgccgggttccttgcttctaactttgctacttatgatgatgcgaacgatttaactcatgagctatttgaacgtcttcagttcggacttattacagcatcagttaagctggccaaagaaaaaggtcgctgtgattatttcgaagaaactcgctggtcccgtggtgaactgccaattgactggtacaacaaacgcgttgactctgttgcagccccgaactatgtttgtgactgggcgtcgttgcgggaagacctggttaagtacggcatccgtaacagtaccctttctgcacttatgccatgcgaatcatccagtcaggtatctaactctaccaacggtattgaaccaccgcgtggtccagtaagtgttaaagaatctaaagaaggttcctttaatcaggtcgtgcctaatattgaacacaacatggacttgtatgattatctatggcgtatgaccaaacgcggcatgaaagggtacttgacccaagctgctattatgcagaaatttacatgtcagagtatttccacaaactttagctatgacccggctgtattccctaaaggcaaggttccgatgtctgttatgcttaaagatatgctttacttctggtctttaggtggtaagactgcttattaccataatacccgagatggctctggtaccgacgactacgaaattgaacaaccttcagcagaagaatgttcgtcctgtaaactttaatataatatggggaccttggtccccatttaaggaaataaatgaaaacgtttaaagagtttgttacagaatctgcccaaatcccagggtatgtagttgcaggacaaggtgtacaacttagctataactttgattctaaaaacaaatctgaggttgataaattcatcgctaagatcgttactgaaatgggtaaaaaggtagctgttggtaagaaaggtcgtggtatcaatgttgtagaagttggtgaacaatacgataagaaaggcaagagcctagatactggcatctattgggctttcaaaattaccggacatgatgaagacggtacacttactgcactggtcaaaacctccggcgtatctcgggccgagaaaaaatttgatgttggtaccaagtattctgtaaaagacggtaaggtgctataatctaattaagttacaatcaggctcatggaagagccaattgaggaaatataatgagtacagtttttaataccaaacaagtagatatcatgaccgaacctatgttccttggttcaggactaggcattgcacgttatgaccaacaacgccataaagtttttgaagaccttattgaaaagcagctgtcattcttttggcgtccagaagaagtcaacttaatgatggaccgtggacaatttgaaaagctgccagagcaccaacagaacctgttcacagataacctgaagtatcaaagtcttctcgattcgattcaaggccgtgcgccttcagcagtatttgccccgttgatttctgatccgagccttgatacttggttccagacctggacgttcagtgaaacaattcactctcgttcttatacgcatattatgcgtaacctgttcactgatccggctaaaatctttgacgaaatcgttcttgatgaagccatcatgaaacgtgctgaatcaatcggtgtttactatgatgcggttactgaagcccatcgtcggtaccagaatgcccaagaacgttgctttaatccagataatattgagattaaacaagccaagcatgacctgatgaaggcaatgtacctttgcatgcatgtagtgaacgctcttgaagctattcgcttctatgtttcttttgcttgtactttcaacttccataagaacatggaaatcatggaaggcaacgctaagattatgaagttcatcgctcgtgatgaacagcttcacctgaaaggaacccagtatcagattcgtcaatggcaacttggtactgatggcgatgaatgggtccaagtagcaaaagaatgcgaagaagaagcagttcgtatctttatggaagttaataagcaggaaaaagactgggccatccatcttttccgagctggtggtcttccaggacttaatgttgaaatcttacataagttcattgattaccttactgtttctcgtatgaaggctgtcggtctgccttgctctatcgttcttgaaagtactcaccatcctattccgtggattcgcgaatacttaaatagtgatacagtacaagctgccccgcaagaagtagaaatttcttcttatcttgtagcccaaatcgataatgatgtggattctgacgtactggccggaattcgcaaatctttataaggataggggcttcggcccctttgttatatgaaagcaatcgcagattaatttagcttcattaagtacgttagtctagaacttgacgaatcggactgtactataaagcaattggagataccaaacaaatttaatgtagtatacgcgatatcggttgatgacgagctagtctacatcggtaagactaagaatcttcgcaaacgtattaactactaccggacatcaatcaacagacaagaccagaattcagattcagtcaaatctgctaaaattttagatgcattaatgtctggtaagaaggttgagttctatgcacggcaatgtttcaacctactcattaacaatgaacttggtgagatgtcgatttcgactatggaccttgaagaacctatgcttatcaagaagttcaatccaccatggaacacccaacacaggataaagaaatgatcgaattatacgacaatctcatggcagtctgcaataagactgaagcttccaagttcttttataaagacgttacaggacggtacgggaccaagttccgtatcttcagctatcactatgcctcgtacagtgattggctcttagacggcgctctggaagcccgtggcattatgtttgagatggacgacaatggccccgtgcgaattgcctcgcgccctatggaaaagttctttaacttgaacgaaaatccattgacaatgaacttagacctttcaaaaatccaatacgttatggataaggccgacggttctttgatttcgacttattgcgaaggcgataaagttcttgtgaaatctaaaacgtctactatttctgagcaggcttttgctgcagaacgtctgttgtaccaagataagtaccttcctctatatcttaaaatggaagagttggcgcctaaaggctatacctttaatttagaatacgcagccccaagcaaccgtattgttctgtactatgatgaacctactattttcctgctgaacgtgcgtcataacgataccggcgagtatgttccatatgaggacctttatgctgatgctgttctgcgccagtatctgattccttctcatgttgtcgatgacggtgaagcctgggtcaaagaaacccgcaaagctgaaggcattgaaggctatattgctgttcttgaaagtggccaacgattcaagctcaaaactgattggtactgcttgctccatcataccaaggactcgattacttctaatgaacgtctgtatgaagcaatcgtaggtggtgcatctgacgatcttcgtgcgatgttcgatggtgatacttatgctgtttctaaaattgcggcgtttgaaagcgaatacatccagtatttgtctagtgctctactaacagtccagggctactacaacgctatgcgcggacgagaccgtaaagactacgctatcaaaggccaagaaacagctatcaaaaatggtaagcctgaactattcaatcttatgatgcagatgtatgcaggcactatgtcctctgaagatatggttaaaggcattgaaaaggcttttctgaaagtctggtctaaatacgtgccggccctttatagtaaagaaatccaaatgatcgaagagtagtttacaaccttgattgctcgtgttatgatatccatacacaaacaaacgaggaaccaaaatgaacctacaaaacatttctaatgatgacgttgttgctcactttgaaggtacttctcatgacggaatctacgtctataaaggtcaaaagaagctcggattcatctcagacctgcgcgttaccatgggtcgaaaactggctgctaagtttaagcaaaaacagtattcaaccaagcaagtcgaagagcgccgtgaagcaatgccggcagctgtagaagaaatgcgttcatttcttgaaaatcagatgactaagtatggcgctgaagtgaaaatcaatatcacacagcctaatgttcatctgaatggtcataaatgctacattatttgtgaccctctttctgacaagtttaatcgcttaggtattaagcatttgtataaaactggtgaagatatggcctttgaactcgacggctataaaactcagttagacaaagctcctaagcatattctcgtcaacaacttgtctcgtgatgagattgttgaggtaatcaaaaagttatgtctaaactaaatatttacattatagtcggtttactcattgccggtctatttggtaccacaaagtatctaagttatcgtgtagatactctaaaggaacaagtggcccagatagacggtgtagcaaaagctcagcagctgcagattgatcttattgctacacagttgcaaggtatccaagctcttacagaaaagaacaaggccgctatcgattctcgtaatgcaaaagataaaaccttgaccgctaataaaaagcgtgaagctgaattagtagaccctaaagcagtaaaagaaatagtaaacgatccgtttaatgagttcgctagagagctccaggaagcgaccagatgaaaagaatgataccagctgtatgtttatctcttgctctggtaggatgttctcaggcgcttcctgaagtgaaacctgcgccaagagtagatgctacttggcctgacccgattaaagaatatcaggcagaatggcaagtgcatgaaatcgacggacagaaatgggtcggcatgccgttcaccgagttccaggatttccgtatctggactaacgacattcttcggtatgtacagcagtctaataacgtgatatgttattatcgtgctccgttaaaagagccgaaatgtcagtgaggcttaggcctcactaggatgataaaatttttgaggaatttattatgactcttcgttctaactctttcgttgcagaaccatcctatctgtctaacgtaaaagtcacttctcttcgcaaaatcatgatgattggttcttgcgctcttacaccccttgaaaagaaagcaaccttcagttacgaatggaaaaatatcgacggcattgattatgtcactaaagtgatgtattatgccccaggtcaagattacgtgtctcgtgtatatgatctgaatccgcagacatacattcgcttatccaagtggtatgctatgatgcatccagtgtttgactaccctcagttctatgaagatgccgtagaacgcaaacgtgtccaggatgtagttaaatgctttgaagaagccgctattcagcacggtcagcgtaaagcagcagtggcgaatggctttatttctgatgtagaatcttatcttcgtggatccggtaaaattttgaaagaagcccgcgaagctctgtatgaggaactcttttaatgactaaagaacaacttgaagaactgcgcggccttatttccgaagttgaaatgaaatgtctgatctatggacgcgcctgtgaacgtcatgatttgaatgatgaatattcacggtttccggctttaaagaaataccgagaagataaacaccaagcctggggtgaagcttctactaaattgaataactttttaaacgggttagtaaaatgacagagaatatgcgccagttcttacttgctcttaacaagtatgataaattatggcttacgctgtgtgacctggtagagaaaggctgttctcaagaatgtgagaagagacatttatctcttattgatgcacaaaagactgttattaaaaatatgtttagagaattagaacaatgaaaactattattttgactatcggctgcccaggttcaggtaaaagcacttgggctcgccagtacattgagaagaacccaggttatttcaatattaaccgtgatgactaccgtgcatccattatggctcttgaagcccgtaatgaatacaagtactcaaagaagcgcgaaggcattgtaacttcgatgcagctggaaaccgcccgtactatcttgtacggtggcgattctgttaaaggcgtgatcatctctgatacgaacttgaatcctactacgcgtcaacgctgggttgaatttgccgccgaatataactggactattaagtaccaggtgttcgatgtaccgtggctcgaactgctgaagcgtaaccagtaccgtggcgaccgtacagttcctattgatgttctgcgtcagatgttctggaaattctgtgagtatcagaagaagcctgtttacaatggtacgcctggtaagccaaaggctgtaatttttgacgttgatggcacattagctaaaatggtaggtcgttctccttatgacctggacaagtgtgatacagacgttatcaacccgatggttgtagaactatctcagatgtacgctaaacaaggctaccatattatcgttgtaagtggacgtgaatctggtactattgaagatcccatgaagtattactatatgactcgtagttgggttgaagacatgggtattcctgtagtaatgcactgccagcgcgaacaaggcgattcaagaagtgatatggttgttaaagaagaaatcttctggaacaaaatcgctcctcatttcgatgtgaagctggctgttgatgaccgcaaccaagttgttgagatgtggcgacgtatcggtgttgaatgctggcaagttaactttggtgacttctaatgaaagcaagtgatgcagtaaggctctctgtggcctttaccgaacgaaatactcttatccctaacgatgtttttgagcatattgcgcgtcgagctattaacggggccttctcggctagtttctgcccttttgccgattttggtatcataagtcgtcgttcagaatacgaagaccagcactataaaatggctgacatcattcgccagtttgaagaactcgggtacgaagtagacgttttctatcaaggcggtaatcgctacggtcctatcgattcaatccgagttaagtggtagtttacaacggctggaagtcatgatatgatgtacttccggtctacttgaggaaaatactatgtacgataaacatcatgaagttgaagaagaagcttacaaaatgcttcggaaatgtgttggaatgaacatgaatccagctttgattaatgaactggcgcgtattcgtaccgacctgaacactcgttacaaagacgagtaccatgttgaatttttacctctttctgatgttaatacgcatttcgtcgttaatgttaaagtacacaccgttcattaaggaaaagctatgttccctaaatattctgaagtagttaaagtagttgcattgcaggttgttggcaattactgcgctgataaccttgacaatgctctgaaggctaaaatgcatgcagatttcacaagtgctattaatacgcttattccaaatcatggtaaagtatctctcgaagtaaatagcattggcgttcaggcagttgtcgaaattgtctttgaaggcggtggtggattcgctgttgaagtaacaggcattttcagtacccaagaggttaatgtttatgttcagtaataaaacatttactcttgaagagtttgccgtagttgttcctcagtttgctcaagctttgattaataaagtatctgaatccagcccagatgcaaagctgagcgtaagacaagaaactcctgtgtcatatttgattactattgaagcccgaaagaaagagtattatctgcttgagcttctgactaatgggcacgttcaatccacctcagtgtatccgttctaagggcttcggcccttattggagaataatgtaaataaggaacaatatgagacctttagtcgaaagaatgataagcccggctgaacttgagcaccgttgccagatttcgccagagctgcaaaatcaagtcagtttacatcgccaagaaatcgataagattatgtctggtaatgataaacgtcggctggttgtagtaggaccttgttcaatccatgatgtagttgctgccgttgattacggtgagcgcttggctgaactccaaaaagagcttcctaatcttctgctttgtatgcgcgtttactttgaaaaaccacgtacaacggtcggttggaaaggtcttatcaatgacccgttcttagatggttcatttgaaattaaccacggactaggtattgctcgcacgttatgtcgcaagcttttgcgaatggggctgcctctggcaacagaagttcttgatccgttcactatcaaatatctgtctggtattttctcttgggtcgctatcggcgctcgtactacagaatctcagactcatcgcgaaatcgcctcaggcctgccaatgactgtaggctttaagaatggaacaaatggctctattaaggttgctactgacgctatgcaatctgcagcgttcccacaccattatatgggaatggaccaaaatggcgtagtcggtattgtctcagctccaggtaacaagagcactcatattgttctgcgcggcggttccgaaggaactaactacgataaagaatcagtctttgatgctatcacccaggcacagaaggcaggcgttaaccaatacttgatggttgattgctctcatgctaacgcccaaggttctttcttgaaccaggttgcagtttgtgctgaagcagcagaattggctaacgttcgaggaattatgttagaatctaaccttaaacaaggtaatcaaaagatttctacagaccttgaatatggtgtatcaattactgatgcgtgtattgactgggaaaccactgaaatggttctccgtaatctgaataacaaaggctaaaaatgaaagctagcacttacttacaaatcgcgtatctgatttcccaagaatcaaagtgttgctcttggaaagtaggtgcggttatcgaaaaagatggtcgcatcatttctacagggtataacggttctccggccggtggaacaaactgctgcgaccatgctgcacagcaaggttggactaagtttgttcctaatgctggatccggccgaatttcaggtaaaggcgggcttgttgttctggctaaagaacatcgcccggcccatagtgagtggtcttctaaaaatgaaatccacgccgagttgaacgctattttgttcgccgctcgtaaaggttcttcgattgaaggatccactctgtattgcactgcatctccttgtcctgagtgtaccaaagctatttcgcagtcaggtattaaaaaggttgtttatgcagagcgctacgatcgctctcctgatgactgggccgatattctagttgaagctggaattgaagttattgaatataaccgtaagaatcttaactctctgaactgggaacagattcgtaatttttgtggtgaataatgaatcttaatgaattcgattctgtcgtaaacattaatccgccgatgccggagcctgtagatgcaactgaagaatttgcatttatgctcaaagctgccggtattaatgttattgttatcggcgctccttcgttttctgtacaaactataaaacgtaacgagcgtttcaaagttgatattgcatctttcagagctcaagacctagctcatctagtttctgaaatcgtgaaatttaaaccggatgtggtttatctacgtgaagttaaagacttcacagaaattgacggtaaacctgcattcactgttcgctttaactttattaaggtagaagtatgaaattgactactgaacaaaaaattgctttacgcgaagtcctgaaagtcaaattatccatgggcgtatctgctatcgttttcgaaaaagcagacggcactatccgtactatgaaaggtactcgtgataaagatgtcgtttcttctctggtaggcgctgagatttacgaaagttatgtaaatcctgctaagcctcgtaaagaatctacggatatggttccactgtttgataccgagttgaatcaatggcgtggctttagcatcgacaaactgatctccgttaatggcatgaaagcagaacatctgctacagttcgttggctaatctgttgtatgctttaaaaatgttttgatattattattctatcctaacaacaaacgagataactatgtctgatttaccaattcgtgctcttggtgagtatgttattttagtgtctgagcctgctcaggctggcgatccagaagtttctgctggtggtattatcattgaacagcgacgagctcaaggtgaagttcctgaactgtgcgaaatctattctgttggtgaagacgttcctaaaggtttcgttgaagttggcgatttgacacctactcctttaggtaatatcaaacacgtaactcatcctttggttgctgctggtatagctaaagctaaagatattaaacagaaattcgtcactgtacactataaaaatttgacttgtgtttacaagtgatataaataaaaatatgaagagatgactcttctatgtggtggagatgcgttaagccaaatgggtcttaaggctccaccttttattcacctcacttgaggttctataatgaaagatactatcaaatttgtacttgacatccagcgtcaatcctggaataacggccatgacaactatggcgcaaccatcgacgtcgaagcagcaatgcttgatctgcttggttacttcaaacatctgaatccggcccagaaagctcttgaagctgaactggcggctaaagatgaactgaaatatgctaagccgctgtgtgctaaggctcataaagctgttcgccactttgtggtaacgctgaagtaatttgtacgcccaccatgtgtacgcatgggtaacgtatgatgtggacgttgttggttatccccacgtaaaacatccgaaaccatcggtgatttgctaccaaagtgcagctagaaaataaacggcaagtgcatgctacctcgaggcgatggctaatcgggagtacgccttatggcctatacatccatcggtgtatatcttatccctgagaaatcagacccggaacctttaagctaacggtgtgcaacagataagagtttaaacgtaccccttgagggcttgcgggagctacaaccgaaagaactgtcgaaagaagttgaacctcggaagaacgtgctcccatgtatttctccaaaatggaagactttataatggctaaacaagcaaaagcaaaaactgcagtaaaagaagttgttggtacctctaaacgcgctggctacaagcgtgcgtcgaacaagcgtatcaatcagacggttgagaaaatcatgcgtcgagctcgtgcggttcttcgcgatgatgcttctcgttttggtaagccgaaagcataagttatgggactcctacgggagtcccttttttgtttagaagcggtgtttacaacatctttagggtgtggtatagtactcttacaccaacaaaatggagaataaaatgaaaatcaatctgaatgctactgttaagtgcaaagaccacgacggttataaagctcaggtagttcacgaagcccagtggatgcttactaaagagcaattcgaactggtttactgcatgactcctgaaggcccttcagacgatttctcatggaaaattcgtttgactaacttcttcacaggtgaagagtatgttctgaaaactatcatcgtcggtaaaatcgtttccgagatttacgaagatgaagaagacggctactcagaagatgtaacgtactacaaaaacggccgccaagtggctgatgatctgatcgaaaaaatgaaagctaaaggtgttattgatttaactcattggacctttatcggttaattttaatggggctacggccccttaattgaggaaatagaaatgcgtaattatgaactagacgtagcttacaaactttctgatgaagcagtcaagcgtttctcctaaagtgtccataaagggttgaattactgcctggcccgtgaaattggtaatcgtccttggtttccagtttctcttgattcaagtggttgtgttcaagctattgaaatggccgacaccgggcgagtcattgcccccaaagaccttaactgcgctgatatcaaaggtcgcgagcaatcatatgtgatgttcacttgtggtgagcttgatgtcggtaccgttgagcggcacattgaacgtaaaccggtccaggtagaagttgagccactgtatctagttttagaacgttctaaaaactttgaacgggcttctattgcttatcgcggcaaacagtgtgctttcactcttgaagaagcaaaacaaatggttgcagaactctgtctgattgctccggtcgaaaccacatacgagctgtacaaattccatggcactgcaaagcctgttgttaccaccgtaatcgaagttaaataaggaactaaaatggaactctataaattcgcatcagaagacgctcgccagaaatttgaactcagcagctgggaaatagacaatgcactggctcaactgttaaaaggtaaagtattcagcctcgtcgaaaagaaacctaatggtactgtcataggtatcaaagttgaaggcgaacactataacgctaaacgccttcgtaaagaatacccgggcctaatgatcgatgattatgcggaagctctgttcactggtgacgaaatcggtacactggttgtcaaggttcctgaaccggtagaaaagttctgtgtaatgactgctgatggaaatagtgggttcttgattcgtgctcatgatatgactctggaacgggcagaagaagttgcatctctctacgtacgcgataacgaagactcacaggcaatagttatcaaaaagatttcatcatttactgcaaagaaagaaatcatagtagataaagttcaatactaagtgtttacattggtgtaggagtatggtatagtactcttacaccaacaaatgaggataaaataatgaacttcaccaacttcgaacgcaaatatgttcacgatttcgatcaatcgcctatctgcctgtggaaacataataatggcgttgtagctcagatcgatatgtattgggaagataattatgttttcatcagctttgagaacggtcctactctagatattaagctggaaggttccatgattaaagtcggtttccatgatgatgtgcgggttcgcaactttgctacccacccatcatggcacggtgataatcgcaaaacattggttaagctttatctgcgccatgttctcggtttaagaaccactgaagaacagcgtgaagcaatctgggatgtcgtttcaaacgaattcacaatttaattgaatggggctacggccccatctttgaggaaatcactatgagcgttactggtaaaacatttaaactgactgacgagaagttcaaagaatacctaggtcgctatagtactatagctgaagtttttgttgaatgtaatcctactaatttaccattcaaggttctggcctgtgatgaaggcggcaatattctagctattgagatcccaggtggtaaagtccttcgtgctgatgaagacgagctttccgagtactggtgtatcttcttagatactgcgtataacagagatatgccgggtttccacttggttgaggccgatcctgtggtggctccttctgaagaagaaaccggttattatgttcttaccgttaacaaagactccaaagctattttaggacctttgacttctaaagatgcatacgagttcgctgaacggcaagctcttaacgccgtccctgaagtagttgttcaggttcttaaaatcgaagctactgcatctgtagaggtgaaattcaaatgatcgtatcaagcttctggaacgaaaaacgtaaaggcatcgaaactatcatcagcgataccttcgatggcaaagtacctactgttgatgccgtccgtaagtacatcgacaagtactgtcctgtagagtttcgtcttatcaacggcgttgactctttgcaggtcaattatattaatgcacgtctggaaaaattagttacagaccgtcctgtaggttattctgacgataacggtgataacgccgttatgcctaaagaacctaaattcaaaaccaagttctgaggaaaatatcatgcgtgtttctgttcctaaagcccaagctcctcgtctggctcgttgcctgttgaataacgaaatgatcgaagttctcagctgtaaggttgaaagtaacaaagtagtttatcacgtcgatgccccagagttctttgagttccctaactgggttacagtacaatgattccggtatctaatgggtttaatcatggtgaaatccagctaattatggctggattacctaagtcagggttccatctaaaaatcctgaaaaacggctcgaacaaggagattacattgttatgaccaacttcgagtcatcaatagaccttgaaaaattaaaggaccgctatgcgctttagaagacacgatactgtctcattaggcatctctggtatcccaggtgttgttatctattgtttagaagcaattccacaataccgtatcgaaccatggtataatgtgaactgggttgatggtaatacttcactgcacgctgaaagcgagctttttaaaatatctaaactaagagatttaactaatgaagaaggttattcttactgatatcgatggtgtggttattcagtggcaatcaggcctggcttattttgcacacaaatacggtatcaaaactgacgtcattattgagatgatttcagacgaaaaattccggtgcccttcagatattttccaaactgatgttgctaccggaaagcagcttatggaaaagtacaataactctgattttattcgatatctggccgcatataaagatgctctgaaggttatcaatgatctcaagaaagactacgaatttgtagctataactgcactaggcaacagtattgatgctaagctgaatagacagttcaatctgaattctttgttccctggcgcattccaggagatcctgatgtgcggccattcagagtctaaggacgcccttctttctcaggctaaagaaaagtatggatcccggattgtttgttatatcgatgatctgccttctcactgtgaagctgccctgaaggtattcaaaggaacagaggttgatgtgttctttatgcctcgtggtgaaagaactgagaaaaccggcgcttattttgttaaagactgggctgaagttcagtatatgatttctaagcgttctccgtgtgttgggaacctggctactcttcataaaatagcaggtactcctgggacccgtaaagatcatgaagatgccatttgggaagtaatccgtaaggcccaagaagctaataaaaataagccttgggcccaaacacctttacgcccgtttgtcccatttactccagagccgtacatcaattgcagagacaaatggaccattaaaggcccgtctgctttaagagcatccttctgaggtagctatgttcgtagttcatactcttatagatggaaaaaatactacacgtgacttcggtcacgttaacatgttcttccgtgaaaaccctctgttcagacaagctaaagatgaagaaatctttaaacaatgtgttgagcaagggttcatttatgtaagcgaatatctctgtaatacatttaatggccggcgggctatgattacataccataagaagctagatttgcttctggaagaagtagcttataataagaccatttataacagaaggcgaattaaatgattttagatatccttaacgaactggccgccacagattcaaccaacgaaaagaaagcgattatgaatcgtgaaaaggataacgaacttctgaagcgtgtattcagaatggcttattctcgtcgattgaactatggtattaagaaatggcctcaggctggagaagttactcaagcagccggaatcttaactttagaagattgcttggattttgcagaatttacattagctcctagaaaagtaacaggacacgctgctatagcagaactagcttcttatattgccgattcttcacctgaaactgtagaagtttttagaagggttcttatgcgggaccttgagtgcggtacaggtacaacaattgctaataaaacttggaaaaacatcgttcctgagtacccacagatgttggcttcatctttcgatgaagctaaaattaaaaagcatattaagtttccggcatacgctcaattaaaagccgacggggctcgttgctttgctgaggtacgagatgatggggttctgtttttctccagatctggcaataattatctaggtctagataaattagccgaacagctaatggaaatgactaaagaagcccgtgagaaacatcccggtggggtaatgattgatggtgagctagtttatcatgccccggccaatttcaaagagaaagattctcttgacttcctgttcggcgacgttgttgaagaacaagaagaaactgttgctgaccgcagcacttcaaatgggttagcaaacaaatctctgaaaggcacaattactcctgaagaagccgaaggtatgaaattccaggtctgggattacgttcctctggatgtaatttattctgaaggtaaactcaagagctttaagtatgatgtgcgctttcgtgctctagaacttatggtcaccgggtttagtcagattattctgattgagaatcactgggtcaataaccttgaagaagctcgcgcaatttatcaaaagtatcgtgaccaagacctggaaggtattattcttaagaatatgcacggtctatgggaagatactcgctctaagaaccaattcaagttcaaagcagtaattgacattgctatgcgaatttctggttattatgcgcactctaaagacccaaataagattggtggtgttaatttaatttcagaatgcggacgtattacttgtgactgcggttcaggtttcacagataccacacataagaaaattaatggtgaatgggttctgattcctattgaccagcgtggtgaacttgaccgtgaatctttgatgttagatgctatcaatggtaagttagttgatagaattgctgactgtgaatgtaacggttggactacctctaaaggtcgcaaagatgggactgttggactgttcttaccaattattaaagggttccgtttcgacaaagaaaaagctgatacattcgaagaagtgttcggtgattggccattctaattaaggagccttcgggctcctttttggacataaatagtagtatcttataaggggaataattatgtctgaacaacttaatgaagttttcgactctgatagtgataagaactatcccgtcgtgaacttgaatttgaaatctaaagtaccacagatctggtctattaaagcacctggcaatgaaaacctagtagctcgtatggtgtcatatcagtctgaaggtgatgctattaaacaagttaaaatgggtgacaaatacgctcatttgattttgatgtctcttagtgctaaaggaactccagccgaacttaaaggcggcttaggtagcaatccaattggtacaatcaatactatttttgatacggtctacgagcaagttaaaaagcttcgtatggatgcagtgatgttcaggttcccgactaagaaaatgaaaggtcaaggtcctgttctccagcgtctgattgcccgcctgtgtatggcacgtacaggtggtaaatttaaagtcctggatgcgctctataactttaccggcaagcacacatatattcttatctatcgtaagagcaaacaattagaggatatctctgggattcctgatatcaacactgagttatatactaaagttgagtcggcagttggtgatgtttgggtatctaagaaagatggtttccaagttactaaagacgaagctattgctgggtctattgcagatgtagaaaaaactcgtactgatattgcagtggttcgtcgtactaagatttctcgtcgtcaggtcgcagctagtcaatcaatggaaacagaacgctttgaaggtgaattgttccagaaatatgaagacagtgctgcagagtttagtcgtcctgctacagctgaactaattccagaatctactgaacttgaattagcggttacttctaaagctaagaaaaatcaagctgcttctatttctgccgcaggtattggtaatcgtctgtatgaacgaaaaattctaacaattgatcaagcagaaaaattagaagaacaattaaaagaatctattatacgtaagatgggcgatgctccgcttacttcagtcaaaagtatggaagcttatgttgctacagctattgacctagtagacgaattaaagcctaagtacgtggccagtattagtaaaaatatcagtgattacaacgacccagaagttaaagctgaattaatagagaaccaatggaaagtctttaaatctaaatttatcaaagattctcttaagggctgggctggtattactgcctcagatttatgggctattacccaaaatagaacgcctaagcaatatactagagctgaagttcgtggcattcgggaatacgtgggctctggttattctgatattaacgatatgcttttaggtcgttattctagcgatagaataaacatcctagatgaaaccgaagtaaaaactgctattgataatttggattctgcatttaaacgtggtgacagaattccagagaacattactttatggcgttcacaaagcatcagagctcctatctttgaagccttagttaaaaataaagttttctatttccgcaacttcgtttcgacttcattgtatccgattatttttggcggttggaaaggaaatgctgctattggtctggctccagaagaaactcgccaagagttgaatatcccaggcacttcagatgaaaacgtcgttattcctgacaataacgataatctaattgatgttaaggtaactgttggctgggctatagatggtgctcataaaattaacgtcgtttatccaggtgatttaagtcatcaatcccaagagatggaagtcattctacctcgtggtactatggttcagattaacaaggtcacgaatgcttcttataatgacggtattgactatagcaaccagaagttcatccaagctgaaatcatgacttctgaccagattgaagaatctatggttgtttatgatggtgatgttctggtagaatctggcgaactggtccctatgggtgcgggcactatcgttactgtagatggcgaggatacagatatcagtgcttctacgttcttgactactaaacggaatatccttagcatcttggcttcctgtatagatatcgaagatactccagagaaatttgtacaaggctagtttacaacaccacaaggacgtggtatagtctttattacacaaacaaatgaggaaatcaaaatgcattcattcatccgttttaatggtatcgaagcttctatcgatgacgttatccctgcttcagagcagttcaatgaagttgtagtaaaagagttgcagaaagtctatggcgataaactctttgctttcgccccgatgcagaactttaccaacccagaccaaacagatactatctttgctggtattatatctggccagcttgaatctgaagctccggtacaaattcaggttgctgatgaaactggcttaatcgccacttctaccgcgttttttggtttccgtaaataaacccctaagggagccgtaaggctcccttttttatgttaaaatcttactaagtccattcccaacgcctgcaacacggcttaatctacttgaaatactacctaatccttgagcacttagatttgggttaatagtatcaatcttttgaacagtcttatcttctaaccaatccatcgcagcttgtttaccaacagcaccgatctgtgcaactctgtaagcgaaggtgacttcgaatgtagctatctgattattatcttcatagctaaattggggtgcgctgacgcccacagggatgcatcctgtgtacatacacacagtgtgaggtaacccattacgggcatgcaagttaacttggatatccgcttcaacatcagaaggcagtgctcttagtcctgttacagggtcttcaacggcgtttacccaatcttgcatagcacggaaattactagcttctgaatccatacggaatgttataactaatggagcatattctcgcccggtgattttgatgttaggactgttatggaaggtgtccatttcatgagctaatctattttcaggaatctgaaccgaatggattagtaatcctgtagtagggaaagccatattaaagaagtcaagcagataagtccctacagtgaattcccctaacaatgattggatagtgcgttgagtcattgctcctatcaaaaatttactcacgcctgattttctaaccaaacgctgggtcccagcagtaataagagatgtaacgccacgtgtaaattcaccttgggttaatccgagccagttatcattaagacccacgttattataaagttccccaccaaattcatttagtagggtctgagttttactcgatggcgctgtggcaaaaacaacagagaacatattagttctctggaaatcgctattagccgcctgtgtcacgaattcatctaaagtatacattatgattggaatcctttagaatacagagaactacggttcaaagtcaagatttctctcatggttaattcaagggtaaaagtgctaggcaagttaggagcaatagccagaccattgaaatgtccgttaggtgttttgtcaaacctaagactttggatttggcaaggaccaaatatatcttctcttccgtcataagaactagtgtatccgaagttacgaaccgtccaaatagttgggttgcttacaacaattacattagtaaggaaagaagtaatagactcaaacattgtcccgcttttagaggacccttctggggtggcatactctaaagtgcttttataccattcatctatttgttctttaacttctttagcaaagtctgatgttcctgtggcaccataagaataatagttaaagatctcatagattttaacaatttgaataaggtcttgaggagttcttggggtaagatcccaagtaaaaaccttagttctattttctgcgccggcatacatactgcgggcagtattatagatttgttcccctttatctgccatcactccttgggtgagagattccaacccgccaaatactgcggtagaagccacattacttaaaacgccggtagcagagccgccacctcgtgtgatcagggattcgcctacgtcattaaatttgtgagatacagaatctacatcggacttagacttaggcaacaggatgttagctacaggggctttagaaagtctctgggagccggtgttagttttttccggagcatcaaaaacagatgatttactaacagcacttcggaaatctcgtaaatctggagaaaccctagatttaaagtcgtatgcagtaaacaaaagaccgttctgatacaaatcatgaactctcatatcttgacttgaatcgttcccggcagaacgttctgccgggtattgagctgtcacagtactggcagcattttttaaagtgtggttagacgtaccttgagaaatcttagaaccggatttcttaatattctctagcccatcttttaattcttcgaaaaacataaagaatccttaattagattttgtaaatccacccataccaggagctggagaactagtaaccggctgcataggcataacagtagtattgtttttgttaatagatgtattaacattcataggagcttgagaagccagtttatcgccaactttatgctgagcttcgattttctcaataactttaacttgctgagattcttgcttatcttctgccggctctggactaacagtagggaacctctttttattccactcgtcttgcataaagcggtactgaggttctagttccttagctagactaggattattcttctggaactcttcgaagttcttagaagccttactaacagcattagcagcagactccatacgctcggtattatcagcccgcgtattctgagcatattcagaagttcgttttaattcagcctgcattttgccttgggctttatacatttctaaacgctgctcccgggttttaccttcaaaagcagggtctttagtatcagaatcagctaatttcatacgtttttcgtattcagcatcatcaatcttaccaagtaaatatctatcgccccaagcagtaattttacgttcagttggagaatcttgctcttctttcaagcgctgattatctttatatttagctacgtaatcttggtcttcttcagaaagctcggcaccagtacgatactggaaacgatctgcggcagaaccgcgaatattatcagcggcatcgcccataccaggaactttactaagaacagcagcaaccaatttagaaagaccaaacataattgtttcgcctaaagtatctaaaacattaaccaggcctttaacaatagcttcagctaatccagtccaagagccttcattaaaggtatcttgaatactttgggccatttcactaatcgatgttaatataggagcccaggttccagctaatttagtgaactcttcaaaatttgatttgaataattcggtccagtatttgaaatggattttgataatatcaatagccataatcaaagctaataccatagcagctgttttagccatattagcaacagcggtcacggtataactaaagagcataccagaaattctgtcagttagtgaaattgatttcttaaaccccgttttagtagtatctaaaagagctttaagaaggtccgtgtcggtcttcttgttcttttcatcatcgtctttattttctgggattactctaggttcatcctgcggtaatacatcagaatccggacgtctattttcatgcggtggaataaggtcttgaagaagatcgtctaatggcggaatctctggaactgggtctgggatagaatcctgcactacctcaacagatggaacatttgctgtttgtggcgtggcttgttctacttttactcccagcttttcagccaatttagtaattctgtcacttattaaagaggctgtgctattaagagcacttaattcatccagctggagttcaatacctgcattagtatcagataaagaatttcctacattagcaagaccgttctctgttgcttcggcccccatttcaacagcagcttgaactccttcaagagttccattaatagtgcctagtgaggtgttttgttcttcttgtagtgcttcattgcgtttttgtggtgctgcgtcggcgattacccgacgcagagattgtgttttaccatttacttttctagccattattcaaagagccctattatattgccaattccttttataggcccgttaggaccaggaatagctacagtgttaacgataccgtcaacccatttaattacgaatgctggaagcttcataaaatcaggcttctctgttctgccatcgacagaagaaaaatacttgtcaagaattgcgtcagccgagccaaatttactgaacctttcaggagcgttaaatttatattcttggccatttaaacgatgggttaatatacggcatccgtacacatccgataaacgataagtgaatccatccttttcaacttcttctttaatcttaccgttaaaggccaaaagttgtaatagaacaatatcgctttgtacagcatccagacctggtttaatagaatctaaaagaattgccagattttcatccggcatttttacttctttcattaaattgtggtgcttaagtccaagcttaggtaatgaaatttcaataccatcaattgtaattttcttaataggcagaatcacgtttaagttcattttttaccttcgctatttgaactggttcaatagtagaaccgttagtaaacatatagatgttggtgattgaatggttattactgacttcgtggataacttcgtcaacgtagaaatcagtacggaattgttctttaggatcgtagaagtttaacttcattccaggagtcacttcaaagttaccatatattttagcagacgcgtagccatcgtactgagacatggtaacaatacggttagcttcttcataaccgttacgataaatcatttccgaataaccaccacttctattaatatagacactattagaaccttcgcctacggttatttttgtgatttctttatcgacaaaactttgagcgtagaatgtagcatccttaaacggatttcgagtatatgtattagcttttacaagccattcaaagtcccaagctaaagataccggcaattggtctacgaattgtccaatagtctgaggcgcaccaacaacaaaagatgcggcgttctgagcaatcatcgcgttgtagtccatcatgtttataccggttatatcttcccaagcaaaaacaaattggtcactttctacagacagccctacatcacgaatatattgcatgaattcctttatattagaagtccaaggaattctaggaacataagtgtttattccgtttattggtggtgccaacttagggtatttttgataaatcacacctatcatttcaaccaatgtttcgtaggcagaactaaagaagcaacggctgaacttcaaattcattatatcatggagcgaactaagctggatagctataatattgtcgcccttatcatcgacacggaccgtgaagtgttttatcccataaattcgtgtctgggttcttgatgtattgctattagaaacggagatctggataatttggtctccattcattttgttgtgaatgtttttagaatcatagaactggagcataccttcattaataccggacaaactatctctcatggtcaaggtagtaaatgtggcaccaagttcaatgaaactgttatcaagccatttatcatagttttgataaagcttaatactgatatttggaaatccagggcgttgtgaaatcatttttgtgctagttccttttcaatcagagataacgtgatgcttctctctataggaatcatattcataacggaagaaagatcgtagccttgtttaactatcaagtgattgattttataaaacgtaaatacttcatctgggttcagaagaagtttaaagatatccataaaatttgtgtaatggatctcatgggaatcgcaacatgaatattttagtacaaagtcaaatatgttcaattttttgattataagttctaaggtttctatatcgatagcttcaacaacttgaactttgttttctgtagacaactcagaccattgatataccccattagaatcttctacagacaaaatgttttctgttattagaccagctatattttctttatagtttttagatggatgtttaaatttaagccgaagcccggatacatcaattacaggaagctcaacggcttcttggtggaaattaatcaacaccattttagcctttccacacttagggcattcaaccttaacaggaattttagtcttacctatgctggatagatagacttttaagaatacaaacgccctgtattcttttgggatgtcactgaagtagtcgttttggagttcttcaataagtttttcttgatcttctggagacttagtgttcatgtcgttccgtaccagaagaaaatctctatagtcagagactgtgaacggagagaatcgatgaacaccgtctggtaattcacatcttacgatattagccatagggagtcctcttattttattgtatttataaatacgtctagaggtaacatatgacaacttataattttgacctaactataaatggcaaaaatatttcttgccgtgcatttacactagaagaatatttgaatcttataaaggctaaagccgataaggttcttgacaaaacaattaagcagctgattaaagattgtactaacgctaaaggcttaactaaacatgagtctgaacttcttctagtcaatctatgggcccattcattaggtgaagttaaccatacagccacctgggtgtgcgactgtggtaatgaaatagacgtgcctattaataccaatcgtatccaaatcgtgggctctagcgatctcctgtattcactagacggacttcgtatccagttcaaatacccagagttatttgatgataatgatattgctttaatgatagctaaatctatcgattatatcattgtcaacggcgaacaaatctttgtcgatgatttatcagaccaagagatagacgatctttattctgctataactactgaagacgtcttgaaaataaaagatatgctgctgaagccacaaatacagttggcagttcctatttcttgcaagtgtgggaagaatcatgtgcaccaaatcaccggactcaaagagttctttaaggttatccaatgaatatagacaatatgtattcggatttagacccagaccttagaatggcctgggataaagacgttgcaagaactgtaggtgcaagagcagtaaaaaatagcatcttaggaattatcactacaagaaagggcagtcgcccttttaatccagattttggatgcagcctttcggacgagctgttcgaaaacatgaccccgttaactgctgatacaattcagcgtaatattgtgtcggctattcaaacatatgagccacgggtagaacgtctgcgtgttgaggtcttagcgttatatgatgataatgctgtagtcgttacagtgatgttttctatcgtcgacgacccggatgtcctagaacgcatcaaaatcaagttacgtgcttctcgttagtgatgtgtataatggcttaggtcctttaaattgagattggtgattaaatgaagttggaagatttacaagatgaattagacagagatttagttattgaccagacaaaacttcagtatgaagctgccaataatcctgttgtctacgggcggtgggtacgtaagcactctatatgtcgtaaagaaatgctgcgtattgaagccatgaaaaaacaagccctgaagaaaagacttgattactacacgggacgtgtagagcctggtgaagaagtgtgtatggattcgtatgagaagtctgaactaaaaactgtaatggctgcagatgaaaccatcttaggaactgaaacaaaacttcaatactggggtattttgcttgagttctgtagtgatgctatggatgctattaaagctcgcggtttcgcgataaaagcagttattgaacttcgtaaatttgaagctggcgaagcgtaataaataagtttattaacagaggagacaaccatgtcgtgtatttgtgtcgtctgtaagactccaatcgatgatgtattggttgttcaatcagacaaaggtccggttcatcccggacaatgttataatcatgcagttgagatgccagttactgagaatacagaacagcaacttaacgaagtgcagcttttaatctagtgtgtaaccttattggttatttttggctctccttacggagagccttttttgtatctagagattagaagttttcttcttcttcatcagaatcaatagattctaattcagtgcgtttaccggccaaagcatcacggatagtgatatcatcagaatctttcaaatcggcttcttttacacgctttttgtaatatttttccaattcgctaagaccatcaagggtctggcaagagtttactttagacataaaatcttcaatggcggcttcgtataaaaattgtttaaagtccataataattccttagagttcgacaagtttcatgacgtaatcgaatttctcatcagcataacgctggatacgttctaagccatgtttgagcaaatagttaatgtgggtgtaagtctttttggcattagcagatttaggtttaacaccgcagtcatcgatgatgtcccaaaccgttgctacatctttagagccatgcttacgaagtactcgaccgatagtctgaagtacgataatctttgatttaacaccgtgcgctaaaataacatggtgtaaattcttaatactgattccggtagagaatacaccgtatgatgcgacaacaacaattccagtttcgttttctgccatttttttaagagcagtacgggtatctgtatcaatttcgccggatacataaaacactttgtcgtggccaaggtctttaatcatctggaataattctttaccgtggctgatgttcttgaacatcacaaaggcgttttgatttttactagctaatttaacagccagatttgcaatccatttattacggcgtttaactgaagtgatatatttaatctcttcttggtaagttttacctttcaaagcaacagtaacgttatctggatatctaagtctgatagcattaattttaagctcagtaacagcgccttcttccatcaactgtgaagttgataccggtttaaaaatatcaccaaacagaccttgatactgcatcaaattagctttgccatctttcaaagaacctgaaaggccgaatttgaacatacagttatttagcccggcaataatagtagagatagatttacctgtggcaagatggcactcgtcattcatcataagaccaaactgactgaaccattctttaggctgtttaatagccgtctgccaagtactaacataaaccaccgcagaagaatctttcttagtaccacttctgataccgagcatattatctcttttgaacagacgataatcacagaagtcatcgatcatttggtcaacaagggcggtagtaggaacaattattagaattttgccttcatagttctctacgtaataacgactcaggagtgcctggatcaatgatttacctgctgatgtaggtaaattcaacagagcccttctttctttaagacccttaaacacggcttctttctgataccaatgaggtgtaatcttagtcagcccagaatagatttctttggaatcgagccacttatcaaaagattcttgagtgatttcttctttttcaaaaatcatagggtctgcgtagactttataacccatattgctggcgaatttctgtacctggttcaccagaccaaatggaagtaatctgtcataacctaatagacgaatgcgcccatcccactgcccgtaacgaaattttggattaaacttatagccttctgcttcgaaggagaaaaagtctcttagttcatagaagatgccttcatcgcattcaaccagaacgtgactaaaatcatgaaattttaatttgatgtccacagcgtgtagcctcagttataaatacatacatatttatacactagaaagagagacactatggacaagcaatatatagatgaccttcgtggtcttgaaaataaaaaagaagccaaggctaagcttatcgaatatgccgaaggatttaatatccaattgaaaaagagcaaaggctttgaaaacctagtcttagatatcgaagccgcttttaaaaagatggctgaagagcctatgccggaagataacgaaggtctgagtatcagtgaccttatcgccgccgatgatgaacttgaaggcaagaacgactttgtacttggtgacgttgaagctaagcctgaagctaaactgttatttgattctccagaaattcctgtatctattcacgaaatcaaagaaaccgttcaaaaacaatcagttgttgaagaaaaagctattcatattgaatcacctattggtaaattcgacacgcctcaacctaaactttcagaaaatacctttgaagaagctgtagctaaaattatcagtgacgaaactacttttgaattaccggaaaactttagccctcattttattctgatggggcgtaatcctggttatacgaccttaccttggtggatttacgagtggattaaagacaatccagactggaaatctagacctaccagtttcccgcaccctagcgcacaccagacactgtttagtttaatttattatatcaaaagaaacggatctattatgattcgtgaaacccgtaattcttcgtttgtaactttaaaataagggaccgactatggcttatagcgtatctattgctcctttggctgcttcagcagtcattggggccacaactaattttacagcaaccacctctggtgctacagttgaaggcacagaaacatttgagtggaccgtaaacggtgtaaaacaatcttctgtcaccgcagctatgaattatattgccgcaggacctgctggtagtaagaccgttaaagtagttgctactgttactccagctgaaggtgaccctgaaacggctgaagcagaaactactctgaccgttaataacaaaactatgccggccattactttaactttgagtcctacttctgtttctaaagaaattggccaatcccaggtagttacagctaacgttgctggtgctccagaaggtgcaagcattgcttatgtttggaagcgtggttctagtgttatttcaggccaaacaggtaaaacaattaccttatctgaatccgcggaaaccagctacacattgaattgtgaagtaacagtttctgctccagattataataatggaactgcaactaaaggtatcgctgtggcctttactaaaaagaccatgagcggtgttctgttattttgactcctgagtctattacgactgagcagggatccgaagcatcttttaaagcggatgttattgggccccagaaggcgcaaccggaacttattcatggactaaagacggttcttctatcgaaggttccacgagtactttggcggttgacacttctactatagggtcacaagtaattggagtttctgttgaggtttcagcagaagattacaatccggttacagttaccgcaactggcgatgtaaccattaccaaaagagtagcccctgagccggaaggagaacttccttatatccaccctctccctttccgaggttcagcttatatctggtgtggttggtgggtcatggatgaaattcagcgaatgaccgccgaaggcaaggactggaaacttgacgaccctgatagcgattactacttgcaccggtacactctggctaaaatgttagatgattaccctgaagttgatgtccaagaatctaggaacggcaggattgtccaccggtctgctcttgacgtcggaataatctacgactatatctactaaataatggggcttcggccccataggggcgtaattagctttaaaaatatgtgatttaataggcccatcatcactaagtggagctacccatgcaagcattaaagattcacatgatgcatgaaactggtactagttttattgacatcgctaagcagttcggtatcagtgctaaagaagccatgcaggactggataaaagttgaaaaggctagagagcgggccaaagcccgtgagcgtgtggtttatcgtaaaaggcttattacagaccatactaaattaattgaaaaaatgaggtgttataatgcataattctgacggtatcagatccagacgtatccaaggttgggatgaggcaggacaacctattggcggcctgaagccacacgttaacgagcagcgtgtttctacattagtagaacactatggcgctctagcggactctgtagctactaccgaactgaaaaaagctaaagatgaagttgaactggaatttaaagcacaagctctgaaaggcaaacgtcactttaattggtatccatcatctacagcaaagaaatataaagaccagctgattaaatggatgcaagatgatggtgtactagttaattggaagcacgaccaacgtgatggtgattgggtagaaattaccttctaatatgctttaaattttttcgtgatatatttggtataatggttttaagctaagagtatcggcggtagaagtctttactcttttatttatgtttgttctgatgtttaaaggtgaaatatgtttgagaaatattctagtcttgagaaccactacaacaacaagtttattgaaagaatccgtcaccacggatacgatgtaactgaaggttggtgtgcacgtgaaaagattcatggtaccaacttttctattatcgttgaacgt

(19) United States
(12) Patent Application Publication
(10) Pub. No.: US 2021/0187046 A1
(43) Pub. Date: Jun. 24, 2021

(54) COCKTAIL COMPOSITIONS COMPRISING RESPIRATORY ANTIBACTERIAL PHAGES AND METHODS OF USE THEREOF

(71) Applicants: TECHNOPHAGE, INVESTIGAÇÃO E DESENVOLVIMENTO EM BIOTECNOLOGIA, SA, Lisboa (PT); TECNIFAR - INDUSTRIA TECNICA FARMACEUTICA, S.A., Lisboa (PT)

(72) Inventors: Clara Isabel Rodrigues Leandro, Parede (PT); Ana Raquel Martins Barbosa, Sobreda (PT); Daniela Jones Dias, Lisboa (PT); Ana Filipa Martins, Lisboa (PT); Miguel Ângelo da Costa Garcia, Lisboa (PT); Sofia Volker Côrte-Real, Cruz Quebrada (PT)

(73) Assignees: TECHNOPHAGE, INVESTIGAÇÃO E DESENVOLVIMENTO EM BIOTECNOLOGIA, SA, Lisboa (PT); TECNIFAR - INDUSTRIA TECNICA FARMACEUTICA, S.A., Lisboa (PT)

(21) Appl. No.: 17/127,631

(22) Filed: Dec. 18, 2020

Related U.S. Application Data
(60) Provisional application No. 62/950,850, filed on Dec. 19, 2019.

(51) Int. Cl. A61K 35/76; A61K 9/00; C12Q 1/04; A61P 31/04

(52) U.S. Cl. CPC A61K 35/76; A61K 9/0078; G01N 2800/26; A61P 31/04; C12Q 1/04

(57) ABSTRACT

The present invention is directed to the field of phage therapy for the treatment and control of bacterial infections, in particular respiratory bacterial infections such as bacterial pneumonia. More specifically, the present invention is directed to novel bacteriophage strains and cocktails thereof, as well as variants thereof; and methods of using same in the treatment and prevention of bacterial infections, including respiratory infections caused by, e.g., Pseudomonas aeruginosa and/or Klebsiella pneumoniae. The cocktails are used as pharmaceutical compositions either alone or in further combination with other therapies, e.g., antibiotics or other standard and non-standard therapies for respiratory infections.

### 0. SEQUENCE LISTING

The instant application contains a Sequence Listing which has been submitted electronically in ASCII format and is hereby incorporated by reference in its entirety. Said ASCII copy, created on Nov. 19, 2020, is named 38019.0007U2_Sequence Listing and is 427,434 bytes in size.

### 1. FIELD OF THE INVENTION

The present invention is directed to the field of phage therapy for the treatment and control of bacterial infections, in particular respiratory bacterial infections such as bacterial pneumonia. One aspect of the present invention is directed to a novel bacteriophage strains cocktail including Kle_F391/08 (genome having a nucleotide sequence of SEQ ID NO: 1), Kle_F17/19 (genome having a nucleotide sequence of SEQ ID NO: 2), and Kle_F58/19 (genome having a nucleotide sequence of SEQ ID NO: 3) as well as variants thereof; and methods of using same in the treatment and prevention of bacterial infections, including respiratory infections caused by, e.g., Klebsiella pneumoniae. This cocktail may be administered in combination with other bacteriophage compositions, including cocktails, directed against the same species of pathogen or a different species of pathogen, for example, Pseudomonas aeruginosa. The cocktails are used as pharmaceutical compositions either alone or in further combination with other therapies, e.g., antibiotics or other standard and non-standard therapies for respiratory infections.

### 2. BACKGROUND

Bacteriophage (phage) are viruses that specifically infect and lyse bacteria. Phage therapy, a method of using whole phage viruses for the treatment of bacterial infectious diseases, was introduced in the 1920s by Felix d'Herelle. With the development of antibiotics in the 1940s, however, interest in phage-based therapeutics declined in the Western world. One of the most important factors that contributed to this decline was the lack of standardized testing and methods of production. The failure to develop industry wide standards for the testing of phage therapies interfered with the documentation of study results, leading to a perceived lack of efficacy, as well as problems of credibility, regarding the value of phage therapy. Another problem in phage production related to the purity grade of commercial preparations of phage, with preparations containing undesired bacterial components, e.g., endotoxins. Accordingly, adverse events were often associated with the preparations, particularly in patients receiving them intravenously.

Nevertheless, in Eastern Europe and the former Soviet Union, where access to antibiotics was limited, the development and use of phage therapy continued jointly with, or in place of, antibiotics. Further, with the rise of antibiotic resistant strains of many bacteria, interest in phage-based therapeutics has returned in the Western world. That is, even though novel classes of antibiotics may be developed, the prospect that bacteria will eventually develop resistance to the new drugs has intensified the search for non-chemotherapeutic means for controlling, preventing, and treating bacterial infections.

Phage therapy, and phage cocktails in particular, present an alternative to antibiotics for the treatment of bacterial infections, and in particular, to respiratory infections, including nosocomial pulmonary infections. Respiratory infections account for more than 4 million deaths annually. Hospital-acquired bacterial pneumonia (HABP) is an acute pulmonary infection and is one of the most frequent type of infections acquired in intensive care unit settings and is associated with increased mortality (ranging from 33 to 41%) (Guzman-Herrador B, et al., 2014, J Hosp Infect 86(1):53-56). Nosocomial pulmonary infections are typically caused by methicillin-resistant Staphylococcus aureus (MRSA), Gram-negative Enterobacteriaceae, such as Klebsiella pneumoniae, or Gram-negative non-Enterobacteriacea, such as Pseudomonas aeruginosa and Acinetobacter species (Quartin AA, et al., 2013, BMC Infect Dis 13:561-566; and Di Pasuale M, et al., 2014, Crit Care Med 42(2):303-312).

Antibiotherapy is routinely used in HABP, however the therapeutic options for the multi-resistant (MDR) bacteria, especially Gram-negative bacteria, are scarce. No new classes of drugs have been introduced recently, and the few options currently available include colistin, tigecycline, and fosfomycin. For severe nosocomial infections, there are very few antibiotic options (Orsi G B, et al., 2011, Expert Rev Anti Infect Ther 9(8):653-679).

Aerosolization of antibiotics can lead to higher antibiotic delivery to the lung parenchyma, compared with intravenous administration of the antibiotic (Luyt C E, et al., 2009, Crit Care 13(6):R200). However, to date, there are no clear clinical benefits of using aerosolized antibiotics, like colistin, in the treatment of lung infections, due to the side effects from direct antibiotic toxicity on airways and lung parenchyma. These include, for example, mucosa irritation, as well as side effects caused by systemic absorption of the antibiotics, such as renal toxicity of aminoglycosides and polymyxins (Luyt C E, et al., 2013, Expert Rev Anti Infect Ther 11(5):511-521; and Quon B S, et al., 2014, Ann Am Thorac Soc 11(3):425-434).

Different studies have attempted to treat bacterial lung infections using bacteriophages administered via different routes (Hoe S, et al., 2013, J Aerosol Med Pulm Drug Deliv 26:317-335; Morello E. et al., 2011, PLoS One 6(2): e16963; and Debarbieux L, et al., 2010, J Infect Dis 201(7):1096-1104). However, there is little published evidence of experimental studies with the aerosolized bacteriophages curing established infections (Ryan E M, et al., 2011, J Pharm Pharmacol 63:1253-1264), Previously published studies have not assessed the effects of the aerosolization of bacteriophages in established infections, mostly examining outcomes after only a few hours of infection (Wilson K R, et al., 200, Microbiology 153(Pt 4):968-979; and Alemayehu D. et al., 2012, MBio 3(2):e00029-12). Moreover, there are few phage cocktails with antimicrobial activity against different bacteria, possibly because of the difficulty in combining different specificities of phage while maintaining storage stability.

Thus there remains a need to develop novel phage products as therapeutic and/or prophylactic agents for use in vivo against pathogenic bacteria, in particular, pulmonary bacteria. There also is a need for better treatments, particularly aerosolized treatments, for respiratory infections. In particular, there is a need for bacteriophage cocktails capable of lysing bacteria responsible for nosocomial respiratory infections, including Pseudomonas aeruginosa and/or Klebsiella pneumonia bacteria. This application addresses these and other needs.

### 3. SUMMARY OF THE INVENTION

Provided are novel Klebsiella pneumonia bacteriophage and their use in the treatment of bacterial infections. Pharmaceutical compositions comprising a bacteriophage cocktail disclosed herein, or the combinations of three or more of the bacteriophage strains described herein, may be used in the treatment, management or prevention of a bacterial infection, particularly a Pseudomonas aeruginosa and/or Klebsiella pneumonia infection.

Also, provided are novel Klebsiella pneumonia bacteriophage cocktails and their use in the treatment of bacterial infections. Pharmaceutical compositions comprising a bacteriophage cocktail disclosed herein, or the combinations of three or more of the bacteriophages described herein, may be used in the treatment, management or prevention of a bacterial infection, particularly a Pseudomonas aeruginosa and/or Klebsiella pneumonia infection. Such pharmaceutical compositions may be particularly useful in the treatment, management or prevention of respiratory infections and the compositions may be formulated for pulmonary delivery.

One aspect of the invention relates to novel Klebsiella pneumonia bacteriophages. Provided are purified Klebsiella pneumoniae phages Kle_F17/19 and Kle_F58/19, and variants thereof which maintain the lytic activity of these bacteriophage, which have antibacterial activity against Klebsiella pneumoniae. These bacteriophage have been deposited: Kle_F17/19 has deposit accession number NCIMB 43534 and Kle_F58/19 has deposit accession number NCIMB 43535. These bacteriophage particularly can lyse K. pneumoniae strains 57/15 (NCIMB accession number 43536) and 237/14 (NCIMB accession number 43537).

Another aspect of the invention relates to compositions comprising three or more different purified bacteriophages in a cocktail combination. In one aspect, the bacteriophage cocktail comprises or consists of, three K. pneumoniae bacteriophage, Kle_F391/08, which has a genome with a nucleotide sequence of SEQ ID NO: 1, Kle_F17/19, which has a genome with a nucleotide sequence of SEQ ID NO: 2, and Kle_F58/19, which as a genome with a nucleotide sequence of SEQ ID NO: 3 (including variants thereof that maintain the lytic activity of the bacteriophage) and having antibacterial activity against K. pneumoniae. This cocktail, in other aspects of the invention, may further include or be administered in combination with a cocktail of one, two, three or more additional bacteriophage, for example, against P. aeruginosa. In an aspect, the cocktail comprising the phage Kle_F391/08 (deposited as NCIMB 42918), Kle_F17/19 (having a genome with the nucleotide sequence of SEQ ID NO: 1) and Kle_F58/19 (having a genome with the nucleotide sequence of SEQ ID NO: 3) formulated with and/or administered in combination with a cocktail of Pseudomonas aeruginosa bacteriophage Psa_F99/10 (deposited as NCIMB 42915), Psa_F27/12 (deposited as NCIMB 42916) and Psa_F95/13 (deposited as NCIMB 42917) (also disclosed in US2019/0290709 which is incorporated herein by reference). In other embodiments, the bacteriophage cocktail disclosed herein is administered with one or more other purified bacteriophage that have antibacterial activity against K. pneumoniae, P. aeruginosa, or a bacteria that is not K. pneumoniae or P. aeruginosa. In still more preferred embodiments, the composition is formulated for administration as an aerosol and for pulmonary delivery.

Another aspect of the invention relates to pharmaceutical compositions comprising a bacteriophage or phage product of the invention and a pharmaceutically acceptable carrier. In some embodiments, the pharmaceutical composition further comprises one or more additional bacteriophage or phage products having antibacterial activity against K. pneumonia. In some embodiments, the composition is formulated in a dosage form in which the bacteriophage is present in an amount to provide a multiplicity of infection (MOI) of about 1 to about 10 upon administration of the composition to a subject in need thereof. In certain embodiments, each phage is administered at a dosage of 10⁸ to 10¹¹, including 10⁹ pfu to 10¹⁰ pfu and for, example, is present at 10⁸ to 10¹¹, including 10⁹ or 10¹⁰ pfu/ml in the composition. In certain embodiments, each phage is present in approximately the same amount, including a ratio of 1:1:1 in the composition or dosage form. In preferred embodiments, the composition is formulated for administration as an aerosol.

Another aspect of the invention relates to methods for treating or reducing the occurrence of or managing a bacterial infection in a subject in need thereof comprising administering to the subject a therapeutically or prophylactically effective amount of a pharmaceutical composition of the invention, as well as the use of the pharmaceutical composition in this regard. In some embodiments, the bacterial infection is caused by a K. pneumoniae bacterial strain, and in certain aspects and/or a P. aeruginosa bacterial strain, including a bacterial strain showing resistance to one or more known antibiotics and/or capable of forming a biofilm. In preferred embodiments, the bacterial infection to be treated, or reduced in occurrence, is a respiratory infection, more preferably a hospital-acquired bacterial pneumonia or a respiratory infection associated with cystic fibrosis. In particularly preferred embodiments, the composition is administered as an aerosol to the lungs. In some embodiments, the composition is re-administered about 4, 6 or 8 hours after initial administration.

Another aspect of the invention relates to a method for diagnosing the causative agent of a bacterial infection and/or assessing whether the infectious agent is susceptible to the lytic activity of a bacteriophage, comprising (i) culturing a sample, such as a swab or sputum or other sample appropriate for culturing the bacteria causing the infection, from a patient; (ii) contacting the culture of step (i) with a bacteriophage or phage product disclosed herein; and (iii) monitoring for evidence of growth or lysis of the culture, where evidence of lysis of the culture indicates that the culture comprises a bacterial strain known to be susceptible to the bacteriophage or phage product used in step (ii). In some embodiments, the sample is a tissue biopsy or swab collected from the respiratory tract of the patient. For example, the sample may comprise bronchoalveolar lavage or bronchial secretions.

Still another aspect of the invention provides a method for reducing or inhibiting colonization or growth of bacteria on a surface comprising contacting the surface with a bacteriophage or phage product of the invention. In some embodiments, the surface is a mucus membrane of a mammal, preferably a mucus membrane of the respiratory tract of a human. In some embodiments, the surface is a non-biological surface, preferably the surface of a hospital apparatus or a piece of hospital equipment, more preferably a surgical apparatus or piece of surgical equipment.

### 3.1 DEFINITIONS

As used herein, the term “isolated” in the context of nucleic acid molecules refers to a first nucleic acid molecule which is separated from other nucleic acid molecules which are present in the natural source of the first nucleic acid molecule. An “isolated” nucleic acid molecule, such as an “orf” or a phage genome, is substantially free of other cellular material, or culture medium when produced by recombinant techniques, or substantially free of chemical precursors or other chemicals when chemically synthesized; and may be free of other DNA or other genomic DNA molecules, e.g., where it has been purified and isolated from other clones in a nucleic acid library or from isolated phage. Further, “isolated” genomic DNA is substantially free of other viral or cellular material, or culture medium when produced by recombinant techniques or isolated from phage, or substantially free of chemical precursors or other chemicals when chemically synthesized, and may be free of other DNA or other genomic DNA molecules, e.g., where it has been purified and isolated from preparations containing other bacteriophage or cellular material.

The term “purified” with respect to a bacteriophage means that the phage has been measurably increased in concentration by any purification process, including but not limited to, isolation from the environment or culture, e.g., isolation from culture following propagation and/or amplification, centrifugation, etc., thereby partially, substantially, nearly completely, or completely removing impurities, such as host cells and host cell components. One of skill in the art will appreciate the amount of purification necessary for a given use. For example, a purified phage meant for use in therapeutic compositions intended for administration to humans ordinarily must be of high purity in accordance with regulatory standards and good manufacturing processes.

The term “purified” with respect to a peptide, polypeptide, fusion protein, or nucleic acid molecule means that the peptide, polypeptide, fusion protein, or nucleic acid molecule has been measurably increased in concentration by any purification process, including but not limited to, column chromatography, HPLC, precipitation, electrophoresis, etc., thereby partially, substantially, nearly completely, or completely removing impurities, such as precursors or other chemicals involved in preparing the peptide, polypeptide, fusion protein, or nucleic acid molecule. One of skill in the art will appreciate the amount of purification necessary for a given use. For example, isolated and purified genomic DNA or protein or polypeptides meant for use in therapeutic compositions intended for administration to humans ordinarily must be of high purity in accordance with regulatory standards and good manufacturing processes.

As used herein the terms “bacteriophage products” or “active bacteriophage products” refer to proteins, or fragments or variants thereof, as well as nucleic acids encoding same, which have been isolated or derived from a bacteriophage of the invention and which retain a biological function or activity associated with the bacteriophage from which it was isolated or derived (e.g., antibacterial activity such as lytic cell killing).

As used herein, the term “variant” in the context of nucleotide sequences refers to a nucleotide sequence that comprises or consists of a nucleotide sequence having a sequence identity of at least 70%, 75%, 80%, 85%, 90%, 93%, 94%, 95%, 96%, 97%, 98%, or at least 99% with a reference nucleic acid sequence, e.g., at least 70%, 75%, 80%, 85%, 90%, 93%, 94%, 95%, 96%, 97%, 98%, or at least 99% sequence identity with one of the nucleotide sequences of SEQ ID NOs: 1, 2, or 3. A variant may be selected that maintains one or more functions of the reference nucleic acid sequence. For example, a variant bacteriophage may exhibit at least one biological activity, e.g., antibacterial activity, such as lytic killing activity, of the bacteriophage from which it is derived. One of skill in the art will appreciate that nucleic acid replication in phages is less than 100% accurate, such that a given phage may show at least 1% variation as it replicates, including during its production as an antibiotic agent. The expected genome variation during manufacture and use of phages may result in progeny that are variants having at least about 85%, 90%, 95%, 96%, 97%, 98%, or 99% sequence identity to the nucleotide sequence of the parent genome. It follows that, in certain embodiments, the bacteriophage of the invention comprises or consists of a genome having at least about 85%, 90%, 95%, 96%, 97%, 98%, or at least 99% sequence identity to the nucleotide sequence of the parent phage, while retaining antibacterial activity against the target (host) bacteria of the parent phage. A “variant” in the context of a bacteriophage, is a bacteriophage, the genome of which has at least about 85%, 90%, 95%, 96%, 97%, 98%, or 99% sequence identity to the nucleotide sequence of the parent genome and maintains the lytic activity (i.e., lyses the same strain or strains of bacteria) as the parent bacteriophage. In certain aspects, the “variant” is identified solely by its lytic activity (for example, as described in the Examples herein) against 1, 2 or three strains of bacteria.

For example, in certain embodiments, the bacteriophage is a variant of Kle_F391/08 (having a genome with a nucleotide sequence of SEQ ID NO: 1), Kle_F17/19 (having a genome with a nucleotide sequence of SEQ ID NO: 2), or Kle_F58/19 (having a genome with a nucleotide sequence of SEQ ID NO: 3), and retains antibacterial and/or lytic activity against one, two or three of Klebsiella pneumonia strains 57/17, 237/14 and 397/07.

The term “progeny” with reference to any of the novel phages herein means bacteriophage replicates containing descendants produced according to subculture of a bacteriophage of a specific nucleic acid identified herein, or by a method known to those ordinarily skilled in the art, or bacteriophages having a RFLP (Restriction fragment length polymorphism) DNA profile substantially equivalent to the bacteriophage of a specific nucleic acid identified herein. The term “have a substantially equivalent or equal RFLP” is expressed to represent a variability between organisms according to the method suggested by Tenover et al. (Tenover, F. C. et al. Interpreting Chromosomal DNA Restriction Patterns Produced by Pulsed-Field Gel Electrophoresis: Criteria for Bacterial Strain Typing. J. Clin. Microbiol 33:2233-2239 (1995)). Tenover et al. suggest an acceptable level of variability with a proviso that genome of identical propagated organisms is restricted with restriction enzymes and then electrophoresed. According to the standard suggested by Tenover et al, a progeny having an equivalent RFLP DNA profile may be considered as a bacteriophage substantially equivalent to the bacteriophage of a specific nucleic acid identified herein, that is, substantially the equivalent of a bacteriophage comprising a genome having the nucleotide sequence of any of SEQ ID NOs: 1-3.

As used herein, the term “host cell” refers to the particular subject cell transfected with a nucleic acid molecule and the progeny or potential progeny of such a cell that contains the nucleic acid molecule or chromosomally integrated version thereof. Progeny of such a cell may not be identical to the parent cell transfected with the nucleic acid molecule due to mutations or environmental influences that may occur in succeeding generations or integration of the nucleic acid molecule into the host cell genome. “Host cell” also refers to a cell, such as a bacterial cell, infected with bacteriophages, e.g., whole phages, where the bacteriophages live and replicate. For the generation of bacteriophage, the host cell may or may not be of the same species or strain from which the bacteriophage was isolated or cultured.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a full-length protein. In a specific embodiment, the fragment is a functional fragment in that it retains at least one function of the protein from which it is isolated, e.g., retaining antibacterial activity, such as lytic cell killing.

As used herein, the term “in combination” or “in further combination” or “further in combination” refers to the use of an additional prophylactic and/or therapeutic agent with a bacteriophage or phage product of the invention, including a phage cocktail of different bacteriophages of the invention. The use of the term “in combination” does not restrict the order in which prophylactic and/or therapeutic agents are administered to a subject. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent (different from the first prophylactic or therapeutic agent) to a subject.

As used herein, the term “boost” or “booster” refers to subsequent, repeat use of the same or substantially the same prophylactic and/or therapeutic agent, such as repeat doses of a bacteriophage, phage product, or phage cocktail of the invention. The prophylactic or therapeutic agent can be first administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) the second administration of the same or substantially the same prophylactic or therapeutic agent to a subject.

As used herein, the terms “prophylactic agent” and “prophylactic agents” refer to an agent, such as a bacteriophage, phage product, or phage cocktail of the invention, which can be used in the prevention, management, control, or reduction in the incidence of, one or more symptoms of a disease or disorder, in particular, a disease or disorder associated with a bacterial infection, more particularly, a disease or disorder associated with a respiratory bacterial infection, such as but not limited to a hospital-acquired bacterial pneumonia or a respiratory bacterial infection associated with cystic fibrosis.

As used herein, the terms “therapeutic agent” and “therapeutic agents” refer to an agent, such as a bacteriophage, phage product, or phage cocktail of the invention, that can be used in the treatment, management, or control of one or more symptoms of a disease or disorder, in particular, a disease or disorder associated with a bacterial infection, more particularly, a disease or disorder associated with a respiratory bacterial infection, such as but not limited to hospital-acquired bacterial pneumonia or a respiratory bacterial infection associated with cystic fibrosis.

As used herein, the terms “treat”, “treatment” and “treating” refer to obtaining a therapeutic benefit in a subject receiving a pharmaceutical composition. With respect to achieving a therapeutic benefit, the object is to eliminate, lessen, decrease the severity of, ameliorate, or slow the progression of the symptoms or underlying cause (e.g., bacterial infection) associated with the pathological condition or disorder. A “therapeutically effective amount” refers to that amount of a therapeutic agent, such as a bacteriophage or phage product in a pharmaceutical composition of the invention, sufficient to achieve at least one therapeutic benefit in a subject receiving the pharmaceutical composition.

As used herein, the terms “prevent”, “prevention” and “preventing” refer to obtaining a prophylactic benefit in a subject receiving a pharmaceutical composition. With respect to achieving a prophylactic benefit, the object is to delay, reduce the incidence of, or prevent the symptoms or underlying cause (e.g., bacterial infection) associated with the pathological condition or disorder. A “prophylactically effective amount” refers to that amount of a prophylactic agent, such as a bacteriophage or phage product in a pharmaceutical composition of the invention, sufficient to achieve at least one prophylactic benefit in a subject receiving the pharmaceutical composition.

As used herein, the terms “antibacterial activity” and “antimicrobial activity”, with reference to a bacteriophage or bacteriophage product (e.g., a phage protein), or a variant or fragment thereof, are used interchangeably to refer to the ability to kill and/or inhibit the growth or reproduction of a microorganism, in particular, the bacteria of the species or strain that the bacteriophage infects. In certain embodiments, antibacterial activity is assessed by culturing bacteria, e.g., Gram-negative bacteria (e.g., P. aeruginosa or K. pneumoniae) according to standard techniques (e.g., in liquid culture or on agar plates), contacting the culture with a bacteriophage, phage protein, or variant thereof of the invention, or with a cocktail of bacteriophages, phage proteins, or variants thereof, and monitoring cell growth after said contacting. For example, in a liquid culture, the bacteria may be grown to an optical density (“OD”) representative of a mid-point in exponential growth of the culture; the culture is exposed to one or more concentrations of one or more bacteriophages of the invention, bacteriophage products, or variants thereof, and the OD is monitored relative to a control culture. Decreased OD relative to a control culture is representative of phage(s) or phage product(s) exhibiting antibacterial activity (e.g., lytic killing activity). Similarly, bacterial colonies can be allowed to form on an agar plate, the plate exposed to one or more bacteriophages or phage products of the invention, or variants thereof, and subsequent growth of the colonies evaluated related to control plates. Decreased size of colonies, or decreased total numbers of colonies, indicate phage(s) or phage product(s) with antibacterial activity.

### 4. BRIEF DESCRIPTION OF THE FIGURES

FIG. 1 illustrates morphological features of bacteriophages Kle_F391/08 (A), Kle_F17/19 (B) and Kle_F58/19 (C). These bacteriophages were characterized using transmission electron microscopy (Hitachi H-7650).

FIG. 2 shows schematic organization of the F391/08 genome. The orfs predicted in the approximately 113 kb genome are represented by arrows and numbered in black. The direction of an arrow indicates the direction of transcription. Color coding: black—open reading framed (orfs) for whose products a functional assignment could be made based on homologous proteins; grey—orfs coding for products that are similar to proteins of unknown function; empty arrows—orfs coding for proteins that share no significant homology with proteins in databases.

FIG. 3 shows schematic organization of the Kle_F17/19 genome. The orfs predicted in the approximately 45 kb genome are represented by arrows and numbered in black. The direction of an arrow indicates the direction of transcription. Color coding: black—orfs for whose products a functional assignment could be made based on homologous proteins; grey—orfs coding for products that are similar to proteins of unknown function; empty arrows—orfs coding for proteins that share no significant homology with proteins in databases.

FIG. 4 shows schematic organization of the Kle_F58/19 genome. The orfs predicted in the approximately 170 kb genome are represented by arrows and numbered in black. The direction of an arrow indicates the direction of transcription. Color coding: black—orfs for whose products a functional assignment could be made based on homologous proteins; grey—orfs coding for products that are similar to proteins of unknown function; empty arrows—orfs coding for proteins that share no significant homology with proteins in databases.

FIG. 5 represents a single lysis curves of F391/08 and Kle_F58/19 bacteriophages (MOI 10) with K. pneumoniae 57/17. Viable cell counts were quantified by the 10-fold serial dilution method and monitored at 1 h intervals for an 8 h period and again at 24 h ppi.

FIG. 6 represents a single lysis curve of Kle_F17/19 bacteriophages (MOI 10) with K. pneumoniae 237/14. Viable cell counts were quantified by the 10-fold serial dilution method and monitored at 1 h intervals for an 8 h period and again at 24 h ppi.

FIG. 7 depicts a combined lysis curve of F391/08, Kle_F17/19 and Kle_F58/19 bacteriophages with approximately an MOI of 10. Viable cell counts were quantified by the 10-fold serial dilution method and monitored at 1 hour intervals for an 8 hour period and again at 24 h ppi.

FIG. 8 illustrates bacterial load 24 h after infection with strain 2 and nebulized purified phage cocktail/antibiotic (co)treatment or NaCl (mock) treatment. Bacterial load is given as cfu per precision cut lung slice. Bars represent mean±SD for 2 technical replicates in n=1 experiment.

FIG. 9 depicts tissue viability 24 h after infection with strain 2 and nebulized purified phage cocktail/antibiotic (co)treatment or NaCl (mock) treatment. Tissue viability was assessed by Calcein staining and is given as relative light units. Bars represent mean±SD for 2 technical replicates (each measured in duplicate), in n=1 experiments.

FIG. 10 depicts phage titers in precision-cut lung slices (PCLS) at 1 h and 24 hours post infection using plaque assay with the indicated detection strains. Phage titers are given as plaque forming units (PFU) per PCLS. Bars represent mean±SD for 2-4 technical replicates (1-2 exposure plates with 2 PCLS each, shown as individual dots), for n=1 experiment.

FIG. 11 illustrates the bacterial load 1 h or 24 h after infection followed by treatment with nebulized phage cocktail or NaCl (vehicle) treatment. Bacterial load is given as CFU per PCLS. Bars represent mean±SD for 4 technical replicates (2 exposure plates with 2 PCLS each, mean per plate shown as dots) for n=1 experiment.

FIG. 12 depicts phage titers of inoculum 1 and 24 h after infection with strain 1 and treatment with phage cocktail as measured using plaque formation assay. Phage titers are given as plaque forming units. Bars represent mean±SD for 2 technical replicates (shown as individual dots) for n=3 experiments.

FIG. 13 shows bacterial load 24 h post infection after treatment with nebulized NaCl or phage cocktail, alone or in combination with antibiotic co-treatment. Bacterial load is given as CFU per PCLS. Bars represent mean±SD for 2 technical replicates (shown as individual dots) for n=3 experiments. *** indicates significance with p<0.001 compared to control column (infected+NaCl 24 h p.i.) according to One-way ANOVA and Sidak's multiple comparison post test. ### indicates significance with p<0.001 according to unpaired, two-tailed t test for direct comparison of indicated groups.

FIG. 14 depicts tissue viability 24 h after infection and treatment with NaCl (vehicle), nebulized phage cocktail alone or antibiotic co-treatment. Tissue viability was assessed by Calcein staining and is given as relative light units (RLU). Bars represent mean±SD for 2 technical replicates (shown as individual dots), each measured in duplicate, for n=3 experiments. **; *** indicates significance with p<0.01; 0.001 compared to control column (infected+NaCl 24 h p.i.) according to One-way ANOVA and Sidak's multiple comparison post test.

### 5. DETAILED DESCRIPTION

The present invention is directed to phage therapy for the treatment and control of bacterial infections, in particular respiratory bacterial infections such as bacterial pneumonia and respiratory infections associated with cystic fibrosis. One aspect invention relates to novel bacteriophage strains, including the K. pneumonia phages Kle_F17/19 (having a genome with the nucleotide sequence of SEQ ID NO: 2) and Kle_F58/19 (having a genome with the nucleotide sequence of SEQ ID NO: 3), as well as variants thereof. Another aspect of the invention relates to cocktail compositions of one or more bacteriophage and/or phage products of the invention, as well as combinations with other phage, including Kle_F391/08 (having a genome of the nucleotide sequence of SEQ ID NO: 1) (previously disclosed in PCT/PT2011/000031, published as International Application PCT WO2012/036580, which is herein incorporated by reference). Another aspect of the invention relates to a cocktail compositions of the K. pneumonia phages Kle_F391/08 (SEQ ID NO: 1), Kle_F17/19 (SEQ ID NO: 2) and Kle_F58/19 (SEQ ID NO: 3) and variants thereof (including variants in which the genome has at least 95%, 98% or 99% sequence identity thereto, including where the variation arises from propagation of the phage). Still another aspect relates to pharmaceutical compositions of the phage(s), as well as methods of using same in the treatment and prevention of bacterial infections, in particular, respiratory infections caused by K. pneumoniae. Still other aspects of the invention relate to use of the phages, and combinations thereof, as diagnostic tools and disinfective agents.

### 5.1 BACTERIOPHAGE AND VARIANTS THEREOF

Another aspect of the invention relates to novel Klebsiella pneumoniae bacteriophages that target a number of strains of K. pneumoniae. Klebsiella pneumoniae is a Gram-negative, non-motile, rod-shaped bacterium, found in the normal flora of the mouth, skin, and intestines. As an encapsulated, facultative anaerobe, the bacterium also naturally occurs in the soil. Clinically, it is the most important member of the Klebsiella genus of Enterobacteriaceae. Klebsiella infections tend to occur in people with a weakened immune system from improper diet, e.g. in alcoholics and diabetics. Klebsiella is also an opportunistic pathogen for patients with chronic pulmonary disease, nasal mucosa atrophy, cystic fibrosis, and rhinoscleroma. New antibiotic resistant strains of K. pneumoniae are appearing, and it is increasingly found as a nosocomial infection, for example, due to contact with contaminated instruments.

K. pneumoniae is indeed one of the most important causative pathogens of respiratory tract infections in humans and alone accounts for 25-43% of the nosocomial pneumonias caused by Gram-negative bacteria (Chibber S et al., 2008, J Med Microbiol 57(12):1508-1513). The high incidence of multidrug resistant bacteria has resulted in limited efficacy with current antibiotics, and a high probability of patient colonization by resistant strains. The capsular polysaccharide is an important virulent factor of Klebsiella sp. strains, and a limiting factor for phage infection. Literature has described 78 capsular types (Hus C R, et al., 2013, PLoS One 8(8):e70092), and phages that infect these species have overcome this “barrier”. K. pneumoniae virulent strains have been predominantly associated with the K1 and K2 capsular serotypes (Cleg S et al., 2016, Microbiol Spectr 4(1); and Lin TZ et al., 2014, J Infect Dis 210:1734-1744), such as in pyogenic liver abscess, though the K1 capsular serotype has been associated with community-acquired isolates rather than nosocomial isolates (Tsay R W et al., 2002, Arch Intern Med 162(9):1021-1027). Nonetheless, depending on the type of infection, strains can show a diverse range of capsular serotypes and the distribution of K. pneumoniae capsular serotypes differs worldwide (Hus C R et al., 2013, PLoS One 8(8):e70092).

In one embodiment, provided is the purified bacteriophage F17/19 (a designation used interchangeably with “Kle_F17/19”), which targets a number of strains of K. pneumoniae.

In another embodiment, provided is the purified bacteriophage F58/19 (a designation used interchangeably with “Kle_F58/19”), which also targets a number of K. pneumoniae strains.

Bacteriophage and certain bacterial strains disclosed herein were deposited with the NCIMB (NCIMB Ltd. Ferguson Building, Craibstone Estate, Bucksburn, Aberdeen UK) on Dec. 13, 2019, and these deposits are hereby incorporated by reference:

TABLE 1

| Accession | Bacteriophage or bacterial strain | |
|---|---|---|
| Number | identifier | Species |
| NCIMB | Kle_F391/08 (SEQ ID NO: 1) | Klebsiella pneumoniae |
| 43533 | | bacteriophage |
| NCIMB | Kle_F17/19 (SEQ ID NO: 2) | Klebsiella pneumoniae |
| 43534 | | bacteriophage |
| NCIMB | Kle_F58/19 (SEQ ID NO: 3) | Klebsiella pneumoniae |
| 43535 | | bacteriophage |
| NCIMB | Klebsiella pneumoniae 57/17-B8 | Klebsiella pneumoniae |
| 43536 | | |
| NCIMB | Klebsiella pneumoniae 237/14 - B7 | Klebsiella pneumoniae |
| 43537 | | |
| NCIMB | Klebsiella pneumoniae 397/07 - B6 | Klebsiella pneumoniae |
| 43538 | | |

Disclosed herein are the nucleotide sequences of the genomes of these phage. In particular, provided are phage Kle_F391/08 having a genome with a nucleotide sequence of SEQ ID NO: 1, Kle_F17/19 having a genome with a nucleotide sequence of SEQ ID NO:2, and Kle_58/19 having a genome with a nucleotide sequence of SEQ ID NO:3, (or at least 95%, 98% or 99% identity to SEQ ID NO:1, accounting, for example, for nucleotide sequence modifications that may occur through replication of the parent phage but do not alter the lytic activity of the phage).

### 5.2 COCKTAIL COMPOSITIONS

A particular aspect of the invention relates to cocktail compositions of different bacteriophages. The “cocktail” may comprise at least two different purified bacteriophage, for example, two, three, four, five, six, seven, eight, nine, ten, or more different purified bacteriophages, or variants thereof. The cocktail may be used alone or in further combination with other therapies, e.g., antibiotic agents and/or antifungal agents.

Phage cocktails provide advantages over the use of phages individually, e.g., to increase the lytic activity against a particular species or strain of bacteria and/or to decrease the possibility of emergence of bacteria resistant to an individual bacteriophage. Different bacteriophage also can be mixed as cocktails to broaden their properties, preferably resulting in a collectively greater antibacterial spectrum of activity. However, few phage cocktails exist with antimicrobial activity against different bacteria, probably because of the difficulty in combining different specificities of bacteriophage strains, while maintaining infecting ability and/or lytic activity of the individual bacteriophage in the presence of distinct strains.

In some embodiments, the invention provides cocktail compositions comprising at least three different purified bacteriophages, with antibacterial activity against the same or different bacterial species or strains. In some particular embodiments, the instant invention provides a cocktail composition comprising at least or consisting of purified bacteriophages Kle_F391/08 (genome having a nucleotide sequence of SEQ ID NO: 1), Kle_F17/19 (genome having a nucleotide sequence of SEQ ID NO: 2), and Kle_F58/19 (genome having a nucleotide sequence of SEQ ID NO: 3), or a variant thereof, including variants that have lytic activity against one of all of K. pneumoniae 57/17, 237/14 and/or 397/07.

In some particular embodiments, provided are compositions comprising or consisting of at least two different purified bacteriophages, including at least one of or at least 2 of or all three of F17/19 or F58/19 or Kle_F391/08, or a variant thereof.

In some particular embodiments, the combination does not impair or reduce (or does not substantially or significantly impair or reduce) infection ability or host range and/or lytic activity of the individual bacteriophage in the presence of distinct bacteriophage strains. In some particularly preferred embodiments, the efficacy of at least one phage in the cocktail combination is enhanced or improved due to the presence of at least one other phage in the cocktail combination, producing a synergistic effect.

In some embodiments, the cocktail composition comprises at least three phage showing antibacterial activity against Klebsiella pneumoniae. In some particular embodiments, the invention provides a cocktail composition comprising at least three different purified bacteriophages or consists of three purified bacteriophages F391/08, F17/19, and F58/19, or a variant thereof having antibacterial activity against K. pneumoniae.

In particularly preferred in other embodiments, the composition comprises or consists of purified bacteriophages F391/08, F17/19, and F58/19, or variants thereof and three purified bacteriophages F99/10, F27/12, and F95/13, and having antibacterial activity against Pseudomonas aeruginosa. Alternatively, the compositions comprising or consisting of the purified bacteriophages F391/08, F17/19, and F58/19 are administered in combination with a composition comprising or consisting of the purified bacteriophages F99/10, F27/12, and F 95/13. F99/10, F27/12, and F 95/13 are disclosed in US2019/0290709, which is hereby incorporated by reference in its entirety and specifically for these bacteriophage. In embodiments, one or two of bacteriophages F391/08, F17/19, and F58/19, or variants thereof are administered in a cocktail with one, two or all three of F99/10, F27/12, and F95/1. These bacteriophage and certain P. aeruginosa strains have been deposited with the NCIMB (NCIMB Ltd. Ferguson Building, Craibstone Estate, Bucksburn, Aberdeen UK) on Dec. 1, 2017, and these deposits are hereby incorporated by reference:

TABLE 2

| Accession | Bacteriophage or bacterial strain | |
|---|---|---|
| Number | identifier | Species |
| NCIMB | F99/10 | Pseudomonas aeruginosa |
| 42915 | | bacteriophage |
| NCIMB | F27/12 | Pseudomonas aeruginosa |
| 42916 | | bacteriophage |
| NCIMB | F95/13 | Pseudomonas aeruginosa |
| 42917 | | bacteriophage |
| NCIMB | Pseudomonas aeruginosa 391/08 | Pseudomonas aeruginosa |
| 42918 | | |
| NCIMB | Pseudomonas aeruginosa 92/15 | Pseudomonas aeruginosa |
| 42919 | | |
| NCIMB | Pseudomonas aeruginosa 105/15 | Pseudomonas aeruginosa |
| 42920 | | |
| NCIMB | Pseudomonas aeruginosa 121-15 | Pseudomonas aeruginosa |
| 42913 | | |

In some embodiments, provided is a cocktail composition comprising at least two different purified bacteriophages, one of which is either F17/19 or F58/19, and at least one selected from a purified bacteriophage of F92/15, F105/15, F134/15, and F141/15, against one or more of strains of Klebsiella species, more preferably including K. pneumoniae, all of which bacteriophage are disclosed in US 2019/0290709, which is incorporated by reference herein in its entirety, or a variant thereof.

In some embodiments, the invention provides a cocktail composition comprising at least F17/19 or F58/19 further in combination with at least one additional phage is selected from the group consisting of bacteriophage F168/08 having antibiotic activity against one or more strains of E. faecalis and/or E. faecium (as disclosed in WO 2010/090542), bacteriophage F170/08 having antibiotic activity against one or more strains of E. faecalis and/or E. faecium (as disclosed in WO 2010/090542), bacteriophage F770/05 having antibacterial activity against one or more strains of P. aeruginosa (as disclosed in WO 2010/090542), bacteriophage F197/08 having antibacterial activity against one or more strains of Staphylococcus aureus (as disclosed in WO 2010/090542), bacteriophage F86/06 having antibacterial activity against one or more strains of Staphylococcus aureus (as disclosed in WO 2010/090542), bacteriophage F87s/06 having antibacterial activity against one or more strains of Staphylococcus aureus (as disclosed in WO 2010/090542), bacteriophage F91a/06 having antibacterial activity against one or more strains of Staphylococcus aureus (as disclosed in WO 2010/090542), bacteriophage F1245/05 having antibacterial activity against one or more strains of Acinetobacter baumanni (as disclosed in WO 2010/090542), bacteriophage strain F394/08 having antibacterial activity against one or more strains of Acinetobacter baumanni (as disclosed in WO 2012/036580), bacteriophage F488/08 having antibacterial activity against one or more strains of Escherichia coli (as disclosed in WO 2012/036580), bacteriophage F510/08 having antibacterial activity against one or more strains of P. aeruginosa (as disclosed in WO 2012/036580), bacteriophage F44/10 having antibacterial activity against one or more strains of Staphylococcus aureus (as disclosed in WO 2012/036580), bacteriophage F387/08 having antibacterial activity against one or more strains of Klebsiella pneumoniae (as disclosed in WO 2012/036580), and bacteriophage F125/10 having antibacterial activity against one or more strains of Staphylococcus aureus (as disclosed in WO 2012/036580) (the contents of each are hereby incorporated by reference in their entireties).

The bacteriophage of the invention and/or for use in cocktail compositions of the invention, can be obtained by any methods known in the art and/or disclosed herein. In some embodiments, the invention provides for methods of production and purification of a bacteriophage F391/08, F17/19, and/or F58/19, for example, from the deposited strain disclosed herein.

Further, bacteriophage may be isolated from a bacterial sample using any method described herein or known in the art (see, e.g., Carlson, “Working with bacteriophages: common techniques and methodological approaches,” In, Kutter and Sulakvelidze (Eds) Bacteriophages: Biology and Applications, 5th ed. CRC Press (2005), incorporated herein by reference in its entirety). Specific bacterial strains that may be used include, e.g. Klebsiella pneumoniae 57/17, 397/07, or 237/14 strains (e.g., for isolating phage F391/08, F17/19 or F58/19). Pseudomonas aeruginosa 391/08, 92/15, 105/115 or 121/15 strains (e.g., for isolating phage F99/10, F110/10, F27/12, F83/13, and/or F95/13); or Bacteriophage also may be isolated from any other bacterial strain susceptible to infection by one or more of the bacteriophage, and in which the bacteriophage replicate.

The skilled artisan also may use one or more methods to propagate or amplify a bacteriophage, particularly purified bacteriophage F391/08, F17/19, or F58/19, as well of variants thereof, so as to obtain greater amounts of a given phage. In some embodiments, a method of producing and/or isolating additional phage may comprise (i) obtaining a culture of K. pneumoniae, (ii) infecting it with the bacteriophage F391/08, F17/19, or F58/19, or a variant thereof; (iii) culturing until significant lysis of the culture is observed; and (iv) isolating from the culture the bacteriophage. The host cell used may be any bacterial strain, for example, any K. pneumoniae strain susceptible to infection by the phage that can be used to replicate the phage. In some embodiments, the host cell used may be, for example, K. pneumoniae 57/17 strain, K. pneumoniae 237/14 strain, or K. pneumoniae 397/07 strain.

### 5.3 PHARMACEUTICAL COMPOSITIONS

The purified bacteriophages disclosed herein, including variants thereof, and phage cocktail combinations, may be administered alone or incorporated into a pharmaceutical composition for the use in treatment or prophylaxis of bacterial infections, e.g., infections caused by bacteria including, but not limited to, P. aeruginosa and K. pneumoniae. The bacteriophage(s) or phage product(s) may be combined with a pharmaceutically acceptable carrier, excipient, or stabilizer. Examples of pharmaceutically acceptable carriers, excipients and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as TWEEN™, polyethylene glycol (PEG), and PLUONICS™. The pharmaceutical compositions of the present invention (e.g., antibacterial compositions) can also include a lubricant, a wetting agent, an emulsifier, a suspending agent, and a preservative, e.g., in addition to the above ingredients. In specific aspects, the purified bacteriophage are formulated in an NaCl solution, for example 0.9% NaCl.

In some embodiments, the pharmaceutical compositions are formulated for administration as an aerosol. Formulations for aerosol delivery may be in the form of a dry powder, fine particles, nanoparticles, solutions, lyophilized preparations, liposomal preparations, and the like. Liposomal formulations can protect the bacteriophage from the harsh condition of the sputum, as well as improving penetration into biofilms and/or allowing more sustained release of the agent within airways. Formulations for aerosol delivery typically comprise sterile water and little or no preservatives, to reduce side effects such as bronchial irritation and bronchospasm. Formulations for aerosol delivery preferably have an osmolality the same as, or substantially the same as, the osmolality of airway surface liquid.

The phages and bacteriophage cocktails disclosed herein may be combined with one or more other therapeutic and/or prophylactic agents useful for the treatment of bacterial infection as described herein and/or known in the art, e.g. one or more other bacteriophages or an antibiotic. For example, a pharmaceutical composition of the invention may comprise two or more purified bacteriophage disclosed herein (with antibacterial activity against the same or different bacterial species or strains), and a bacteriophage known in the art. In specific embodiments, the therapeutic components of a combination target two or more species or strains of bacteria.

The pharmaceutical compositions of the present invention also may be combined with one or more non-phage therapeutic and/or prophylactic agents, useful for the treatment and/or prevention of bacterial infections, as described herein and/or known in the art (e.g. one or more traditional antibiotic agents). Other therapeutic and/or prophylactic agents that may be used in combination with the phage(s) or phage product(s) of the invention include, but are not limited to, antibiotic agents, anti-inflammatory agents, antiviral agents, antifungal agents, or local anesthetic agents. In some preferred embodiments, the pharmaceutical composition is formulated for treatment and/or prevention of pulmonary infections and comprises one or more additional therapeutic and/or prophylactic agents selected from antibiotic agents, antifungal agents, and local anesthetic agents. In some embodiments, the pharmaceutical composition comprises a phage cocktail combination of the invention, which is administered in the absence of a standard or traditional antibiotic agent.

In particular embodiments, the bacteriophage cocktail is administered with a broad spectrum antibiotic, particularly with activity against gram negative bacteria, such as an anti-pseudomonal β-lactam (for example, piperacillin, tazobactam, ceftazidime or meropenem) and/or an aminoglycoside (for example, amikacin), and/or in combination with an antibiotic against gram positive bacteria, such as vancomycin or an oxazolidinone (for example, linesolid), and/or colistin. Standard or traditional antibiotic agents include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef. ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolami de, benzolamide, bumetanide, celecoxib, chlorthalidone, clopami de, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof

In some embodiments, the pharmaceutical composition of the invention comprises an antibiotic agent having antibacterial activity against P. aeruginosa and/or K. pneumoniae. In some other embodiments, the pharmaceutical composition of the invention comprises an antibiotic agent having antibacterial activity against bacteria other than P. aeruginosa and/or K. pneumoniae. In preferred embodiments, the antibiotic agent is used in an amount effective to additively or synergistically enhance the therapeutic and/or prophylactic effect of a phage, phage product, or phage cocktail of the present invention for a given infection.

Standard antifungal agents include amphotericin B such as liposomal amphotericin B and non-liposomal amphotericin B.

In some preferred embodiments, the pharmaceutical composition of the invention is formulated for administration as an aerosol and further comprises one or more antibiotics also for aerosol delivery. Antibiotics for aerosol delivery include, e.g., inhaled aminoglycosides, such as tobramycin like tobramycin solution or tobramycin dry powder, gentamicin, amikacin, inhaled polymyxins, such as colistin solution or colistin dry powder and colistimethate sodium; and inhaled monobactams, such as aztreonam solution or nebulized aztreonam lysine; as well as aerosolized levofloxacin, ceftazidime, fosfomycin, gentamicin, vancomycin, amphotericin, capreomycin, fifampin, isoniazid, and ciproflaxin (Quon BS et al., 2014, Annals ATS 11(3):425-434.) In some embodiments, the aerosolized pharmaceutical composition of the invention is further comprises one or more antifungal agents also for aerosol delivery, such as liposomal amphotericin B.

In some embodiments, the pharmaceutical composition of the invention is formulated for use in treating and/or preventing bacterial infections caused by Klebsiella species, such as K. pneumonae. In some such embodiments, the pharmaceutical composition comprises a cocktail composition comprising one or more purified bacteriophage of F17/19 or F58/19, and optionally F391/08, or variants thereof, and in an embodiment, comprises or consists of F17/19, F58/19 and F391/08, or variants thereof. The composition may further comprise or consist of purified bacteriophage F99/10, F27/12 and F95/13 or variants thereof with activity against P. aeruginosa. In some embodiments, the pharmaceutical composition may further comprise an additional agent, e.g., an antibiotic agent having antibacterial activity against K. pneumoniae and/or P. aeruginosa and/or an antibiotic agent having antibacterial activity against bacteria other than K. pneumoniae or P. aeruginosa. In some embodiments, the composition is formulated in a dosage form in which the bacteriophage is present in an amount to provide a multiplicity of infection (MOI) of about 1 to about 10 upon administration of the composition to a subject in need thereof In certain embodiments, each phage is administered at a dosage of 10⁹ pfu to 10¹⁰ pfu and for, example, is present at 10⁹ pfu/ml in the composition In preferred embodiments, the composition is formulated for administration as an aerosol.

Pharmaceutical compositions comprising the purified bacteriophage cocktail of the present invention can be formulated in a unit dose or multi-dose formulation. Preferred formulations are formulations that can be delivered as an aerosol, as discussed above. Other suitable formulations include a suspension, emulsion, lotion, solution, cream, ointment, or dusting powder, or in a skin patch.

In addition or alternatively, the pharmaceutical compositions provided herein can be administered in the form of a suppository or pessary, orally (e.g., as a tablet, which may contain excipients such as starch or lactose, as a capsule, ovule, elixir, solution, or suspension, each optionally containing flavoring, coloring agents, and/or excipients), or they can be injected parenterally (e.g., intravenously, intramuscularly, or subcutaneously). For parenteral administration, the compositions may be used in the form of a sterile aqueous solution which may contain other substances, for example enough salts or monosaccharides to make the solution isotonic with blood. For buccal or sublingual administration, the compositions may be administered in the form of tablets or lozenges, which can be formulated in a conventional manner. Topical formulations generally include a sterile buffer, such as a sterile PBS, water, or saline buffer, or a sterile SM buffer.

Modes of administration described herein and/or known in the art may be used to deliver desired dosages of the phages, phage products, and/or phage cocktails of the invention and in accordance with suitable dosage regimens. Dosages and dosage regimens may vary depending on the particular formulation, route of administration, condition being treated, and other factors. Animal experiments can provide reliable guidance for the determination of effective doses in human therapy, e.g., as within the skill of the ordinary physician. Interspecies scaling of effective doses can be performed by one of ordinary skill in the art following the principles described, e.g., by Mordenti, J. et al. “The use of interspecies scaling in toxicokinetics” in Toxicokinetics and New Drug Development, Yacobi et al., Eds., Pergamon Press, New York 1989, pp 42-96. For example, a murine model of acute pulmonary infection can be used to evaluate efficacy of pharmaceutical compositions of the invention, as detailed in the Examples below.

The pharmaceutical compositions of the invention can be administered according to a dosage regimen. In some embodiments, the dosage regime involves administration of a cocktail composition of the invention every 6 or 8 hours (for example, a multiple dosing regimen for a topical phage cocktail on diabetic cutaneous wounds (Mendes J J, et al., 2013, Wound Repair Regen 21:595-603)). In preferred embodiments, initial administration is followed by a second or “booster” dose, involving re-administration of the pharmaceutical composition. For example, the booster may follow an initial dose after about 1 hour, 2 hours, 3 hours, 4, hours, 5, hours, 6 hours, 7 hours, 8 hours, 10 hours, 12 hours, 1 day, or 2 days. In preferred embodiments, e.g., in treating respiratory or pulmonary infections, including but not limited to hospital-acquired bacterial pneumonia (HABP), a booster dose is use about 4, about 5, about 6 hours or about 8 hours after the initial dose.

### 5.4 THERAPEUTIC USE

Another aspect relates to the use of the purified bacteriophages and bacteriophage cocktails in pharmaceutical compositions for preventing, reducing the incidence of and/or treating bacterial infections. Phage present great potential for treating bacterial infections, due to their specificity and effectiveness in lysing pathogenic bacteria, including those associated with multidrug resistance (Larche J, et al., 2012, Antimicrob Agents Chemother 56(12):6175-6180), their potential efficiency against bacteria in biofilms (Phee A et al., 2013, J Endod 39(3):364-369); their lack of pathogenicity towards human and animal cells (Abedon ST et al., 2011, Bacteriophage 1(2):66-85), and their activity in microaerophilic environments even with high bacterial load (Azeredo J, et al. 2008. Curr Pharm Biotechnol 9:261-266). Phage cocktails in particular can provide additional advantages over the use of individual phages, e.g., to increase lytic activity against a particular bacterial strain, to increase host range, and/or to decrease the possibility of bacterial resistance emerging to an individual bacteriophage. Indeed, different bacteriophage are mixed as cocktails to broaden their properties, preferably resulting in a collectively greater antibacterial spectrum, such as an expanded host range, which makes development of resistance less likely in the subject receiving the agent.

In specific embodiments, the subject receiving a pharmaceutical composition of the invention is a mammal (e.g., bovine, ovine, caprine, equid, primate (e.g., human), rodent, lagomorph or avian (e.g., chicken, duck, goose)). In preferred embodiments, the subject receiving a pharmaceutical composition of the invention is a human, and particularly a patient that suffers from or is at risk of suffering from respiratory or pulmonary infections, including hospital-acquired bacterial pneumonia (HABP), ventilator acquired pneumonia (VAP) and health care associated pneumonia (HCAP) or cystic fibrosis-associated infection. In certain embodiments, the infection is caused by MDR, XDR or PDR infectious agents. In certain embodiments, the infection is refractory to one or more non-phage antibiotic treatments.

In preferred embodiments, pharmaceutical compositions of the invention have activity against a plurality of bacterial strains. In some preferred embodiments, the pharmaceutical composition comprises a phage cocktail combination having activity against a plurality of strains of P. aeruginosa and/or K. pneumonae. Accordingly, the invention provides methods of treating and/or preventing infections associated with P. aeruginosa and/or K. pneumonae in both humans and animals using a phage, phage product, or phage cocktail composition of the invention. In other aspects, the invention provides methods of treating and/or preventing infections associated with related species or strains of these bacteria.

P. aeruginosa and K. pneumonae are responsible for many severe opportunistic infections, particularly in individuals with compromised immune systems. The pharmaceutical compositions of the present invention are contemplated for treating and/or preventing any infection associated with P. aeruginosa and/or K. pneumonae, or associated with other species or strains of bacteria, including, but not limited to, infections of the lungs and respiratory tract, post-operative infections, infections associated with catheters and surgical drains, and infections of the blood. In preferred embodiments, the pharmaceutical compositions of the invention find use in treating and/or preventing and/or reducing the risk or incidence of or the severity of bacterial infections associated with the lungs and respiratory tract.

Respiratory and pulmonary infections include, but are not limited to, infections associated with cystic fibrosis, such as cystic fibrosis bronchiectasis; pneumonia, including hospital-acquired bacterial pneumonia, ventilator-associated pneumonia, and bronchopneumonia; non-cystic fibrosis bronchiectasis; bronchitis; chronic obstructive pulmonary disease; mycobacterial disease, post-lung transplant infection; infections associated with tuberculosis; empyema with thoracic fistula; pleuritis with fistula, lung abscesses; rhinitis; purulent cysts; and lung-derived septicemia. Symptoms of respiratory or pulmonary infections include, e.g., cough, wheezing, production of sputum, dyspnea (difficulty breathing), dysphonia (difficulty speaking), and overall decreased quality of life. In particularly preferred embodiments, the respiratory or pulmonary infection is hospital-acquired bacterial pneumonia (HABP).

Regarding HABP, the time of onset during hospitalization is an indicator of risk for specific pathogens and outcomes. With early onset, e.g., within the first 4 days of hospitalization, the most frequent agents are endogenous microbiota like Streptococcus pneumonia and Haemophilus influenzae, as well as Gram negative and community S. aureus sensitive to antibiotics. With late onset, e.g., onset occurring more than 5 days after being hospitalized, gram-negative bacteria account for the majority of cases, many of which are resistant to antibiotics, such as certain strains of P. aeruginosa, Klebsiella pneumonia, Enterobacter spp., and Acinetobacter spp., as well as certain S. aureus infections, particularly those in neurosurgical patients, diabetics, and patients with chronic renal problems (2005, Am J Respir Crit Care Med Vol 171(4):388:416). Strains of P. aeruginosa, K. pneumonia are especially relevant to late-onset HABP.

K. pneumonae and P. aeruginosa and also are associated with infections that involve other organ systems that have a high fluid content, and it is contemplated that the phage cocktails of the invention have therapeutic and/or prophylactic use with respect to, including to reduce the risk or incidence of, such infections. For example, the pharmaceutical compositions of the invention may be used for the prevention or treatment of infections of the cerebrospinal fluid, of peritoneal fluid, and of the urinary tract.

In some embodiments, the invention provides methods of treating and/or preventing and/or reducing the incidence or severity of respiratory or pulmonary infection, comprising administering to a subject in need thereof a therapeutically or prophylactically effective amount of a pharmaceutical composition of the phage cocktail provided herein. In particular embodiments, the pharmaceutical compositions comprises or consists of one, two or all three of F391/08, F17/19 and F58/19. In preferred embodiments, administration results in an improvement in breathing, e.g., returning labored or rapid breathing to normal.

In a particularly preferred embodiment, the invention provides methods of treating a surprising range of K. pneumonae and, in certain embodiments, P. aeruginosa bacterial strains, using a phage cocktail of the invention. For example, a phage cocktail comprising or consisting of the K. pneumonae phages F391/08, F17/19 and F58/19 shows efficacy against a highly diverse range of K. pneumonae clinical strains (53% infection), e.g., when compared to homologous K. pneumonae phage. In other embodiments, the phage cocktail further comprising or consisting of the P. aeruginosa phages F99/10, F27/12 and F95/13 shows efficacy against a highly diverse range of P. aeruginosa strains clinical strains, presenting varied capsular serotype (57%), e.g., when compared to certain other P. aeruginosa phages.

In preferred embodiments, administration comprises administration of the pharmaceutical composition via an aerosol into one or more airways of the subject, e.g., administration by inhalation. Administration by inhalation can improve drug delivery to the target site of infection (i.e., the airways) and/or limit potential for systemic side effects. Administration of the pharmaceutical composition as an aerosol includes, but is not limited to, administration by inhalation, intranasal instillation, catheterization of the trachea, delivery to the pleural cavity of the lungs, or bronchoscopy (Abedon ST, 2015, Bacteriophage, 5(1):e1020260-1 to e1020260-13). During administration of the pharmaceutical composition as an aerosol, the bacteriophage remain viable and may be contained in particles of suitable size to reach the lower airways. For example, in particularly preferred embodiments, the majority of aerosolized particles are less than 5 μm in diameter, e.g., at least 50%, 60%, 70%, or 80% of the particles are less than 5 μm in diameter, and more preferably are about 2 μm in diameter.

For intranasal administration or administration by inhalation, the bacteriophage and/or phage product of the invention may be delivered in the form of a dry powder, fine particles, nanoparticles, solutions, lyophilized preparations, liposomal preparations, and the like. Typically the formulation comprising the phage, phage product, and/or phage cocktail of the invention is in the form of a dry powder inhaler or an aerosol spray delivered from a pressurized container, pump, spray, or nebulizer with the use of a suitable propellant, e.g., dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (FIFA 227EA™), carbon dioxide, or other suitable gas.

In the case of a pressurized aerosol, the dosage unit may be determined by providing a valve to deliver a metered amount. The pressurized container, pump, spray, or nebulizer may contain a solution or suspension of the active compound, e.g., using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of the phage, phage product, and/or phage cocktail of the invention and a suitable powder base such as lactose or starch.

Nebulization may be achieved using any known means in the art or as described herein. Typically, nebulization is achieved by jet nebulizers, which use air or oxygen under high pressure to generate the aerosol. Other nebulizers include vibrating mesh nebulizers, driven by piezoelectric actuators, which reduce size variability and reduce nebulization time (see, for example, Aeroneb, Nebutec 4, Nebutec 6). Another approach comprises mechanical ventilation, where the nebulizer is connected to the inspiratory limb of the ventilator circuit. Still another approach uses emulsion-based “spray-drying” to transform a solution or emulsion from a fluid state into fine particles with uniform size distribution (about 1-5 μm). Two typical classes of nebulizers include the AeroEclips (Trudell Medical International), a jet nebulizer where nebulization only occurs when the patient inhales; and the Omron (Omron, MicroAir U22), a battery-powered mesh nebulizer that relies on the vibration of a piezoelectirc crystal to force the agents through a fine mesh, creating an aerosol (Sahota et al., 2015, J. Aerosol Medicine and Pulmonary Drug Delivery 28(0): 1-8). In some preferred embodiments, a SYSTAM L290 (SYSTAM, Villeneuve Sur Lot, France) nebulizer is used. This nebulizer produces an ultrasonic aerosol where about 70% of the particles are less than 5 μm diameter.

Pharmaceutical compositions of the invention will comprise a therapeutically and/or prophylactically effective amount of one of more phages or phage products, as described herein. A therapeutically and/or prophylactically effective amount refers to an amount required to bring about a therapeutic and/or prophylactic benefit, respectively, in a subject receiving said amount. A therapeutically and/or prophylactically effective amount will depend on the particular formulation, route of administration, condition being treated, whether other agents or therapies are used in combination with methods of the invention, and other factors.

In some embodiments, the pharmaceutical composition is delivered to a subject in need thereof so as to provide one or more bacteriophage in an amount corresponding to a multiplicity of infection (MOI) of about 1 to about 10. MOI is determined by assessing the approximate bacterial load in the lungs, or calculating the bacterial load in the lungs of a particular patient, or using an estimate for a given type of respiratory infection; and then providing phage in an amount calculated to give the desired MOI (e.g., 2×10⁷ pfu/g of lung gives a MOI of 10). MOI may be selected based on the “multiplicity of 10 rule,” which states that where there are on average in order of 10 phages adsorbed per bacterium, bacterial density reduces significantly (Abedon ST, 2009, Foodborne Pathog Dis 6:807-815; and Kasman L M, et al., 2002, J Virol 76:5557-5564); whereas lower-titer phage administration (e.g., using a MOI lower than 10) is unlikely to be successful (Goode D, et al., 2003, App Environ Microbiol 69:5032-5036; Kumari S, et al., 2010, J Infect Dev Ctries 4:367-377).

In some preferred embodiments, a phage cocktail comprising F391/08, F17/19, and F58/19 is delivered to provide a MOI between 1 and 10 of each phage, results in a decrease in K. pneumonae in the lungs by about 80%, about 85%, about 95%, about 97%, about 98%, or by as much as about 100% (where viable cell count decreases to zero). In some preferred embodiments, the phage cocktail further comprising (or administering in combination with) purified bacteriophage F99/10, F27/12, and F95/13, delivered to provide a MOI between 1 and 10 of each phage, results in a decrease in P. aeruginosa in the lungs by about 80%, about 85%, about 95%, about 97%, about 98%, or by as much as about 100%. In some particularly preferred embodiments, the phage cocktail surprisingly shows synergistic bacteriolytic action as compared to each bacteriophage on its own.

In some embodiments, lower doses surprisingly may provide advantages over higher doses. For example, in some embodiments, a MOI at or about 1 maintains low levels of bacteria in the lungs for longer periods of time than a MOI at or about 10. For example, lower MOI's F391/08, F17/19, and F58/19 may achieve lower K. pneumonae load, and lower MOI's of F99/10, F110/10, and/or F27/12 may achieve lower P. aeruginosa load, in the lungs of infected animals for longer periods of time post-treatment, e.g., for 12 hours, 15 hours, 18 hours, 24 hours, 30 hours, 36 hours, or longer post-treatment. Without wishing to be bound by theory, this may be due to delay in the appearance of bacterial resistance in response to the lower doses of phage.

In certain embodiments, each phage is administered at a dosage of 10⁸ to 10¹¹, including 10⁹ pfu to 10¹⁰ pfu and for, example, is present at 10⁸ to 10¹¹, including 10⁹ or 10¹⁰ pfu/ml in the composition. In certain embodiments, each phage is present in approximately the same amount, including a ratio of 1:1:1 in the composition or dosage form.

In some other embodiments, a MOI as low as about 0.2 to 0.4 may result in efficacy, e.g., statistically significant reductions in K. pneumonae and/or P. aeruginosa load in the lungs of infected animals. Without being bound to a particular theory, efficacy may be due to active therapy. That is, phage doses at a MOI of 10 provide phage sufficiently in excess of the target bacteria population to reduce bacterial load without the need for phage replication or life cycle completion. Lower phage doses may rely on active therapy, which involves phage infection/replication cycles to reduce the target bacterium (Loc Carrillo C, et al., 2005, Appl Environ Microbiol 71:6554-6563; see also Cairns B J, et al., 2009, PLoS Pathog 5:e1000253; and Hooton S P, et al., 2011, Int J Food Microbiol 151:157-163).

In certain embodiments, a purified bacteriophage or bacteriophage cocktail composition of the invention is used as a single agent for treating or preventing infections caused by P. aeruginosa and/or K. pneumonae, such as respiratory or pulmonary infections. In other embodiments, a bacteriophage or bacteriophage cocktail disclosed herein is used in further combination with other agents, including standard antibiotics that target the same or different kinds of bacteria, including bacteria selected from any gram-positive bacteria, any gram-negative bacteria, and any other groups of bacteria that is not classified as gram-positive or gram-negative. The compositions of the invention may also be used in combination with any other means of treating bacterial infection known to one of skill in the art, in particular, any other means of treating respiratory infections.

In some particularly preferred embodiments, the invention provides methods of treating and/or preventing respiratory or pulmonary infections comprising administering a phage cocktail of the invention in combination with a standard and/or non-standard therapy. Standard therapies for respiratory infections includes inhalation and/systemic administration of antibiotic agents such as tobramycin, amikacin, colistin, aztreonam, as well as levofloxacin, ceftazidime, fosfomycin, gentamicin, vancomycin, amphotericin, capreomycin, fifampin, isoniazid, and ciproflaxin; and inhalation and/or systemic administration of antifungal agents such as amphotericin B.

In some embodiments, the phage, phage product, or phage cocktail composition of the invention is administered as an aerosol, while an additional agent is administered systemically. For example, in some preferred embodiments, a phage cocktail composition of the invention is administered by inhalation while an antibiotic agent is administered systemically, such as an antibiotic agent having activity against P. aeruginosa and/or K. pneumonea. In some embodiments, the phage cocktail composition of the invention is administered via inhalation along with an additional agent that also is administered as an aerosol. For example, in some preferred embodiments, the phage cocktail pharmaceutical composition of the invention is administered along with another antibiotic agent or an antifungal agent as an aerosol into the lungs.

In some embodiments, the invention provides methods of treating and/or preventing respiratory or pulmonary infections comprising administering a phage, phage product, or phage cocktail composition of the invention in combination with a non-standard therapy for respiratory infections. Non-standard therapies generally are used where the respiratory infection is refractory to one or more standard therapies.

### 5.5 DISINFECTANT AND ANTI-INFECTIVE USE

Bacterial pathogens most often infect at mucus membranes (e.g., through mucus membranes of the upper or lower respiratory tract, though the urogenital system, ocular structures, and the like). The mucus membranes themselves are often a reservoir, sometimes the only reservoir, for pathogenic bacteria found in the environment. There are very few anti-infectives designed to control this reservoir for pathogenic bacteria, though studies have shown that reducing or eliminating this reservoir, especially in environments such as hospitals and nursing homes, markedly reduces the incidence of infections.

The phages, phage products, and phage cocktails of the invention can be used in anti-infective compositions for controlling the growth of bacteria, in particular K. pneumoniae and P. aeruginosa, in order to prevent or reduce the incidence of nocosomial infections. The anti-infective compositions find use in reducing or inhibiting colonization or growth of bacterial on a surface contacted therewith. The phages, phage products, and phage cocktails of the invention may be incorporated into compositions that are formulated for application to biological surfaces, such as the skin and mucus membranes, as well as for application to non-biological surfaces.

Anti-infective formulations for use on biological surfaces include, but are not limited to, gels, creams, ointments, sprays, and the like. In particular embodiments, the anti-infective formulation is used to sterilize a surgical field, or the hands and/or exposed skin of healthcare workers and/or patients. In preferred embodiments, the biological surface is a mucus membrane of a mammal, more preferably, the mucus membrane of a human. In particularly preferred embodiments, the biological surface is a mucus membrane of the respiratory tract, such as the nasal mucosa, linings of the pharynx, larynx, trachea, bronchi, and/or lungs.

Anti-infective formulations for use on non-biological surfaces include sprays, solutions, suspensions, wipes impregnated with a solution or suspension, and the like. In particular embodiments, the anti-infective formulation is used on solid surfaces in hospitals, nursing homes, ambulances, etc., including, e.g., appliances, countertops, and medical devices, hospital equipment. In preferred embodiments, the non-biological surface is a surface of a hospital apparatus or piece of hospital equipment. In particularly preferred embodiments, the non-biological surface is a surgical apparatus or piece of surgical equipment.

### 5.6 DIAGNOSTIC METHODS

The present invention also encompasses diagnostic methods for determining the causative agent in a bacterial infection. In certain embodiments, the diagnosis of the causative agent of a bacterial infection is performed by (i) culturing a sample from a patient, e.g., a swab, sputum, or other sample appropriate for culturing the bacteria causing the infection; (ii) contacting the culture with one or more phages, phage products, and phage cocktails of the invention; and (iii) monitoring for evidence of cell growth and/or lysis of the culture. Because the activity of phages and/or their isolated products (e.g., polypeptides, biologically active fragments or variants thereof, or nucleic acids encoding same) tends to be species or strain specific, susceptibility, or lack of susceptibility, to one or more phages, phage products, and phage cocktails of the invention can indicate the species or strain of bacteria causing the infection.

In some embodiments, a test culture is obtained from a patient and contacted with one or more of F391/08, F17/19, or F58/19, or a variant thereof. Decreased growth and/or lysis of the culture can indicate that the test sample comprises K. pneumonae, in particular, a strain of K. pneumonae susceptible to infection by the bacteriophage or bacteriophage cocktail used, as disclosed herein, thereby identifying the infective agent and allowing appropriate diagnosis and/or treatment.

The sample may be a tissue biopsy or swab collected from the patient, or a fluid sample, such as blood, tears, or urine. In preferred embodiments, the tissue sample is obtained from the respiratory tract of the patient, e.g., a mucus sample, sputum, or a swab from a nostril.

### 6. EXAMPLES

It is understood that the following examples and embodiments described herein are for illustrative purposes only and that various modifications or changes in light thereof will be suggested to persons skilled in the art and are to be included within the spirit and purview of this application and the scope of the appended claims.

Unless otherwise indicated, specific bacteriophage disclosed herein were isolated, processed and analyzed according to the following methods. Further, the study described below was approved locally by the Animal Ethics Committee of the Instituto de Medicina Molecular and approved nationally by the Portuguese General Directorate of Veterinary Services (Direcção Geral de Veterinária), in accordance with Portuguese law. All animals in the study were maintained in accordance with European Directive 86/609/EC (Council of the European Communities. Council Directive 86/609/EEC of 24 Nov. 1986 on the approximation of laws, regulations and administrative provisions of the Member States regarding the protection of animals used for experimental and other scientific purposes. Off J Eur Communities L358:1-28), Portuguese law (Portaria 1005/92) (Portuguese Agricultural Ministry. Portaria no. 1005/92 of 23 October on the protection of animals used for experimental and other scientific purposes. Diário da República I—Série B 245:4930-4942), and the Guide for the Care and Use of Laboratory Animals (NRC 2011) (Institute for Laboratory Animal Research. 2011. Guide for the care and use of laboratory animals. Washington (DC): National Academies Press.).

One aim of this study was to investigate the antimicrobial activity of a nebulized bacteriophage cocktail against Pseudomonas aeruginosa and Klebsiella pneumoniae in a murine experimental model of acute pulmonary infection. Nebulization of aerosolized bacteriophage allowed delivery directly to affected lung areas, overcoming certain side effects of nebulized antibiotics.

### 6.1. EXAMPLE 1

### Bacterial Strains

A selected group of K. pneumoniae bacterial isolates were collected between 2005 and 2019 (n=36), in at least 7 different health care facilities. Overall, these isolates were collected from hospital settings (n=25), outpatients (n=9), or from unknown origin (n=2), and from a diversity of biological products [urine (n=10), respiratory secretions (n=5), unknown (n=8), abdominal fluids (n=5) and others (n=8)]. This panel was evaluated with regards to antibiotic susceptibility testing by disk diffusion method against a selected panel of clinically important antibiotics (β-lactam antibiotics—ampicillin, ceftazidime, piperacillin with tazobactam, and meropenem; fluoroquinolones—ciprofloxacin; aminoglycosides—gentamicin; and sulphonamides—trimethoprim with sulfamethoxazole). Interpretation of results was performed according to the cut-off values recommended by the European Committee on Antimicrobial Susceptibility Testing (EUCAST, http://mic.eucast.org/Eucast2/). Isolates were considered multidrug resistant (MDR) when presenting non-susceptibility to three or more structurally unrelated classes of antibiotics (Magiorakos A P, Srinivasan A, Carey R B., Carmeli Y, Falagas M E, et al. 2011. Clin Microbiol Infect 2012; 18: 268-281).

This set of K. pneumoniae bacterial isolates (n=36) was also characterized by MLST, as previously reported to assess genetic diversity, clonal group and worldwide prevalence. The resulting sequences were then analyzed with Finch TV and assigned to respective STs using the tools available on the MLST website (https://pubmlst.org) Each bacterial isolate was streaked onto tryptone soy agar media plates (TSA, Biokar Diagnostics, Pantin Cedex, France) incubated at +37° C. for 18 h. All clinical strains were stored in tryptone soy broth (TSB, Biokar Diagnostics, Pantin Cedex, France) with 15% glycerol (w/v) at −70° C. until needed.

For in vitro experiments cryopreserved strains at −70° C. were grown overnight on TSA at 37° C. for 18h. Single colonies were posteriorly grown in TSB during overnight at 37° C. with agitation. A new bacterial suspension (a dilution of the overnight culture) was prepared and incubated at 37° C. with agitation. Bacteria were harvested when reached the exponential growth phase (optical density at 600 nm 0.3-0.5). An inoculum of approximately 2.0×10⁶ cfu/mL was used in the lysis curves.

### 6.2. EXAMPLE 2

### Bacteriophage Origin, Amplification, and Cocktail

### 6.2.1 Bacteriophage Origin

Klebsiella pneumoniae F391/08, Kle_F17/19 and Kle_F58/19 virulent bacteriophages were isolated from sewage water from the Lisbon area and amplified in K. pneumoniae 397/07 (F391/08), K. pneumoniae 237/14 (Kle_F17/19) and K. pneumoniae 57/17 (Kle_F58/19) clinical strains.

To isolate lytic bacteriophages against K. pneumoniae several clinical strains were used. Sewage water from different origins of the Lisbon urban area were tested to determine the presence of bacteriophages by the ability to infect K. pneumoniae clinical strains by double agar overlay plaque assay (Kropinski A, Mazzocco A, Waddell T E, Lingohr E, Johnson RP. 2009. Enumeration of bacteriophages by double agar overlay plaque assay. Methods Mol Biol 501:69-76.).

Briefly, the bacterial strains were grown overnight in TSB at +37° C. with agitation. A new bacterial suspension (dilution of the overnight culture) was prepared, incubated at +37° C. with agitation and harvested when reached the exponential growth phase (optical density at 600 nm 0.3-0.5). Each culture was added to water sample and the mixture was incubated at +37° C. during 30 minutes to which was added 3 ml of soft agar 0.7% pre-equilibrated. The agar-water-bacterial suspension was overlaid onto TSA plates 1.5%, allowed to solidify at room temperature and incubated at +37° C. After 18 hours' incubation the plates were checked for the presence of phage plaques (clearing zones) within the bacterial lawn, indicating the presence of bacteriophages. Bacteriophage plaques were picked using sterile pipette tips, transferred to SM buffer and stored at +4° C.

### 6.2.2 Bacteriophage Amplification

New isolated bacteriophages were subject to a process of propagation, amplification, purification (3 consecutive elutions) in the indicator strains, before the evaluation of the host range. Susceptibility of 36 K. pneumoniae bacterial isolates to a particular bacteriophage infection was performed using the double agar overlay plaque assay. The sensibility of 36 bacterial isolates against a particular bacteriophage was determined by the observation of phage plaques within the bacterial lawn. The bacteriophages with the highest percentage of infection in host range were selected and proceed to a new process of amplification, concentration by high speed centrifugation, purification in cesium chloride (CsCl) gradient, extraction of bacteriophage genomic DNA and restriction fragment length polymorphism analysis.

The phages with distinct restriction profiles and broader host range were selected for sequencing. Complete genome sequences were determined by pyro sequencing using the Illumina HiSeq2000 genome analyzer.

After bioinformatics analysis the most promising bacteriophages were selected for the composition of a therapeutic cocktail. The morphology of K. pneumoniae F391/08, Kle_F17/19 and Kle_F58/19 bacteriophages was analyzed at the Histology and Comparative Pathology Laboratory of Institute of Molecular Medicine, Lisbon, Portugal. These data were integrated with the genomic analysis, and these bacteriophages were classified according to the Ackermann classification (Ackermann HW. 2009. Phage classification and characterization. Methods Mol Biol 501:127-140.).

### 6.3. EXAMPLE 3

### Bacteriophage Analysis

### 6.3.1 Phenotypic Characterization

Phenotypic characterization of the collected bacteria showed that among β-lactam antibiotics, 100% of the isolates were non-susceptible to ampicillin, 97.2% was non-susceptible to piperacillin plus tazobactam, 86.1% were non-susceptible to ceftazidime, and 77.8% were non susceptible to meropenem. Regarding non β-lactam antibiotics, this group of bacterial isolates showed 91.7% of non-susceptibility to ciprofloxacin, 72.2% showed non-susceptibility to gentamicin, and 88.9% showed non-susceptibility to trimethoprim with sulfamethoxazole. Overall, 33/36 (91.7%) of the isolates were MDR (Table 3).

TABLE 3
Infection by K. pneumoniae phages, phenotypic, and genotypic characterization
of the selected subset of bacterial isolates (n = 36).

| Strain | Year | Infection | MDR | AR profile | Carbapenemase | ST |
|---|---|---|---|---|---|---|
| 1633 | 05 | F391/08 | + | AMP; TZP; CZD; | — | 35 |
| | | | | CIP; CN; SXT | | |
| 397 | 07 | F391/08 | − | AMP; TZP; CIP | — | New |
| | | | | | | ST |
| 488 | 07 | No infection | + | AMP; TZP; CIP; CN; | — | 1822 |
| | | | | SXT | | |
| 236 | 14 | F391/08 | + | AMP; TZP; CZD; | — | 423 |
| | | | | CIP; CN; SXT | | |
| 237 | 14 | F17/19 | − | AMP; CAZ; CN | — | 66 |
| 241 | 14 | F17/19 | − | AMP; TZP; CIP | — | 449 |
| 17 | 17 | No infection | + | AMP; TZP; CZD; | — | 11 |
| | | | | MEM; CIP; SXT | | |
| 24 | 17 | F58/19 | + | AMP; TZP; CZD; | — | 11 |
| | | | | MEM; CIP | | |
| 26 | 17 | F58/19; | + | AMP; TZP; CZD; | — | 15 |
| | | F391/08 | | MEM; CIP; CN; SXT | | |
| 28 | 17 | No infection | + | AMP; TZP; CZD; | — | 17 |
| | | | | MEM; CIP; CN; SXT | | |
| 29 | 17 | F17/19 | + | AMP; TZP; CZD; | — | 449 |
| | | | | MEM; CIP; SXT | | |
| 30 | 17 | F391/08 | + | AMP; TZP; CZD; | — | 13 |
| | | | | MEM; CIP; CN; SXT | | |
| 41 | 17 | F58/19 | + | AMP; TZP; CZD; | KPC, SHV | 258 |
| | | | | MEM; CIP; SXT | | |
| 43 | 17 | F391/08 | + | AMP; TZP; CIP; | — | 147 |
| | | | | SXT | | |
| 57 | 17 | F58/19; | + | AMP; TZP; CZD; | — | 15 |
| | | F391/08 | | MEM; CIP; CN; SXT | | |
| 63 | 17 | F58/19 | + | AMP; TZP; CZD; | — | 348 |
| | | | | MEM; CIP; CN; | | |
| | | | | SXT | | |
| 05 | 18 | F58/19 | + | AMP; TZP; CZD; | — | 348 |
| | | | | MEM; CIP; CN; SXT | | |
| 82 | 18 | No infection | + | AMP; TZP; CZD; | KPC | 258 |
| | | | | MEM; CIP; SXT | | |
| 127 | 18 | F17/19 | + | AMP; TZP; CIP; CN; | — | 14 |
| | | | | SXT | | |
| 128 | 18 | No infection | + | AMP; TZP; CZD; | — | 231 |
| | | | | MEM; CIP; CN; SXT | | |
| 6 | 19 | F58/19 | + | AMP; TZP; CZD; | — | 134 |
| | | No infection | | MEM; CIP; CN; SXT | | |
| 9 | 19 | No infection | + | AMP; TZP; CZD; | — | 48 |
| | | | | MEM; CIP; CN; SXT | | |
| 13 | 19 | No infection | + | AMP; TZP; CZD; | OXA, CTX-M | 101 |
| | | | | MEM; CIP; CN; SXT | | |
| 14 | 19 | No infection | + | AMP; TZP; CZD; | KPC | 113 |
| | | | | MEM; CIP; CN; SXT | | |
| 15 | 19 | No infection | + | AMP; TZP; CZD; | KPC | 258 |
| | | | | MEM; CIP; CN; SXT | | |
| 16 | 19 | No infection | + | AMP; TZP; CZD; | KPC | 258 |
| | | | | MEM; CIP; CN; SXT | | |
| 17 | 19 | F17/19; | + | AMP; TZP; CZD; | KPC | 14 |
| | | F391/08 | | MEM; CIP; CN; SXT | | |
| 18 | 19 | No infection | + | AMP; TZP; CZD; | KPC | 258 |
| | | | | MEM; CIP; CN; SXT | | |
| 20 | 19 | F58/19 | + | AMP; TZP; CZD; | KPC | 1272 |
| | | | | MEM; CIP; CN; SXT | | |
| 23 | 19 | No infection | + | AMP; TZP; CZD; | KPC | 258 |
| | | | | MEM; CIP; CN; SXT | | |
| 26 | 19 | No infection | + | AMP; TZP; CZD; | KPC | NA |
| | | | | MEM; CIP; SXT | | |
| 27 | 19 | No infection | + | AMP; TZP; CZD; | NDM | NA |
| | | | | MEM; CIP; CN; SXT | | |
| 28 | 19 | No infection | + | AMP; TZP; CZD; | NDM | NA |
| | | | | MEM; CIP; SXT | | |
| 29 | 19 | F58/19 | + | AMP; TZP; CZD; | KPC, VIM | NA |
| | | | | MEM; CIP; CN; SXT | | |
| 30 | 19 | No infection | + | AMP; TZP; CZD; | VIM | NA |
| | | | | MEM; CIP; CN; SXT | | |
| 31 | 19 | No infection | + | AMP; TZP; CZD; | KPC | NA |
| | | | | MEM; CIP; CN; SXT | | |

Additionally, interpretative reading of the antibiotic susceptibility testing suggested the production of an extended-spectrum β-lactamases (ESBL) or carbapenemases (Table 1). Overall, the majority of the isolates presented non-susceptibility to last resource antibiotics, such as carbapenems (23/30, 76.6%), MDR (n=22/30, 73.3%), and production of clinically important carbapenemases or ESBL (16/30, 53.3%), such as the worldwide spread VIM-2, KPC-2 or even NDM-1.

Identification of clonal lineages showed high genetic diversity represented through 19 different STs, and specifically with detection of high risk clones (ST11, ST13, ST14, ST15 or ST258), which show a widespread prevalence worldwide, and are in fact responsible for outbreaks and high morbidity and mortality (Brink, A. J., 2019. Epidemiology of carbapenem-resistant Gram-negative infections globally. Curr Opin Infect Dis 32, 609-616.; Karakonstantis, S., Kritsotakis, E. I., Gikas, A., 2019. Pandrug-resistant Gram-negative bacteria: a systematic review of current epidemiology, prognosis and treatment options. J Antimicrob Chemother.; Li, J., Li, Y., Song, N., Chen, Y., 2019. Risk factors for carbapenem-resistant Klebsiella pneumoniae infection: a meta-analysis. J Glob Antimicrob Resist.; Navon-Venezia, S., Kondratyeva, K., Carattoli, A., 2017. Klebsiella pneumoniae: a major worldwide source and shuttle for antibiotic resistance. FEMS Microbiol Rev 41, 252-275.; Sarda, C., Fazal, F., Rello, J., 2019. Management of ventilator-associated pneumonia (VAP) caused by resistant gram-negative bacteria: which is the best strategy to treat? Expert Rev Respir Med 13, 787-798.).

### 6.3.2 Bacteriophage Morphology

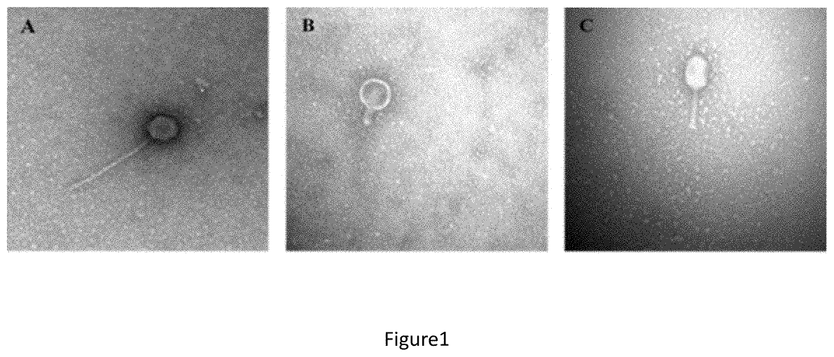

Transmission electron microscopy was used to classify the purified bacteriophages based on their virion morphology. K. pneumoniae F391/08, Kle_F17/19 and Kle_F58/19 bacteriophages belong to the order Caudovirales. F391/08 bacteriophage presented an icosahedral head with long, non-contractile, thin tail, which is often flexible. These features, along with its genomic properties allowed us to classify this bacteriophage as member of the Siphoviridae family. Kle_F17/19 and Kle_F58/19 bacteriophages were classified as members of the Myoviridae family presenting a contractile tail and icosahedral heads (capsids), with a baseplate structure and tail fibers (FIG. 1).

### 6.3.3 Bacteriophage Host Range

The Klebsiella pneumoniae selected bacteriophages were tested in 36 clinical strains isolated from human clinical samples collected and identified in hospitals from the Lisbon area. Among this panel of diverse clinically important K. pneumoniae isolates, 53.8% were infected by at least one of the phages composing the cocktail (Table 4).

TABLE 4
Percentage of infected strains in the host-range by each
of the K. pneumoniae phages and the total coverage.

| | Individual percentage of | Global percentage of |
|---|---|---|
| Phage | infection (%) | infection (%) |
| Kle_F391/08 | 22.2 | |
| Kle_F17/19 | 13.9 | 53.8 |
| Kle_F58/19 | 25.0 | |

Indeed, K. pneumoniae is an important MDR pathogen affecting humans and a major source for hospital infections associated with high morbidity and mortality due to limited treatment options. These strains may be practically resistant to all available classes of antibiotics, posing a great challenge to clinicians due to limited available treatment options (Brink, A. J., 2019. Epidemiology of carbapenem-resistant Gram-negative infections globally. Curr Opin Infect Dis 32, 609-616; Navon-Venezia, S., Kondratyeva, K., Carattoli, A., 2017. Klebsiella pneumoniae: a major worldwide source and shuttle for antibiotic resistance. FEMS Microbiol Rev 41, 252-275).

Since the first emergence of carbapenemase-producing K. pneumoniae, nearly 100 international outbreaks caused by high-risk clones were reported. The most significant outbreaks have been caused by clonal group 258, responsible for 68% of all outbreaks and consisting of three STs: ST258, ST11 and ST512. The second most prevalent clonal group, responsible for about 20% of all outbreaks was clonal group 15, consisting of ST14 and ST15. Other important STs contribute to outbreaks and clinically important infection to a lesser extent (ST147, ST37, ST101, ST17) (Navon-Venezia, S., Kondratyeva, K., Carattoli, A., 2017. Klebsiella pneumoniae: a major worldwide source and shuttle for antibiotic resistance. FEMS Microbiol Rev 41, 252-275).

Overall, infections caused by carbapenem resistant K. pneumoniae high risk clones are a major health concern and are many times responsible for high mortality rates worldwide. K. pneumoniae phages were able to effectively eliminate isolates exhibiting not only carbapenem resistant caused by acquired carbapenemases, but also belonging to five (ST11, ST14, ST15, ST258, and ST147) out the nine above mentioned high risk clones showing a worldwide prevalence.

### 6.3.4 Genomic Analysis

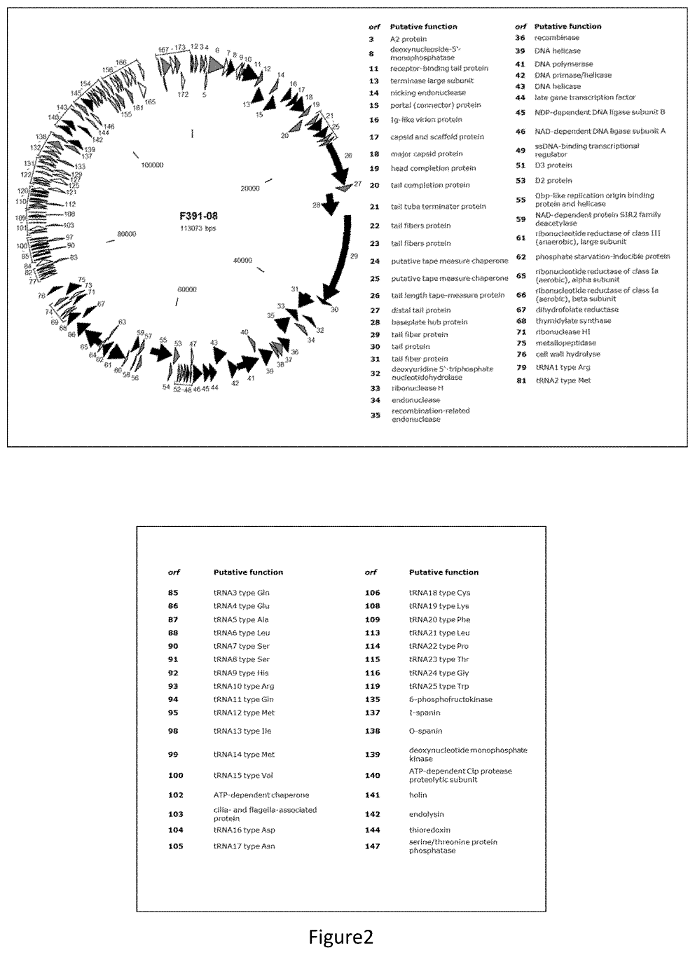

Whole-genome sequencing of K. pneumoniae F391/08, Kle_F17/19 and Kle_F58/19 bacteriophages genomic DNA was carried out. Upon annotation, a circular map for each of the genomes was prepared, indicating the predicted ORFs encoding hypothetical proteins and their putative functions (FIGS. 2-4). Kle_F391/08 has a genome of nucleotide sequence of SEQ ID NO: 1, Kle_F17/19 has a genome with a nucleotide sequence of SEQ ID NO:2, and Kle_F58/19 has a genome with a nucleotide sequence of SEQ ID NO:3. An initial NCBI nucleotide blast analysis (blastn) of the complete genome sequence of the selected bacteriophages was performed. The K. pneumoniae bacteriophage F391/08 revealed significant homology with Klebsiella phage vB_Kpn_IME260 (NCBI Reference Sequence: KX845404.2). Kle_F17/19 bacteriophage showed the highest similarity with bacteriophage Klebsiella phage KOX1 (NCBI Reference Sequence: KY780482.1). The bacteriophage Kle_F58/19 genomic sequence presented homologies with other sequences from the NCBI database. The highest similarity was found for Klebsiella phage vB_KpnM_Potts1 (NCBI Reference Sequence: MN013081.1).

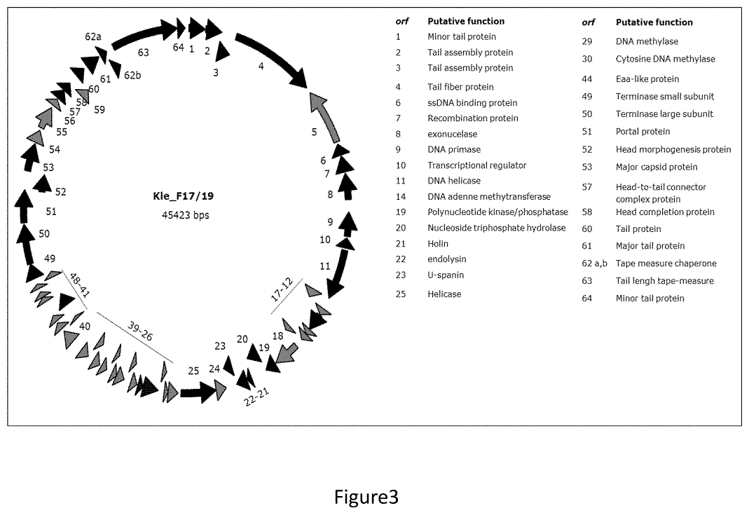

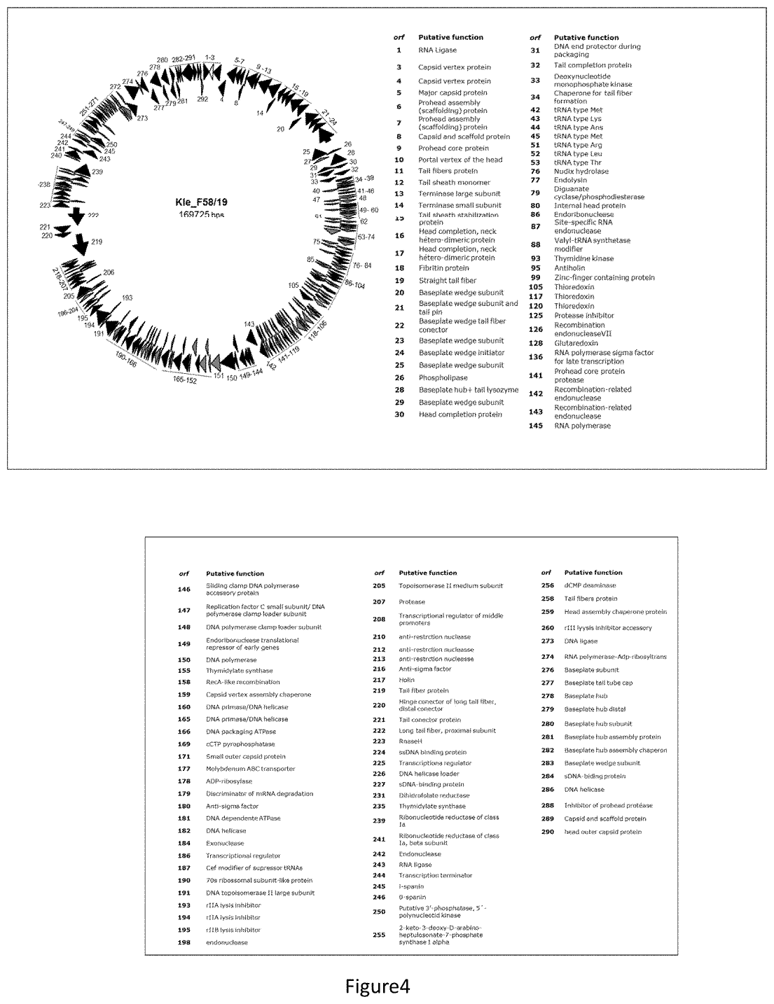

The bacteriophage F391/08 with a genome size of 113073 bp (FIG. 2), shared up to 95% sequence identity in 96% of genome coverage with Klebsiella phage vB_Kpn_IME260. One hundred seventy-three ORFs were predicted with 28% putative function assigned of which 25 were tRNA genes. One of the predicted ORFs had no significant homology with any sequence from the NCBI non-redundant protein sequence database. Ninety of the predicted ORFS presented homology with proteins from the NCBI non-redundant protein sequence database however no putative function could be assigned. Genome analysis of the Kle_F17/19 bacteriophage revealed that the 45423 bp sequence (FIG. 3) exhibited in 94% of the genome sequence identities up to 96% with the Myoviridae Klebsiella phage KOX1 (Brown T L, Petrovski S, Hoyle D, Chan H T, Lock P, Tucci J. 2017. Characterization and formulation into solid dosage forms of a novel bacteriophage lytic against Klebsiella oxytoca. PLoS ONE 12(8): e0183510). The 64 predicted ORFs presented putative function assigned in 50%. In thirty-two of the ORFs no putative function could be assigned. The bacteriophage Kle_F58/19 with a 169725 bp genome (FIG. 4) presented high similarities with the Myoviridae Klebsiella phage vB_KpnM_Potts1. The sequence identity was up to 97.5% in 98% of the genome sequence. Two hundred ninety-two ORFs were predicted with 45.9% putative function assigned within which 7 were tRNA genes. Approximate 45% of the predicted ORFs had homology with sequences from the NCBI non-redundant protein sequence database however without function assigned. Only one ORF had no significant homology with any sequence from the NCBI non-redundant protein sequence database.

No significant similarity with known virulence or toxin proteins or with elements typically associated with lysogeny (integrases, repressors, and anti-repressors) could be found in the sequences of these bacteriophages.

### 6.3.5 Lytic Activity of Bacteriophage Cocktail

The lytic activity of the new Klebsiella pneumoniae F391/08, Kle_F17/19 and Kle_F58/19 bacteriophages was evaluated against planktonic cultures in order to characterize the bacteriophage cocktail.

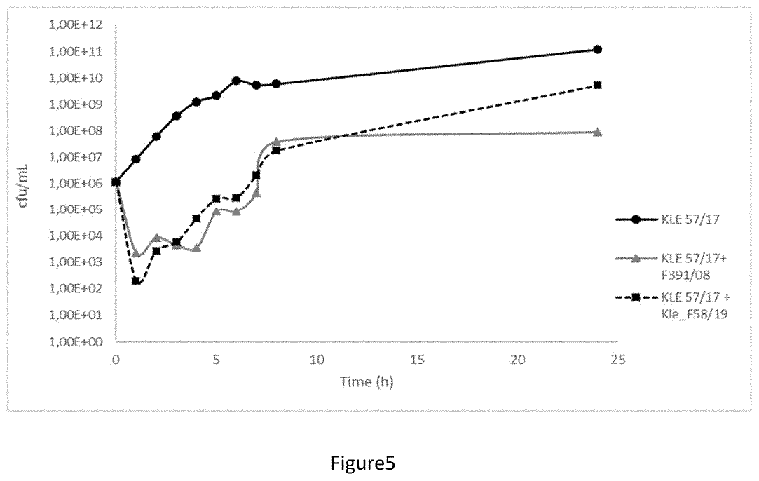

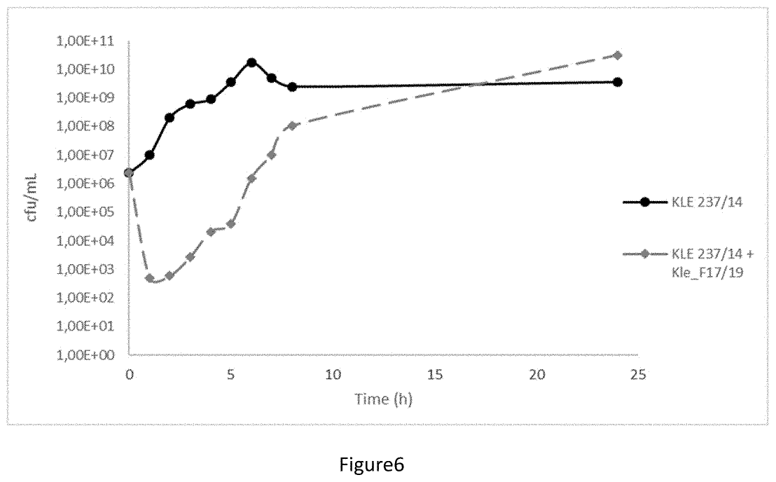

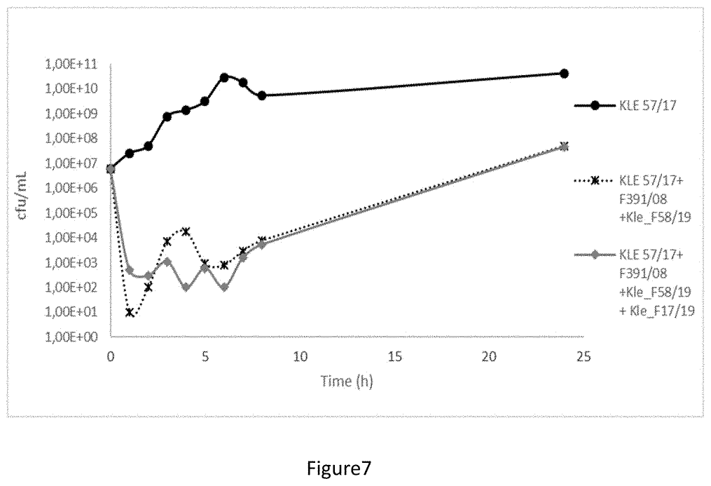

Conventional lysis curves were performed in controlled conditions using a previously determined bacterial inoculum. Cultures were prepared with an inoculum of approximately 2×10⁶ cfu/mL. Two different K. pneumoniae strains 57/17 and 237/14 were used because it was not possible to find in our bacterial collection a highly virulent strain that at the same time was infected by the 3 bacteriophages. Each bacteriophage was tested individually (FIGS. 5 and 6) and in combination (FIG. 7), with MOI approximate to 10. Viable bacteria counts were monitored at 1 h intervals for an 8 h period and again at 24 h post bacteriophage inoculation (ppi).

Bacteriophage F391/08 was tested individually at MOI approximate to 10 (FIG. 5) and at 1 h post bacteriophage inoculation (ppi) the culture presented a decrease in the bacterial load of ˜3 log (2.3×10³ cfu/mL) compared with the bacteria control culture (8.4×10⁶ cfu/mL). Within the first 4 hours' F391/08 was able to maintain the viable bacteria counts below 10⁴ cfu/mL. Bacterial load started to increase in culture and 8 h ppi the culture exceeded the initial bacterial load (3.8×10⁷ cfu/mL). However, at 24 h ppi, viable counts were at 8.8×10⁷ cfu/mL, an extremely significant reduction in comparison with the control culture (1.2×10¹¹ cfu/mL) demonstrating that F391/08 retains the ability to infect KLE 57/17 even at a stationary phase.

Bacteriophage Kle_F17/19 was tested at MOI˜10 against K. pneumoniae 237/14 and in 1 hour reduced the viable counts of bacteria in approximately 5 log units (5.0×10² cfu/mL) when compared with the control culture of bacteria (1.0×10⁷ cfu/mL). Until 4 h ppi Kle_F17/19 bacteriophage was able to maintain the bacterial load in culture below 10⁴ cfu/mL as observed for F391/08 in its host. At 8 h ppi viable bacteria counts had increased rapidly to 1.1×10⁸ cfu/mL and continued until the end of the culture incubation (24 h ppi) with viable bacteria counts at 3.1×10¹⁰ cfu/mL, almost 1 log higher than the values determined for the control culture.

Kle_F58/19 bacteriophage (FIG. 6) action against strain 57/17 was more rapid than phage F391/08. At 1 h ppi viable bacteria counts were reduced by approximately 4 log units compared with the control culture of bacteria reaching 2.0×10² cfu/mL. By 8 h ppi viable bacteria were increased with a count of 1.8×10⁷ cfu/mL at that time point. At 24 h ppi bacteriophage Kle_F58/19 was not so effective against the host such as F391/08, nevertheless was able to achieve a 96% reduction of viable cells, a highly significant decrease compared with the control culture of bacteria.

Individually the 3 bacteriophages, although with a highly lytic capacity, showed slightly differences in efficacy over time in the respective hosts that probably reflects the differences in their adsorption rates, latent periods and burst sizes (data not shown).

FIG. 7 depicts the combined lysis curves of bacteriophages F391/08 and Kle_F58/19 with approximate MOI 10 in K. pneumoniae 57/17. A marked decrease was observed on the activity of the bacteriophages at 1 h ppi (1.0×101 cfu/mL). During the next 8 hours the combined action of the bacteriophages, although with some variation, maintained the viable cell count below 10⁴ cfu/mL, extending the time of action observed for each of the bacteriophages individually. Despite bacterial regrowth, the cell counts at 24 h ppi were 5.1×10⁷ cfu/mL, a significant reduction (˜3 log units) when compared with the bacteria load of the control culture (4.2×10¹⁰ cfu/mL).

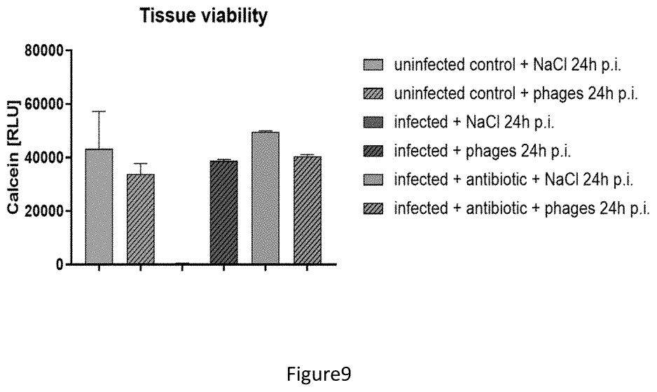

In addition to the lysis curve with F391/08 and Kle_F58/19, a second combined culture was prepared with the 3 bacteriophages F391/08, Kle_F58/19 and Kle_F17/19 (MOI˜10) aiming to observe any action of Kle_F17/19 on the effectiveness of F391/08 and Kle_F58/19. Although K. pneumoniae 57/17 is not susceptible to infection by the bacteriophage Kle_F17/19, the presence of this phage seemed to induce a stabilizing effect to the efficacy of bacteriophages F391/08 and Kle_F58/19 when combined in the first 6 hours of culture (FIG. 9). At 1 h ppi the decrease in the cell counts was not as pronounced compared with the curve of F391/08 and Kle_F58/19, however the behaviour in the following hours suggests an interaction with Kle_F17/19. At 8 h ppi the bacteria load was low with 5.3×10³cfu/mL compared with the control culture with 5.3×10⁹cfu/mL. At the end of the culture incubation the determined cell counts were 4.7×10⁷ cfu/mL a concentration close to the bacteria input in the beginning of the culture.

As previously reported by Loc-Carrillo and Abedon in 2011 (Loc-Carrillo C, Abedon S T. 2011. Pros and cons of phage therapy. Bacteriophage 1(2):111-114) different bacteriophages can be mixed in cocktails to expand their efficiency, typically resulting in a collectively greater antibacterial spectrum of activity. This study demonstrated precisely that.

### 6.3.6 Conclusions

The bacteriophage cocktail characterized and tested in this study is composed of three Klebsiella pneumoniae bacteriophages F391/08, Kle_F17/19 and Kle_F58/19. Genome sequence analysis didn't identify any known genes related to integration, toxins or antibiotic resistances, which are important features regarding the bacteriophages safe use. Comparative genomic analysis revealed that the K. pneumoniae bacteriophages F391/08, Kle_F17/19 and Kle_F58/19 share similarity with Klebsiella phages vB_Kpn_IME260, KOX1 and vB_KpnM_Potts1, respectively.

Klebsiella pneumoniae bacteriophage vB_Kpn_IME260 is a T5-like virus (Xing S, Pan X, Sun Q, Pei G, An X, Mi Z, Huang Y, Zhao B, Tong Y. 2017. Complete genome sequence of a novel multidrug-resistant Klebsiella pneumonia phage, vB_Kpn_IME260. Genome Announc 5:e00055-170). K. oxytoca bacteriophage KOX1 is the most homologous to Kle_F17/19 and Brown et al (Brown T L, Petrovski S, Hoyle D, Chan H T, Lock P, Tucci J. 2017. Characterization and formulation into solid dosage forms of a novel bacteriophage lytic against Klebsiella oxytoca. PLoS ONE 12(8): e0183510) described KOX1 as a Myoviridae. No information regarding vB_KpnM_Potts1 bacteriophage was available in the literature besides the morphology. The transmission electron microscopy analysis confirmed that bacteriophages Kle_F58/19 and Kle_F17/19 belong to the Myoviridae family.

Another important selection criteria for bacteriophages is their host range, which should be as broad as possible, particularly including clinically prevalent bacterial species (Gill J J, Hyman P. 2010. Phage choice, isolation, and preparation for phage therapy. Curr Pharm Biotechnol 11(1):2-14). The extent of the lytic activity of K. pneumoniae bacteriophages F391/08, Kle_F17/19 and Kle_F58/19 was tested against 36 clinical strains, with highly genomic diversity, isolated during the last years in hospitals from the metropolitan area of Lisbon.

Time-kill curves are often used to study the antibacterial effect of single and combination drug compounds and dosing regimens before in vivo efficacy studies (NCCLS. 1999. Methods for determining bactericidal activity of antimicrobial agents: approved guideline. 940 West Valley Road, Suite 1400, Wayne, Pennsylvania 19087 USA: NCCLS). In the current study, lysis curves were prepared to subject bacterial cultures to the bacteriophages lytic ability, individually and combined. A single bacterial inoculum was used. This value was carefully selected based on previous evidence (Mendes J J, Leandro C, Motolla C, Barbosa R, Silva F, Oliveira M, Vilela C, Melo-Cristino J, Gorski A, Pimentel M, São-José C, Cavaco-Silva P, Garcia M. 2014. In vitro design of a novel lytic bacteriophage cocktail with therapeutic potential against organisms causing diabetic foot infections. J Med Microbiol 63:1055-1065). All cultures had an initial bacterial reduction between the first- and second-hour post phage inoculation, followed by re-growth that was noticeable after 6 hours and even more pronounced after 24 hours. Individually all the three bacteriophages showed a high lytic efficacy against planktonic cells. Ninety-five to 99% bacterial reductions at 24 h culture showed their individual increased ability to control K. pneumoniae load, except for Kle_F17/19. Combined activity of two of the 3 phages that compose the cocktail, presented a synergistic bacteriolytic action against the bacterial strain. The chosen MOI was selected based on the “multiplicity of 10 rule,” which states that if the goal is significant reduction in bacterial density, then one should strive for on the order of 10 bacteriophages adsorbed to the average bacterium (Abedon ST. 2009. Kinetics of phage-mediated biocontrol of bacteria. Foodborne Pathog Dis 6:807-815; Kasman L M, Kasman A, Westwater C, Dolan J, Schmidt M G, Norris J S. 2002. Overcoming the phage replication threshold: a mathematical model with implications for phage therapy. J Virol 76:5557-5564). Re-growth was observed in planktonic cells exposed to bacteriophages.

### 6.3.7 EXAMPLE 4

### In vitro Interaction of P. aeruginosa Bacteriophage Cocktail With Antibiotics

An in vitro technique was performed to assess potential interactions between a bacteriophage cocktail of F99/10, F27/12 and F95/13, and a selection of antibiotics frequently used as empiric therapy for nosocomial pneumonia. Although the cocktail addresses Gram negative bacteria, empiric antibiotic treatment often includes drugs for the treatment of pneumonia caused by Gram positive pathogens; thus, molecules that address the treatment of S. aureus were also addressed.

Briefly, broth microdilution method was used to determine Minimum Inhibitory Concentration (MIC) against β-lactams (piperacillin with tazobactam, ceftazidime, and meropenem), aminoglycosi des (amikacin), glycopeptides (vancomycin) and oxazolidinones (linezolid), with and without presence of single and combined bacteriophages, at different MOI (0.1, 1 and 10). The method was developed and optimized as a proof of concept for P. aeruginosa using a single strain infected by all three phages (PSA_1992/05; NCIMB deposit 42914)). The selected MIC range was 0.016 to 32 mg/L, allowing the breakpoints for susceptibility and resistance to be included and detected for all seven antibiotics. Different time points (4 h, 6 h, 12 h and 18 h) were used for optimization of the technique, but final readings were determined at 18 h. All experiments were conducted in duplicates. The study was optimized for the individual bacteriophages and a final assay is presented here containing data for the complete cocktail.

Briefly, the final tests performed with the cocktail (bacteriophages Psa_F99/10, Psa_F27/12 and Psa_F95/13) corroborate the results obtained for each individual phage (data not shown). The efficacy of the cocktail was sufficient to produce a shift in the MIC of PSA_1992/05 from a much higher MIC value to ≤0.016 mg/L, for all tested antibiotics, and for all the three MOI tested during this study (0.1, 1 and 10) (Table 5).

TABLE 5
MIC for P. aeruginosa PSA_1992/05 in the presence of
bacteriophage cocktail at MOI 0.1, 1 and 10.
MIC (mg/L) and clinical breakpoint

| | TZP | C/ZD | MEM | AK | | | CS |
|---|---|---|---|---|---|---|---|
| | (S ≤ 16; | (S ≤ 8; | (S ≤ 2; | (S ≤ 8; | VA | LZD | (S ≤ 2; |
| TP-122A | R > 16) | R > 8) | R > 8) | R > 16) | (NA) | (NA) | R > 2) |
| No Phage | 1 | .5 | .25 | .5 | >32 | >32 | 0.25 |
| MOI 0.1 | ≤0.016 | ≤0.016 | ≤0.016 | ≤0.016 | ≤0.016 | ≤0.016 | ≤0.016 |
| MOI 1 | ≤0.016 | ≤0.016 | ≤0.016 | ≤0.016 | ≤0.016 | ≤0.016 | ≤0.016 |
| MOI 10 | ≤0.016 | ≤0.016 | ≤0.016 | ≤0.016 | ≤0.016 | ≤0.016 | ≤0.016 |

PSA_1992/05 infection with the bacteriophage cocktail (cocktail activity control reading) at 18 h revealed no inhibition of the bacterial growth.
TZP, piperacillin with tazobactam; CZP, ceftazidime; MEM, meropenem; AK, amikacin; VA, vacomycin; LZD, linezolid; CS, colistin.
NA, Non applicable.

This data demonstrates an increased combined efficacy of the cocktail of F99/10, F27/12 and F95/13 in the presence of these antibiotics. No negative effect produced by the addition of the phage cocktail was observed.

Overall, although vancomycin and linezolid are antibiotics that do not show any activity against Gram negative bacteria, their presence did not inhibit the activity the cocktail. The main classes of antibiotics used to treat nosocomial pneumonia have been assessed in combination with phages alone for proof of concept and optimization purposes, and as a cocktail. Overall, no interaction that may result in inhibition of phages or in the significant decrease of the antibiotic activity have been detected.

### 6.3.8 EXAMPLE 5

### Feasibility Study of Inhalative Phage Application

Nebulization have been successively used for inhalation therapy, namely using bacteriophages in animal models by means of powder formulations (Chang, R. Y. K., Chen, K., Wang, J., Wallin, M., Britton, W., Morales, S., Kutter, E., Li, J., Chan, H. K., 2018. Proof-of-Principle Study in a Murine Lung Infection Model of Antipseudomonal Activity of Phage PEV20 in a Dry-Powder Formulation. Antimicrob Agents Chemother 62, 2). In here, a feasibility study of an inhalative phage application was developed with a liquid formulation, using different options of vibrating mesh nebulizers. Those consist of hospital-grade nebulizers, used not only in hospital wards, but also in intensive care units that can be adequately attached to any standard ventilator circuit.

For comparison of three vibrating mesh nebulizer devices (Aeroneb, Nebutec 4, Nebutec 6), a cocktail containing 10⁹ pfu/ml of each phage F99/10, F27/12 and F95/13 was prepared using phage suspensions and DPBS (first experiment) or 0.9% NaCl solution (second experiment) as the diluent. For each diluent, three independent nebulization experiments were performed using all three devices. Per nebulization, 1 ml of phage cocktail was nebulized within a timeframe of 1 minute, followed by a sedimentation time of 60 minutes. Phages were collected within a petri dish containing 15 ml of the respective collection buffer. Titers of phages were determined using specific detection (titration) strains for each phage, respectively. Droplet size for each device was determined experimentally by nebulization and laser diffraction, and was in line with manufacturer information. Furthermore, aerosol delivery rate was determined experimentally, and the pulmonary deposition probability was calculated based on the measured data, suggesting that both Aeroneb and Nebutec 4 achieve comparable deposition, and are superior to Nebutec 6 (data now shown). Of note, only 75%±8.6% of nebulized liquid was successfully deposited onto the petri dish due to adhesion of droplets to the nebulization cylinder. Overall, phage recoveries were higher with NaCl as diluent, compared to DPBS. Furthermore, the Aeroneb device yielded the most uniform recovery rates across all three phages (data now shown).

Based on the results, the Aeroneb device and NaCl as vehicle were selected. Overall, the obtained results allowed the selection of vibrating mesh nebulizer device Aeroneb, which was then applied to remaining efficacy and toxicity studies conducted in precision-cut lung slices (PCLS) ex vivo model (See Example 6). The recovery and log reduction of phage titers were consistent between two formulations, between different phages, and were also consistent with expected phage losses throughout the system.

### 6.3.9 EXAMPLE 6

### Efficacy and Toxicity Study in Precision Cut Lung Slices (PCLS) Ex vivo Model

For assessment of efficacy and toxicity of bacteriophages F99/10, F27/12 and F95/13, a comprehensive ex vivo study was carefully designed in PCLS P. aeruginosa infection model. The system was planned and optimized in rat PCLS that were subjected to treatment with the cocktail of purified F99/10, F27/12 and F95/13 by submersion and nebulisation, in the presence and absence of antibiotic co-treatment. The system was then transferred to a final setup of human PCLS. To evaluate bacteriophage efficacy and toxicity after inhalative application on P. aeruginosa infected human lung tissue, alone or in combination with antibiotics, the experiments were carried out with human PCLS. P. aeruginosa, and nebulized phage cocktail was applied directly onto the air-liquid interface cultivated tissue, while antibiotics (at subinhibitory concentration 0.5 μg/ml) were applied submerged (i.e. into the culture medium). In addition, uninfected controls were treated with nebulized phage cocktail to exclude cytotoxic effects of the phages on human lung tissue. Similarly, to the results obtained for rat PCLS (data not shown), both the nebulized phage treatment and the antibiotic reduced bacterial load of infected human PCLS to similar extent, while their co-treatment resulted in a strong synergistic effect in bacterial reduction.

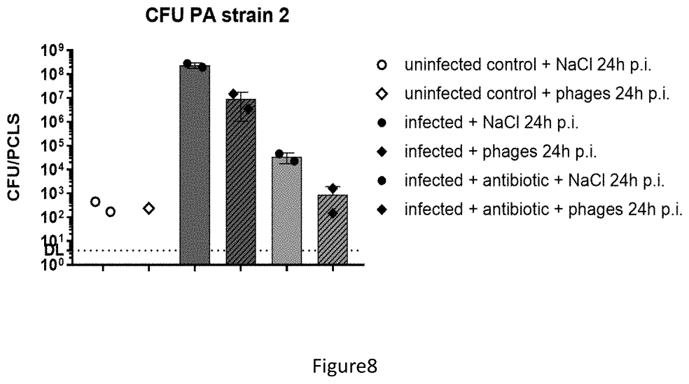

Phage treatment alone reduced bacterial load by more than 1 log, while antibiotic treatment alone led to an about 4 log reduction of cfu. In combination, a synergistic effect was observed, leading to a more than 5 log reduction of bacterial load (FIG. 8). Upon analysis of tissue viability, no cytotoxic effect of the purified phages on uninfected tissue could be observed. Treatment of infected PCLS with purified phages resulted in a complete rescue of the loss of viability induced by to P. aeruginosa infection. Antibiotic treatment alone or in combination with phage treatment restored tissue viability to a comparable extent (FIG. 9). In uninfected human PCLS, phages could be detected at 24 h post treatment at numbers comparable to the inoculum. In contrast, phage replication was observed in infected samples, but not in infected samples co-treated with antibiotics, which is in line with the observed differences in bacterial load (data not shown).

Overall, the study conducted in P. aeruginosa PCLS ex vivo model showed that treatment of infected rat/human slices with the cocktail of F99/10, F27/12 and F95/13, not only showed efficacy trough submersion and nebulization treatments, but also restored lung tissue viability while showing no toxic effects, even in the presence of combination antibiotic therapy (meropenem, amikacin and vancomycin). Analysis of uninfected slices treated with the cocktail, in multiples conditions (submerged vs nebulized; rat vs human; phage lysates vs purified phages), demonstrated no negative effect of the cocktail treatment on lung tissue viability, reinforcing that there is no predictable toxicity, and specifically cytotoxicity, caused by phage treatment in the lung. This evidence is also in accordance with previous studies that also showed no adverse effects of phage treatment in other infection models and compassionate use cases (McCallin, S., Sacher, J. C., Zheng, J., Chan, B. K., 2019. Current State of Compassionate Phage Therapy. Viruses 11, 343; Oliveira, A., Sereno, R., Nicolau, A., Azeredo, J., 2009. In vivo toxicity study of phage lysate in chickens. Br Poult Sci 50, 558-563.

With regards to different administration routes, the nebulised phage treatment successfully reduced the bacterial load of infected PCLS, thus showing comparable efficacy to the submerged treatment, although expected bacteriophage losses occur in the nebulizer. An increase in phage titer towards 24 h was observed. Determination of phage titers in the inoculum revealed that phage numbers in samples with or without antibiotic mix were comparable, thus presence of antibiotic did not impact on phage titer or activity. On the other hand, it was also possible to confirm that the cocktail did not interfere with the activity of the antibiotics. Indeed, phage/antibiotic co-treatment completely restored tissue viability. Overall, this combination is also supported by all safety endpoints in infected and uninfected slices of rat/human lung.

### 6.3.10. EXAMPLE 7

### Nebulization Studies in K. pneumoniae Infection Model of PCLS

The efficacy of the inhalative application of the Klebsiella pneumoniae F391/08, Kle_F17/19 and Kle_F58/19 bacteriophages was investigated on respiratory bacterial infection in an ex vivo model of K. pneumoniae infection in precision-cut lung slices (PCLS). The study included the confirmation of biological activity of bacteriophages after nebulization, the setup of the K. pneumoniae infection model and evaluation of phage activity after nebulization in rat PCLS.

Material and Methods

The experiments were performed at Fraunhofer ITEM, department of Preclinical Pharmacology (Hannover, Germany). The sacrifice of animals to remove organs for the preparation of PCLS was registered at the responsible authority (Lower Saxony Federal State Office for Consumer Protection and Food Safety), and performed in accordance with the Regulations of the German Animal Protection Law (Tierschutzgesetz of 18 May 2006, BGB1. I S. 1206, 1313; adopted 28 Jul. 2014, BGB1.I S. 1308) and the Directive 2010/63/EU of the European Parliament and of the Council on the protection of animals used for scientific purposes.

Female rats (Wistar W U, age at experiment 8-21 weeks) were obtained from Charles River (Sulzfeld, Germany). Animals were kept under conventional housing conditions (22° C., 55% humidity, and 12-h day/night rhythm) until use for PCLS preparation. Rats were sacrificed by an overdose of pentobarbital sodium (Narcoren, Merial GmbH, Hallbergmoos, Germany) and exsanguination via the vena cava. Whole rat lungs were filled with 37 C warm low-melting agarose/medium solution (1.5% (v/v) final concentration). Filled lobes were cooled in ice-cold PBS to polymerize the agarose. Cylinders of 10 mm diameter were drilled out of the lung tissue and cut into approximately 300 μm thick slices using a Krumdieck microtome (Alabama Research and Development, Munford, Ala., USA). Slices were collected in 4° C. cold EBSS, then transferred to petri dishes with PCLS culture medium and incubated under cell culture conditions (37° C., 5% CO2) for approximately 30 min. Subsequently, slices were washed four times for 30 minutes with PCLS culture medium and finally incubated overnight under cell culture conditions prior to infection experiments.

K. pneumoniae test infection and detection strains (57/17, 131/15, 130/14 and 25/H) were grown overnight in TSB medium at 37° C. and 150 rpm. Overnight cultures were diluted 1:30 and incubated for another 1 h at 37° C. and 150 rpm to yield cultures in the exponential growth phase. Detection strains were used at an OD600 of 0.3-0.5 for the plaque forming assay. Cultures of infection strain 57/17 were adjusted to 2×10⁶ CFU/ml and samples of the inoculum were plated to confirm the cfu concentration.

Bacteriophage nebulization was performed using a vibrating mesh nebulizer (AeroNeb). Briefly, a cocktail containing 10⁹PFU/ml of each phage was prepared in 0.9% NaCl and the aerosol was generated Per nebulization, 1 mL of phage cocktail was nebulized within a timeframe of 1 minute, followed by a sedimentation time of 60 minutes. For the nebulization pre-test (without PCLS), a phage cocktail of phages F391/08, Kle_F17/19 and Kle_F58/19 was prepared and phages were nebulized and collected in a petri dish containing 15 ml vehicle as collection buffer (0.9% NaCl). The biological activity of phages was determined by double agar overlay plaque assay.

The efficacy of the nebulized phages in rat PCLS was performed to evaluate if the phage nebulization on PCLS was efficient in the K. pneumoniae infection model. Therefore, rat PCLS were cultured at the air-liquid-interface (ALI) in 12-well transwell plates (500 μl reservoir volume) and infected with K. pneumoniae strain 57/17 (1×10⁵ CFU/PCLS applied directly onto the tissue. One-hour post infection (p.i.), the inoculum was removed, PCLS were washed and the transwells transferred onto new companion plate wells with 500 μl of fresh medium in the lower compartment. A phage cocktail containing 1×10¹⁰ PFU/mL per phage was prepared (diluent: 0.9% NaCl). Using the Aeroneb device and the exposure system with customized adapter for 4 transwells, 1 ml of phage cocktail or vehicle (0.9% NaCl) was nebulized onto PCLS (nebulization time: 1 min, sedimentation time: 60 min). Separate exposure systems and adapters were used for nebulization of phage or vehicle to preclude contamination. PCLS were post-incubated overnight at 37° C., 5% CO2, and samples were harvested and analyzed at 24 h p.i.. Additionally, in this first experiment separate samples were harvested directly after phage nebulization to determine the actual deposited PFU per well.

After confirmation of the efficacy of bacteriophage nebulization in the K. pneumoniae PCLS model, the experiments were extended to test the efficacy of the bacteriophage cocktail alone and in combination with antibiotic co-treatment. The preparation, cultivation and infection of rat PCLS as well as phage treatment by nebulization were performed as described before. For combination treatment with antibiotics, the medium of the respective PCLS was supplied with meropenem, vancomycin and amikacin at final concentrations of 8, 8, or 1 μg/ml, respectively. Antibiotic treatment was performed simultaneously with phage treatment at one hour p.i.. Therefore, the washed PCLS transwell was transferred to a new companion plate, with the basolateral compartment containing medium or medium with antibiotic cocktail, and exposed at the apical side to bacteriophage or saline aerosol as described above.

PCLS were analyzed for cfu load, tissue viability and phage replication. For analysis of the bacterial load and phage titer, PCLS were lysed with 1% Triton X-100/PBS for 30 min at 4° C. Tissue residues were removed and the lysate centrifuged at 8000×g for 10 min at 4° C. to pellet bacteria. The cleared lysates were used for determination of phage titer. The pellets were resuspended in 250 μl PBS/Tween and used for determination of bacterial load.

Phage titers were determined by plaque assay, using K. pneumoniae strains 131/15, 130/14 and 25/14 for specific detection of the phages F391/08, Kle_F17/19 and Kle_F58/19, respectively. Each sample treated with phage cocktail was co-plated with each detection strain. Phages were released from PCLS by tissue lysis, and separated from bacteria by centrifugation, as described above. Series of 1:10 dilutions of phage samples in PBS/Tween were prepared in Eppendorf tubes and mixed by inversion, to avoid mechanical disturbance. 100 μl of each phage dilution together with 100 μl of detection strain culture was added to 3 ml of soft-agar tempered to 48° C. and the mixture spread onto one TSA plate. At the end of every PFU experiment, all detection strains were plated to exclude contamination by phages of the bacterial cultures. After incubation of agar plates overnight at 37° C., phage plaques were counted. For calculation of PFU per PCLS, plaque counts were multiplied by the respective dilution factor and by a factor of 2.5 which reflects the ratio of total volume of phage sample (250 μl) to plated volume (100 μl).

For the quantification of the bacterial load from the PCLS lysates, series of 1:10 dilutions in PBS/Tween of the respective samples were prepared and 50 μl of each dilution was plated onto one half of a TSA plate. After incubation of agar plates overnight at 37° C., colonies were counted. For calculation of CFU per PCLS, colony counts were multiplied by the respective dilution factor and by a factor of 5, reflecting the ratio of total volume of bacterial suspension (250 μl) to plated volume (50 μl).

Viability of uninfected and infected tissue was assessed by Calcein staining. Calcein AM permeates intact cells and is intracellularly hydrolyzed to produce Calcein, a fluorescent compound that is retained in the cell cytoplasm. An increase of dead or damaged cells within PCLS results in a decrease of Calcein staining intensity. PCLS incubated in 70% ethanol for 15 minutes at RT were used as reference (“dead control”). All PCLS were washed once with warm medium and incubated with Calcein AM staining solution (4 μM in DMEM/F12) for 45 minutes at 37° C. and 150 rpm in the dark. After washing 3× with PBS, PCLS were lysed with 1% Triton-X100 for 30 minutes at 4° C. in the dark. Subsequently, 50 μl of tissue lysate were transferred into a black-walled 96-well plate and fluorescence (excitation wavelength: 485 nm, emission wavelength: 535 nm) was measured using a microplate reader. All samples were measured in duplicates.

Results

Prior to the first nebulization tests the biological activity of the phages after nebulization was assayed. A phage cocktail with 1×10⁶ PFU/ml was prepared in 0.9% NaCl. After nebulization, the phage titers recovered indicated a high loss during deposition and thus a new cocktail was prepared with 1×10⁹ PFU/ml of each phage. Additional nebulization tests were performed to estimate the expected phage titers after nebulization. The detailed results are given in Table 6.

TABLE 6
Phage recovery after nebulization with Aeroneb nebulizer.

| | F391/08 | Kle_F17/19 | Kle_F58/19 |
|---|---|---|---|
| Working solution | 10⁹ PFU/ml | 10⁹ PFU/ml | 10⁹ PFU/ml |

After nebulization:

| | | | |
|---|---|---|---|
| Phage (Det. Strain) | F391/08 | Kle_F17/19 | Kle_F58/19 |
| | (131/15) | (130/14) | (25/14) |
| | 1.20 × 10⁷ | 2.58 × 10⁷ | 5.4 × 10⁶ |
| | PFU total | PFU total | PFU total |
| Total log reduction | ~2 log | ~1.5 log | ~2.5 log |

In summary, with an initial phage cocktail of 1×10⁹PFU/ml after nebulization a remaining activity of approx. 1.5 to 2.5 log lower can be expected (phage 391/08 ˜2 log, phage Kle_F17/19 ˜1.5 log, phage Kle_F58/19 ˜2.5 log). To accommodate for this reduction, it was decided to run the efficacy experiments with a higher phage cocktail titer of 1×10¹⁰ PFU/ml per phage.

Based on these results it was concluded, that, despite the partial loss of activity in the phage titer after nebulization, these results should be sufficient to show efficacy in the PCLS infection model since bacteriophage activity after nebulization was confirmed.

For the treatment with nebulized bacteriophages, rat PCLS were cultivated at the air-liquid interface in transwells, and the bacterial inoculum as well as the nebulized phage (or NaCl as vehicle treatment) were deposited directly onto the tissue. Nebulization was performed as described previously.

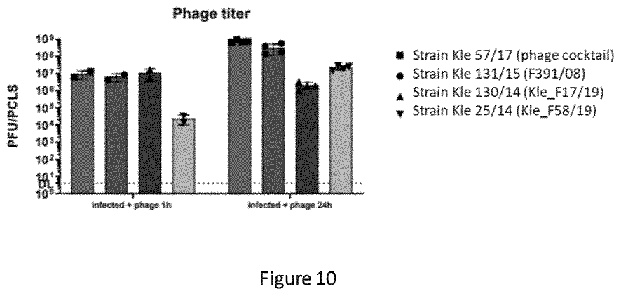

The nebulization of the phage cocktail containing 1×10¹⁰ PFU/ml of each phage resulted in a deposited titer of approx. 1×10⁷ PFU per well (per PCLS) for phage 391/08 and Kle_F17/19 and approx. 1×10⁴ PFU/ PCLS for phage Kle_F58/19 directly after nebulization (1 h p.i.). Phage 391/08 and Kle_F58/19 strongly replicated in K. pneumoniae 57/17 infected PCLS, reaching a titer of approx. 3×10⁸ PFU and 2×10⁷ pfu, respectively, while phage Kle_F17/19 even slightly decreased in titer (2×10⁶ PFU) (FIG. 10). This was expected since phage Kle_F17/19 is not able to infect K. pneumoniae 57/17. Nevertheless, it was decided to test the cocktail of the 3 phages in this strain to gather results from any toxic effects that might be induced by this phage treatment.

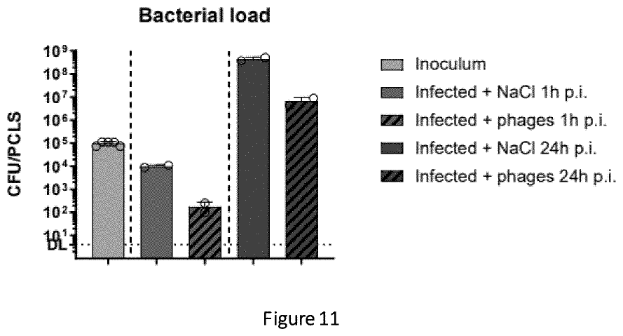

PCLS were infected with an inoculum of 1×10⁵ CFU Klebsiella pneumoniae for 1 h followed by removal of the inoculum, washing and transfer to a new well to avoid overgrowth of non-tissue attached planktonic bacteria. With this procedure, the actual bacterial load directly after exposure to vehicle (NaCl) was approx. 10⁴ CFU/PCLS. If exposed to nebulized phage cocktail for 1 h, the CFU load was already decreased to 10² CFU/PCLS indicating rapid lysis of the bacteria by the bacteriophages (FIG. 8). Strong bacterial growth resulted in bacterial load of approx. 4×10⁸ CFU at 24 h p.i. in vehicle-treated PCLS. Treatment with nebulized phage cocktail resulted in an approx. 2 log reduced bacterial load at 24 h p.i. Thus, the nebulized phage treatment successfully reduced the bacterial load of K. pneumoniae infected PCLS (FIG. 11).

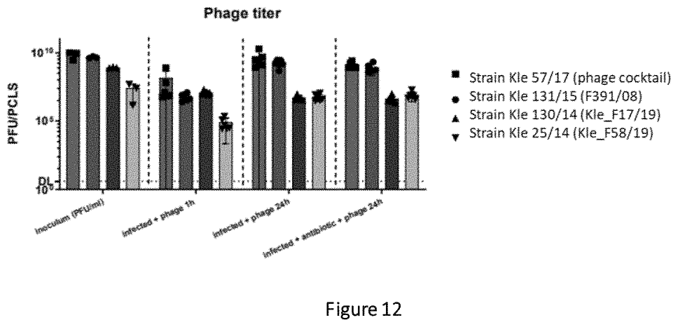

To achieve the proof-of-concept for bacteriophage efficacy after inhalative application alone or in combination with antibiotics, rat PCLS cultivated at the air-liquid interface were infected with K. pneumoniae strain 57/17 and nebulized with the phage cocktail directly onto the air-liquid interface cultivated tissue. For co-treatment, antibiotics (at subinhibitory concentration) were applied submerged (i.e. into the culture medium in the basolateral compartment of the transwell) at the time of phage treatment. Per condition 1 plate with 4 replicate wells was used. From those 2 wells were used for determination of CFU/PFU and the other 2 wells were used for determination of tissue viability (Calcein staining). Nebulization of the phage cocktail resulted in deposition of approximately 10⁷ PFU for phages 391/08 and Kle_F17/19, and approx. 10⁵ PFU for phage Kle_F58/19, directly after nebulization (1 h p.i.) (FIG. 12).

Subsequently, phage 391/08 and Kle_F58/19 replicated in the K. pneumonia-infected PCLS, resulting in 2-3 log increased phage titers at 24 h p.i., while phage Kle_F17/19 titer remained stable. Samples with antibiotic co-treatment showed similar phage titers, as can be expected due to the unaffected bacterial load that was observed.

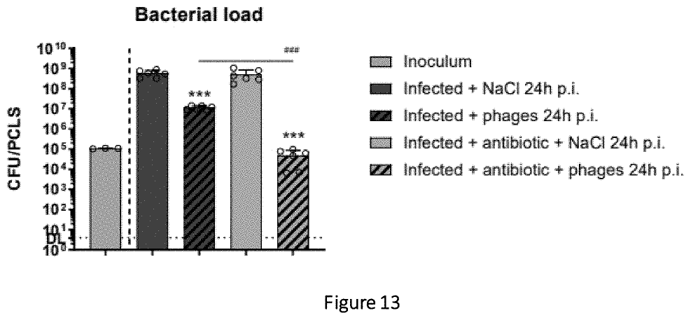

The nebulized phage treatment successfully reduced the CFU load of the infected PCLS about approx. 1.5 log (FIG. 13) while the (submerged) treatment with a subinhibitory dose of antibiotics did not reduce the CFU. Notably, the combined treatment of antibiotics with nebulized bacteriophage cocktail showed a strong synergistic effect, as it reduced CFU load by approx. 4 log (FIG. 13).

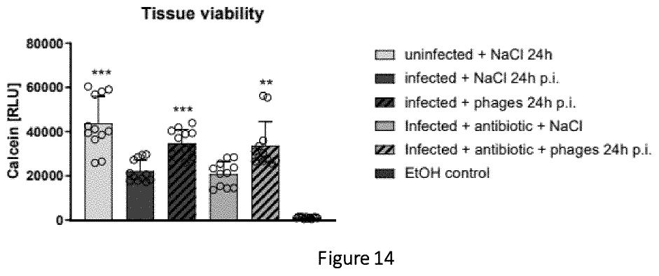

The infection with K. pneumoniae induced a significant loss of tissue viability in PCLS at 24 h p.i. (FIG. 14). Treatment with nebulized phage cocktail significantly inhibited this infection-induced loss of tissue viability (FIG. 14). Treatment with subinhibitory doses of antibiotics had no effect on tissue viability, as to be expected from the unaltered CFU. In the phage/antibiotic co-treatment the synergistic effect on bacterial load was not reflected in a further increase of tissue viability, as phage treatment alone already significantly improved tissue viability.

Discussion

Precision cut lung slices (PCLS) are fresh lung tissue sections and, as an organotypic model, contain all cell types present in the lung and allow to investigate the response of lung tissue in detail. The high degree of complexity provides a physiologically relevant model. The data obtained from the PCLS studies demonstrated a clear efficacy of the nebulized bacteriophage treatment of K. pneumoniae infected lung tissue slices. The phage cocktail administration reduced bacterial load and restored tissue viability. Additional synergistic effects on CFU reduction, of the antibiotic resistant K. pneumoniae strain tested, were observed in combination treatment with sub-inhibitory doses of antibiotics.

Certain modifications and improvements will occur to those skilled in the art upon a reading of the foregoing description. It should be understood that all such modifications and improvements have been deleted herein for the sake of conciseness and readability but are properly within the scope of the following claims.

All publications, patents, and patent applications cited herein are hereby incorporated by reference in their entirety for all purposes.

What is claimed is:

1. A pharmaceutical composition comprising purified bacteriophage F17/19 or F58/19 and a pharmaceutically acceptable carrier.

2. The pharmaceutical composition of claim 1, which comprises purified bacteriophage F17/19 and F58/19.

3. The pharmaceutical composition of claim 1, further comprising the purified bacteriophage F391/08.

4. The pharmaceutical composition of claim 1 which comprises the purified bacteriophages F17/19, F58/19 and F391/08.

5. The pharmaceutical composition of claim 1 which consists of the purified bacteriophages F17/19, F58/19 and F391/08.

6. The pharmaceutical composition of claim 1 which further comprises one or more of the purified bacteriophage F99/10, F27/12 and F95/13.

7. The pharmaceutical composition of claim 5, which further comprises the purified bacteriophage F99/10, F27/12 and F/95/13.

8. The pharmaceutical composition of claim 1, which further comprises one or more additional purified bacteriophage having antibacterial activity against Klebsiella pneumonae.

9. The pharmaceutical composition of claim 1, which further comprises one or more additional purified bacteriophage having antibacterial activity against Pseudomonas aeruginosa.

10. The pharmaceutical composition of claim 1, which further comprises at least one of the purified bacteriophage F99/10, F110/10, F27/12, F83/13, F95/13, F92/15, F105/15, F134/15, or F141/15.

11. The pharmaceutical composition of claim 1, wherein said composition is formulated for administration as an aerosol.

12. The pharmaceutical composition of claim 1, wherein each said bacteriophage is present in an amount to provide a multiplicity of infection (MOI) of about 1 to about 10 upon administration of said composition to a subject in need thereof

13. The pharmaceutical composition of claim 1, wherein each said bacteriophage is present at approximately 10⁸ to 10¹¹ pfu.

14. The pharmaceutical composition of claims 1, wherein said composition is formulated for administration as an aerosol.

15. A method of treating or reducing the occurrence of a bacterial infection in a subject in need thereof comprising administering to said subject a therapeutically or prophylactically effective amount of the pharmaceutical composition of claim 1.

16. The method of claim 15 wherein the pharmaceutical composition comprises the purified bacteriophages F17/19, F58/19 and F391/08 and is administered in combination with a pharmaceutical composition comprising the purified bacteriophages F99/10, F27/12 and F/95/13 and a pharmaceutically acceptable carrier.

17. The method according to claim 15 wherein said bacterial infection is caused by a Pseudomonas aeruginosa or Klebsiella pneumoniae bacterial strain,

18. The method of claim 17, wherein said bacterial infection is a respiratory infection, preferably a hospital-acquired bacterial pneumonia or infection associated with cystic fibrosis.

19. The method of claim 18 , wherein said composition is administered as an aerosol to the lungs.

20. A method for diagnosing the causative agent of a bacterial infection comprising
(i) culturing a tissue sample from a patient;
(ii) contacting the culture of step (i) with purified bacteriophage F17/19 or F58/19;
and
(iii) monitoring for evidence of growth or lysis of the culture
wherein evidence of lysis of the culture indicates that the culture comprises a bacterial strain known to be susceptible to the bacteriophage or protein used in step (ii).
